(19) United States
(12) Patent Application Publication
(10) Pub. No.: US 2010/0286147 A1
(43) Pub. Date: Nov. 11, 2010

(54) FUNGICIDAL AMIDES

(75) Inventors: Mary Ann Hanagan, Newark, DE (US); Robert James Pasteris, Newark, DE (US)

Correspondence Address: E I DU PONT DE NEMOURS AND COMPANY;LEGAL PATENT RECORDS CENTER, BARLEY MILL PLAZA 25/1122B, 4417 LANCASTER PIKE, WILMINGTON, DE, 19805, US

(73) Assignee: E.I. DUPONT DE NEMOURS AND COMPANY, Wilmington, DE (US)

(21) Appl. No.: 12/811,126

(22) PCT Filed: Jan. 22, 2009
(86) PCT No.: PCT/US09/31618

§ 371 Date: Jun. 29, 2010

Related U.S. Application Data
(60) Provisional application No. 61/062,367, filed on Jan. 25, 2008.

(51) Int. Cl. A61K 31/5377; A61K 31/497; A61K 31/454; C07D 417/14

(52) U.S. Cl. 514/235.8; 514/253.1; 514/326; 544/129; 544/364; 546/209

(57) ABSTRACT

Disclosed are compounds of Formulae 1 and 1A (including all geometric and stereoisomers), N-oxides, and salts thereof,

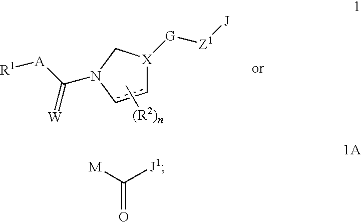

wherein
R¹, R², A, G, M, W, Z¹, X, J, J¹ and n are as defined in the disclosure.

Also disclosed are compositions containing the compounds of Formula 1 and methods for controlling plant disease caused by a fungal pathogen comprising applying an effective amount of a compound or a composition of the invention.

### FIELD OF THE INVENTION

This invention relates to certain carboxamides, their N-oxides, salts and compositions, and methods of their use as fungicides.

### BACKGROUND OF THE INVENTION

The control of plant diseases caused by fungal plant pathogens is extremely important in achieving high crop efficiency. Plant disease damage to ornamental, vegetable, field, cereal, and fruit crops can cause significant reduction in productivity and thereby result in increased costs to the consumer. Many products are commercially available for these purposes, but the need continues for new compounds which are more effective, less costly, less toxic, environmentally safer or have different sites of action.

World Patent Publication WO 2005/003128 discloses certain thiazolylpiperidines of Formula i and their use as microsomal triglyceride transfer protein inhibitors.

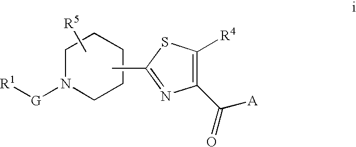

World Patent Publication WO 2004/058751 discloses certain piperidinyl-thiazole carboxamides for altering vascular tone.

PCT Patent Publication WO 2007/014290 discloses certain azocyclic amides of Formula ii

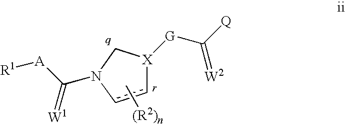

and their use as fungicides.

### SUMMARY OF THE INVENTION

This invention relates to compounds of Formula 1 (including all geometric and stereoisomers), N-oxides, and salts thereof, agricultural compositions containing them and their use as fungicides:

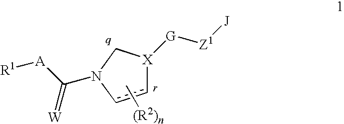

wherein
R¹ is an optionally substituted phenyl or 5- or 6-membered heteroaromatic ring or optionally substituted naphthalenyl;
A is CHR¹⁵ or NR¹⁶;
R¹⁵ is H, halogen, cyano, hydroxy, —CHO, C₁-C₄ alkyl, C₂-C₄ alkenyl, C₂-C₄ alkynyl, C₁-C₄ haloalkyl, C₂-C₄ haloalkenyl, C₂-C₄ haloalkynyl, C₂-C₄ alkoxyalkyl, C₂-C₄ alkylthioalkyl, C₂-C₄ alkylsulfinylalkyl, C₂-C₄ alkylsulfonylalkyl, C₂-C₄ alkylcarbonyl, C₂-C₄ haloalkylcarbonyl, C₂-C₅ alkoxycarbonyl, C₃-C₅ alkoxycarbonylalkyl, C₂-C₅ alkylaminocarbonyl, C₃-C₅ dialkylaminocarbonyl, C₁-C₄ alkoxy, C₁-C₄ haloalkoxy, C₁-C₄ alkylthio, C₁-C₄ haloalkylthio, C₁-C₄ alkylsulfinyl, C₁-C₄ haloalkylsulfinyl, C₁-C₄ alkylsulfonyl or C₁-C₄ haloalkylsulfonyl;
R¹⁶ is H, C₁-C₄ alkyl, C₂-C₄ alkenyl, C₂-C₄ alkynyl, C₁-C₄ haloalkyl, C₂-C₄ haloalkenyl, C₂-C₄ haloalkynyl, C₂-C₄ alkoxyalkyl, C₂-C₄ alkylthioalkyl, C₂-C₄ alkylsulfinylalkyl, C₂-C₄ alkylsulfonylalkyl, C₂-C₄ alkylcarbonyl, C₂-C₄ haloalkylcarbonyl, C₂-C₅ alkoxycarbonyl, C₃-C₅ alkoxycarbonylalkyl, C₂-C₅ alkylaminocarbonyl, C₃-C₅ dialkylaminocarbonyl, C₁-C₄ alkylsulfonyl or C₁-C₄ haloalkylsulfonyl;
W is O or S;
X is a radical selected from

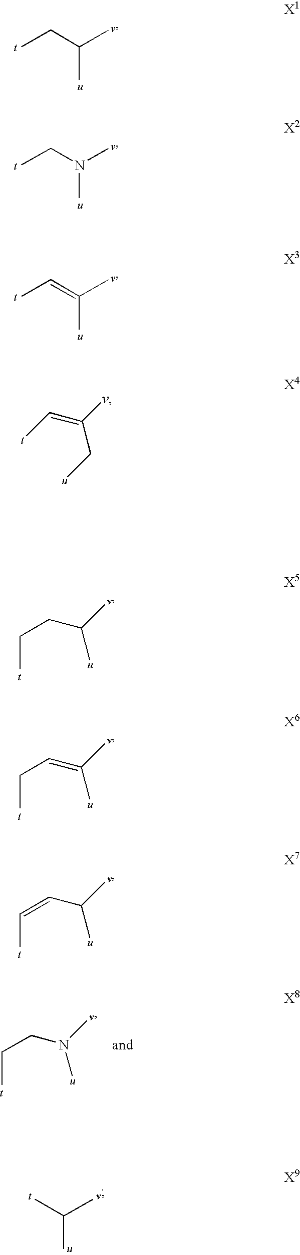

wherein the bond of X¹, X², X³, X⁴, X⁵, X⁶, X⁷, X⁸ or X⁹ which is identified with “t” is connected to the carbon atom identified with “q” of Formula 1, the bond which is identified with “u” is connected to the carbon atom identified with “r” of Formula 1, and the bond which is identified with “v” is connected to G;
each R² is independently C₁-C₄ alkyl, C₁-C₄ alkenyl, C₁-C₄ haloalkyl, C₁-C₄ alkoxy, halogen, cyano or hydroxy; or
two R² are taken together as C₁-C₄ alkylene or C₂-C₄ alkenylene to form a bridged bicyclic or fused bicyclic ring system; or
two R² attached to adjacent ring carbon atoms joined by a double bond are taken together as —CH═CH—CH═CH— optionally substituted with 1 to 3 substituents selected from C₁-C₄ alkyl, C₁-C₄ haloalkyl, C₁-C₄ alkoxy, C₁-C₄ haloalkoxy, halogen, hydroxy, amino, cyano and nitro;
G is an optionally substituted 5-membered heterocyclic ring;
J is a 5-, 6- or 7-membered ring, a 8- to 11-membered bicyclic ring system or a 7- to 11-membered spirocyclic ring system, each ring or ring system containing ring members selected from carbon, up to 4 heteroatoms selected from up to 2 O, up to 2 S and up to 4 N, and up to 3 ring members selected from C(═O), C(═S), S(═O)ₐ(═NR²³)_{b} and SiR¹⁷R¹⁸, each ring or ring system substituted with 1 to 2 substituents independently selected from —Z²Q and optionally substituted with 1 to 5 substituents independently selected from R⁵;
each R⁵ is independently H, halogen, cyano, hydroxy, amino, nitro, —CHO, —C(═O)OH, —C(═O)NH₂, —NR²⁵R²⁶, C₁-C₆ alkyl, C₂-C₆ alkenyl, C₂-C₆ alkynyl, C₁-C₆ haloalkyl, C₂-C₆ haloalkenyl, C₂-C₆ haloalkynyl, C₃-C₈ cycloalkyl, C₃-C₈ halocycloalkyl, C₄-C₁₀ alkylcycloalkyl, C₄-C₁₀ cycloalkylalkyl, C₆-C₁₄ cycloalkylcycloalkyl, C₄-C₁₀ halocycloalkylalkyl, C₅-C₁₀ alkylcycloalkylalkyl, C₃-C₈ cycloalkenyl, C₃-C₈ halocycloalkenyl, C₂-C₆ alkoxyalkyl, C₄-C₁₀ cycloalkoxyalkyl, C₃-C₈ alkoxyalkoxyalkyl, C₂-C₆ alkylthioalkyl, C₂-C₆ alkylsulfinylalkyl, C₂-C₆ alkylsulfonylalkyl, C₂-C₆ alkylaminoalkyl, C₃-C₈ dialkylaminoalkyl, C₂-C₆ haloalkylaminoalkyl, C₄-C₁₀ cycloalkylaminoalkyl, C₂-C₆ alkylcarbonyl, C₂-C₆ haloalkylcarbonyl, C₄-C₈ cycloalkylcarbonyl, C₂-C₆ alkoxycarbonyl, C₄-C₈ cycloalkoxycarbonyl, C₅-C₁₀ cycloalkylalkoxycarbonyl, C₂-C₆ alkylaminocarbonyl, C₃-C₈ dialkylaminocarbonyl, C₄-C₈ cycloalkylaminocarbonyl, C₂-C₆ haloalkoxyalkyl, C₁-C₆ hydroxyalkyl, C₁-C₆ alkoxy, C₁-C₆ haloalkoxy, C₃-C₈ cycloalkoxy, C₃-C₈ halocycloalkoxy, C₄-C₁₀ cycloalkylalkoxy, C₂-C₆ alkenyloxy, C₂-C₆ haloalkenyloxy, C₂-C₆ alkynyloxy, C₂-C₆ haloalkynyloxy, C₂-C₆ alkoxyalkoxy, C₂-C₆ alkylcarbonyloxy, C₂-C₆ haloalkylcarbonyloxy, C₄-C₈ cycloalkylcarbonyloxy, C₃-C₆ alkylcarbonylalkoxy, C₁-C₆ alkylthio, C₁-C₆ haloalkylthio, C₃-C₈ cycloalkylthio, C₁-C₆ alkylsulfinyl, C₁-C₆ haloalkylsulfinyl, C₁-C₆ alkylsulfonyl, C₁-C₆ haloalkylsulfonyl, C₃-C₈ cycloalkylsulfonyl, C₃-C₁₀ trialkylsilyl, C₁-C₆ alkylsulfonylamino or C₁-C₆ haloalkylsulfonylamino;
R²⁵ is H, C₁-C₆ alkyl, C₁-C₆ haloalkyl, C₃-C₈ cycloalkyl, C₂-C₆ alkylcarbonyl, C₂-C₆ haloalkylcarbonyl, C₂-C₆ alkoxycarbonyl or C₂-C₆ haloalkoxycarbonyl;
R²⁶ is C₁-C₆ alkyl, C₁-C₆ haloalkyl, C₃-C₈ cycloalkyl, C₂-C₆ alkylcarbonyl, C₂-C₆ haloalkylcarbonyl, C₂-C₆ alkoxycarbonyl, C₂-C₆ haloalkoxycarbonyl or —Z⁴Q;
each R¹⁷ and R¹⁸ is independently C₁-C₅ alkyl, C₂-C₅ alkenyl, C₂-C₅ alkynyl, C₃-C₅ cycloalkyl, C₃-C₆ halocycloalkyl, C₄-C₁₀ cycloalkylalkyl, C₄-C₇ alkylcycloalkyl, C₅-C₇ alkylcycloalkylalkyl, C₁-C₅ haloalkyl, C₁-C₅ alkoxy or C₁-C₅ haloalkoxy;
each Q is independently phenyl, benzyl, naphthalenyl, a 5- or 6-membered heteroaromatic ring or an 8- to 11-membered heteroaromatic bicyclic ring system, each substituted with 1 to 2 substituents independently selected from R⁷ on carbon or nitrogen atom ring members, and each optionally substituted with 1 to 5 substituents independently selected from R^{7a} on carbon atom ring members and R¹² on nitrogen atom ring members; or
   a 3- to 7-membered nonaromatic carbocyclic ring, a 5-, 6- or 7-membered nonaromatic heterocyclic ring or an 8- to 11-membered nonaromatic bicyclic ring system, each optionally including ring members selected from C(═O), C(═S), S(═O)ₐ(═NR²³)_{b} and SiR¹⁷R¹⁸, and each ring or ring system substituted with 1 to 2 substituents independently selected from R⁷ on carbon or nitrogen atom ring members, and each optionally substituted with 1 to 5 substituents independently selected from R^{7a} on carbon atom ring members and R¹² on nitrogen atom ring members;
each R⁷ is independently —Z³G^{A}, —Z³G^{N} or —Z³G^{P};
each G^{A} is independently a phenyl or 5- or 6-membered heteroaromatic ring, each ring substituted with up to 5 substituents independently selected from R^{v} on carbon atom ring members and R²² on nitrogen atom ring members;
each G^{N} is independently a 3- to 7-membered nonaromatic ring including ring members selected from (CR^{v})₂, O, S, NR²², —C(R^{v})═C(R^{v})—, —C(R^{v})═N—, —N═N—, C(═O), C(═S), C(═NR²³), S(═O)ₐ(═NR²³)_{b} and SiR¹⁷R¹⁸;
each G^{P} is independently an 8- to 10-membered aromatic or 7- to 11-membered nonaromatic bicyclic ring system, said ring system including ring members selected from (CR^{v})₂, O, S, NR²², —C(R^{v})═C(R^{v})—, —C(R^{v})═N—, —N═N—, C(═O), C(═S), C(═NR²³), S(═O)ₐ(═NR²³)_{b} and SiR¹⁷R¹⁸;
each R^{v} is independently H, halogen, cyano, hydroxy, amino, nitro, —CHO, —C(═O)OH, —C(═O)NH₂, —SO₂NH₂, —C(═S)NH₂, —C(═O)NHCN, —C(═O)NHOH, —SH, —SO₂NHCN, —SO₂NHOH, —OCN, —SCN, —SF₅, —NHCHO, —NHNH₂, —N₃, —NHOH, —NHCN, —NHC(═O)NH₂, —N═C═O, —N═C═S, C₁-C₆ alkyl, C₂-C₆ alkenyl, C₂-C₆ alkynyl, C₁-C₆ haloalkyl, C₂-C₈ alkylcarbonyl, C₂-C₈ haloalkylcarbonyl, C₂-C₈ alkoxycarbonyl, C₄-C₁₀ cycloalkoxycarbonyl, C₅-C₁₂ cycloalkylalkoxycarbonyl, C₂-C₈ alkylaminocarbonyl, C₃-C₁₀ dialkylaminocarbonyl, C₂-C₆ haloalkenyl, C₂-C₆ haloalkynyl, C₃-C₈ cycloalkyl, C₃-C₈ halocycloalkyl, C₄-C₁₀ alkylcycloalkyl, C₄-C₁₀ cycloalkylalkyl, C₆-C₁₄ cycloalkylcycloalkyl, C₄-C₁₀ halocycloalkylalkyl, C₅-C₁₂ alkylcycloalkylalkyl, C₃-C₈ cycloalkenyl, C₃-C₈ halocycloalkenyl, C₂-C₈ alkoxyalkyl, C₄-C₁₀ cycloalkoxyalkyl, C₃-C₁₀ alkoxyalkoxyalkyl, C₂-C₈ alkylthioalkyl, C₂-C₈ alkylsulfinylalkyl, C₂-C₈ alkylsulfonylalkyl, C₂-C₈ alkylaminoalkyl, C₃-C₁₀ dialkylaminoalkyl, C₂-C₈ haloalkylaminoalkyl, C₄-C₁₀ cycloalkylaminoalkyl, C₄-C₁₀ cycloalkylcarbonyl, C₄-C₁₀ cycloalkylaminocarbonyl, C₂-C₇ cyanoalkyl, C₁-C₆ hydroxyalkyl, C₄-C₁₀ cycloalkenylalkyl, C₂-C₈ haloalkoxyalkyl, C₂-C₈ alkoxyhaloalkyl, C₂-C₈ haloalkoxyhaloalkyl, C₄-C₁₀ halocycloalkoxyalkyl, C₄-C₁₀ cycloalkenyloxyalkyl, C₄-C₁₀ halocycloalkenyloxyalkyl, C₃-C₁₀ dialkoxyalkyl, C₄-C₁₂ trialkoxyalkyl, C₃-C₈ alkoxyalkenyl, C₃-C₈ alkoxyalkynyl, C₃-C₁₀ halodialkylaminoalkyl, C₅-C₁₂ cycloalkyl(alkyl)aminoalkyl, C₂-C₈ alkyl(thiocarbonyl), C₃-C₁₀ alkoxyalkylcarbonyl, C₃-C₁₀ alkoxycarbonylalkyl, C₂-C₈ haloalkoxycarbonyl, C₃-C₁₀ alkoxyalkoxycarbonyl, C₂-C₈ (alkylthio)carbonyl, C₂-C₈ alkoxy(thiocarbonyl), C₂-C₈ alkylthio(thiocarbonyl), C₂-C₈ alkylamino(thiocarbonyl), C₃-C₁₀ dialkylamino(thiocarbonyl), C₃-C₁₀ alkoxy(alkyl)aminocarbonyl, C₂-C₈ alkylsulfonylaminocarbonyl, C₂-C₈ haloalkylsulfonylaminocarbonyl, C₂-C₈ alkylamidino, C₃-C₁₀ dialkylamidino, C₁-C₆ alkoxy, C₁-C₆ haloalkoxy, C₂-C₈ alkylcarbonyloxy, C₁-C₆ alkylthio, C₁-C₆ haloalkylthio, C₁-C₆ alkylsulfinyl, C₁-C₆ haloalkylsulfinyl, C₁-C₆ alkylsulfonyl, C₁-C₆ haloalkylsulfonyl, C₁-C₆ alkylaminosulfonyl, C₂-C₈ dialkylaminosulfonyl, C₃-C₁₀ trialkylsilyl, C₃-C₈ cycloalkoxy, C₃-C₈ halocycloalkoxy, C₄-C₁₀ cycloalkylalkoxy, C₂-C₆ alkenyloxy, C₂-C₆ haloalkenyloxy, C₂-C₆ alkynyloxy, C₃-C₆ haloalkynyloxy, C₂-C₈ alkoxyalkoxy, C₂-C₈ halo alkylcarbonyloxy, C₄-C₁₀ cycloalkylcarbonyloxy, C₃-C₁₀ alkylcarbonylalkoxy, C₃-C₈ cycloalkylthio, C₃-C₈ cycloalkylsulfonyl, C₃-C₈ cycloalkenyloxy, C₃-C₈ halocycloalkenyloxy, C₂-C₈ haloalkoxyalkoxy, C₂-C₈ alkoxyhaloalkoxy, C₂-C₈ haloalkoxyhaloalkoxy, C₃-C₁₀ alkoxycarbonylalkoxy, C₂-C₈ alkyl(thiocarbonyl)oxy, C₂-C₈ alkylcarbonylthio, C₂-C₈ alkyl(thiocarbonyl)thio, C₃-C₈ cycloalkylsulfinyl, C₃-C₁₀ halotrialkylsilyl, C₁-C₆ alkylamino, C₂-C₈ dialkylamino, C₂-C₈ alkylcarbonylamino, C₁-C₆ alkylsulfonylamino, C₁-C₆ haloalkylamino, C₂-C₈ halodialkylamino, C₃-C₈ cycloalkylamino, C₂-C₈ haloalkylcarbonylamino, C₁-C₆ haloalkylsulfonylamino, C₄-C₁₀ cycloalkylalkylamino, C₄-C₁₀ cycloalkyl(alkyl)amino, C₃-C₁₀ alkoxycarbonylalkylamino, C₁-C₆ alkoxyamino, C₁-C₆ haloalkoxyamino, C₄-C₁₂ dialkylimido, C₂-C₈ alkoxycarbonylamino, C₂-C₈ halo alkoxycarbonylamino, C₂-C₈ alkylaminocarbonylamino, C₃-C₁₀ dialkylaminocarbonylamino, C₃-C₁₀ alkylaminocarbonylalkylamino, C₄-C₁₂ dialkylaminocarbonylalkylamino, C₂-C₈ alkylamino(thiocarbonyl)amino, C₃-C₁₀ dialkylamino(thiocarbonyl)amino, C₃-C₁₀ alkylamino(thiocarbonyl)alkylamino or C₄-C₁₂ dialkylamino(thiocarbonyl)alkylamino;
each R^{7a} is independently C₁-C₆ alkyl, C₂-C₆ alkenyl, C₂-C₆ alkynyl, C₃-C₆ cycloalkyl, C₄-C₁₀ cycloalkylalkyl, C₄-C₁₀ alkylcycloalkyl, C₅-C₁₀ alkylcycloalkylalkyl, C₁-C₆ haloalkyl, C₂-C₆ haloalkenyl, C₂-C₆ haloalkynyl, C₃-C₆ halocycloalkyl, halogen, hydroxy, amino, cyano, nitro, C₁-C₄ alkoxy, C₁-C₄ haloalkoxy, C₁-C₄ alkylthio, C₁-C₄ alkylsulfinyl, C₁-C₄ alkylsulfonyl, C₁-C₄ haloalkylthio, C₁-C₄ haloalkylsulfinyl, C₁-C₄ haloalkylsulfonyl, C₁-C₄ alkylamino, C₂-C₈ dialkylamino, C₃-C₆ cycloalkylamino, C₂-C₄ alkoxyalkyl, C₁-C₄ hydroxyalkyl, C₂-C₄ alkylcarbonyl, C₂-C₆ alkoxycarbonyl, C₂-C₆ alkylcarbonyloxy, C₂-C₆ alkylcarbonylthio, C₂-C₆ alkylaminocarbonyl, C₃-C₈ dialkylaminocarbonyl or C₃-C₆ trialkylsilyl; or
R⁵ and R^{7a} are taken together with the atoms linking R⁵ and R^{7a} to form an optionally substituted 5- to 7-membered ring containing ring members selected from carbon, up to 3 heteroatoms selected from up to 1 O, up to 1 S and up to 1 N, and up to 3 ring members selected from C(═O), C(═S), S(═O)ₐ(═NR²³)_{b} and SiR¹⁷R¹⁸;
R¹² is H, C₁-C₃ alkyl, C₁-C₃ alkylcarbonyl, C₁-C₃ alkoxy or C₁-C₃ alkoxycarbonyl;
each Z¹ and Z² is independently a direct bond, O, C(═O), S(O)ₘ, CHR²⁰ or NR²¹;
each Z³ is independently a direct bond, O, NR²², C(═O), C(═S), S(O)ₘ, CHR²⁰, CHR²⁰—CHR²⁰, CR²⁴═CR²⁷, C≡C, OCHR²⁰ or CHR²⁰O;
each Z⁴ is independently O, C(═O), S(O)ₘ or CHR²⁰;
each R²⁰ is independently H, C₁-C₄ alkyl or C₁-C₄ haloalkyl;
each R²¹ is independently H, C₁-C₆ alkyl, C₁-C₆ haloalkyl, C₃-C₈ cycloalkyl, C₂-C₆ alkylcarbonyl, C₂-C₆ haloalkylcarbonyl, C₂-C₆ alkoxycarbonyl or C₂-C₆ haloalkoxycarbonyl;
each R²² is independently H, C₁-C₄ alkyl or C₁-C₄ haloalkyl;
each R²³ is independently H, cyano, C₁-C₆ alkyl, C₁-C₆ haloalkyl, C₃-C₈ cycloalkyl, C₃-C₈ halocycloalkyl, C₁-C₆ alkoxy, C₁-C₆ haloalkoxy, C₁-C₆ alkylamino, C₂-C₈ dialkylamino, C₁-C₆ haloalkylamino or phenyl;
each R²⁴ and R²⁷ is independently H, C₁-C₄ alkyl or C₁-C₄ haloalkyl;
each m is independently 0, 1 or 2;
n is 0, 1 or 2; and
a and b are independently 0, 1 or 2 in each instance of S(═O)ₐ(═NR²³)_{b}, provided that the sum of a and b is 1 or 2.

More particularly, this invention pertains to a compound selected from compounds of Formula 1 (including all geometric and stereoisomers) and N-oxides and salts thereof. This invention also relates to a compound selected from compounds of Formula 1A and N-oxides and salts thereof

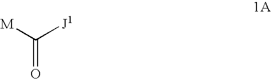

wherein
M is C₁-C₃ alkyl, C₁-C₃ haloalkyl, hydroxy, C₁-C₄ alkoxy, C₁-C₂ haloalkoxy, C₁-C₄ alkylamino, C₂-C₈ dialkylamino, 1-piperidinyl, 1-pyrrolidinyl or 4-morpholinyl; and
J¹ is any one of J-29-1 through J-29-60 depicted in Exhibit A as described below wherein the bond shown projecting to the left is bonded to —C(═O)M of Formula 1A.

More particularly, this invention pertains to a compound of Formula 1A (including all geometric and stereoisomers), an N-oxide or salt thereof (except that the compounds of Formula 1A of this invention are limited to those stereoisomer embodiments defined for J¹ in the Summary of Invention as depicted in Exhibit A below).

This invention also relates to a fungicidal composition comprising a compound of Formula 1 (including all geometric and stereoisomers, N-oxides, and salts thereof) (i.e. in a fungicidally effective amount) and at least one additional component selected from the group consisting of surfactants, solid diluents and liquid diluents.

This invention also relates to a fungicidal composition comprising a mixture of a compound of Formula 1 (including all geometric and stereoisomers, N-oxides, and salts thereof) and at least one other fungicide (e.g., at least one other fungicide having a different site of action).

This invention further relates to a method for controlling plant diseases caused by fungal plant pathogens comprising applying to the plant or portion thereof, or to the plant seed, a fungicidally effective amount of a compound of Formula 1 (including all geometric and stereoisomers, N-oxides, and salts thereof) (e.g., as a composition described herein).

This invention additionally relates to fungicidal compositions and methods of controlling plant diseases as described above.

### DETAILS OF THE INVENTION

As used herein, the terms “comprises,” “comprising,” “includes,” “including,” “has,” “having” or any other variation thereof, are intended to cover a non-exclusive inclusion. For example, a composition, process, method, article, or apparatus that comprises a list of elements is not necessarily limited to only those elements but may include other elements not expressly listed or inherent to such composition, process, method, article, or apparatus. Further, unless expressly stated to the contrary, “or” refers to an inclusive or and not to an exclusive or. For example, a condition A or B is satisfied by any one of the following: A is true (or present) and B is false (or not present), A is false (or not present) and B is true (or present), and Both A and B are true (or present).

Also, use of “a” or “an” are employed to describe elements and components of the invention. This is done merely for convenience and to give a general sense of the invention. This description should be read to include one or at least one and the singular also includes the plural unless it is obvious that it is meant otherwise.

As referred to in the present disclosure and claims, “plant” includes members of Kingdom Plantae, particularly seed plants (Spermatopsida), at all life stages, including young plants (e.g., germinating seeds developing into seedlings) and mature, reproductive stages (e.g., plants producing flowers and seeds). Portions of plants include geotropic members typically growing beneath of the surface of the growing medium (e.g., soil), such as roots, tubers, bulbs and corms, and also members growing above the growing medium, such as foliage (including stems and leaves), flowers, fruits and seeds. The term “seedling”, used either alone or in a combination of words means a young plant developing from the embryo of a seed.

In the above recitations, the term “alkyl”, used either alone or in compound words such as “alkylthio” or “haloalkyl” includes straight-chain or branched alkyl, such as, methyl, ethyl, n-propyl, i-propyl, or the different butyl, pentyl or hexyl isomers. “Alkenyl” includes straight-chain or branched alkenes such as ethenyl, 1-propenyl, 2-propenyl, and the different butenyl, pentenyl and hexenyl isomers. “Alkenyl” also includes polyenes such as 1,2-propadienyl and 2,4-hexadienyl. “Alkynyl” includes straight-chain or branched alkynes such as ethynyl, 1-propynyl, 2-propynyl and the different butynyl, pentynyl and hexynyl isomers. “Alkynyl” can also include moieties comprised of multiple triple bonds such as 2,5-hexadiynyl. “Alkylene” denotes a straight-chain or branched alkanediyl. Examples of “alkylene” include CH₂, CH₂CH₂, CH(CH₃), CH₂CH₂CH₂, CH₂CH(CH₃) and the different butylene isomers. “Alkenylene” denotes a straight-chain or branched alkenediyl containing one olefinic bond. Examples of “alkenylene” include CH═CH, CH₂CH═CH, CH═C(CH₃), CH₂CH═CH and CH₂CH═CHCH₂.

“Cycloalkyl” includes, for example, cyclopropyl, cyclobutyl, cyclopentyl, and cyclohexyl. “Cycloalkenyl” includes groups such as cyclopentenyl and cyclohexenyl as well as groups with more than one double bond such as 1,3- and 1,4-cyclohexadienyl. The term “alkylcycloalkyl” denotes alkyl substitution on a cycloalkyl moiety and includes, for example, ethylcyclopropyl, i-propylcyclobutyl, 3-methylcyclopentyl and 4-methylcyclohexyl. The term “cycloalkylalkyl” denotes cycloalkyl substitution on an alkyl moiety. Examples of “cycloalkylalkyl” include cyclopropylmethyl, cyclopentylethyl, and other cycloalkyl moieties bonded to straight-chain or branched alkyl groups. “cycloalkylcycloalkyl” denotes an cycloalkyl group substituted with other cycloalkyl group. Examples of “cycloalkylcycloalkyl” include 2-cyclopropylcyclopropyl and 3-cyclopropylcyclopentyl. “Halocycloalkylalkyl” denotes halogen substitution on the cycloalkyl moiety, the alkyl moiety or both of the cycloalkyl and alkyl moieties. Examples of “halocycloalkylalkyl” include (2-chlorocyclopropyl)methyl, 2-cyclopentyl-1-chloroethyl, and 2-(3-chlorocyclopentyl)-1-chloroethyl.

“Alkoxy” includes, for example, methoxy, ethoxy, n-propyloxy, isopropyloxy and the different butoxy, pentoxy and hexyloxy isomers. “Alkoxyalkoxy” denotes at least one straight-chain or branched alkoxy substitution on a straight-chain or branched alkoxy. Examples of “alkoxyalkoxy” include CH₃OCH₂O—, CH₃OCH₂(CH₃O)CHCH₂O— and (CH₃)₂CHOCH₂CH₂O—. The term “haloalkoxyalkoxy” denotes an alkoxyalkoxy group substituted with a haloalkoxy moiety. Examples of “haloalkoxyalkoxy” include CF₃OCH₂O—, ClCH₂CH₂OCH₂CH₂O— and Cl₃CCH₂OCH₂O— as well as branched alkyl derivatives. The term “alkoxyhaloalkoxy” denotes a haloalkoxy group further substituted with an alkoxy moiety. Examples of “alkoxyhaloalkoxy” include CH₃OCHClO—, CH₃CH₂OCH₂CHClO— and CH₃CH₂OCCl₂O— as well as branched alkyl derivatives. The term “haloalkoxyhaloalkoxy” denotes a haloalkoxy group further substituted with a haloalkoxy moiety. Examples of “haloalkoxyhaloalkoxy” include CF₃OCHClO—, ClCH₂CH₂OCHClCH₂O— and Cl₃CCH₂OCHClO— as well as branched alkyl derivatives. “Alkoxyalkyl” denotes alkoxy substitution on alkyl. Examples of “alkoxyalkyl” include CH₃OCH₂, CH₃OCH₂CH₂, CH₃CH₂OCH₂, CH₃CH₂CH₂CH₂OCH₂ and CH₃CH₂OCH₂CH₂. The term “cycloalkoxyalkyl” denotes cycloalkoxy substitution on an alkyl moiety. Examples of “cycloalkoxyalkyl” include cyclopropoxymethyl, cyclopentoxyethyl, and other cycloalkoxy moieties bonded to straight-chain or branched alkyl groups. “Alkoxyalkoxyalkyl” denotes at least one straight-chain or branched alkoxy moiety bonded to a straight-chain or branched alkoxy moiety bonded to an alkyl moiety. Examples of “alkoxyalkoxyalkyl” include CH₃OCH₂OCH₂—, CH₃CH₂O(CH₃)CHOCH₂— and (CH₃O)₂CHOCH₂—. “Alkenyloxy” includes straight-chain or branched alkenyloxy moieties. Examples of “alkenyloxy” include H₂C═CHCH₂O, (CH₃)₂C═CHCH₂O, (CH₃)CH═CHCH₂O, (CH₃)CH═C(CH₃)CH₂O and CH₂═CHCH₂CH₂O. “Alkynyloxy” includes straight-chain or branched alkynyloxy moieties. Examples of “alkynyloxy” include HC≡CCH₂O, CH₃C≡CCH₂O and CH₃C≡CCH₂CH₂O.

“Alkylthio” includes branched or straight-chain alkylthio moieties such as methylthio, ethylthio, and the different propylthio, butylthio, pentylthio and hexylthio isomers. “Alkylthioalkyl” denotes alkylthio substitution on alkyl. Examples of “alkylthioalkyl” include CH₃SCH₂, CH₃SCH₂CH₂, CH₃CH₂SCH₂, CH₃CH₂CH₂CH₂SCH₂ and CH₃CH₂SCH₂CH₂. “Alkylsulfinyl” includes both enantiomers of an alkylsulfinyl group. Examples of “alkylsulfinyl” include CH₃S(O), CH₃CH₂S(O), CH₃CH₂CH₂S(O), (CH₃)₂CHS(O) and the different butylsulfinyl, pentylsulfinyl and hexylsulfinyl isomers. “Alkylsulfinylalkyl” denotes alkylsulfinyl substitution on alkyl. Examples of “alkylsulfinylalkyl” include CH₃S(═O)CH₂, CH₃S(═O)CH₂CH₂, CH₃CH₂S(═O)CH₂ and CH₃CH₂S(═O)CH₂CH₂. Examples of “alkylsulfonyl” include CH₃S(O)₂, CH₃CH₂S(O)₂, CH₃CH₂CH₂S(O)₂, (CH₃)₂CHS(O)₂ and the different butylsulfonyl, pentylsulfonyl and hexylsulfonyl isomers. “Alkylsulfonylalkyl” denotes alkylsulfinyl substitution on alkyl. Examples of “alkylsulfonylalkyl” include CH₃S(═O)₂CH₂, CH₃S(═O)₂CH₂CH₂, CH₃CH₂S(═O)₂CH₂ and CH₃CH₂S(═O)₂CH₂CH₂.

Examples of “alkylcarbonyl” include CH₃C(O), CH₃CH₂CH₂C(O) and (CH₃)₂CHC(O). Examples of “alkoxycarbonyl” include CH₃OC(═O), CH₃CH₂OC(═O), CH₃CH₂CH₂OC(═O), (CH₃)₂CHOC(═O) and the different butoxy- or pentoxycarbonyl isomers. Examples of “alkylaminocarbonyl” include CH₃NHC(═O)—, CH₃CH₂NHC(═O)—, CH₃CH₂CH₂NHC(═O)—, (CH₃)₂CHNHC(═O)— and the different butylamino- or pentylaminocarbonyl isomers. Examples of “dialkylaminocarbonyl” include (CH₃)₂NC(═O)—, (CH₃CH₂)₂NC(═O)—, CH₃CH₂(CH₃)NC(═O)—, (CH₃)₂CHN(CH₃)C(═O)— and CH₃CH₂CH₂(CH₃)NC(═O)—. “Cycloalkylalkoxycarbonyl” denotes cycloalkyl substituted on the alkoxy moiety of an alkoxycarbonyl group. Examples of “cycloalkylalkoxycarbonyl” include cyclopropyl-CH₂OC(═O)—, cyclopropyl-CH(CH₃)OC(═O)— and cyclopentyl-CH₂OC(═O)—. “Alkoxy(alkyl)aminocarbonyl” denotes straight-chain or branched alkyl and alkoxy moieties bonded to the nitrogen atom of an aminocarbonyl group. Examples of “Alkoxy(alkyl)aminocarbonyl” include CH₃O(CH₃)NC(═O)—, CH₃CH₂O(CH₃)NC(═O)— and (CH₃)₂CHO(CH₃)NC(═O)—. The terms “haloalkylsulfonylaminocarbonyl” denotes halogen substitution on either the alkyl moiety or the nitrogen atom of an aminocarbonyl group or both the alkyl moiety and the nitrogen atom. Examples of “haloalkylsulfonylaminocarbonyl” include CF₃SO₂NH(C═O)— and CF₃SO₂NCl(C═O)—. The term “alkylcarbonyloxy” denotes straight-chain or branched alkyl bonded to a C(═O)O moiety. Examples of “alkylcarbonyloxy” include CH₃CH₂C(═O)O and (CH₃)₂CHC(═O)O. “Alkoxycarbonylalkyl” denotes alkoxycarbonyl substitution on straight-chain or branched alkyl. Examples of “alkoxycarbonylalkyl” include CH₃OC(═O)CH₂CH(CH₃), CH₃CH₂OC(═O)CH₂CH₂, (CH₃)₂CHOC(═O)CH₂. The term “alkylcarbonylalkoxy” denotes alkylcarbonyl bonded to an alkoxy moiety. Examples of “alkylcarbonylalkoxy” include CH₃C(═O)CH₂CH₂O and CH₃CH₂C(═O)CH₂O. Examples of “alkoxycarbonyloxy” include CH₃CH₂CH₂OC(═O)O and (CH₃)₂CHOC(═O)O.

“Alkyl(thiocarbonyl)” denotes straight-chain or branched alkyl moieties bonded to a C(═S) moiety. Examples of “alkyl(thiocarbonyl)” include CH₃C(═S)—, CH₃CH₂CH₂C(═S)— and (CH₃)₂CHC(═S)—. “Alkoxy(thiocarbonyl)” denotes straight-chain or branched alkoxy moieties bonded to a C(═S) moiety. Examples of “alkoxy(thiocarbonyl)” include CH₃OC(═S)—, CH₃CH₂CH₂OC(═S)— and (CH₃)₂CHOC(═S)—. “Alkylthio(thiocarbonyl)” denotes a straight-chain or branched alkylthio moiety bonded to a C(═S) moiety. Examples of “alkylthio(thiocarbonyl)” include CH₃SC(═S)—, CH₃CH₂CH₂SC(═S)— and (CH₃)₂CHSC(═S)—. “Alkylamino(thiocarbonyl)” denotes a straight-chain or branched alkylamino moiety bonded to a C(═S) moiety. Examples of “alkylamino(thiocarbonyl)” include CH₃NHC(═S)—, CH₃CH₂CH₂NHC(═S)— and (CH₃)₂CHNHC(═S)—. “Dialkylamino(thiocarbonyl)” denotes a straight-chain or branched dialkylamino moiety bonded to a C(═S) moiety. Examples of “dialkylamino(thiocarbonyl)” include (CH₃)₂NC(═S)—, CH₃CH₂CH₂(CH₃)NC(═S)— and (CH₃)₂C(CH₃)NC(═S)—.

“Alkylamidino” denotes a straight-chain or branched alkylamino moiety bonded to a carbon atom of a C(═N) moiety, or an unsubstituted amino moiety bonded to the carbon atom of a C(═N) moiety and a straight-chain or branched alkyl moiety bonded to the nitrogen atom of the C(═N) moiety. Examples of “alkylamidino” include CH₃NHC(═NH)—, CH₃CH₂NHC(═NH)— and H₂NC(═NCH₃)—. “Dialkylamidino” denotes a straight-chain or branched dialkylamino moiety bonded to the carbon atom of a C(═N) moiety, or a straight-chain or branched alkylamino moiety bonded to the carbon atom of a C(═N) moiety and a straight-chain or branched alkyl moiety bonded to the nitrogen atom of the C(═N) moiety. Examples of “dialkylamidino” include (CH₃)₂NC(═NH)—, CH₃CH₂(CH₃)NC(═NH)— and CH₃NHC(═NCH₃)—.

“Alkylamino”, “dialkylamino” and the like, are defined analogously to the above examples. The term “halodialkylamino” denotes a dialkylamino group substituted on at least one alkyl moiety with one or more halogenatoms which may be the same or different. Examples of “halodialkylamino” include CF₃(CH₃)N—, (CF₃)₂N— and CH₂Cl(CH₃)N—. “Cycloalkylamino” means the amino nitrogen atom is attached to a cycloalkyl radical and a hydrogen atom and includes groups such as cyclopropylamino, cyclobutylamino, cyclopentylamino and cyclohexylamino. “Cycloalkyl(alkyl)amino” means a cycloalkylamino group wherein the amino hydrogen atom is replaced by an alkyl radical. Examples of “cycloalkyl(alkyl)amino” include groups such as cyclopropyl(methyl)amino, cyclobutyl(butyl)amino, cyclopentyl(propyl)amino, cyclohexyl(methyl)amino and the like. “Haloalkylaminoalkyl” denotes an alkylaminoalkyl group substituted on the amino nitrogen or either alkyl moiety or a combination thereof with one or more halogen atoms which may be the same or different. “Haloalkylaminoalkyl” includes a halogen group attached to any alkyl groups as well as nitrogen. Examples of “haloalkylaminoalkyl” include CH₃NHCHCl—, (CH₃)₂CClNHCH₂— and CH₃NClCH(CH₃)—.

The term “dialkylimido” denotes two independent straight-chain or branched alkylcarbonyl moieties bonded to the nitrogen atom of an amino group. Examples of “dialkylimido” include (CH₃C(═O))₂N— and CH₃CH₂C(═O)(CH₃C(═O))N—. The term “alkoxycarbonylamino” denotes a straight-chain or branched alkoxy moiety bonded to the C(═O) moiety of a carbonylamino group. Examples of “alkoxycarbonylamino” include CH₃OC(═O)NH— and CH₃CH₂OC(═O)NH—. The term “alkylaminocarbonylamino” denotes a straight-chain or branched alkylamino moiety bonded to the C(═O) moiety of a carbonylamino group. Examples of “alkylaminocarbonylamino” include CH₃NHC(═O)NH— and CH₃CH₂NHC(═O)NH—. The term “dialkylaminocarbonylamino” denotes a straight-chain or branched dialkylamino moiety bonded to the C(═O) moiety of a carbonylamino group. Examples of “dialkylaminocarbonylamino” include (CH₃)₂NC(═O)NH— and CH₃CH₂(CH₃)NC(═O)NH—. The term “alkylaminocarbonylalkylamino” denotes a straight-chain or branched alkylamino moiety bonded to the C(═O) moiety of a carbonylamino group and a straight-chain or branched alkyl moiety bonded to the amino nitrogen of a carbonylamino group. Examples of “alkylaminocarbonylalkylamino” include CH₃NHC(═O)N(CH₃)— and CH₃CH₂NHC(═O)N(CH₃)—. The term “dialkylaminocarbonylalkylamino” denotes a straight-chain or branched dialkylamino moiety bonded to the C(═O) moiety of a carbonylamino group and a straight-chain or branched alkyl moiety bonded to the amino nitrogen of a carbonylamino group. Examples of “dialkylaminocarbonylalkylamino” include (CH₃)₂NC(═O)N(CH₃)— and CH₃CH₂(CH₃)NC(═O)N(CH₃)—. The terms “alkylamino(thiocarbonyl)amino” denotes straight-chain or branched alkylamino moieties bonded to a C(═S) moiety of carbonylamino group. Examples of “alkylamino(thiocarbonyl)amino” include CH₃NHC(═S)NH— and CH₃CH₂NHC(═S)NH—.

“Trialkylsilyl” includes 3 branched and/or straight-chain alkyl radicals attached to and linked through a silicon atom, such as trimethylsilyl, triethylsilyl and tert-butyldimethylsilyl. The terms “halotrialkylsilyl” denotes one or more halogen atoms substituted on at least one alkyl moiety of the trialkylsilyl group. Examples of “halotrialkylsilyl” include CF₃(CH₃)₂Si—, (CF₃)₃Si—, and CH₂Cl(CH₃)₂Si—.

“Hydroxyalkyl” denotes an alkyl group substituted with one hydroxy group. Examples of “hydroxyalkyl” include HOCH₂CH₂, CH₃CH₂(OH)CH and HOCH₂CH₂CH₂CH₂.

The term “halogen”, either alone or in compound words such as “haloalkyl”, includes fluorine, chlorine, bromine or iodine. Furthermore, when used in compound words such as “haloalkyl”, said alkyl may be partially or fully substituted with halogen atoms which may be the same or different. Examples of “haloalkyl” include F₃C, ClCH₂, CF₃CH₂ and CF₃CCl₂. The terms “haloalkenyl”, “haloalkynyl”, “halocycloalkyl”, “haloalkoxy”, “haloalkylthio”, and the like, are defined analogously to the term “haloalkyl”. Examples of “haloalkenyl” include (Cl)₂C═CHCH₂ and CF₃CH₂CH═CHCH₂. Examples of “haloalkynyl” include HC≡CCHCl, CF₃C≡C, CCl₃C≡C and FCH₂C≡CCH₂. Examples of “haloalkoxy” include CF₃O, CCl₃CH₂O, HCF₂CH₂CH₂O and CF₃CH₂O. Examples of “haloalkylthio” include CCl₃S, CF₃S, CCl₃CH₂S and ClCH₂CH₂CH₂S. Examples of “haloalkylsulfinyl” include CF₃S(O), CCl₃S(O), CF₃CH₂S(O) and CF₃CF₂S(O). Examples of “haloalkylsulfonyl” include CF₃S(O)₂, CCl₃S(O)₂, CF₃CH₂S(O)₂ and CF₃CF₂S(O)₂.

Unless otherwise indicated, a “ring” or “ring system” as a component of Formula 1 (e.g., substituent J and Q) is carbocyclic or heterocyclic. The term “ring system” denotes two or more connected rings. The term “spirocyclic ring system” denotes a ring system consisting of two rings connected at a single atom (so the rings have a single atom in commonality). The term “bicyclic ring system” denotes a ring system consisting of two rings sharing two or more common atoms. In a “fused bicyclic ring system” the common atoms are adjacent, and therefore the rings share two adjacent atoms and bond connecting them. In a “bridged bicyclic ring system” the common atoms are not adjacent (i.e. there is no bond between the bridgehead atoms). A “bridged bicyclic ring system” is conceptually formed by bonding a segment of one or more atoms to nonadjacent ring members of a ring.

A ring, a bicyclic ring system or spirocyclic ring system can be part of an extended ring system containing more than two rings wherein substituents on the ring, bicyclic ring system or spirocyclic ring system are taken together to form the additional rings, which may be in bicyclic and/or spirocyclic relationships with other rings in the extended ring system. For example, the particular J or J¹ moiety J-29-59 depicted in Exhibit A consists of a dihydro isoxazoline ring having one R⁵ substituent as Z²Q, which is a phenyl ring substituted with a phenyl group (as Z³G^{A}) and also one R^{7a} group taken together with another R⁵ substituent on the dihydro isoxazoline ring as —CH₂CH₂CH₂— to form the additional six-membered ring component in the ring system.

The term “ring member” refers to an atom (e.g., C, O, N or S) or other moiety (e.g., C(═O), C(═S) or S(═O)ₐ(═NR²³)_{b}) forming the backbone of a ring or ring system. The term “carbocyclic ring” denotes a ring wherein the atoms forming the ring backbone are selected only from carbon. The term “carbocyclic ring system” denotes two or more fused rings wherein the atoms forming the backbone of the rings are selected only from carbon. The term “heterocyclic ring” denotes a ring wherein at least one of the atoms forming the ring backbone is other than carbon. The term “heterocyclic ring system” denotes two or more fused rings wherein at least one of the atoms forming the backbone of the rings is other than carbon. “Aromatic” indicates that each of the ring atoms is essentially in the same plane and has a p-orbital perpendicular to the ring plane, and in which (4n+2)π electrons, where n is a positive integer, are associated with the ring to comply with Hückel's rule. The term “heteroaromatic ring” refers to a heterocyclic ring that is aromatic. The term “saturated heterocyclic ring” denotes a heterocyclic ring containing only single bonds between ring members. The term “partially saturated heterocyclic ring” denotes a heterocyclic ring containing at least one double bond but which is not aromatic.

The dotted line in Formula 1 and in other rings depicted in the present description (e.g., J-44, J-45, J-48 and J-49 in Exhibit 3) represents that the bond indicated can be a single bond or double bond. Unless otherwise indicated, heterocyclic rings and ring systems are attached to the remainder of Formula 1 through any available carbon or nitrogen by replacement of a hydrogen on said carbon or nitrogen, and all substituents on the heterocyclic rings and ring systems are attached through any available carbon or nitrogen by replacement of a hydrogen on said carbon or nitrogen.

As already described, J is a 5-, 6- or 7-membered ring, a 8- to 11-membered bicyclic ring system or a 7- to 11-membered spirocyclic ring system, each ring or ring system containing ring members selected from carbon, up to 4 heteroatoms selected from up to 2 O, up to 2 S and up to 4 N, and up to 3 ring members selected from C(═O), C(═S), S(═O)ₐ(═NR²³)_{b} and SiR¹⁷R¹⁸, each ring or ring system substituted with 1 to 2 substituents independently selected from —Z²Q and optionally substituted with 1 to 5 substituents independently selected from R⁵. As the heteroatoms are optional, 0 to 4 heteroatoms may be present. In this description the heteroatoms selected from up to 2 S are atoms and not the moieties S(═O)ₐ(═NR²³)_{b}. The heteroatoms selected from up to 4 N may be oxidized as N-oxides, because the present invention also relates to N-oxide derivatives of the compounds of Formula 1. Therefore the optional 1 to 3 ring members selected from C(═O), C(═S), S(═O)ₐ(═NR²³)_{b} and SiR¹⁷R¹⁸ are in addition to the optional 1 to 4 heteroatoms selected from up to 2 O, up to 2 S and up to 4 N. Of note is when the total number of unoxidized sulfur atoms (i.e. S) and oxidized sulfur moieties (i.e. S(═O)ₐ(═NR²³)_{b}) does not exceed 2, so that at most two ring members selected from S and S(═O)ₐ(═NR²³)_{b} are present in the ring or ring system. When none of the optional heteroatoms and none of the optional ring members selected from S(═O)ₐ(═NR²³)_{b} and SiR¹⁷R¹⁸ are present, the ring or ring system is carbocyclic. The R⁵ substituents may be attached to carbon atom ring members and to nitrogen atom ring members having an available point of attachment. The carbon-based ring members C(═O) and C(═S) do not have available points of attachment. Furthermore in SiR¹⁷R¹⁸ ring members, the substituents R¹⁷ and R¹⁸ are otherwise separately defined, and these ring members cannot be further substituted with R⁵. As the R⁵ substituents are optional, 0 to 5 substituents may be present, limited by the number of available points of attachment.

Similarly, R⁵ and R^{7a} may be taken together with the atoms linking R⁵ and R^{7a} to form an optionally substituted 5- to 7-membered ring containing ring members selected from carbon, up to 3 heteroatoms selected from up to 1 O, up to 1 S and up to 1 N, and up to 3 ring members selected from C(═O), C(═S), S(═O)ₐ(═NR²³)_{b} and SiR¹⁷R¹⁸. As the heteroatoms are optional, 0 to 3 heteroatoms may be present. In this description the heteroatom selected from up to 1 S is an atom and not the moiety S(═O)ₐ(═NR²³)_{b}. The heteroatom selected from up to 1 N may be oxidized as an N-oxide, because the present invention also relates to N-oxide derivatives of the compounds of Formula 1. Therefore the optional 1 to 3 ring members selected from C(═O), C(═S), S(═O)ₐ(═NR²³)_{b} and SiR¹⁷R¹⁸ are in addition to the optional 1 to 3 heteroatoms selected from up to 1 O, up to 1 S and up to 1 N. Of note is when the total number of unoxidized sulfur atoms (i.e. S) and oxidized sulfur moieties (i.e. S(═O)ₐ(═NR²³)_{b}) does not exceed 1, so that at most one ring member selected from S and S(═O)ₐ(═NR²³)_{b} is present in the ring. When none of the optional heteroatoms and none of the optional ring members selected from S(═O)ₐ(═NR²³)_{b} and SiR¹⁷R¹⁸ are present, the ring is carbocyclic. The 5- to 7-membered ring is optionally substituted. The substituents on the atoms linking R⁵ and R^{7a} are described in the definition of the components linking R⁵ and R^{7a}. For example, when linking component Z² is CHR²⁰, the substituent R²⁰ is defined to be H, C₁-C₄ alkyl or C₁-C₄ haloalkyl. Regarding optional substituents attached to the portion of the ring consisting of R⁵ and R^{7a} taken together, an optional substituent is a non-hydrogen substituent that does not extinguish fungicidal activity. Optional substituents may be attached to carbon atom ring members and to nitrogen atom ring members having an available point of attachment. The carbon-based ring members C(═O) and C(═S) do not have available points of attachment. Furthermore in SiR¹⁷R¹⁸ ring members, the substituents R¹⁷ and R¹⁸ are otherwise separately defined, and these ring members cannot be further substituted. Likewise in S(═O)ₐ(═NR²³)_{b} ring members, the substituent R²³ is otherwise separately defined, and these ring members cannot be further substituted.

The total number of carbon atoms in a substituent group is indicated by the “Cᵢ-Cⱼ” prefix where i and j are numbers from 1 to 10. For example, C₁-C₄ alkylsulfonyl designates methylsulfonyl through butylsulfonyl; C₂ alkoxyalkyl designates CH₃OCH₂; C₃ alkoxyalkyl designates, for example, CH₃CH(OCH₃), CH₃OCH₂CH₂ or CH₃CH₂OCH₂; and C₄ alkoxyalkyl designates the various isomers of an alkyl group substituted with an alkoxy group containing a total of four carbon atoms, examples including CH₃CH₂CH₂OCH₂ and CH₃ CH₂OCH₂CH₂.

When a compound is substituted with a substituent bearing a subscript that indicates the number of said substituents can vary, then when the number of said substituents is greater than 1, said substituents are independently selected from the group of defined substituents. Furthermore when a range is indicated (e.g., i-j substituents), then the number of substituents may be selected from the integers between i and j inclusive. When a group (e.g., J) contains a substituent (e.g., R⁵) which can be hydrogen, then when this substituent is taken as hydrogen, it is recognized that this is equivalent to said group being unsubstituted. When a variable group is shown to be optionally attached to a position, for example, (R²)ₙ wherein n may be 0, or as a further example (R⁴)ₖ wherein k may be 0 in U-17 of Exhibit 1, then hydrogen may be at the position even if not recited in the definition of the variable group (e.g., R² and R⁴). When a position on a group is said to be “not substituted” or “unsubstituted”, then hydrogen atoms are attached to take up any free valency. The term “optionally substituted” in connection with groups listed for R¹, R², R⁵, R^{7a}, G, J and Q refers to groups that are unsubstituted or have at least 1 non-hydrogen substituent. Unless otherwise indicated, these groups may be substituted with as many optional substituents as can be accommodated by replacing a hydrogen atom with a non-hydrogen substituent on any available carbon or nitrogen atom. Commonly, the number of optional substituents (when present) ranges from 1 to 3. When a range specified for the number of substituents (e.g., x being an integer from 0 to 5 in Exhibit 3) exceeds the number of positions available for substituents on a ring (e.g., there is only 1 position available if s is 1 (s cannot equal 0) or no positions available if s is 2 for (R⁵)ₓ on J-1 in Exhibit 3), the actual higher end of the range is recognized to be the number of available positions. The term “optionally substituted” means that the number of substituents can be zero. For example, the phrase “optionally substituted with up to 2 substituents selected from R³ on carbon ring members and selected from R¹¹ on nitrogen ring members” means that 0, 1 or 2 substituents can be present (if the number of potential connection points allows), and thus the number of R³ and R¹¹ substituents can be zero. Similarly, the phrase “optionally substituted with 1 to 5 substituents” means that 0, 1, 2, 3, 4 or 5 substituents can be present if the number of available connection points allows. The term “unsubstituted” in connection with a group such as a ring or ring system means the group does not have any substituents other than its one or more attachments to the remainder of Formula 1. The term “meta-substituted phenyl” means a phenyl ring substituted with a non-hydrogen substituent at a meta position relative to attachment of the phenyl ring to the remainder of Formula 1.

As noted above, R¹ is an optionally substituted phenyl, or 5- or 6-membered heteroaromatic ring or optionally substituted naphthalenyl; G is an optionally substituted 5-membered heterocyclic ring; R⁵ and R^{7a} may be taken together with the atoms linking R⁵ and R^{7a} to form an optionally substituted 5- to 7-membered ring containing ring members selected from carbon, up to 3 heteroatoms selected from up to 1 O, up to 1 S and up to 1 N, and up to 1 to 3 ring members selected from C(═O), C(═S), S(═O)ₐ(═NR²³)_{b} and SiR¹⁷R¹⁸. The term “substituted” in connection with the definitions of R¹, G, R⁵ and R^{7a} refers to groups that have at least one non-hydrogen substituent that does not extinguish fungicidal activity. Since these groups are optionally substituted, they need not have any non-hydrogen substituents. As these groups are “optionally substituted” without the number of substituents indicated, these groups may be substituted with as many optional substituents as can be accommodated by replacing a hydrogen atom with a non-hydrogen substituent on any available carbon or nitrogen atom.

When Z³ is CR²⁴═CR²⁷, OCHR²⁰ or CHR²⁰O, the left end of the radicals are connected to Q and the right end of the radicals are connected to G^{A}, G^{N} or G^{P}.

Naming of substituents in the present disclosure uses recognized terminology providing conciseness in precisely conveying to those skilled in the art the chemical structure. For sake of conciseness, locant descriptors may be omitted; “pyrazol-1-yl” means “1H-pyrazol-1-yl” according to the Chemical Abstracts system of nomenclature. The term “pyridyl” is synonymous with “pyridinyl”. The order of listing substituents may be different from the Chemical Abstracts system if the difference does not affect the meaning.

Compounds of this invention can exist as one or more stereoisomers. The various stereoisomers include enantiomers, diastereomers, atropisomers and geometric isomers. One skilled in the art will appreciate that one stereoisomer may be more active and/or may exhibit beneficial effects when enriched relative to the other stereoisomer(s) or when separated from the other stereoisomer(s). Additionally, the skilled artisan knows how to separate, enrich, and/or to selectively prepare said stereoisomers. The compounds of the invention may be present as a mixture of stereoisomers, individual stereoisomers, or as an optically active form. For example, when J is J-29 (see Exhibit 3) bonded at the 3-position to the remainder of Formula 1 and J-29 has one Q substituent other than H at the 5-position (Z² being a direct bond, s being 1, and x being 0), then Formula 1 possesses a chiral center at the carbon atom to which Q is bonded. The two enantiomers are depicted as Formula 1′ and Formula 1″ with the chiral center identified with an asterisk (*).

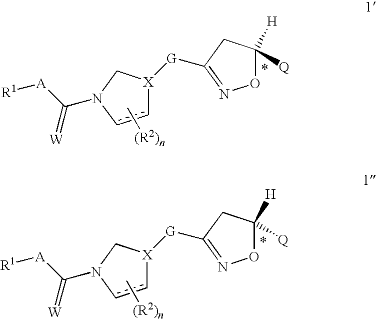

This invention comprises racemic mixtures, for example, equal amounts of the enantiomers of Formulae 1′ and 1″. In addition, this invention includes compounds that are enriched compared to the racemic mixture in an enantiomer of Formula 1. Also included are the essentially pure enantiomers of compounds of Formula 1, for example, Formula 1′ and Formula″.

When enantiomerically enriched, one enantiomer is present in greater amounts than the other, and the extent of enrichment can be defined by an expression of enantiomeric excess (“ee”), which is defined as (2x−1)·100%, where x is the mole fraction of the dominant enantiomer in the mixture (e.g., an ee of 20% corresponds to a 60:40 ratio of enantiomers).

Preferably the compositions of this invention have at least a 50% enantiomeric excess; more preferably at least a 75% enantiomeric excess; still more preferably at least a 90% enantiomeric excess; and the most preferably at least a 94% enantiomeric excess of the more active isomer. Of particular note are enantiomerically pure embodiments of the more active isomer.

Compounds of Formula 1 can comprise additional chiral centers. For example, substituents and other molecular constituents such as R⁴, R⁵, R^{7a}, G, J, Q and X¹ through X⁹ may themselves contain chiral centers. This invention comprises racemic mixtures as well as enriched and essentially pure stereoconfigurations at these additional chiral centers.

Compounds of this invention can exist as one or more conformational isomers due to restricted rotation about the amide bond (e.g., C(W)—N) in Formula 1. This invention comprises mixtures of conformational isomers. In addition, this invention includes compounds that are enriched in one conformer relative to others.

Some of the unsaturated rings and ring systems depicted in Exhibits 1, 2, 3, 4 and 5 can have an arrangement of single and double bonds between ring members different from that depicted. Such differing arrangements of bonds for a particular arrangement of ring atoms correspond to different tautomers. For these unsaturated rings and ring systems, the particular tautomer depicted is to be considered representative of all the tautomers possible for the arrangement of ring atoms shown. The tables listing particular compounds incorporating the ring and ring systems depicted in the Exhibits may involve a tautomer different from the tautomer depicted in the Exhibits.

The compounds of the invention include N-oxide derivatives. One skilled in the art will appreciate that not all nitrogen-containing heterocycles can form N-oxides since the nitrogen requires an available lone pair of electrons for oxidation to the oxide; one skilled in the art will recognize those nitrogen-containing heterocycles which can form N-oxides. One skilled in the art will also recognize that tertiary amines can form N-oxides. Synthetic methods for the preparation of N-oxides of heterocycles and tertiary amines are very well known by one skilled in the art including the oxidation of heterocycles and tertiary amines with peroxy acids such as peracetic and m-chloroperbenzoic acid (MCPBA), hydrogen peroxide, alkyl hydroperoxides such as tent-butyl hydroperoxide, sodium perborate, and dioxiranes such as dimethyldioxirane. These methods for the preparation of N-oxides have been extensively described and reviewed in the literature; see, for example, T. L. Gilchrist in Comprehensive Organic Synthesis, vol. 7, pp 748-750, S. V. Ley, Ed., Pergamon Press; M. Tisler and B. Stanovnik in Comprehensive Heterocyclic Chemistry, vol. 3, pp 18-20, A. J. Boulton and A. McKillop, Eds., Pergamon Press; M. R. Grimmett and B. R. T. Keene in Advances in Heterocyclic Chemistry, vol. 43, pp 149-161, A. R. Katritzky, Ed., Academic Press; M. Tisler and B. Stanovnik in Advances in Heterocyclic Chemistry, vol. 9, pp 285-291, A. R. Katritzky and A. J. Boulton, Eds., Academic Press; and G. W. H. Cheeseman and E. S. G. Werstiuk in Advances in Heterocyclic Chemistry, vol. 22, pp 390-392, A. R. Katritzky and A. J. Boulton, Eds., Academic Press.

The present compounds of Formula 1 can be in the form of agriculturally suitable salts. One skilled in the art recognizes that because in the environment and under physiological conditions salts of chemical compounds are in equilibrium with their corresponding nonsalt forms, salts share the biological utility of the nonsalt forms. Thus a wide variety of salts of the compounds of Formula 1 are useful for control of plant diseases caused by fungal plant pathogens (i.e. are agriculturally suitable). The salts of the compounds of Formula 1 include acid-addition salts with inorganic or organic acids such as hydrobromic, hydrochloric, nitric, phosphoric, sulfuric, acetic, butyric, fumaric, lactic, maleic, malonic, oxalic, propionic, salicylic, tartaric, 4-toluenesulfonic or valeric acids. When a compound of Formula 1 contains an acidic moiety such as a carboxylic acid or phenol, salts also include those formed with organic or inorganic bases such as pyridine, triethylamine or ammonia, or amides, hydrides, hydroxides or carbonates of sodium, potassium, lithium, calcium, magnesium or barium.

Compounds selected from Formula 1 and 1A (including geometric and stereoisomers), N-oxides, and salts thereof, typically exist in more than one form, and Formula 1 or 1A thus includes all crystalline and non-crystalline forms of the compounds that Formula 1 or 1A represents. Non-crystalline forms include embodiments which are solids such as waxes and gums as well as embodiments which are liquids such as solutions and melts. Crystalline forms include embodiments which represent essentially a single crystal type and embodiments which represent a mixture of polymorphs (i.e. different crystalline types). The term “polymorph” refers to a particular crystalline form of a chemical compound that can crystallize in different crystalline forms, these forms having different arrangements and/or conformations of the molecules in the crystal lattice. Although polymorphs can have the same chemical composition, they can also differ in composition due the presence or absence of co-crystallized water or other molecules, which can be weakly or strongly bound in the lattice. Polymorphs can differ in such chemical, physical and biological properties as crystal shape, density, hardness, color, chemical stability, melting point, hygroscopicity, suspensibility, dissolution rate and biological availability. One skilled in the art will appreciate that a polymorph of a compound represented by Formula 1 or 1A can exhibit beneficial effects (e.g., suitability for preparation of useful formulations, improved biological performance) relative to another polymorph or a mixture of polymorphs of the same compound represented by Formula 1 or 1A. Preparation and isolation of a particular polymorph of a compound represented by Formula 1 or 1A can be achieved by methods known to those skilled in the art including, for example, crystallization using selected solvents and temperatures.

Embodiments of the present invention as described in the Summary of the Invention include those described below. In the following Embodiments, Formulae 1 and 1A include N-oxides and salts thereof, and reference to “a compound of Formula 1” or “a compound of Formula 1A” includes the definitions of substituents specified in the Summary of the Invention unless further defined in the Embodiments.

Embodiments of the present invention include:

### Embodiment 1

A compound of Formula 1 wherein A is CHR¹⁵.

### Embodiment 1a

A compound of Formula 1 or Embodiment 1 wherein R¹⁵ is H, halogen, cyano, hydroxy, —CHO, C₁-C₄ alkyl, C₁-C₄ haloalkyl or C₂-C₅ alkoxycarbonyl.

### Embodiment 1b

A compound of Embodiment 1a wherein R¹⁵ is H, cyano, hydroxy, methyl or methoxycarbonyl.

### Embodiment 1c

A compound of Embodiment 1b wherein R¹⁵ is H.

### Embodiment 2

A compound of Formula 1 wherein A is NR¹⁶.

### Embodiment 2a

A compound of Formula 1 or any one of Embodiments 1 through 2 wherein R¹⁶ is H, C₁-C₄ alkyl, C₁-C₄ haloalkyl, C₂-C₄ alkylcarbonyl, C₂-C₄ haloalkylcarbonyl or C₂-C₄ alkoxycarbonyl.

### Embodiment 2b

A compound of Embodiment 2a wherein R¹⁶ is H, methyl, methylcarbonyl or methoxycarbonyl.

### Embodiment 2c

A compound of Embodiment 2b wherein R¹⁶ is H.

### Embodiment 3

A compound of Formula 1 or any one of Embodiments 1 through 2c wherein W is O.

### Embodiment 4

A compound of Formula 1 or any one of Embodiments 1 through 2c wherein W is S.

### Embodiment 5

A compound of Formula 1 wherein
each R² is independently C₁-C₄ alkyl, C₁-C₄ alkenyl, C₁-C₄ haloalkyl, C₁-C₄ alkoxy, halogen, cyano or hydroxy; or
two R² are taken together as C₁-C₃ alkylene or C₂-C₃ alkenylene to form a bridged bicyclic ring system; or
two R² attached to adjacent ring carbon atoms joined by a double bond are taken together as —CH═CH—CH═CH— optionally substituted with 1 to 3 substituents selected from C₁-C₄ alkyl, C₁-C₄ haloalkyl, C₁-C₄ alkoxy, C₁-C₄ haloalkoxy, halogen, hydroxy, amino, cyano and nitro.

### Embodiment 5a

A compound of Embodiment 5 wherein each R² is independently C₁-C₂ alkyl, C₁-C₂ haloalkyl, C₁-C₂ alkoxy, halogen, cyano or hydroxy.

### Embodiment 5b

A compound of Embodiment 5a wherein each R² is independently methyl, methoxy, cyano or hydroxy.

### Embodiment 5c

A compound of Embodiment 5b wherein each R² is methyl.

### Embodiment 6

A compound of Formula 1 or any one of Embodiments 1 through 5c wherein n is 0 or 1.

### Embodiment 7

A compound of Embodiment 6 wherein n is 0.

### Embodiment 7a

A compound of Embodiment 6 wherein n is 1.

### Embodiment 8

A compound of Formula 1 or any one of Embodiments 1 through 7a wherein X is X¹, X² or X³.

### Embodiment 9

A compound of Embodiment 8 wherein X is X¹ or X².

### Embodiment 10

A compound of Embodiment 9 wherein X is X¹.

### Embodiment 11

A compound of Formula 1 or any one of Embodiments 1 through 10 wherein the ring comprising X is saturated (i.e. contains only single bonds).

### Embodiment 12

A compound of Formula 1 or any one of Embodiments 1 through 11 wherein R¹ is a phenyl or 5- or 6-membered heteroaromatic ring optionally substituted with substituents that do not link together to make R¹ a fused ring system.

### Embodiment 12a

A compound of Embodiment 12 wherein R¹ is a phenyl or 5- or 6-membered heteroaromatic ring optionally substituted with 1-3 substituents independently selected from R^{4a} on carbon ring members and R^{4b} on nitrogen ring members;
each R^{4a} is independently C₁-C₆ alkyl, C₂-C₆ alkenyl, C₂-C₆ alkynyl, C₃-C₆ cycloalkyl, C₄-C₁₀ cycloalkylalkyl, C₄-C₁₀ alkylcycloalkyl, C₅-C₁₀ alkylcycloalkylalkyl, C₁-C₆ haloalkyl, C₂-C₆ haloalkenyl, C₂-C₆ haloalkynyl, C₃-C₆ halocycloalkyl, halogen, hydroxy, amino, cyano, nitro, C₁-C₄ alkoxy, C₁-C₄ haloalkoxy, C₁-C₄ alkylthio, C₁-C₄ alkylsulfinyl, C₁-C₄ alkylsulfonyl, C₁-C₄ haloalkylthio, C₁-C₄ haloalkylsulfinyl, C₁-C₄ haloalkylsulfonyl, C₁-C₄ alkylamino, C₂-C₈ dialkylamino, C₃-C₆ cycloalkylamino, C₂-C₄ alkoxyalkyl, C₁-C₄ hydroxyalkyl, C₂-C₄ alkylcarbonyl, C₂-C₆ alkoxycarbonyl, C₂-C₆ alkylcarbonyloxy, C₂-C₆ alkylcarbonylthio, C₂-C₆ alkylaminocarbonyl, C₃-C₈ dialkylaminocarbonyl or C₃-C₆ trialkylsilyl; and
each R^{4b} is independently C₁-C₆ alkyl, C₃-C₆ alkenyl, C₃-C₆ alkynyl, C₃-C₆ cycloalkyl, C₁-C₆ haloalkyl, C₃-C₆ haloalkenyl, C₃-C₆ haloalkynyl, C₃-C₆ halocycloalkyl or C₂-C₄ alkoxyalkyl.

### Embodiment 12b

A compound of Embodiment 12a wherein R¹ is a phenyl or 5- or 6-membered heteroaromatic ring optionally substituted with 1-2 substituents independently selected from R^{4a} on carbon ring members and R^{4b} on nitrogen ring members.

### Embodiment 13

A compound of any one of Embodiments 12a through 12b wherein each R^{4a} is independently C₁-C₃ alkyl, C₂-C₃ alkenyl, C₂-C₃ alkynyl, cyclopropyl, C₁-C₃ haloalkyl, C₂-C₃ haloalkenyl, C₂-C₃ haloalkynyl, halocyclopropyl, halogen, cyano, nitro, C₁-C₂ alkoxy, C₁-C₂ haloalkoxy, C₁-C₂ alkylthio, C₁-C₂ haloalkylthio, C₂-C₃ alkoxyalkyl, C₂-C₃ alkylcarbonyl, C₂-C₃ alkoxycarbonyl, C₂-C₃ alkylaminocarbonyl or C₃-C₄ dialkylaminocarbonyl.

### Embodiment 14

A compound of Embodiment 13 wherein each R^{4a} is independently C₁-C₃ alkyl, C₂-C₃ alkenyl, C₂-C₃ alkynyl, cyclopropyl, C₁-C₃ haloalkyl, C₂-C₃ haloalkenyl, C₂-C₃ haloalkynyl, halocyclopropyl, halogen, cyano, nitro, C₁-C₂ alkoxy or C₁-C₂ haloalkoxy.

### Embodiment 15

A compound of Embodiment 14 wherein each R^{4a} is independently halogen, C₁-C₃ alkyl, C₁-C₃ haloalkyl, C₁-C₂ alkoxy or C₁-C₂ haloalkoxy.

### Embodiment 15a

A compound of Embodiment 15 wherein each R^{4a} is independently C₁-C₂ alkyl, C₁-C₂ haloalkyl, halogen, C₁-C₂ alkoxy or C₁-C₂ haloalkoxy;

### Embodiment 16

A compound of Embodiment 15a wherein each R^{4a} is independently halogen, C₁-C₂ alkyl, C₁-C₂ haloalkyl or C₁-C₂ alkoxy.

### Embodiment 17

A compound of Embodiment 16 wherein each R^{4a} is independently C₁-C₂ alkyl, trifluoromethyl, Cl, Br, I or methoxy.

### Embodiment 18

A compound of Embodiment 17 wherein each R^{4a} is independently C₁-C₂ alkyl, trifluoromethyl, Cl or Br.

### Embodiment 19

A compound of any one of Embodiments 12a through 18 wherein each R^{4b} is independently C₁-C₃ alkyl, C₃ alkenyl (e.g., allyl), C₃ alkynyl (e.g., propargyl), cyclopropyl, C₁-C₃ haloalkyl, C₃ haloalkenyl, C₃ haloalkynyl, halocyclopropyl or C₂-C₃ alkoxyalkyl.

### Embodiment 20

A compound of Embodiment 19 wherein each R^{4b} is independently C₁-C₃ alkyl, C₃ alkenyl, C₃ alkynyl, cyclopropyl, C₁-C₃ haloalkyl, C₃ haloalkenyl or halocyclopropyl.

### Embodiment 21

A compound of Embodiment 20 wherein each R^{4b} is independently C₁-C₂ alkyl or C₁-C₂ haloalkyl.

### Embodiment 22

A compound of Embodiment 21 wherein each R^{4b} is independently C₁-C₂ alkyl or trifluoromethyl.

### Embodiment 23

A compound of Embodiment 22 wherein each R^{4b} is independently C₁-C₂ alkyl.

### Embodiment 24

A compound of any one of Embodiments 12a through 23 wherein R¹ is one of U-1 through U-50 depicted in Exhibit 1;

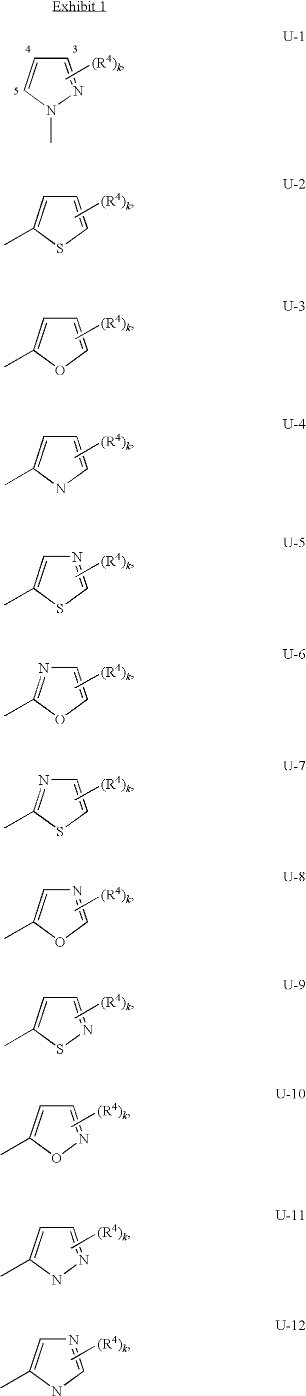

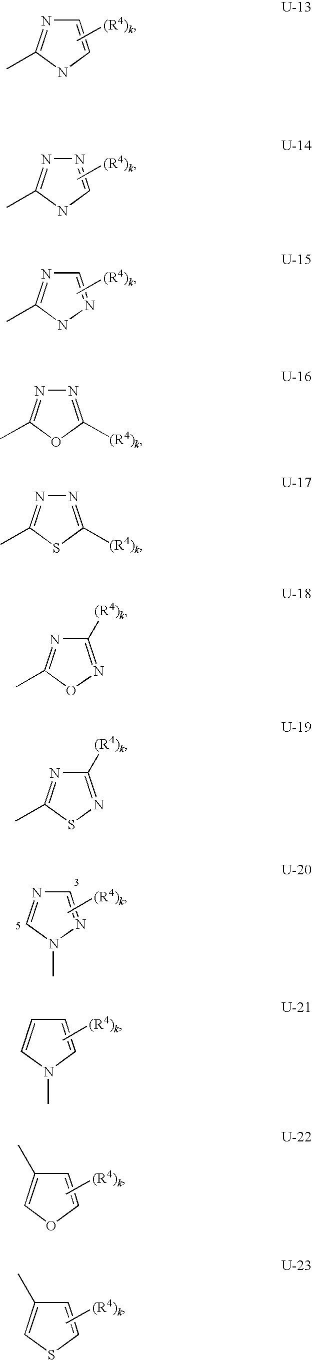

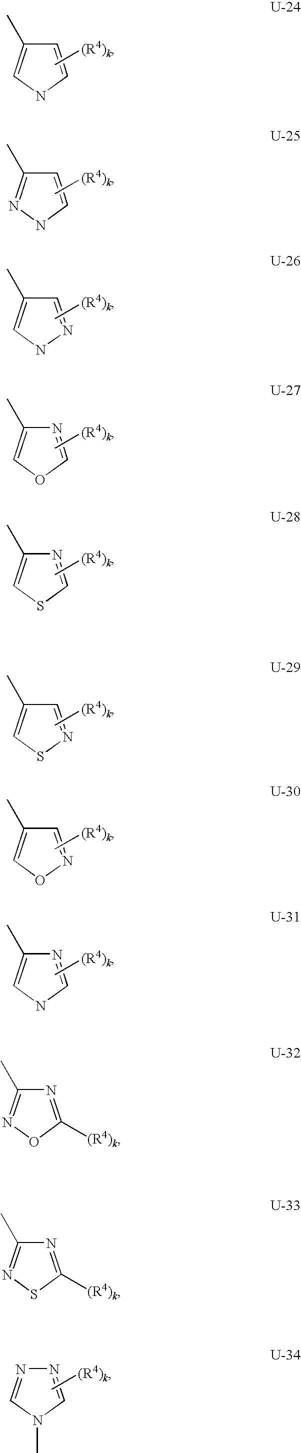

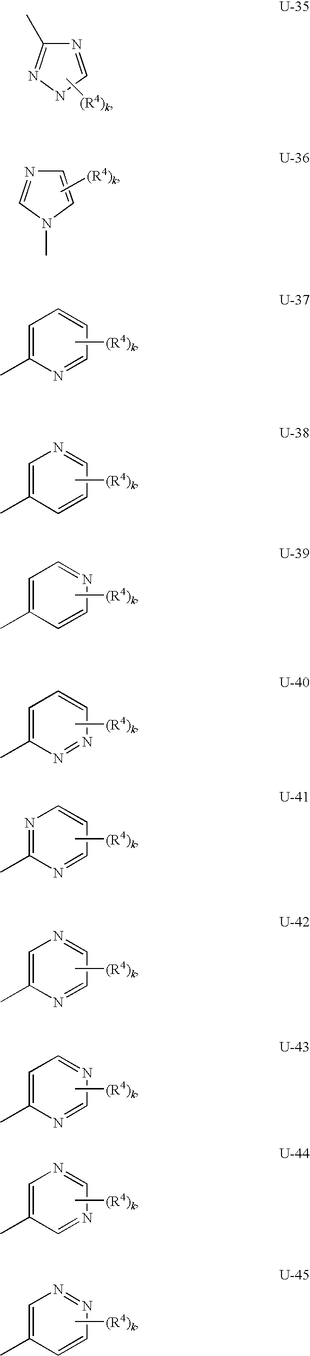

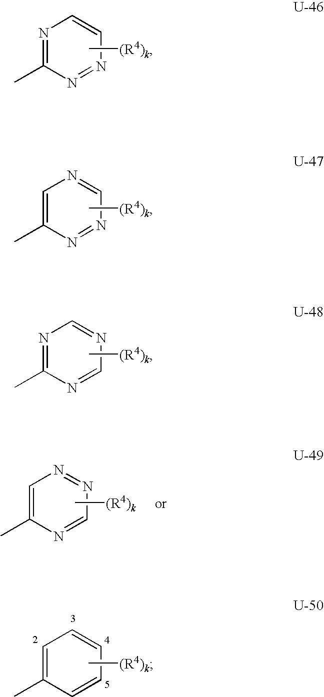

wherein
when R⁴ is attached to a carbon ring member, said R⁴ is selected from R^{4a}, and when R⁴ is attached to a nitrogen ring member (e.g., in U-4, U-11 through U-15, U-24 through U-26, U-31 or U-35), said R⁴ is selected from R^{4b}; and
k is 0, 1 or 2.

### Embodiment 24a

A compound of Embodiment 24 wherein k is 1 or 2.

### Embodiment 25

A compound of Embodiment 24 wherein k is 1 or 2 and at least one R⁴ is Cl.

### Embodiment 26

A compound of Embodiment 24 wherein k is 1 or 2 and at least one R⁴ is Br.

### Embodiment 27

A compound of Embodiment 24 wherein k is 1 or 2 and at least one R⁴ is methyl.

### Embodiment 28

A compound of Embodiment 24 wherein k is 1 or 2 and at least one R⁴ is ethyl.

### Embodiment 29

A compound of Embodiment 24 wherein k is 1 or 2 and at least one R⁴ is trifluoromethyl.

### Embodiment 30

A compound of Embodiment 24 wherein k is 1 or 2 and at least one R⁴ is methoxy.

### Embodiment 31

A compound of any one of Embodiments 24 through 30 wherein R¹ is selected from U-1 through U-5, U-8, U-11, U-13, U-15, U-20 through U-28, U-31, U-36 through U-39 and U-50.

### Embodiment 32

A compound of Embodiment 31 wherein R¹ is selected from U-1 through U-3, U-5, U-8, U-11, U-13, U-20, U-22, U-23, U-25 through U-28, U-36 through U-39 and U-50.

### Embodiment 33

A compound of Embodiment 32 wherein R¹ is selected from U-1 through U-3, U-11, U-13, U-20, U-22, U-23, U-36 through U-39 and U-50.

### Embodiment 34

A compound of Embodiment 33 wherein R¹ is U-1, U-20 or U-50.

### Embodiment 35

A compound of Embodiment 34 wherein R¹ is U-1.

### Embodiment 35a

A compound of Embodiment 34 wherein R¹ is U-20.

### Embodiment 36

A compound of Embodiment 34 wherein R¹ is U-50.

### Embodiment 37

A compound of Embodiment 35 wherein k is 1 and R⁴ is connected to the 3- or 5-position of U-1.

### Embodiment 37a

A compound of Embodiment 35 wherein k is 2 and one R⁴ is connected to the 3-position and the other R⁴ is connected to the 5-position of U-1.

### Embodiment 38

A compound of Embodiment 35a wherein k is 1 and R⁴ is connected to the 3- or 5-position of U-20.

### Embodiment 38a

A compound of Embodiment 35a wherein k is 2 and one R⁴ is connected to the 3-position and the other R⁴ is connected to the 5-position of U-20.

### Embodiment 39

A compound of Embodiment 36 wherein k is 1 and R⁴ is connected to the 2- or 5-position of U-50.

### Embodiment 40

A compound of Embodiment 36 wherein k is 2 and one R⁴ is connected to the 2-position and the other R⁴ is connected to the 5-position of U-50.

### Embodiment 41

A compound of Formula 1 or any one of Embodiments 1 through 40 wherein G is a 5-membered heterocyclic ring optionally substituted with up to 2 substituents selected from R³ on carbon ring members and selected from R¹¹ on nitrogen ring members;
each R³ is independently C₁-C₃ alkyl, C₁-C₃ haloalkyl or halogen; and
each R¹¹ is independently C₁-C₃ alkyl.

### Embodiment 41a

A compound of Embodiment 41 wherein each R³ is independently C₁-C₃ alkyl or halogen.

### Embodiment 41b

A compound of Embodiment 41a wherein each R³ is independently methyl or halogen.

### Embodiment 41c

A compound of Embodiment 41b wherein each R³ is methyl.

### Embodiment 42

A compound of any one of Embodiments 41 through 41c wherein G is one of G-1 through G-59 depicted in Exhibit 2;

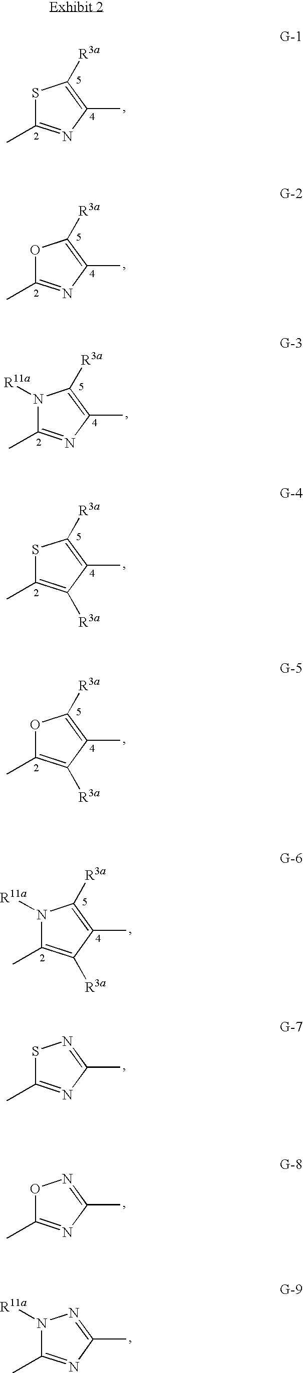

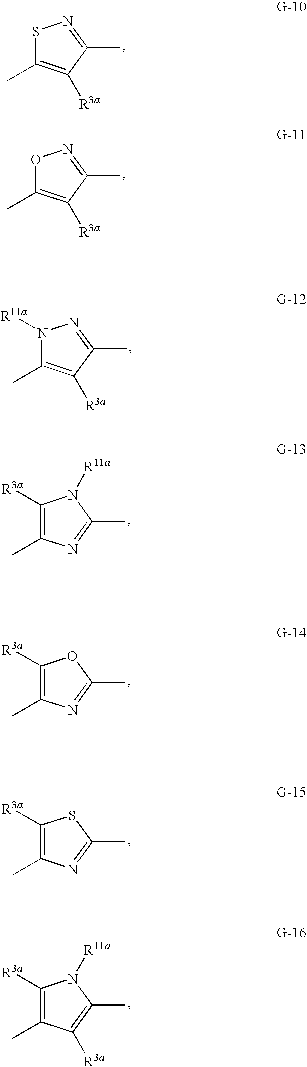

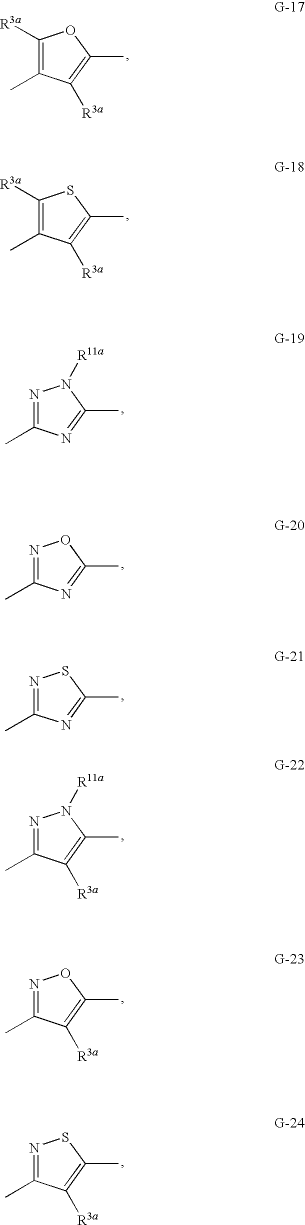

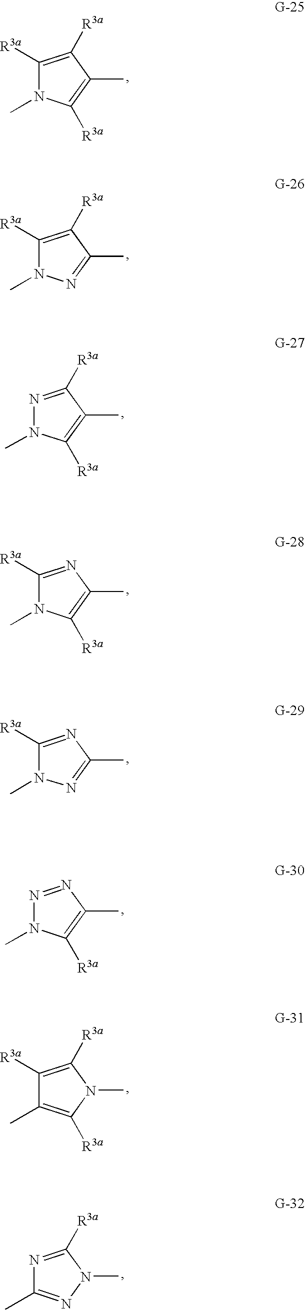

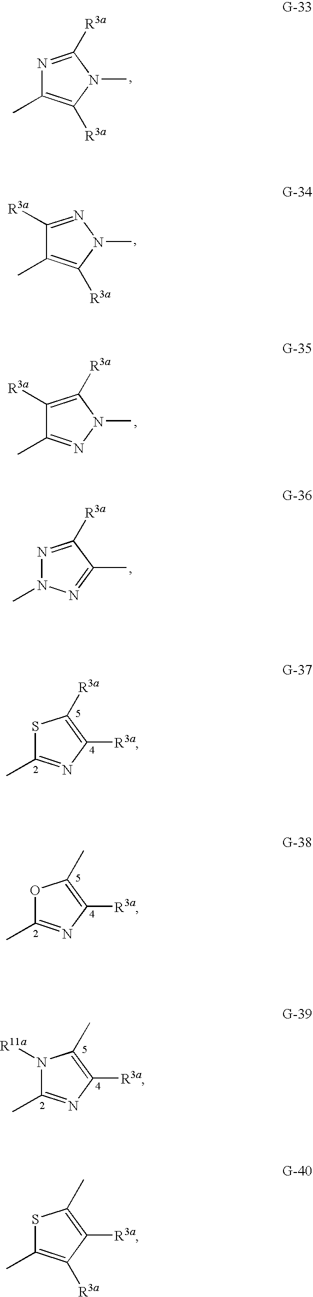

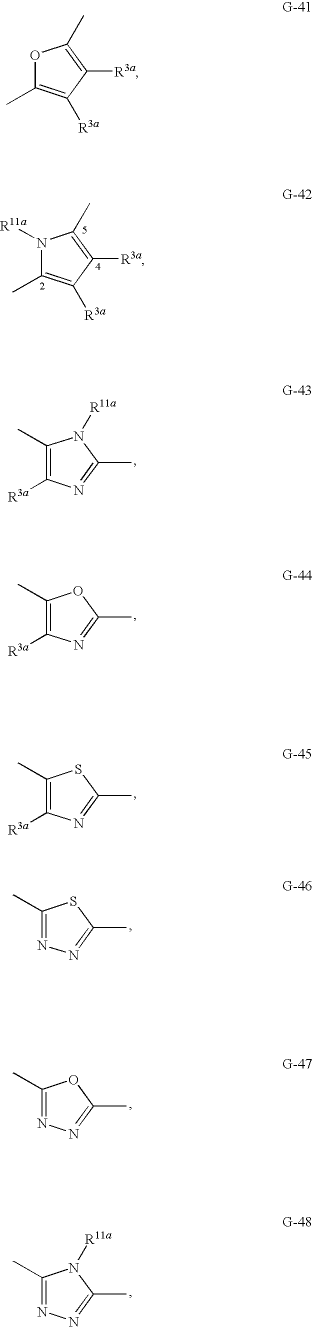

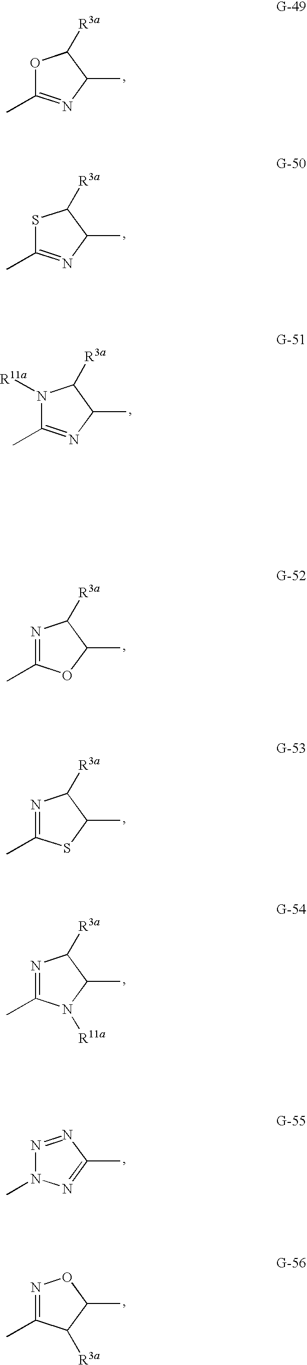

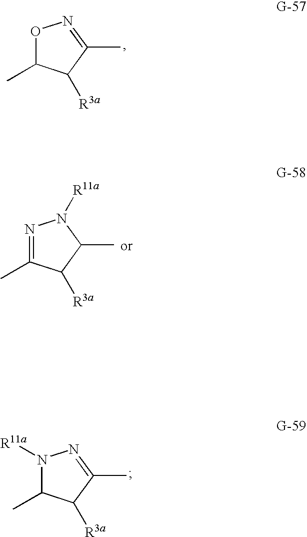

wherein the bond projecting to the left is bonded to X, and the bond projecting to the right is bonded to Z¹; each R^{3a} is independently selected from H or R³; and R^{11a} is selected from H and R¹¹.

### Embodiment 43

A compound of Embodiment 42 wherein G is selected from G-1 through G-3, G-7, G-8, G-10, G-11, G-14, G-15, G-23, G-24, G-26 through G-28, G-30, G-36 through G-38 and G-49 through G-55.

### Embodiment 44

A compound of Embodiment 43 wherein G is selected from G-1, G-2, G-7, G-8, G-14, G-15, G-23, G-24, G-26, G-27, G-36, G-37, G-38, G-49, G-50 and G-55.

### Embodiment 45

A compound of Embodiment 44 wherein G is selected from G-1, G-2, G-15, G-26, G-27, G-36, G-37 and G-38.

### Embodiment 46

A compound of Embodiment 45 wherein G is selected from G-1, G-2, G-15, G-26 and G-36.

### Embodiment 47

A compound of Embodiment 46 wherein G is G-1. Of note are embodiments of these compounds within Embodiments 1 through 40, Embodiments 52 through 83, and Embodiments A1 through A5.

### Embodiment 48

A compound of Embodiment 46 wherein G is G-2. Of note are embodiments of these compounds within Embodiments 1 through 40, Embodiments 52 through 83, and Embodiments A1 through A5.

### Embodiment 49

A compound of Embodiment 46 wherein G is G-15. Of note are embodiments of these compounds within Embodiments 1 through 40, Embodiments 52 through 83, and Embodiments A1 through A5.

### Embodiment 50

A compound of Embodiment 46 wherein G is G-26. Of note are embodiments of these compounds within Embodiments 1 through 40, Embodiments 52 through 83, and Embodiments A1 through A5.

### Embodiment 51

A compound of Embodiment 46 wherein G is G-36. Of note are embodiments of these compounds within Embodiments 1 through 40, Embodiments 52 through 83, and Embodiments A1 through A5.

### Embodiment 52

A compound of any one of Embodiments 42 through 51 wherein each R^{3a} is independently H, C₁-C₃ alkyl or halogen.

### Embodiment 53

A compound of Embodiment 52 wherein each R^{3a} is independently H or methyl.

### Embodiment 54

A compound of any one of Embodiments 42 through 51 wherein each R^{3a} is H and each R^{11a} is independently H or methyl.

### Embodiment 55

A compound of Formula 1 or any one of Embodiments 41 through 51 wherein G is unsubstituted.

### Embodiment 56

A compound of Formula 1 or any one of Embodiments 1 through 55 wherein each R⁵ is independently H, cyano, C₁-C₆ alkyl, C₁-C₆ haloalkyl, C₃-C₈ cycloalkyl, C₃-C₈ halocycloalkyl, C₂-C₆ alkoxyalkyl, C₁-C₆ alkoxy, C₁-C₆ haloalkoxy, C₃-C₈ cycloalkoxy, C₂-C₆ alkenyloxy, C₂-C₆ haloalkenyloxy, C₂-C₆ alkynyloxy, C₂-C₆ alkoxyalkoxy, C₂-C₆ alkylcarbonyloxy, C₂-C₆ haloalkylcarbonyloxy, C₁-C₆ alkylthio, C₁-C₆ haloalkylthio, C₃-C₁₀ trialkylsilyl, —NR²⁵R²⁶ or halogen.

### Embodiment 57

A compound of Embodiment 56 wherein each R⁵ is independently H, cyano, C₁-C₆ alkyl, C₁-C₆ haloalkyl, C₁-C₆ alkoxy, C₁-C₆ haloalkoxy, —NR²⁵R²⁶ or halogen.

### Embodiment 57a

A compound of Embodiments 56 or 57 wherein R⁵ is other than halogen.

### Embodiment 58

A compound of Embodiment 57 wherein each R⁵ is independently H, cyano, C₁-C₄ alkyl, C₁-C₄ haloalkyl, C₁-C₄ alkylcarbonyl or halogen.

### Embodiment 59

A compound of Embodiment 58 wherein each R⁵ is independently H and C₁-C₃ alkyl.

### Embodiment 60

A compound of Formula 1 or any one of Embodiments 1 through 59 wherein J is one of J-1 through J-82 depicted in Exhibit 3;

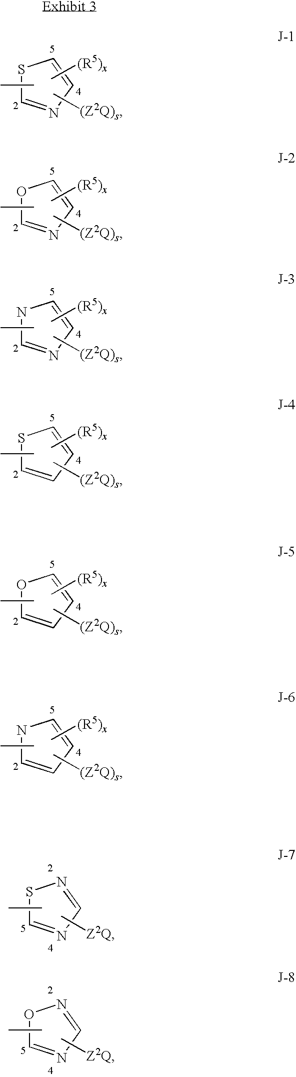

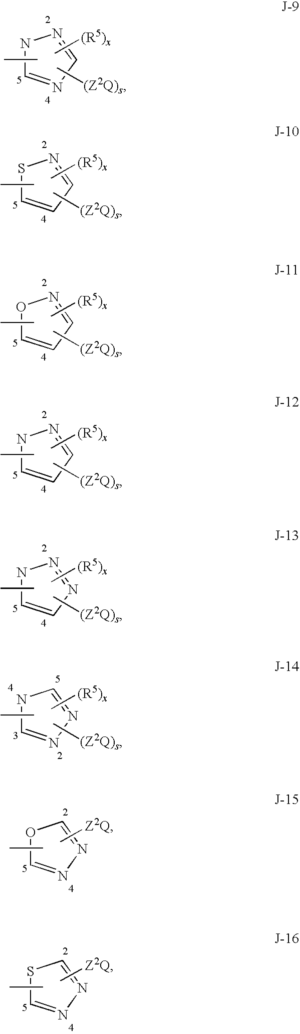

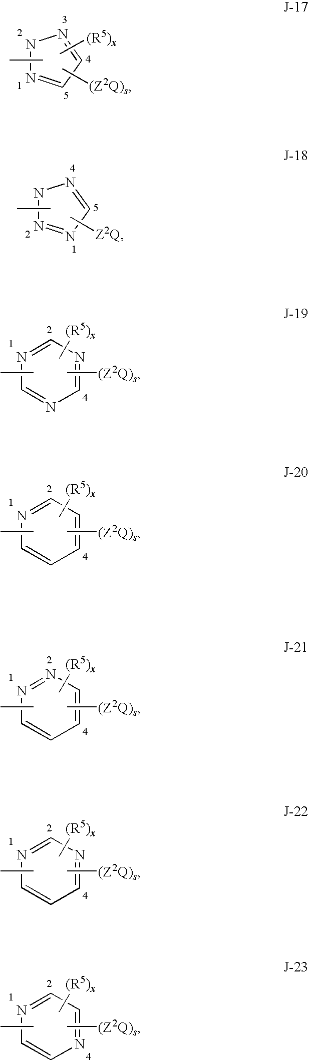

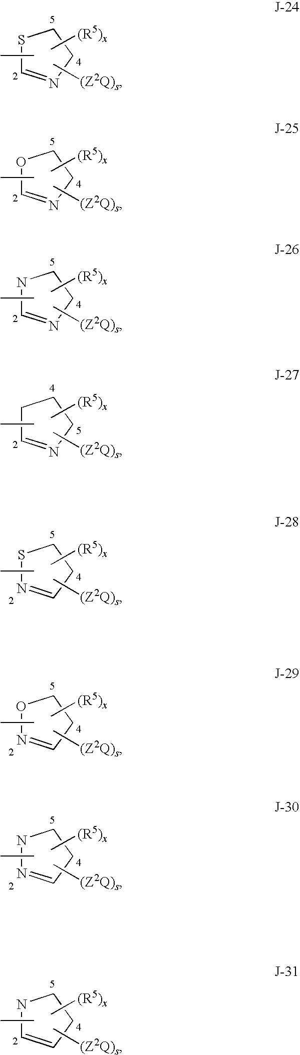

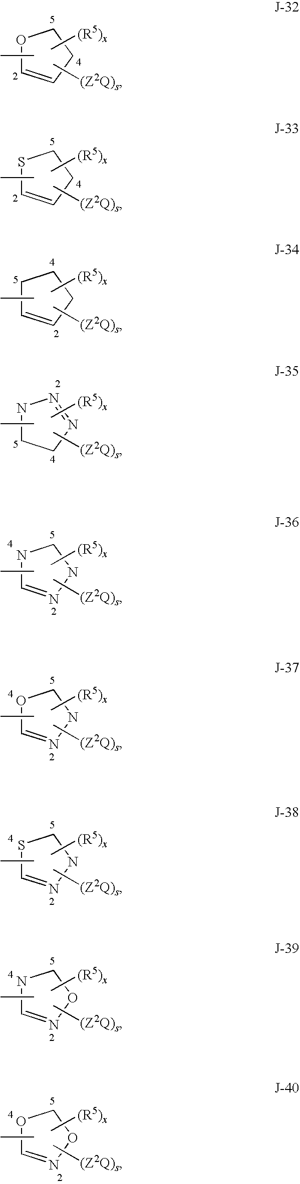

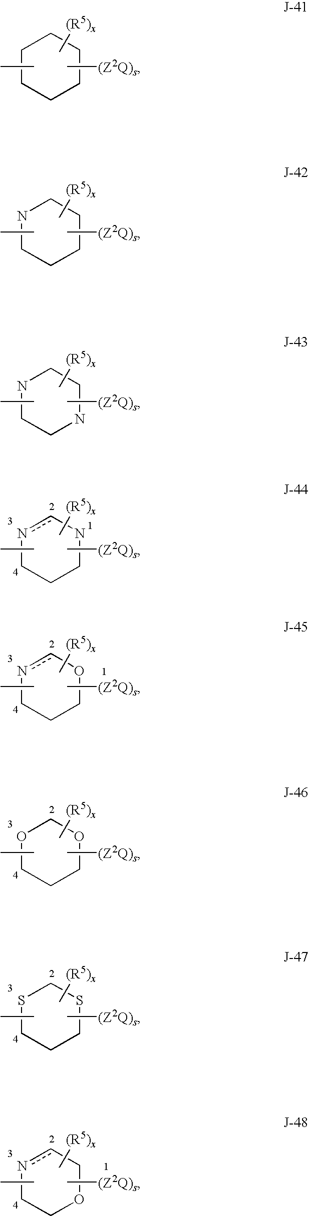

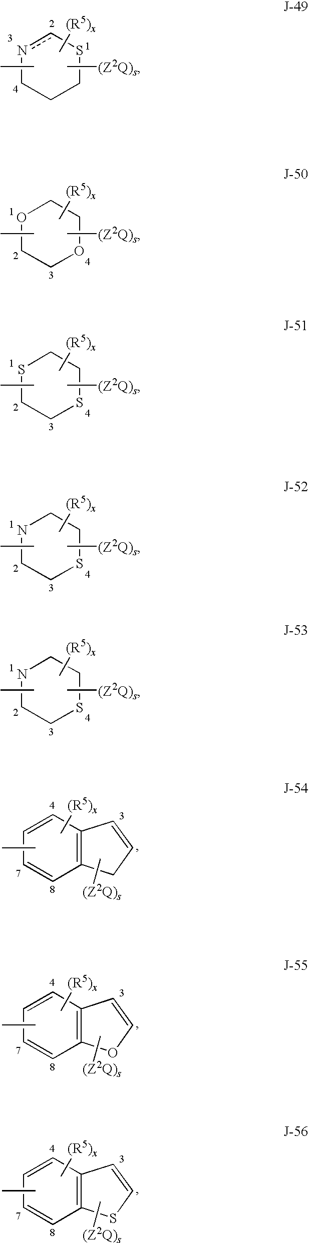

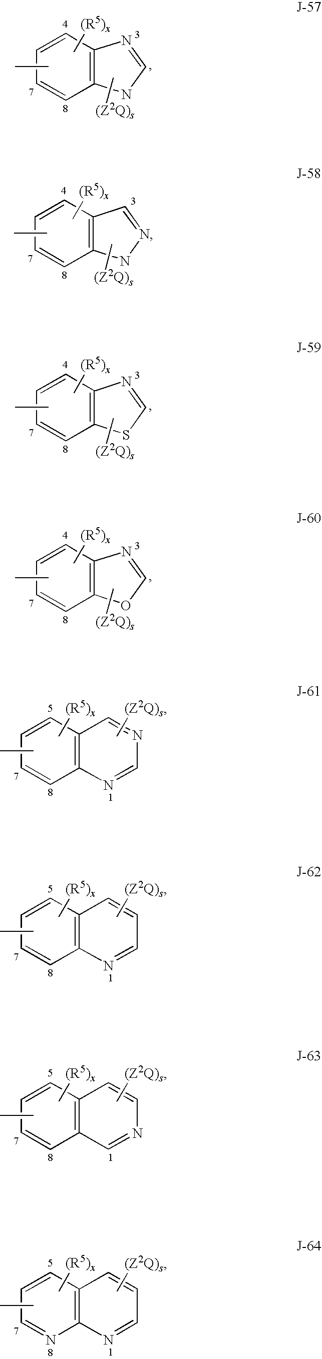

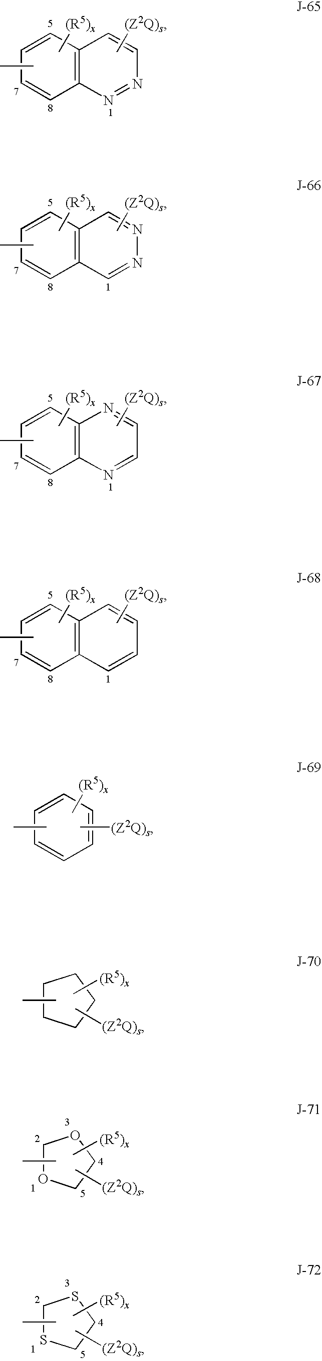

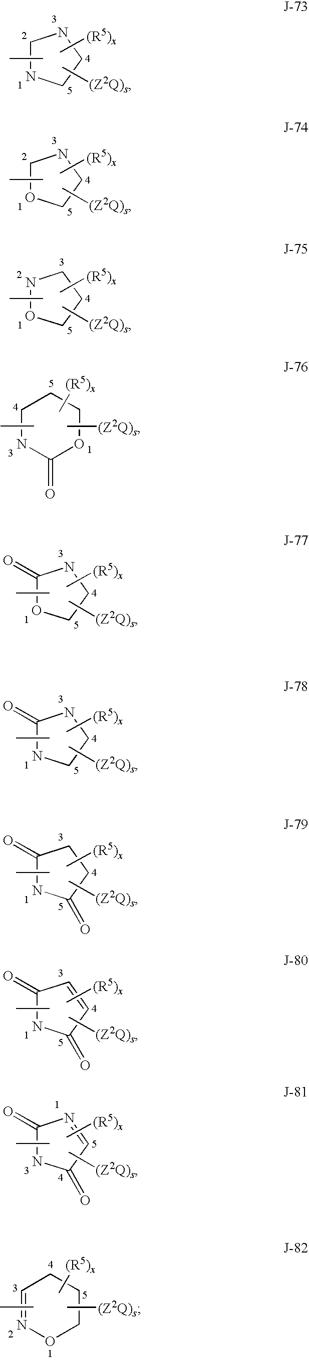

wherein the bond shown projecting to the left is bonded to Z¹; x is an integer from 0 to 5; and s is an integer from 1 to 2.

### Embodiment 61

A compound of Embodiment 60 wherein x is 0 or 1.

### Embodiment 61a

A compound of Embodiment 61 wherein x is 0.

### Embodiment 62

A compound of Embodiment 61a wherein s is 1 or 2.

### Embodiment 63

A compound of Embodiment 62 wherein s is 1.

### Embodiment 64

A compound of any one of Embodiments 60 through 63 wherein J is selected from J-1, J-2, J-3, J-4, J-5, J-7, J-8, J-9, J-10, J-11, J-12, J-14, J-15, J-16, J-20, J-24, J-25, J-26, J-29, J-30, J-37, J-38, J-45 and J-69.

### Embodiment 65

A compound of Embodiment 64 wherein J is selected from J-4, J-5, J-8, J-11, J-15, J-16, J-20, J-29, J-30, J-37, J-38, and J-69.

### Embodiment 66

A compound of Embodiment 65 wherein J is selected from J-4, J-5, J-11, J-20, J-29, J-37, J-38, and J-69.

### Embodiment 67

A compound of Embodiment 66 wherein J is J-11.

### Embodiment 68

A compound of Embodiment 66 wherein J is J-29.

### Embodiment 69

A compound of Embodiment 59 wherein J is J-69.

### Embodiment 70

A compound of Embodiment 67 wherein the 3-position of J-11 is connected to Z¹ and the 5-position of J-11 is connected to Z²Q.

### Embodiment 71

A compound of Embodiment 68 wherein the 3-position of J-29 is connected to Z¹ and the 5-position of J-29 is connected to Z²Q.

### Embodiment 72

A compound of Formula 1 or any one of Embodiments 1-through 71 wherein the ring or ring system of J directly connected to Z¹ is substituted with one —Z²Q.

### Embodiment 72a

A compound of Embodiment 68 wherein J is one of J-29-1 through J-29-60 depicted in Exhibit A;

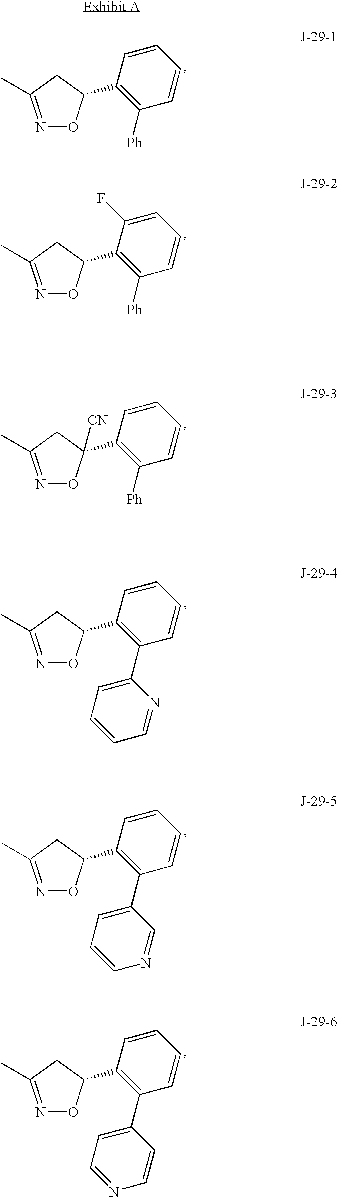

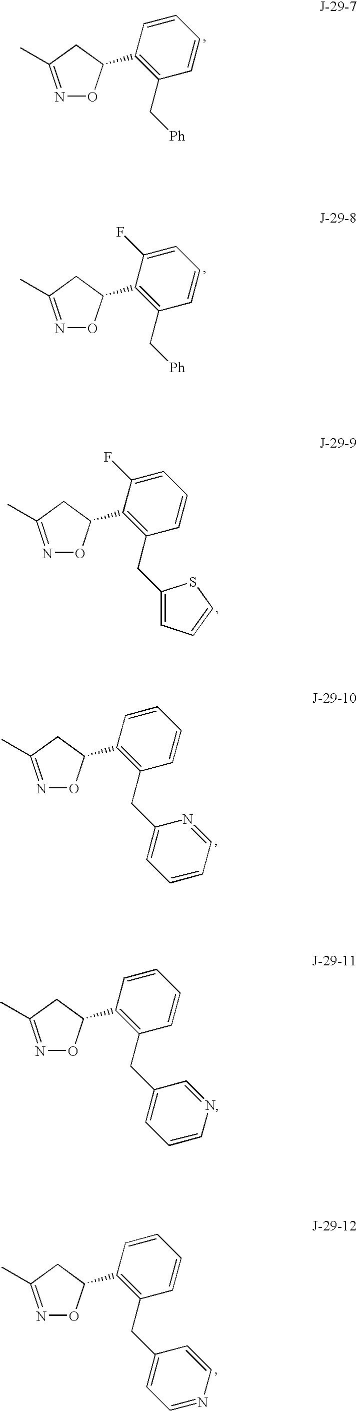

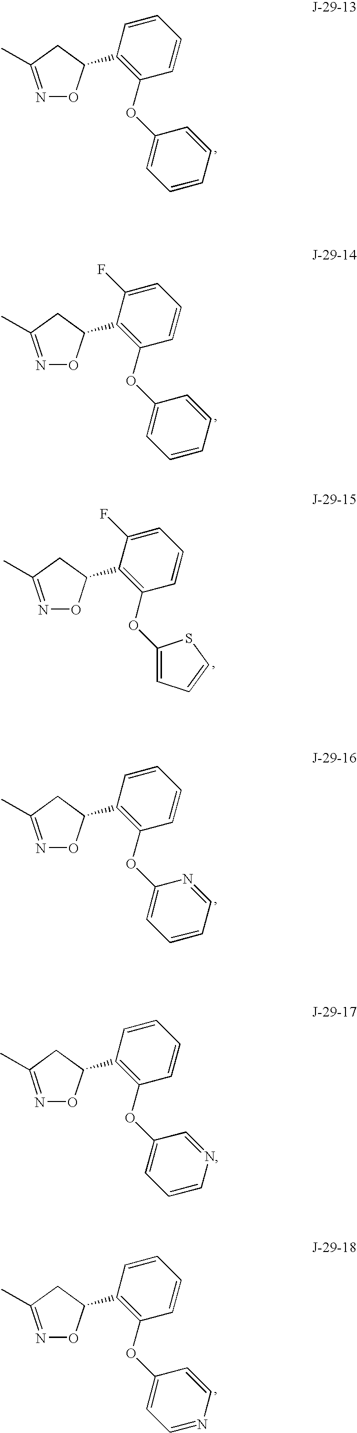

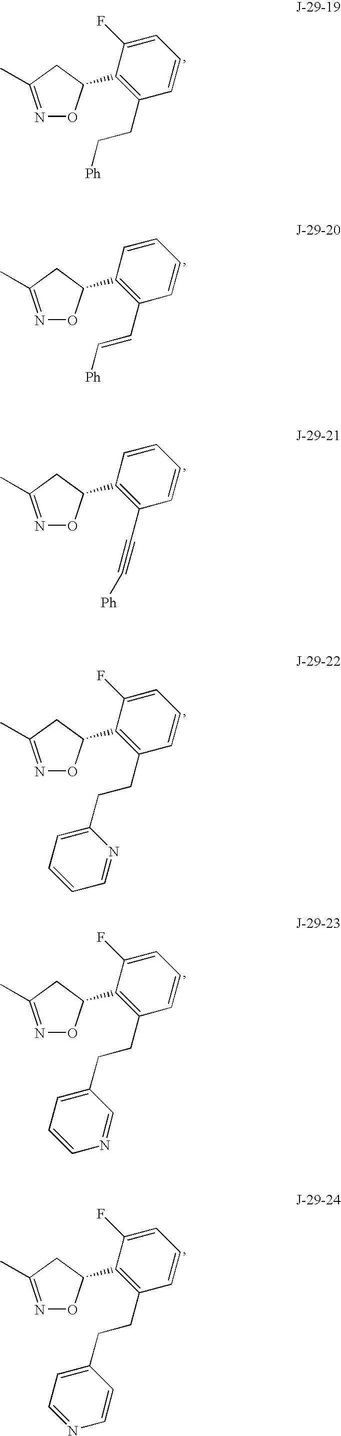

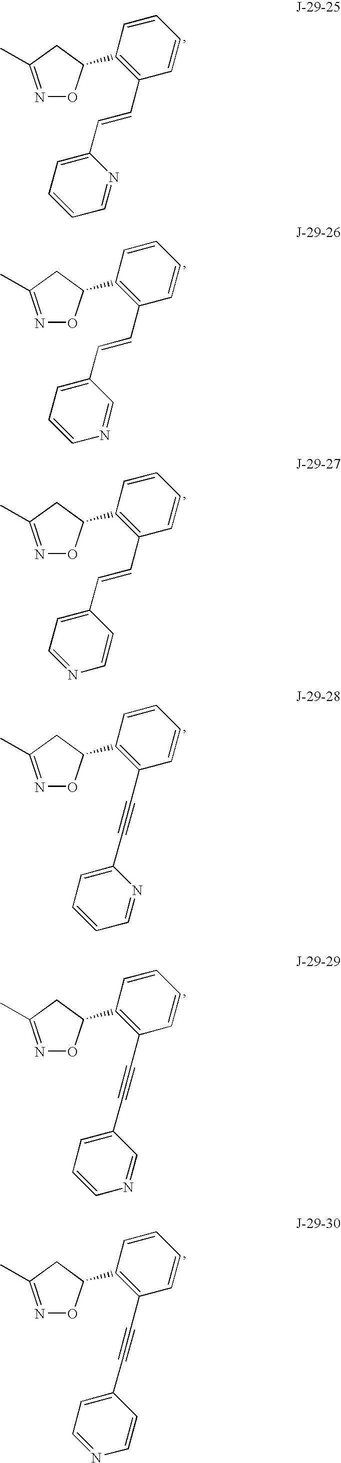

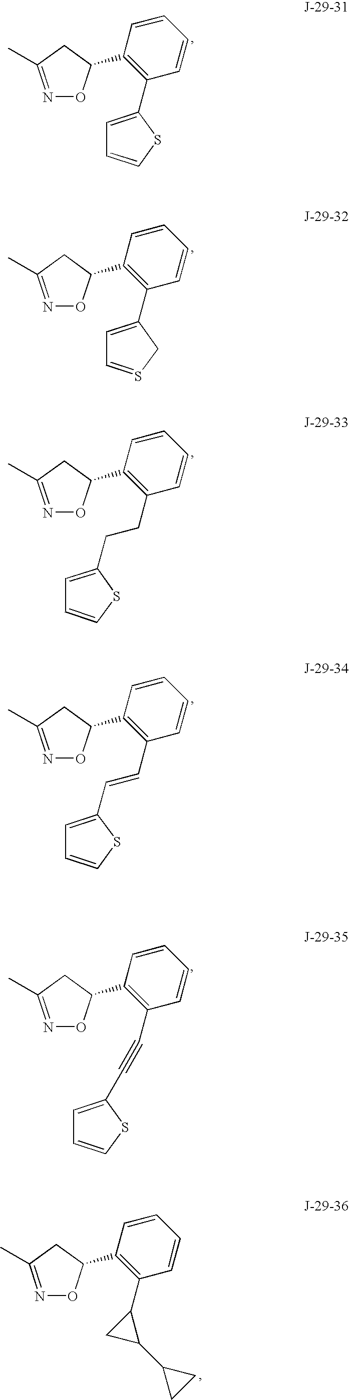

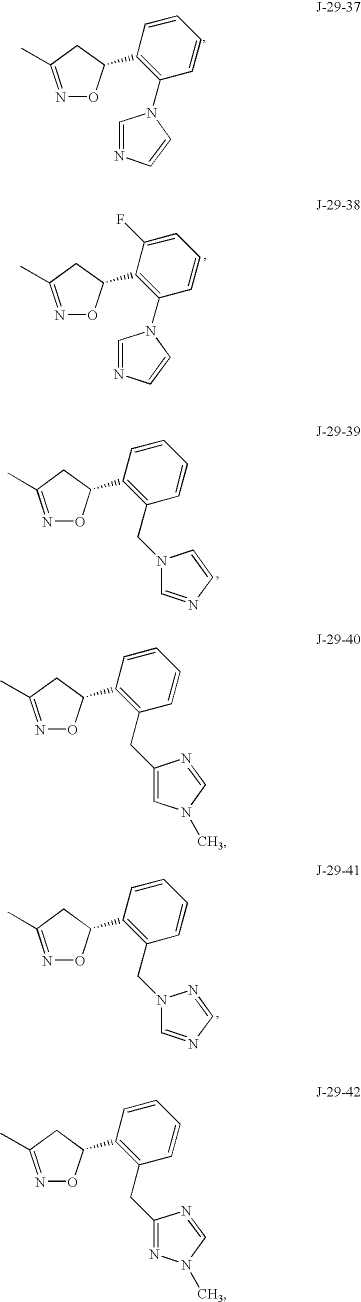

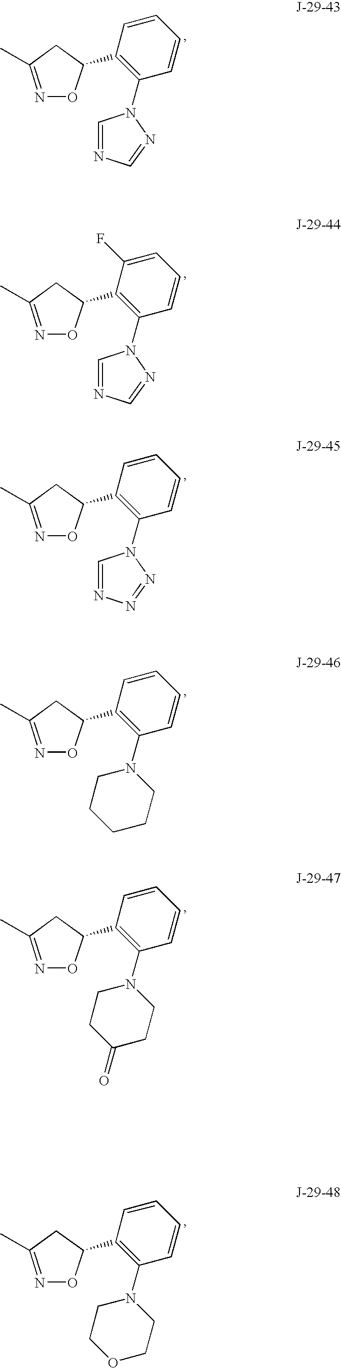

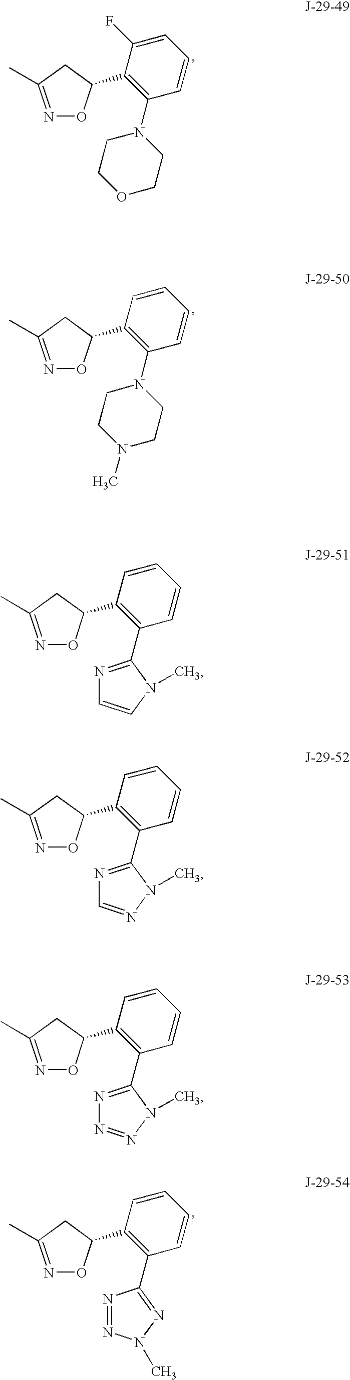

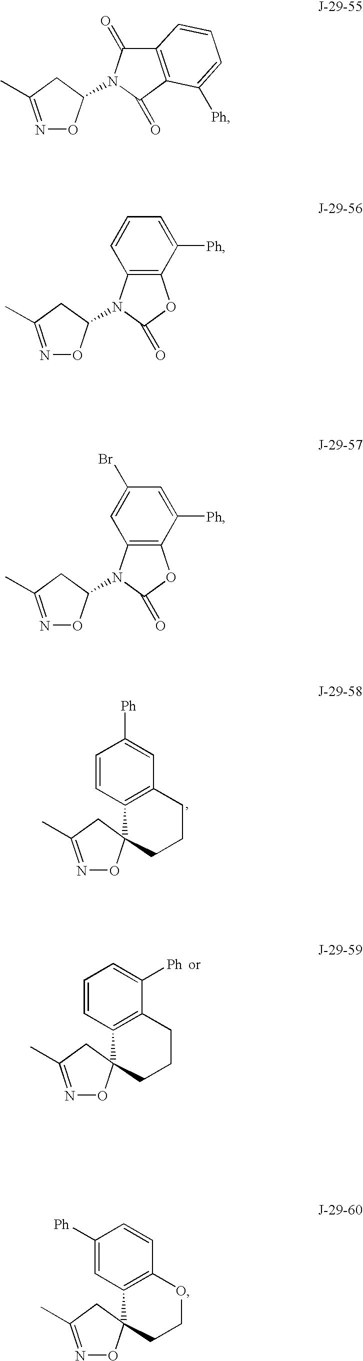

wherein Ph is phenyl, and the bond shown projecting to the left is bonded to Z¹ in Formula 1.

### Embodiment 72b

A compound of Embodiment 72a wherein J is one of J-29-1 through J-29-57.

### Embodiment 73

A compound of Formula 1 or any one of Embodiments 1 through 72b wherein Z¹ is a direct bond, O, C(═O), S(O)ₘ, CHR²⁰ or NR²¹.

### Embodiment 73a

A compound of Embodiment 73 wherein Z¹ is a direct bond.

### Embodiment 74

A compound of Formula 1 or any one of Embodiments 1 through 73a wherein Z² is a direct bond, O, C(═O), S(O)ₘ, CHR²⁰ or NR²¹.

### Embodiment 74a

A compound of Embodiment 74 wherein Z² is a direct bond or NR²¹.

### Embodiment 74b

A compound of Embodiment 74a wherein Z² is a direct bond.

### Embodiment 75

A compound of Formula 1 or any one of Embodiments 1 through 74b wherein Q is one of Q-1 through Q-106 depicted in Exhibit 4;

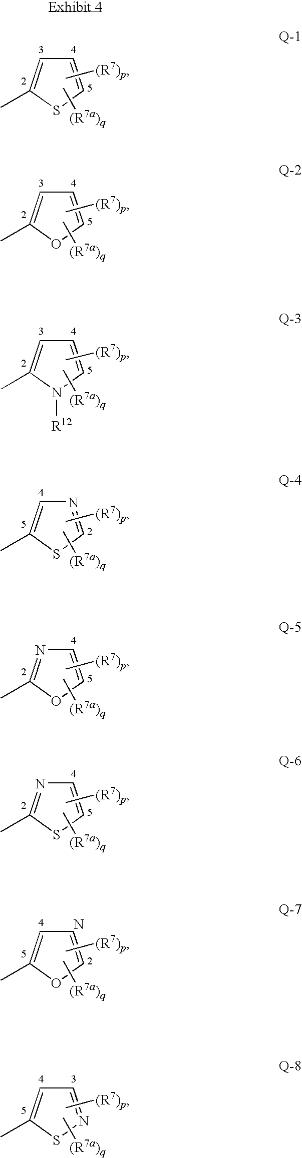

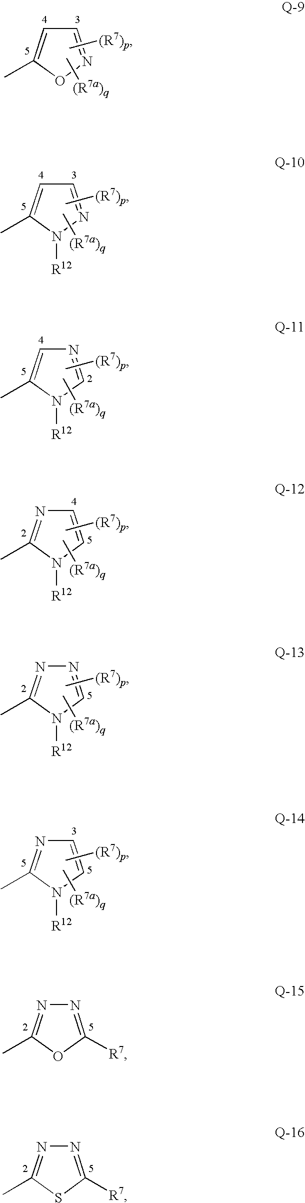

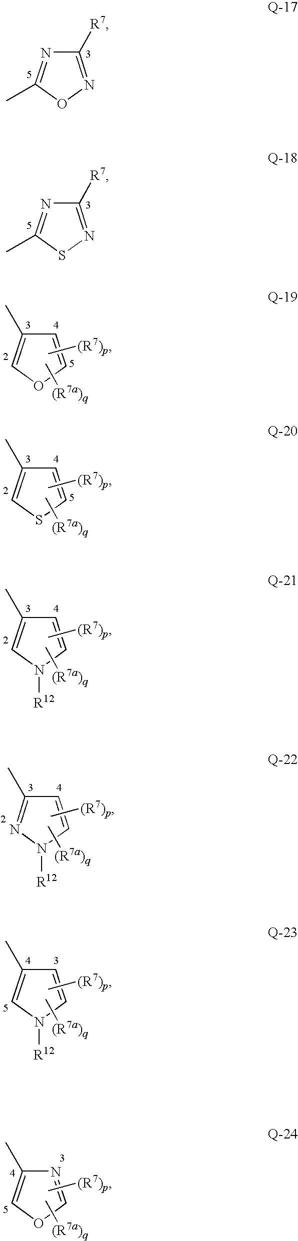

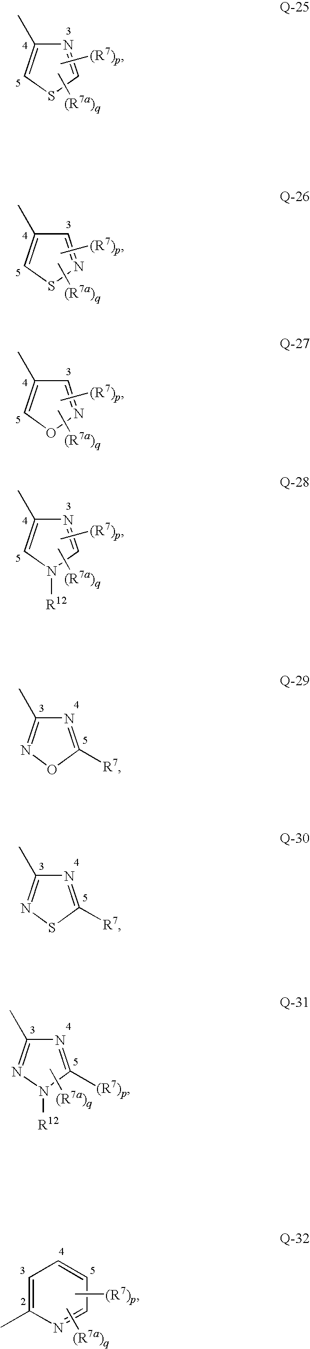

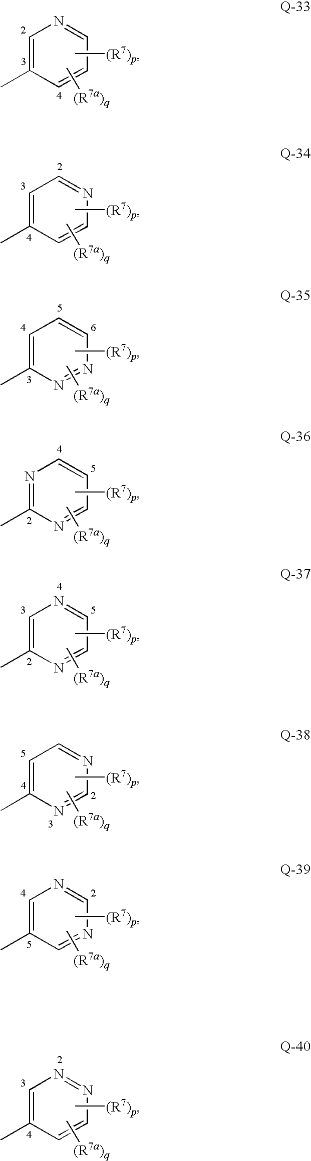

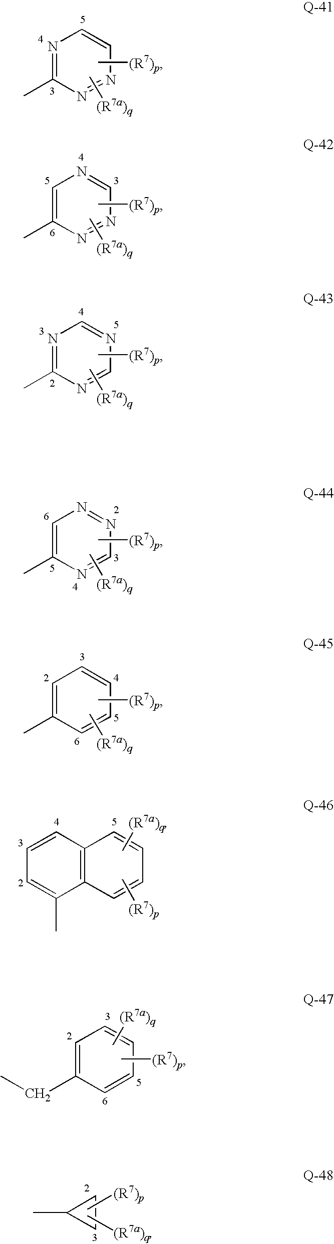

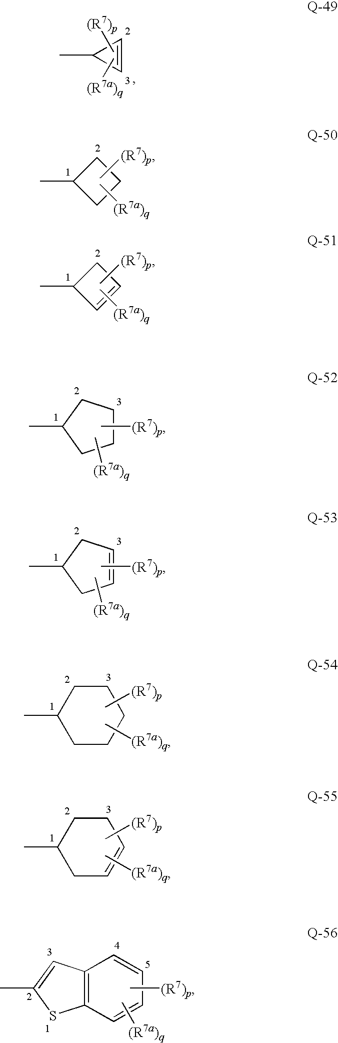

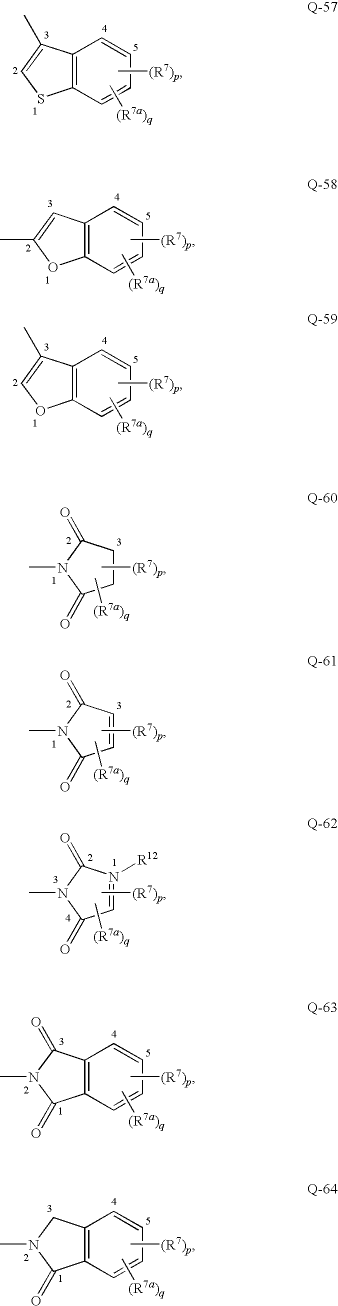

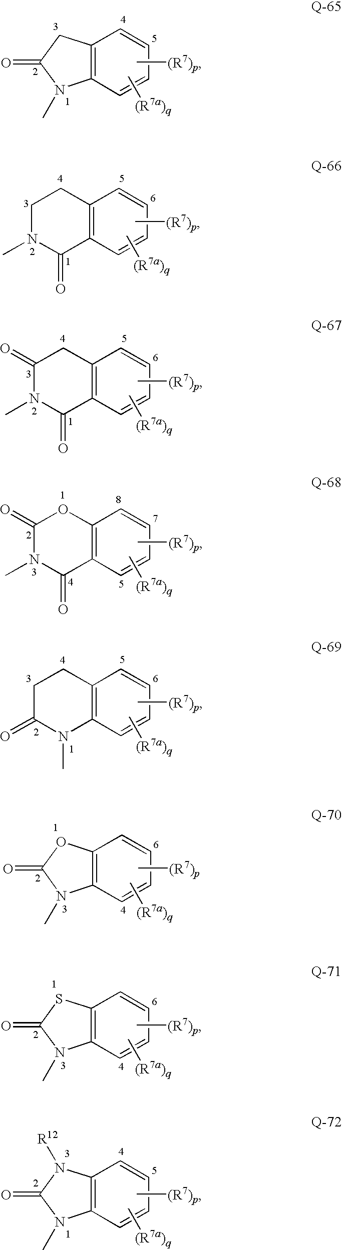

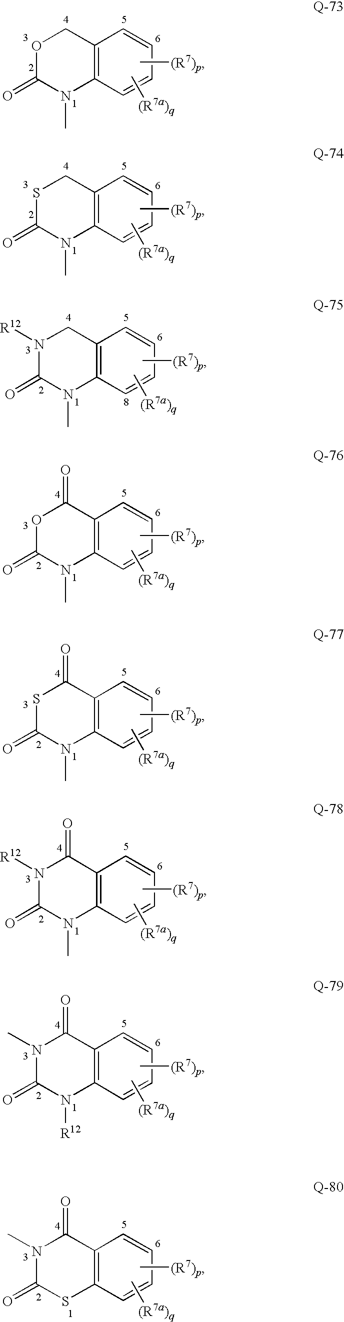

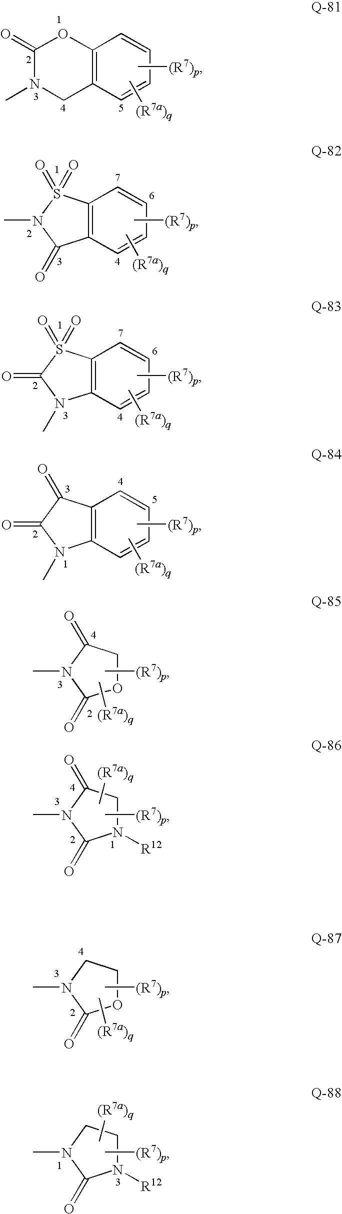

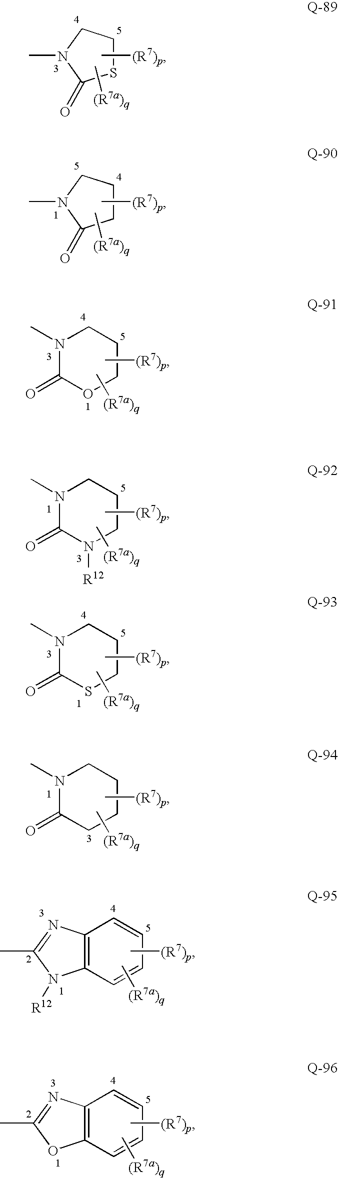

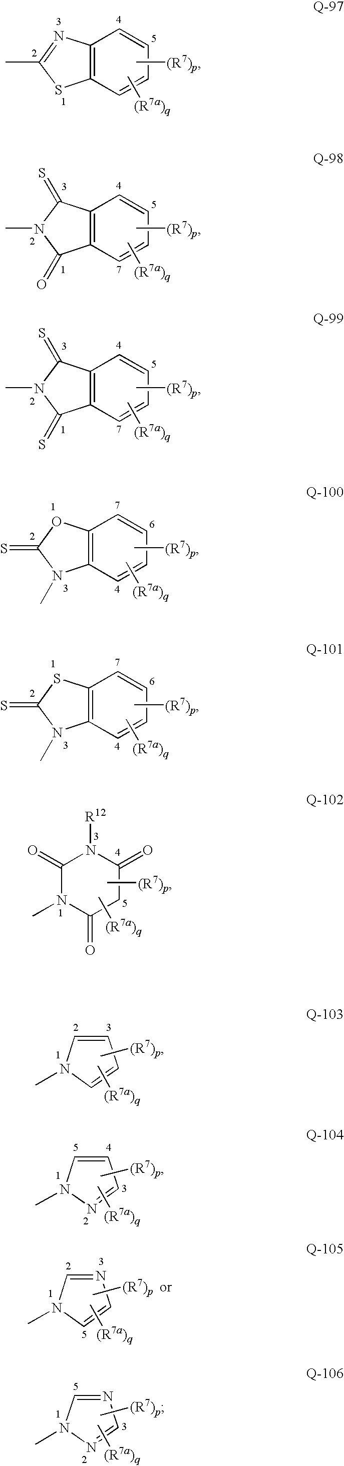

wherein the bond shown projecting to the left is bonded to Z²; R¹² attached to a nitrogen ring member is optionally replaced by R⁷ (e.g., Q-3, Q-10 through Q-14, Q-21 through Q-23, Q-28, Q-31, Q-62, Q-75, Q-78, Q-79, Q-86, Q-88, Q-92 or Q-95); p is 1 or 2; and q is 0, 1, 2, 3, 4 or 5.

### Embodiment 76

A compound of Embodiment 75 wherein Q is selected from Q-1, Q-20, Q-32 through Q-34, Q-45 through Q-47, Q-60 through Q-73, Q-76 through Q-79, Q-84 through Q-94 and Q-98 through Q-106.

### Embodiment 77

A compound of Embodiment 76 wherein Q is Q-1, Q-45, Q-62, Q-63, Q-64, Q-65, Q-68, Q-69, Q-70, Q-71, Q-72, Q-73, Q-76, Q-78, Q-79, Q-84, Q-85, Q-98, Q-99, Q-100, Q-101 through Q-106.

### Embodiment 78

A compound of Embodiment 77 wherein Q is Q-45, Q-62, Q-63, Q-64, Q-65, Q-68, Q-69, Q-70, Q-71, Q-72, Q-85 or Q-104.

### Embodiment 79

A compound of Embodiment 78 wherein Q is Q-45, Q-62, Q-63, Q-65, Q-70, Q-71, Q-72, Q-85 or Q-104.

### Embodiment 80

A compound of Embodiment 79 wherein Q is Q-45, Q-62, Q-63, Q-65, Q-70 or Q-104.

### Embodiment 80a

A compound of any one of Embodiments 77 through 80 wherein Q is other than Q-62 or Q-104.

### Embodiment 80b

A compound of Embodiment 80 wherein Q is Q-45.

### Embodiment 80c

A compound of Embodiment 80 wherein Q is Q-62.

### Embodiment 80d

A compound of Embodiment 80 wherein Q is Q-104.

### Embodiment 81

A compound of Formula 1 or any one of Embodiments 1 through 74b wherein each Q is independently phenyl, benzyl, naphthalenyl, a 5- or 6-membered heteroaromatic ring or an 8- to 11-membered heteroaromatic bicyclic ring system, each ring or ring system substituted with 1 substituent selected from R⁷ on carbon or nitrogen atom ring members.

### Embodiment 82

A compound of Embodiment 81 wherein Q is phenyl substituted with one R⁷.

### Embodiment 83

A compound of Embodiment 81 wherein Q is benzyl substituted with one R⁷.

### Embodiment 84

A compound of Embodiment 81 wherein Q is an 8- to 11-membered heteroaromatic bicyclic ring system substituted with one R⁷.

### Embodiment 85

A compound of Formula 1 or any one of Embodiments 1 through 84 wherein each Z³ is independently a direct bond, O, NR²², C(═O), C(═S), S(O)ₘ, CHR²⁰, CHR²⁰—CHR²⁰, CR²⁴═CR²⁷, C≡C or OCHR²⁰.

### Embodiment 85a

A compound of Embodiment 85 wherein each Z³ is a C(═O).

### Embodiment 86

A compound of Embodiment 85 wherein each Z³ is independently a direct bond, O, NR²², S(O)ₘ, CHR²⁰, CHR²⁰—CHR²⁰, CR²⁴═CR²⁷, C≡C or OCHR²⁰.

### Embodiment 87

A compound of Embodiment 86 wherein each Z³ is independently a direct bond, O, NR²², S(O)ₘ, CHR²⁰, CHR²⁰—CHR²⁰, CR²⁴═CR²⁷ or C≡C.

### Embodiment 88

A compound of Embodiment 87 wherein each Z³ is independently a direct bond, O, NR²², CHR²⁰ or CHR²⁰—CHR²⁰.

### Embodiment 88a

A compound of Embodiment 88 wherein each Z³ is CH₂.

### Embodiment 89

A compound of Embodiment 88 wherein each Z³ is independently a direct bond, O or NR²².

### Embodiment 90

A compound of Embodiment 89 wherein each Z³ is a direct bond.

### Embodiment 91

A compound of Embodiment 89 wherein each Z³ is O.

### Embodiment 92

A compound of Formula 1 or any one of Embodiments 1 through 91 wherein R⁷ is —Z³G^{A}.

### Embodiment 93

A compound of Embodiment 92 wherein G^{A} is phenyl.

### Embodiment 94

A compound of Embodiment 92 wherein G^{A} is a 5- or 6-membered heteroaromatic ring.

### Embodiment 95

A compound of Formula 1 or any one of Embodiments 1 through 91 wherein R⁷ is —Z³G^{N}.

### Embodiment 96

A compound of Formula 1 any one of Embodiments 1 through 91 wherein R⁷ is —Z³G^{P}.

### Embodiment 97

A compound of Formula 1 or any one of Embodiments 1 through 96 wherein each G^{A} is independently one of G^{A}-1 through G^{A}-49, each G^{N} is independently one of G^{N}-1 through G^{N}-32, and each G^{P} is independently one of G^{P}-1 through G^{P}-35 respectively, as depicted in Exhibit 5.

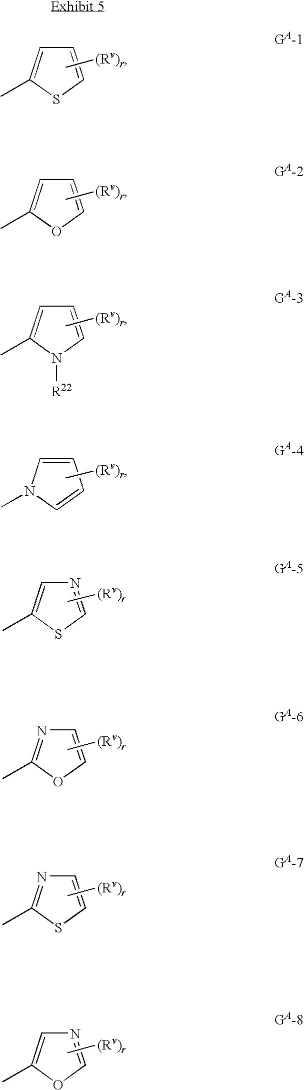

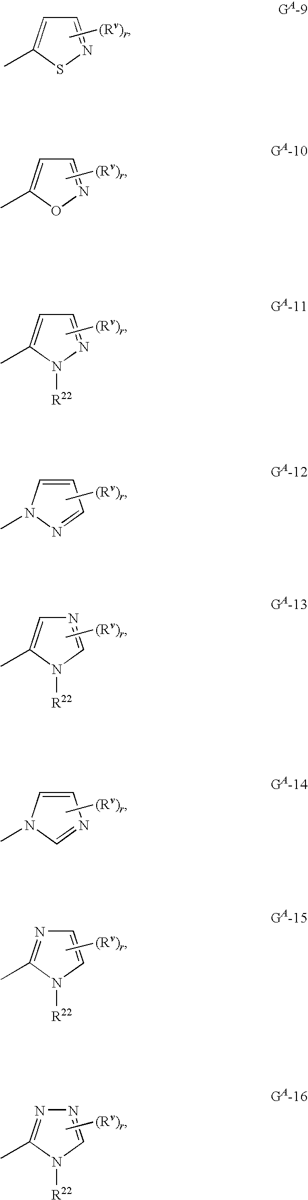

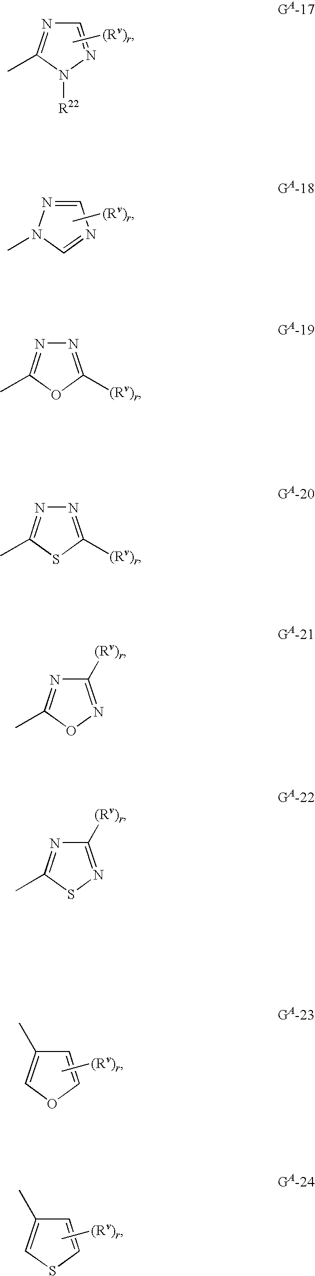

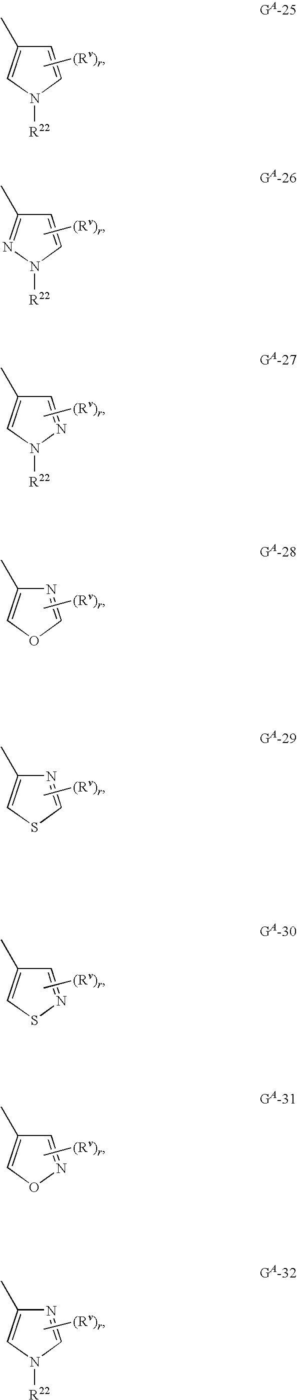

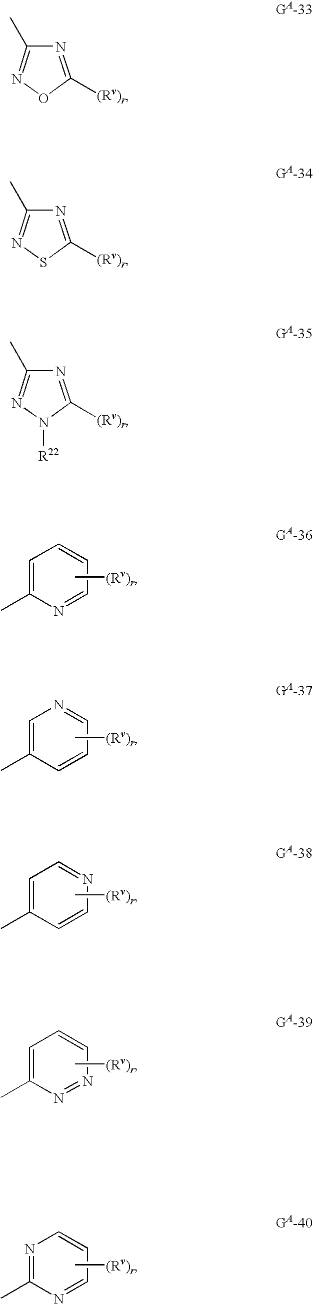

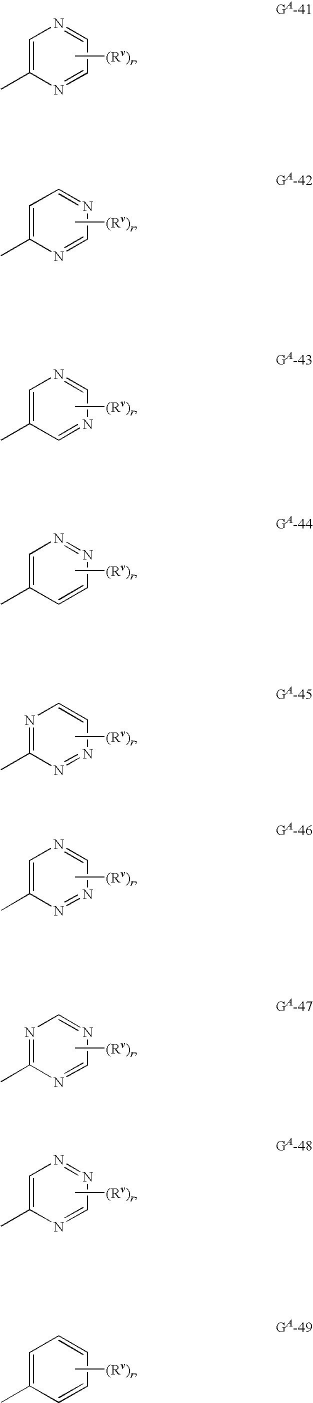

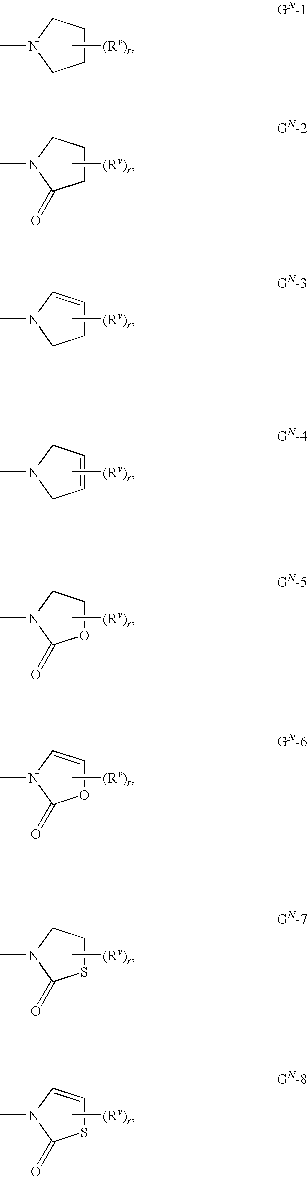

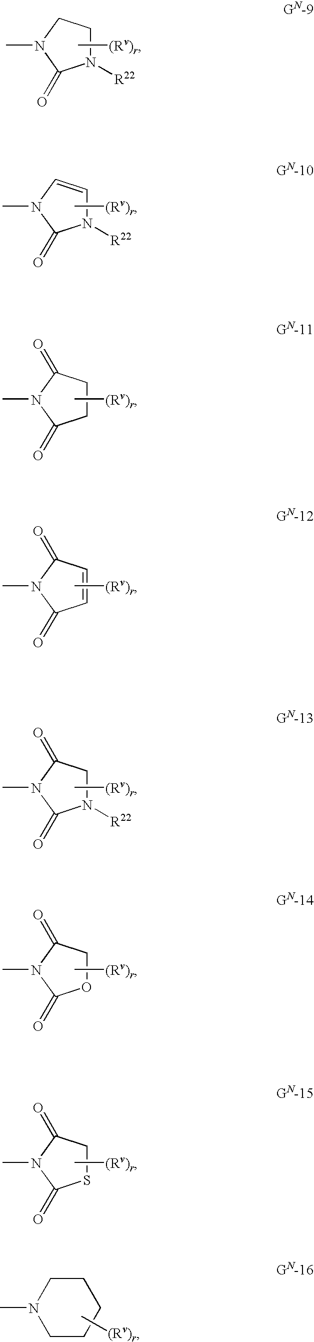

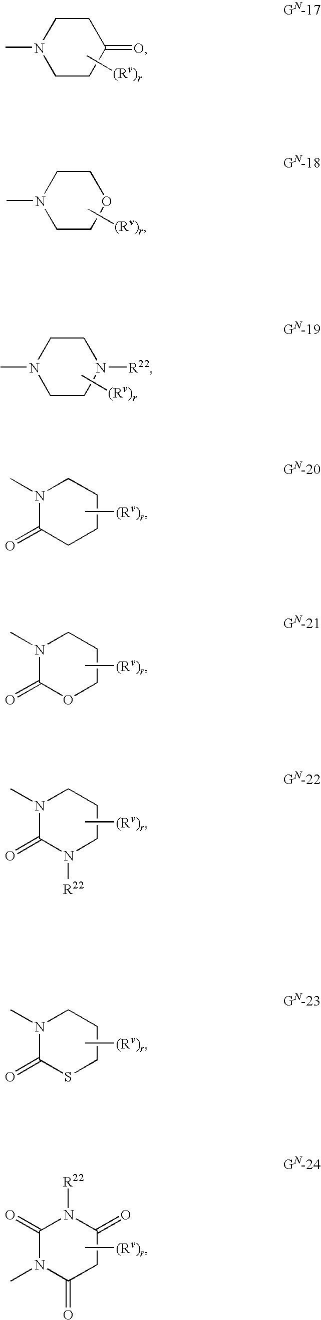

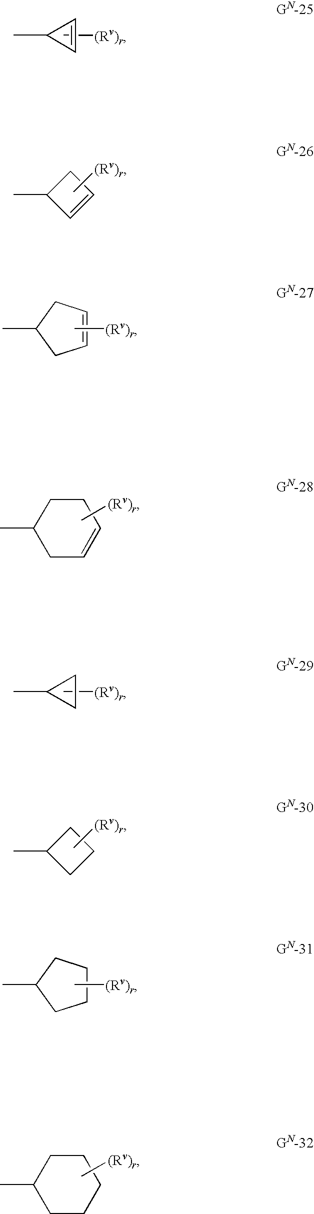

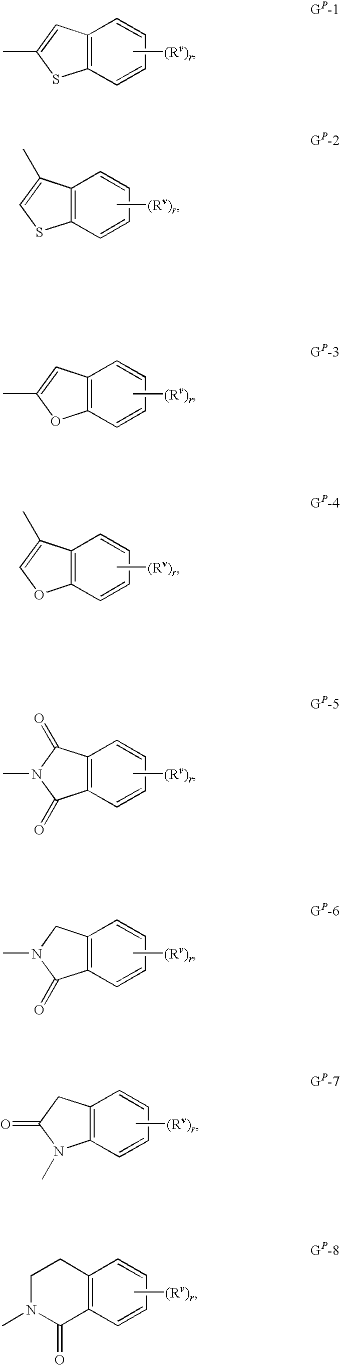

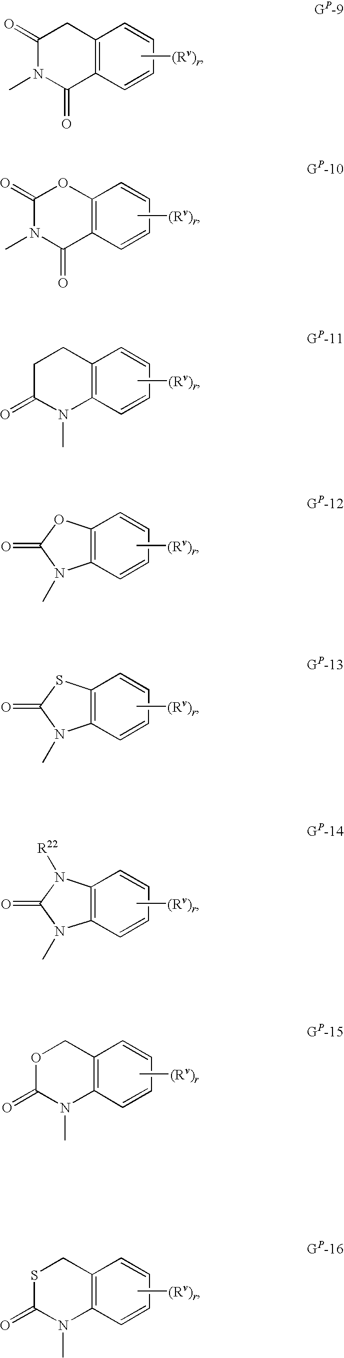

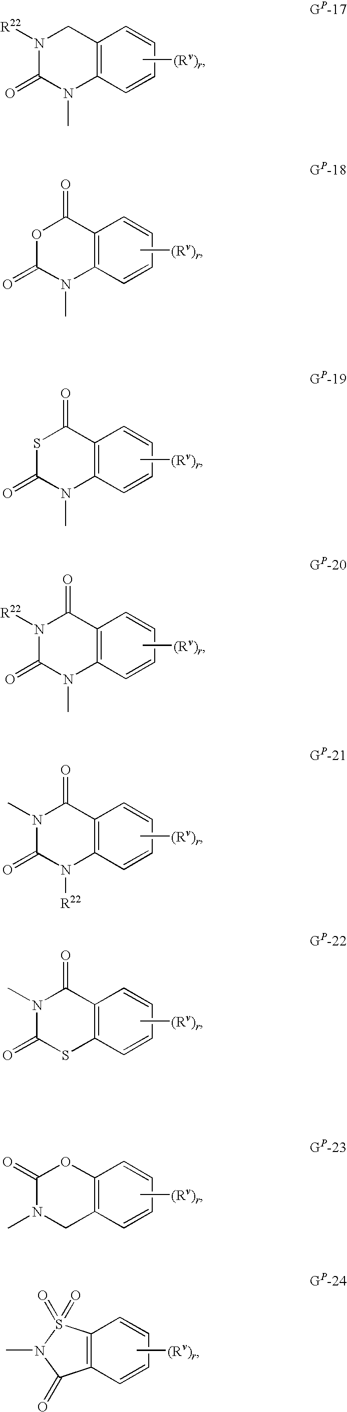

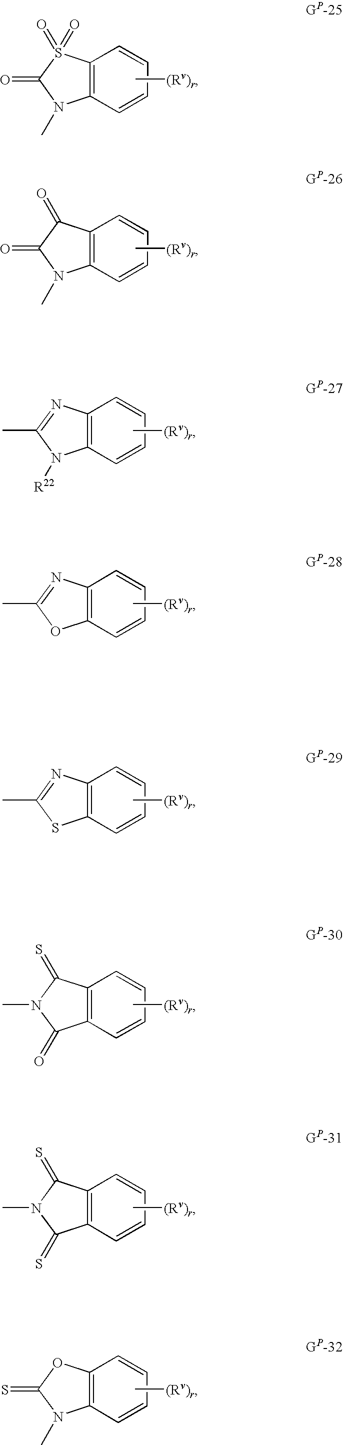

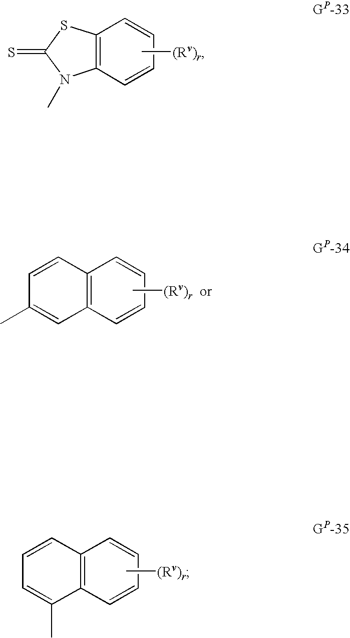

wherein the bond shown projecting to the left is bonded to Z³; and r is 0, 1, 2, 3, 4 or 5.

### Embodiment 97a

A compound of Embodiment 97 wherein r is 0, 1, 2 or 3.

### Embodiment 97b

A compound of Embodiment 97 or 97a wherein G^{A} is selected from G^{A}-1 through G^{A}-18, G^{A}-23 through G^{A}-38 and G^{A}-49, G^{N} is selected from G^{N}-1, G^{N}-2, G^{N}-5, G^{N}-6, G^{N}-9 through G^{N}-16 and G^{N}-29, and G^{P} is selected from G^{P}-1 through G^{P}-6, G^{P}-34 and G^{P}-38.

### Embodiment 98

A compound of Embodiment 97b wherein G^{A} is selected from G^{A}-1 through G^{A}-18, G^{A}-23 through G^{A}-38 and G^{A}-49, and G^{N} is selected from G^{N}-1, G^{N}-2, G^{N}-5, G^{N}-6, G^{N}-9 through G^{N}-16 and G^{N}-29.

### Embodiment 99

A compound of Embodiment 98 wherein G^{A} is selected from G^{A}-18 and G^{A}-49.

### Embodiment 100

A compound of Embodiment 99 wherein G^{A} is G^{A}-18.

### Embodiment 101

A compound of Embodiment 99 wherein G^{A} is G^{A}-49.

### Embodiment 102

A compound of Formula 1 or any one of Embodiments 1 through 101 wherein each R^{v} is independently H, halogen, cyano, hydroxy, —C(═O)OH, —C(═O)NH₂, —SO₂NH₂, —SH, C₁-C₆ alkyl, C₂-C₆ alkenyl, C₂-C₆ alkynyl, C₁-C₆ haloalkyl, C₂-C₈ alkylcarbonyl, C₂-C₈ alkoxycarbonyl, C₄-C₁₀ cycloalkoxycarbonyl, C₅-C₁₂ cycloalkylalkoxycarbonyl, C₂-C₈ alkylaminocarbonyl, C₃-C₁₀ dialkylaminocarbonyl, C₂-C₆ haloalkenyl, C₂-C₆ haloalkynyl, C₃-C₈ cycloalkyl, C₃-C₈ halocycloalkyl, C₄-C₁₀ alkylcycloalkyl, C₄-C₁₀ cycloalkylalkyl, C₆-C₁₄ cycloalkylcycloalkyl, C₄-C₁₀ halocycloalkylalkyl, C₅-C₁₂ alkylcycloalkylalkyl, C₂-C₈ alkoxyalkyl, C₄-C₁₀ cycloalkoxyalkyl, C₃-C₁₀ alkoxyalkoxyalkyl, C₂-C₈ alkylthioalkyl, C₂-C₈ alkylsulfinylalkyl, C₂-C₈ alkylsulfonylalkyl, C₂-C₈ alkylaminoalkyl, C₃-C₁₀ dialkylaminoalkyl, C₂-C₈ haloalkylaminoalkyl, C₄-C₁₀ cycloalkylaminoalkyl, C₄-C₁₀ cycloalkylcarbonyl, C₄-C₁₀ cycloalkylaminocarbonyl, C₂-C₇ cyanoalkyl, C₁-C₆ hydroxyalkyl, C₄-C₁₀ cycloalkenylalkyl, C₂-C₈ haloalkoxyalkyl, C₂-C₈ alkoxyhaloalkyl, C₃-C₁₀ alkoxyalkylcarbonyl, C₃-C₁₀ alkoxycarbonylalkyl, C₃-C₁₀ alkoxy(alkyl)aminocarbonyl, C₂-C₈ alkylamidino, C₃-C₁₀ dialkylamidino, C₁-C₆ alkoxy, C₁-C₆ haloalkoxy, C₂-C₈ alkylcarbonyloxy, C₁-C₆ alkylthio, C₁-C₆ haloalkylthio, C₁-C₆ alkylsulfinyl, C₁-C₆ alkylsulfonyl, C₁-C₆ alkylaminosulfonyl, C₂-C₈ dialkylaminosulfonyl, C₃-C₁₀ trialkylsilyl, C₂-C₈ alkoxyalkoxy, C₁-C₆ alkylamino, C₂-C₈ dialkylamino, C₂-C₈ alkylcarbonylamino, C₁-C₆ alkylsulfonylamino or C₁-C₆ halo alkylamino.

### Embodiment 103

A compound of Embodiment 102 wherein each R^{v} is independently H, halogen, cyano, hydroxy, C₁-C₆ alkyl, C₂-C₆ alkenyl, C₂-C₆ alkynyl, C₁-C₆ haloalkyl, C₂-C₈ alkylcarbonyl, C₂-C₈ alkoxycarbonyl, C₃-C₈ cycloalkyl, C₄-C₁₀ alkylcycloalkyl, C₄-C₁₀ cycloalkylalkyl, C₆-C₁₄ cycloalkylcycloalkyl, C₂-C₈ alkoxyalkyl, C₃-C₁₀ dialkylaminoalkyl, C₂-C₇ cyanoalkyl, C₁-C₆ hydroxyalkyl, C₂-C₈ haloalkoxyalkyl, C₃-C₁₀ alkoxyalkylcarbonyl, C₃-C₁₀ alkoxycarbonylalkyl, C₁-C₆ alkoxy, C₁-C₆ haloalkoxy, C₂-C₈ alkylcarbonyloxy, C₁-C₆ alkylthio, C₁-C₆ haloalkylthio, C₁-C₆ alkylsulfinyl, C₁-C₆ alkylsulfonyl, C₁-C₆ alkylamino or C₂-C₈ dialkylamino.

### Embodiment 104

A compound of Embodiment 103 wherein each R^{v} is independently H, halogen, cyano, hydroxy, C₁-C₂ alkyl, C₁-C₂ haloalkyl, C₁-C₂ alkoxy or C₁-C₂ haloalkoxy.

### Embodiment 104a

A compound of Embodiment 104 wherein each R^{v} is independently

H, halogen, hydroxy, or methyl.

### Embodiment 105

A compound of Formula 1 or any one of Embodiments 1 through 104 wherein each R^{7a} is independently C₁-C₆ alkyl, C₃-C₆ cycloalkyl, C₁-C₆ haloalkyl, halogen, cyano, C₁-C₄ alkoxy, C₁-C₄ haloalkoxy or C₂-C₆ alkoxycarbonyl.

### Embodiment 106

A compound of Embodiment 105 wherein each R^{7a} is independently methyl, CF₃, halogen or methoxy.

### Embodiment 107

A compound of Formula 1 or any one of Embodiments 1 through 106 wherein R²¹ is H, C₁-C₃ alkyl, C₁-C₃ alkylcarbonyl or C₂-C₃ alkoxycarbonyl.

### Embodiment 108

A compound of Formula 1 or any one of Embodiments 1 through 107 wherein each Z⁴ is independently C(═O) or S(O)₂.

### Embodiment 109

A compound of Embodiment 108 wherein each Z⁴ is C(═O).

### Embodiment 110

A compound of Formula 1 or any one of Embodiments 1 through 109 wherein when G is an optionally substituted thiazole ring connected at its 2-position to X and at its 4-position to Z¹ in Formula 1, A is CHR¹⁵, and J is a substituted isoxazole ring connected at its 4-position to Z¹, then Z¹ is O, C(═O), S(O)ₘ, CHR²⁰ or NR²¹.

### Embodiment 111

A compound of Formula 1 or any one of Embodiments 1 through 110 wherein when G is an optionally substituted thiazole ring connected at its 2-position to X and at its 4-position to Z¹ in Formula 1, and J is a substituted isoxazole ring connected at its 4-position to Z¹, then Z¹ is O, C(═O), S(O)ₘ, CHR²⁰ or NR²¹.

### Embodiment 112

A compound of Formula 1 or any one of Embodiments 1 through 111 wherein when G is an optionally substituted thiazole ring connected at its 2-position to X and at its 4-position to Z¹ in Formula 1, A is CHR¹⁵, Z¹ is a direct bond, and J is a substituted isoxazole ring, then J is connected to the remainder of the Formula 1 at the 3- or 5-position of the isoxazole ring.

### Embodiment 113

A compound of Formula 1 or any one of Embodiments 1 through 112 wherein when G is an optionally substituted thiazole ring connected at its 2-position to X and at its 4-position to Z¹ in Formula 1, A is CHR¹⁵, Z¹ is a direct bond, and J is a substituted isoxazole ring, then J is connected to the remainder of the Formula 1 at the 3-position of the isoxazole ring.

### Embodiment 114

A compound of Formula 1 or any one of Embodiments 1 through 113 wherein when G is an optionally substituted thiazole ring connected at its 2-position to X and at its 4-position to Z¹ in Formula 1, Z¹ is a direct bond, and J is a substituted isoxazole ring, then J is connected to the remainder of the Formula 1 at the 3-position of the isoxazole ring.

### Embodiment 115

A compound of Formula 1 or any one of Embodiments 1 through 114 wherein when X is X¹ and the ring containing X is saturated, A is NH, G is an optionally substituted thiazole ring connected at its 2-position to X and at its 4-position to Z¹ in Formula 1, and J is a substituted imidazole ring connected at its 2-position to the remainder of Formula 1, then Z¹ is O, C(═O), S(O)ₘ, CHR²⁰ or NR²¹.

### Embodiment 116

A compound of Formula 1 or any one of Embodiments 1 through 115 wherein when X is X¹ and the ring containing X is saturated, A is NR¹⁶, G is an optionally substituted thiazole ring connected at its 2-position to X and at its 4-position to Z¹ in Formula 1, and J is a substituted imidazole ring connected at its 2-position to the remainder of Formula 1, then Z¹ is O, C(═O), S(O)ₘ, CHR²⁰ or NR²¹.

### Embodiment 117

A compound of Formula 1 or any one of Embodiments 1 through 116 wherein when G is an optionally substituted thiazole ring connected at its 2-position to X and at its 4-position to Z¹ in Formula 1, then J is other than substituted imidazolyl.

Combinations of Embodiments 1-117 are illustrated by:

### Embodiment A1

A compound of Formula 1 wherein
R¹ is a phenyl or 5- or 6-membered heteroaromatic ring optionally substituted with 1-3 substituents independently selected from R^{4a} on carbon ring members and R^{4b} on nitrogen ring members;
G is a 5-membered heterocyclic ring optionally substituted with up to 2 substituents selected from R³ on carbon ring members and selected from R¹¹ on nitrogen ring members;
J is one of J-1 through J-82 (as depicted in Exhibit 3) wherein the bond shown projecting to the left is bonded to Z¹;
each R² is independently C₁-C₂ alkyl, C₁-C₂ haloalkyl, C₁-C₂ alkoxy, halogen, cyano or hydroxy;
each R³ is independently C₁-C₃ alkyl, C₁-C₃ haloalkyl or halogen;
each R^{4a} is independently C₁-C₆ alkyl, C₂-C₆ alkenyl, C₂-C₆ alkynyl, C₃-C₆ cycloalkyl, C₄-C₁₀ cycloalkylalkyl, C₄-C₁₀ alkylcycloalkyl, C₅-C₁₀ alkylcycloalkylalkyl, C₁-C₆ haloalkyl, C₂-C₆ haloalkenyl, C₂-C₆ haloalkynyl, C₃-C₆ halocycloalkyl, halogen, hydroxy, amino, cyano, nitro, C₁-C₄ alkoxy, C₁-C₄ haloalkoxy, C₁-C₄ alkylthio, C₁-C₄ alkylsulfinyl, C₁-C₄ alkylsulfonyl, C₁-C₄ haloalkylthio, C₁-C₄ haloalkylsulfinyl, C₁-C₄ haloalkylsulfonyl, C₁-C₄ alkylamino, C₂-C₈ dialkylamino, C₃-C₆ cycloalkylamino, C₂-C₄ alkoxyalkyl, C₁-C₄ hydroxyalkyl, C₂-C₄ alkylcarbonyl, C₂-C₆ alkoxycarbonyl, C₂-C₆ alkylcarbonyloxy, C₂-C₆ alkylcarbonylthio, C₂-C₆ alkylaminocarbonyl, C₃-C₈ dialkylaminocarbonyl or C₃-C₆ trialkylsilyl;
each R^{4b} is independently C₁-C₆ alkyl, C₃-C₆ alkenyl, C₃-C₆ alkynyl, C₃-C₆ cycloalkyl, C₁-C₆ haloalkyl, C₃-C₆ haloalkenyl, C₃-C₆ haloalkynyl, C₃-C₆ halocycloalkyl or C₂-C₄ alkoxyalkyl;
each R¹¹ is independently C₁-C₃ alkyl;
R¹⁵ is H, halogen, cyano, hydroxy, —CHO, C₁-C₄ alkyl, C₁-C₄ haloalkyl or C₂-C₅ alkoxycarbonyl;
R¹⁶ is H, C₁-C₄ alkyl, C₁-C₄ haloalkyl, C₂-C₄ alkylcarbonyl, C₂-C₄ haloalkylcarbonyl or C₂-C₄ alkoxycarbonyl;
x is an integer from 0 to 5; and
s is an integer from 1 to 2.

### Embodiment A2

A compound of Embodiment A1 wherein
G is one of G-1 through G-59 (as depicted in Exhibit 2) wherein the bond projecting to the left is bonded to X, and bond projecting to the right is bonded to Z¹;
J is selected from J-1, J-2, J-3, J-4, J-5, J-7, J-8, J-9, J-10, J-11, J-12, J-14, J-15, J-16, J-20, J-24, J-25, J-26, J-29, J-30, J-37, J-38, J-45 and J-69;
Q is one of Q-1 through Q-106 (as depicted in Exhibit 4);
R¹ is one of U-1 through U-50 (as depicted in Exhibit 1) wherein when R⁴ is attached to a carbon ring member, said R⁴ is selected from R^{4a}, and when R⁴ is attached to a nitrogen ring member (e.g., in U-4, U-11 through U-15, U-24 through U-26, U-31 or U-35), said R⁴ is selected from R^{4b};
each R² is independently methyl, methoxy, cyano or hydroxy;
each R^{1a} is independently selected from H and R³;
each R⁵ is independently H, cyano, C₁-C₆ alkyl, C₁-C₆ haloalkyl, C₃-C₈ cycloalkyl, C₃-C₈ halocycloalkyl, C₂-C₆ alkoxyalkyl, C₁-C₆ alkoxy, C₁-C₆ haloalkoxy, C₃-C₈ cycloalkoxy, C₂-C₆ alkenyloxy, C₂-C₆ haloalkenyloxy, C₂-C₆ alkynyloxy, C₂-C₆ alkoxyalkoxy, C₂-C₆ alkylcarbonyloxy, C₂-C₆ halo alkylcarbonyloxy, C₁-C₆ alkylthio, C₁-C₆ haloalkylthio, C₃-C₁₀ trialkylsilyl or —NR²⁵R²⁶;
R^{11a} is selected from H and R¹¹;
R¹⁵ is H, cyano, hydroxy, methyl or methoxycarbonyl;
R¹⁶ is H, methyl, methylcarbonyl or methoxycarbonyl;
each Z⁴ is C(═O);
k is 0, 1 or 2;
p is 1 or 2;
q is 0, 1, 2, 3, 4 or 5; and
s is 1.

### Embodiment A3

A compound of Embodiment A2 wherein
G is selected from G-1, G-2, G-7, G-8, G-14, G-15, G-23, G-24, G-26, G-27, G-36, G-37, G-38, G-49, G-50 and G-55;
J is selected from J-4, J-5, J-8, J-11, J-15, J-16, J-20, J-29, J-30, J-37, J-38 and J-69;
each Q is independently Q-1, Q-20, Q-32 through Q-34, Q-45 through Q-47, Q-60 through Q-73, Q-76 through Q-79, Q-84 through Q-94 and Q-98 through Q-106;
A is CH₂ or NH;
W is O;
X is X¹, X² or X³;
Z¹ is a direct bond;
Z² is a direct bond or NR²¹;
R¹ is selected from U-1 through U-3, U-11, U-13, U-20, U-22, U-23, U-36 through U-39 and U-50;
each R³ is independently methyl or halogen;
each R^{4a} is independently C₁-C₂ alkyl, C₁-C₂ haloalkyl, halogen, C₁-C₂ alkoxy or C₁-C₂ haloalkoxy;
each R^{4b} is independently C₁-C₂ alkyl or C₁-C₂ haloalkyl;
each R^{7a} is independently C₁-C₆ alkyl, C₃-C₆ cycloalkyl, C₁-C₆ haloalkyl, halogen, cyano, C₁-C₄ alkoxy, C₁-C₄ haloalkoxy or C₂-C₆ alkoxycarbonyl;
k is 1 or 2; and
n is 0.

### Embodiment A4

A compound of Embodiment A3 wherein
A is CH₂;
G is selected from G-1, G-2, G-15, G-26, G-27, G-36, G-37 and G-38; and G is unsubstituted;
J is J-29;
Q is selected from Q-1, Q-45, Q-63, Q-64, Q-65, Q-68, Q-69, Q-70, Q-71, Q-72, Q-73, Q-76, Q-78, Q-79, Q-84, Q-85, Q-98, Q-99, Q-100 and Q-101 through Q-106;
X is X¹ or X²; and the ring comprising X is saturated;

R¹ is U-1, U-20 or U-50;
each R^{4a} is independently C₁-C₂ alkyl, trifluoromethyl, Cl, Br, I or methoxy;
each R^{4b} is independently C₁-C₂ alkyl or trifluoromethyl; and
each R⁵ is independently H, cyano, C₁-C₆ alkyl, C₁-C₆ haloalkyl, C₁-C₆ alkoxy, C₁-C₆ haloalkoxy or —NR²⁵R²⁶.

### Embodiment A5

A compound of Embodiment A4 wherein
G is selected from G-1, G-2, G-15, G-26 and G-36;
J is any one of J-29-1 to J-29-60 (depicted in Exhibit A);
Q is selected from Q-45, Q-63, Q-64, Q-65, Q-68, Q-69, Q-70, Q-71, Q-72 and Q-85; and
X is X¹.

Embodiments of the present invention also include:

### Embodiment B1

A compound of Formula 1A wherein M is C₁-C₂ alkyl, C₁-C₂ haloalkyl, hydroxy, C₁-C₄ alkoxy, C₁-C₂ haloalkoxy, C₁-C₃ alkylamino, C₂-C₆ dialkylamino, 1-piperidinyl, 1-pyrrolidinyl or 4-morpholinyl.

### Embodiment B2

A compound of Formula 1A wherein M is C₁-C₃ alkyl, C₁-C₃ haloalkyl, hydroxy, C₂-C₈ dialkylamino, 1-piperidinyl, 1-pyrrolidinyl or 4-morpholinyl.

### Embodiment B3

A compound of Embodiment B2 wherein M is methyl, halomethyl, hydroxy, C₂-C₈ dialkylamino, 1-piperidinyl, 1-pyrrolidinyl or 4-morpholinyl.

### Embodiment B4

A compound of Embodiment B3 wherein M is C₂-C₈ dialkylamino, 1-piperidinyl, 1-pyrrolidinyl or 4-morpholinyl.

### Embodiment B5

A compound of Formula 1A or any one of Embodiments B1 through B4 wherein J¹ is any one of J-29-1 through J-29-57 (as depicted in Exhibit A).

With regards to the compounds of Formula 1A of this invention, it is noted that various embodiments of J-29 can be present in two or more enantiomeric forms. The enantiomeric forms of J-29 embodiments for compounds of Formula 1A of this invention are those depicted in Exhibit A above. All J-29 enantiomers are included in the Formula 1A compounds in this invention for embodiments where no specific J-29 enantiomeric form is depicted.

Specific embodiments include compounds of Formula 1 selected from the group consisting of:
1-[4-[4-[4,5-dihydro-5-[3-(1H-1,2,4-triazol-1-yl)phenyl]-3-isoxazolyl]-2-thiazolyl]-1-piperidinyl]-2-[5-methyl-3-(trifluoromethyl)-1H-pyrazol-1-yl]ethanone,
1-[4-[4-(5-[1,1′-biphenyl]-4-yl-4,5-dihydro-3-isoxazolyl)-2-thiazolyl]-1-piperidinyl]-2-[5-methyl-3-(trifluoromethyl)-1H-pyrazol-1-yl]ethanone,
4-[4-(5-[1,1′-biphenyl]-2-yl-4,5-dihydro-3-isoxazolyl)-2-thiazolyl]-N-(2,5-dimethylphenyl)-1-piperidinecarboxamide,
4-[4-(4,5-dihydro-5-[2-(1H-1,2,4-triazol-1-yl)phenyl]-3-isoxazolyl)-2-thiazolyl]-N-(2,5-dimethylphenyl)-1-piperidinecarboxamide,
1-[4-[4-[4,5-dihydro-5-[2-(1H-1,2,4-triazol-1-yl)phenyl]-3-isoxazolyl]-2-thiazolyl]-1-piperidinyl]-2-[5-methyl-3-(trifluoromethyl)-1H-pyrazol-1-yl]ethanone,
1-[4-[4-[5-[2-fluoro-6-(1H-1,2,4-triazol-1-yl)phenyl]-4,5-dihydro-3-isoxazolyl]-2-thiazolyl]-1-piperidinyl]-2-[5-methyl-3-(trifluoromethyl)-1H-pyrazol-1-yl]ethanone, and
1-[4-[4-(5-[1,1′-biphenyl]-2-yl-4,5-dihydro-3-isoxazolyl)-2-thiazolyl]-1-piperidinyl]-2-[5-methyl-3-(trifluoromethyl)-1H-pyrazol-1-yl]ethanone.

This invention provides a fungicidal composition comprising a compound selected from compounds of Formula 1 (including all geometric and stereoisomers) and N-oxides and salts thereof, and at least one other fungicide. Of note as embodiments of such compositions are compositions comprising a compound corresponding to any of the compound embodiments described above.

This invention provides a fungicidal composition comprising a fungicidally effective amount of a compound selected from compounds of Formula 1 (including all geometric and stereoisomers) and N-oxides and salts thereof, and at least one additional component selected from the group consisting of surfactants, solid diluents and liquid diluents. Of note as embodiments of such compositions are compositions comprising a compound corresponding to any of the compound embodiments described above.

This invention provides a method for controlling plant diseases caused by fungal plant pathogens comprising applying to the plant or portion thereof, or to the plant seed, a fungicidally effective amount of a compound selected from compounds of Formula 1 (including all geometric and stereoisomers) and N-oxides and salts thereof. Of note as embodiments of such methods are methods comprising applying a fungicidally effective amount of a compound corresponding to any of the compound embodiments described above. Of particular note are embodiments where the compounds are applied as compositions of this invention.

Also of note are the above embodiments, including Embodiments 1 through 117, A1 through A5, and B1 through B5 wherein Formulae 1 and 1A do not include N-oxides thereof, do not include salts thereof, or do not include N-oxides and salts thereof.

The compounds of Formulae 1 and 1A can be prepared by one or more of the following methods and variations as described in Schemes 1-29. The definitions of A, G, J, W, X, Q, Z¹, Z², Z³, R¹, R², R¹⁵, R¹⁶ and n in the compounds of Formulae 1-48 and Formulae 1Ba and 1Bb below are as defined above in the Summary of the Invention unless otherwise noted. Formulae 1a-1i are various subsets of Formula 1; Formulae 37a is an alternative depiction of Formula 37.

As shown in Scheme 1, compounds of Formula 1a (Formula 1 wherein A is CHR¹⁵) wherein W is O can be prepared by coupling of an acid chloride of Formula 2 with an amine of Formula 3 in the presence of an acid scavenger. Typical acid scavengers include amine bases such as triethylamine, N,N-diisopropylethylamine and pyridine. Other scavengers include hydroxides such as sodium and potassium hydroxide and carbonates such as sodium carbonate and potassium carbonate. In certain instances it is useful to use polymer-supported acid scavengers such as polymer-bound N,N-diisopropylethylamine and polymer-bound 4-(dimethylamino)pyridine. Acid salts of the Formula 3 amines can also be used in this reaction, provided that at least 2 equivalents of the acid scavenger is present. Typical acids used to form salts with amines include hydrochloric acid, oxalic acid and trifluoroacetic acid. In a subsequent step, amides of Formula 1a wherein W is O can be converted to thioamides of Formula 1a wherein W is S using a variety of standard thiating reagents such as phosphorus pentasulfide or 2,4-bis(4-methoxyphenyl)-1,3-dithia-2,4-diphosphetane-2,4-disulfide (Lawesson's reagent).

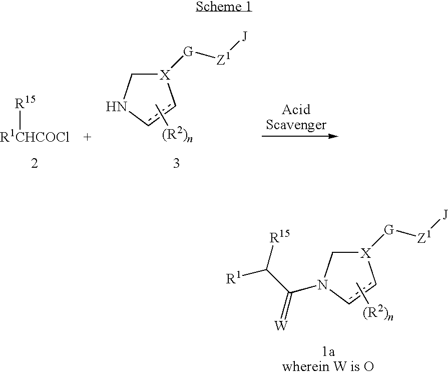

An alternate procedure for the preparation of compounds of Formula 1a wherein W is O is depicted in Scheme 2 and involves coupling of an acid of Formula 4 with an amine of Formula 3 (or its acid salt) in the presence of a dehydrative coupling reagent such as dicyclohexylcarbodiimide (DCC), 1-(3-dimethylaminopropyl)-3-ethylcarbodiimide hydrochloride (EDC) or O-benzotriazol-1-yl-N,N,N′,N′-tetramethyluronium hexafluoro-phosphate (HBTU). Polymer-supported reagents are again useful here, such as polymer-bound cyclohexylcarbodiimide. These reactions are typically run at 0-40° C. in a solvent such as dichloromethane or acetonitrile in the presence of a base such as triethylamine or N,N-diisopropylethylamine. The acids of Formula 4 are known or can be prepared by methods known to one skilled in the art. For example, R¹CH₂COOH where R¹ is a heteroaromatic ring linked through nitrogen can be prepared by reacting the corresponding R¹H compound with a haloacetic acid or ester in the presence of base; see, for example, U.S. Pat. No. 4,084,955. R¹CH₂COOH wherein R¹ is a phenyl or a heteroaromatic ring linked through carbon can be prepared from the corresponding R¹CH₂-halogen compounds by displacement of the halogen with cyanide followed by hydrolysis; see, for example, K. Adachi, Yuki Gosei Kagaku Kyokaishi 1969, 27, 875-876; from R¹C(═O)CH₃ by the Willgerodt-Kindler reaction; see, for example, H. R. Darabi et al., Tetrahedron Letters 1999, 40, 7549-7552 and M. M. Alam and S. R. Adapa, Synthetic Communications 2003, 33, 59-63 and references cited therein; or from R¹Br or R¹I by palladium-catalyzed coupling with tent-butyl acetate or diethyl malonate followed by ester hydrolysis; see, for example, W. A. Moradi and S. L. Buchwald, J. Am. Chem. Soc. 2001, 123, 7996-8002 and J. F. Hartwig et al., J. Am. Chem. Soc. 2002, 124, 12557-12565.

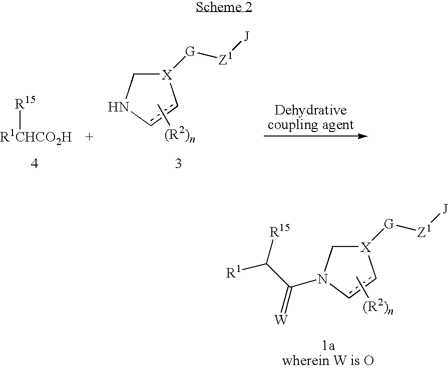

As the synthetic literature includes many amide-forming methods, the synthetic procedures of Schemes 1 and 2 are simply representative examples of an wide variety of methods useful for the preparation of Formula 1 compounds. One skilled in the art also realizes that acid chlorides of Formula 2 can be prepared from acids of Formula 4 by numerous well-known methods.

Certain compounds of Formula 1b (Formula 1 wherein A is CHR¹⁵ and W is O) wherein R¹ is a 5-membered nitrogen-containing heteroaromatic ring linked through the nitrogen atom can be prepared by reaction of the parent heterocycle of Formula 5 and a haloacetamide of Formula 6 as shown in Scheme 3. The reaction is carried out in the presence of a base such as sodium hydride or potassium carbonate in a solvent such as tetrahydrofuran, N,N-dimethylformamide or acetonitrile at 0 to 80° C. The haloacetamide of Formula 6 can be prepared by the reaction of an amine of Formula 3 with an α-halo carboxylic acid halide or an α-halo carboxylic acid or its anhydride, analogous to the amide-forming reactions described in Schemes 1 and 2, respectively.

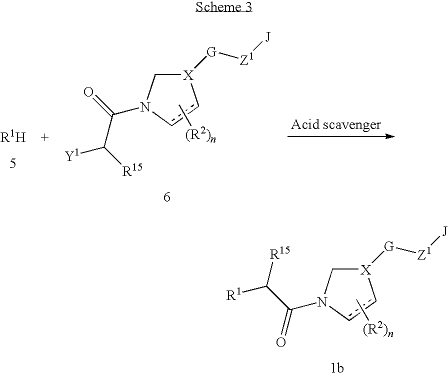

wherein R¹ is a 5-membered nitrogen-containing heteroaromatic ring unsubstituted on N; and Y¹ is Cl, Br or I.

Compounds of Formulae 1c (Formula 1 wherein A is NH), wherein R¹ is phenyl, naphthalenyl or a 5- or 6-membered heteroaromatic ring, and W is O or S, can be prepared by reaction of an amine of Formula 3 with an isocyanate or isothiocyanate, respectively, of Formula 7 as depicted in Scheme 4. This reaction is typically carried out at an ambient temperature in an aprotic solvent such as dichloromethane or acetonitrile.

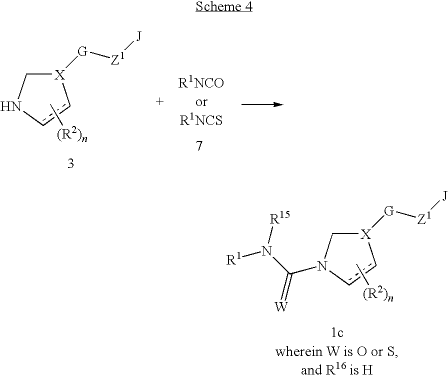

Compounds of Formulae 1c can also be prepared by the reaction of an amine of Formula 8 with a carbamoyl or thiocarbamoyl chloride or imidazole of Formula 9 as shown in Scheme 5. When Y is chlorine, the reaction is typically carried out in the presence of an acid scavenger. Typical acid scavengers include amine bases such as triethylamine, N,N-diisopropylethylamine and pyridine. Other scavengers include hydroxides such as sodium and potassium hydroxide and carbonates such as sodium carbonate and potassium carbonate. The carbamoyl or thiocarbamoyl chlorides of Formula 9 (wherein Y is Cl) can be prepared from amines of Formula 3 by treatment with phosgene or thiophosgene, respectively, or their equivalents, while carbamoyl or thiocarbamoyl imidazoles of Formula 9 (wherein Y is imidazol-1-yl) can be prepared from amines of Formula 3 by treatment with 1,1′-carbonyldiimidazole or 1,1′-thiocarbonyldiimidazole, respectively, according to general methods known to one skilled in the art.

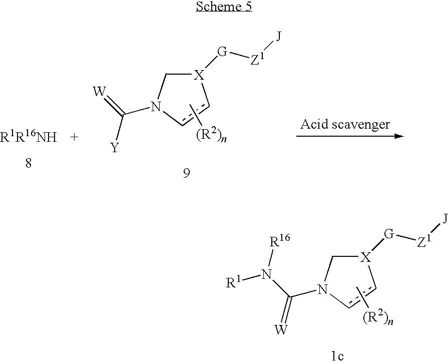

wherein W is O or S; and Y is Cl or imidazol-1-yl.

Certain compounds of Formula 1d (i.e. Formula 1 in which the ring containing X is saturated) can be prepared from compounds of Formula 1e where the ring containing X is unsaturated by catalytic hydrogenation as shown in Scheme 6. Typical conditions involve exposing a compound of Formula 1e to hydrogen gas at a pressure of 70 to 700 kPa, preferably 270 to 350 kPa, in the presence of a metal catalyst such as palladium supported on an inert carrier such as activated carbon, in a weight ratio of 5 to 20% of metal to carrier, suspended in a solvent such as ethanol at an ambient temperature. This type of reduction is very well known; see, for example, Catalytic Hydrogenation, L. Cerveny, Ed., Elsevier Science, Amsterdam, 1986. One skilled in the art will recognize that other certain functionalities that may be present in compounds of Formula 1e can also be reduced under catalytic hydrogenation conditions, thus requiring a suitable choice of catalyst and conditions

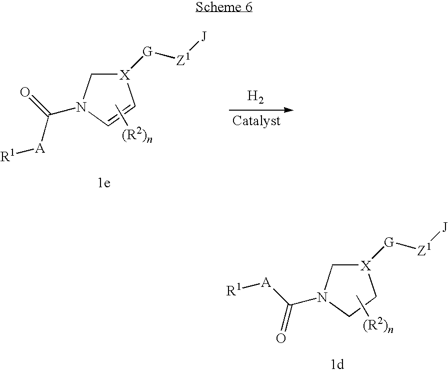

wherein X is X¹, X², X⁵, X⁸ or X⁹.

Certain compounds of Formula 1 wherein X is X¹, X⁵, X⁷ or X⁹, and G is linked to the ring containing X via a nitrogen atom, can be prepared by displacement of an appropriate leaving group Y² on the ring containing the X of Formula 10 with a nitrogen-containing heterocycle of Formula 11 in the presence of a base as depicted in Scheme 7. Suitable bases include sodium hydride or potassium carbonate, and the reaction is carried out in a solvent such as N,N-dimethylformamide or acetonitrile at 0 to 80° C. Suitable leaving groups in the compounds of Formula 10 include bromide, iodide, mesylate (OS(O)₂CH₃), triflate (OS(O)₂CF₃) and the like, and compounds of Formula 10 can be prepared from the corresponding compounds wherein Y² is OH, using general methods known in the art.

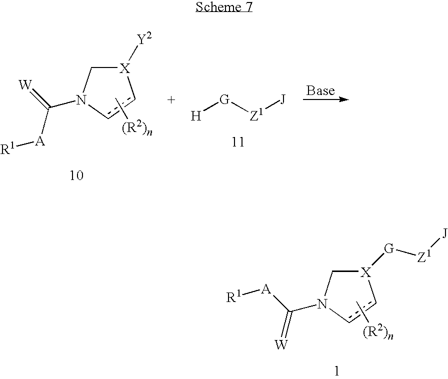

wherein W is O or S; X is X¹, X⁵, X⁷ or X⁹; and Y² is a leaving group such as Br, I, OS(O)₂Me or OS(O)₂CF₃.

Compounds of Formula 1 wherein X is X² or X⁸ can be prepared by reaction of a compound of Formula 12 with a heterocyclic halide or triflate (OS(O)₂CF₃) of Formula 13 as shown in Scheme 8. The reaction is carried out in the presence of a base such as potassium carbonate in a solvent such as dimethylsulfoxide, N,N-dimethylformamide or acetonitrile at 0 to 80° C. Compounds of Formula 13 wherein Y² is triflate can be prepared from corresponding compounds wherein Y² is OH by methods known to one skilled in the art.

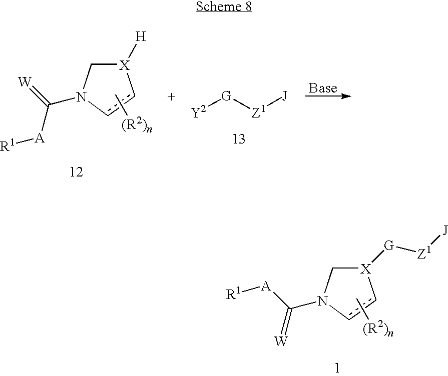

wherein W is O or S; X is X² or X⁸; and Y² is a leaving group such as Br, I OS(O)₂Me or OS(O)₂CF₃.

The amine compounds of Formula 3 can be prepared from the protected amine compounds of Formula 14 where Y³ is an amine-protecting group as shown in Scheme 9. A wide array of amine-protecting groups are available (see, for example, T. W. Greene and P. G. M. Wuts, Protective Groups in Organic Synthesis, 2nd ed.; Wiley: New York, 1991), and the use and choice of the appropriate protecting groups will be apparent to one skilled in chemical synthesis. The protecting group can be removed and the amine isolated as its acid salt or the free amine by general methods known in the art. One skilled in the art will also recognize that the protected amines of Formula 14 can be prepared by methods analogous to those described in Schemes 6, 7, and 8 above where the group R¹AC(═W) is replaced by Y³ to give useful intermediates of Formula 14 for the preparation of compounds of Formula 1.

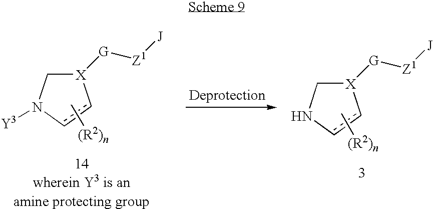

The compounds of Formula 14 can also be prepared by reaction of a suitably functionalized compound of Formula 15 with a suitably functionalized compound of Formula 16 as shown in Scheme 10. The functional groups Y⁴ and Y⁵ are selected from, but not limited to, moieties such as aldehydes, ketones, esters, acids, amides, thioamides, nitriles, amines, alcohols, thiols, hydrazines, oximes, amidines, amideoximes, olefins, acetylenes, halides, alkyl halides, methanesulfonates, trifluoromethanesulfonates, boronic acids, boronates, and the like, which under the appropriate reaction conditions, will allow the construction of the various heterocyclic rings G. As an example, reaction of a compound of Formula 15 where Y⁴ is a thioamide group with a compound of Formula 16 where Y⁵ is a bromoacetyl or chloroacetyl group will give a compound of Formula 14 where G is a thiazole ring. The synthetic literature describes many general methods for forming 5-membered heteroaromatic rings and 5-membered partially saturated heterocyclic rings (e.g., G-1 through G-59); see, for example, Comprehensive Heterocyclic Chemistry, Vol. 4-6, A. R. Katritzky and C. W. Rees editors, Pergamon Press, New York, 1984; Comprehensive Heterocyclic Chemistry II, Vol. 2-4, A. R. Katritzky, C. W. Rees, and E. F. Scriven editors, Pergamon Press, New York, 1996; and the series, The Chemistry of Heterocyclic Compounds, E. C. Taylor, editor, Wiley, New York. The use of intermediates of Formula 15 where X is X¹ and Y⁴ is Br, I, methanesulfonate or trifluoromethanesulfonate to prepare organozinc reagents for use in cross-coupling reactions with aromatic rings has been described; see, for example, S. Bellotte, Synlett 1998, 379-380, and M. Nakamura et al., Synlett 2005, 1794-1798. One skilled in the art knows how to select the appropriate functional groups to construct the desired heterocyclic rings such as G. Compounds of Formula 15 and 16 are known or can be prepared by general methods known in the art. For example, compounds of Formula 15 wherein Y⁴ is a thioamide group can be prepared from corresponding compounds wherein Y⁴ is cyano by treatment with sodium hydrosulfide, analogous to the method demonstrated in Example 2, Step B.

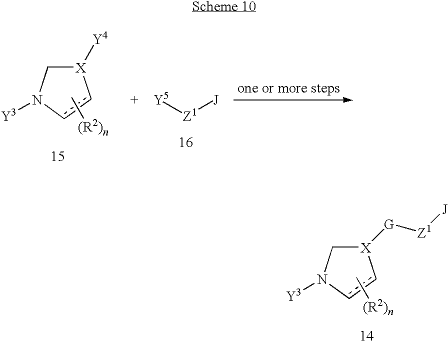

wherein Y⁴ and Y⁵ are functional groups suitable for construction of the desired heterocycle G.

One skilled in the art recognizes that the methods particularly described herein are illustrative of a wide variety of methods known in the synthetic organic chemistry art that are useful for preparing compounds of Formula 1. The order of assembling the molecular components of Formula 1 can be varied, and analogous starting compounds and reagents can be selected to prepare the various compounds within the scope of Formula 1. For example, the method of Scheme 10 involves forming the G ring from precursor groups Y⁴ and Y⁵ before removing protecting group Y³ as shown in Scheme 9 and attaching the left portion of the molecule (R¹AC(═W)—) as shown in Schemes 1 through 5. Alternatively, a method analogous to Scheme 10 can be used to form the G ring from precursor groups Y⁴ and Y⁵ after attaching the left portion of molecule using methods analogous to Schemes 1 through 5. This alternate synthetic route is demonstrated in Example 2 wherein Step A is analogous to Scheme 4, Step B is analogous to a method for preparing a starting compound for Scheme 10, Step C corresponds to Scheme 28, Step D is analogous to Scheme 20 and Step E is analogous to Scheme 10.

Certain compounds of Formula 14 where Z¹ is O, S, or NR²¹ can be prepared by displacement of an appropriate leaving group Y² on G of Formula 17 with a compound of Formula 18 in the presence of a base as depicted in Scheme 11. Suitable bases include sodium hydride or potassium carbonate, and the reaction is carried out in a solvent such as N,N-dimethylformamide or acetonitrile at 0 to 80° C. Suitable leaving groups in the compounds of Formula 17 include bromide, iodide, mesylate (OS(O)₂CH₃), triflate (OS(O)₂CF₃) and the like. Compounds of Formula 17 can be prepared from corresponding compounds wherein Y² is OH by general methods known in the art. The compounds of Formula 18 are known or can be prepared by general methods known in the art.

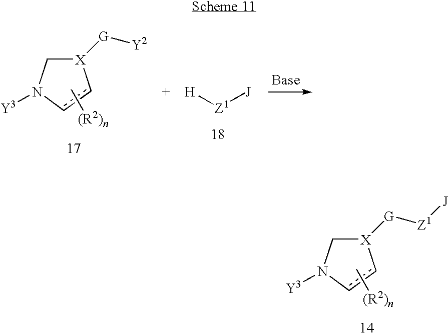

wherein Y² is a leaving group such as Br, I, OS(O)₂Me or OS(O)₂CF₃; and Z¹ is O, S or NR²¹.

Certain compounds of Formula 14 where Z¹ is O, S, or NR²¹ can also be prepared by displacement of an appropriate leaving group Y² on J of Formula 20 with a compound of Formula 19 in the presence of a base as depicted in Scheme 12. Suitable bases include sodium hydride or potassium carbonate, and the reaction is carried out in a solvent such as N,N-dimethylformamide or acetonitrile at 0 to 80° C. Suitable leaving groups in the compounds of Formula 20 include bromide, iodide, mesylate (OS(O)₂CH₃), triflate (OS(O)₂CF₃) and the like. Compounds of Formula 20 can be prepared from corresponding compounds wherein Y² is OH using general methods known in the art.

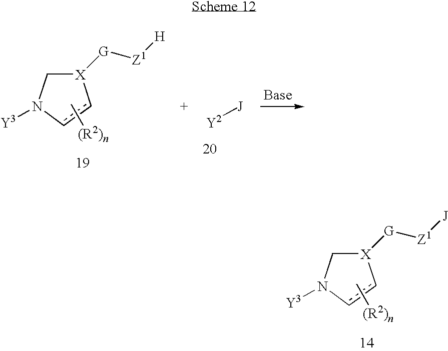

wherein Y² is a leaving group such as Br, I, OS(O)₂Me or OS(O)₂CF₃; and Z¹ is O, S or NR²¹.

Compounds of Formula 14 can also be prepared by reaction of a suitably functionalized compound of Formula 21 with a suitably functionalized compound of Formula 22 as shown in Scheme 13. The functional groups Y⁶ and Y⁷ are selected from, but not limited to, moieties such as aldehydes, ketones, esters, acids, amides, thioamides, nitriles, amines, alcohols, thiols, hydrazines, oximes, amidines, amide oximes, olefins, acetylenes, halides, alkyl halides, methanesulfonates, trifluoromethanesulfonates, boronic acids, boronates, and the like, which, under the appropriate reaction conditions will allow the construction of the various heterocyclic rings J. As an example, reaction of a compound of Formula 21 where Y⁶ is a chloro oxime moiety with a compound of Formula 22 where Y⁷ is a vinyl or acetylene group in the presence of base will give a compound of Formula 14 where J is an isoxazoline or isoxazole, respectively. The synthetic literature includes many general methods for the formation of carbocyclic and heterocyclic rings and ring systems (for example, J-1 through J-82); see, for example, Comprehensive Heterocyclic Chemistry, Vol. 4-6, A. R. Katritzky and C. W. Rees editors, Pergamon Press, New York, 1984; Comprehensive Heterocyclic Chemistry II, Vol. 2-4, A. R. Katritzky, C. W. Rees, and E. F. Scriven editors, Pergamon Press, New York, 1996; the series, The Chemistry of Heterocyclic Compounds, E. C. Taylor, editor, Wiley, New York, and Rodd's Chemistry of Carbon Compounds, Vol. 2-4, Elsevier, N.Y. General procedures for cycloaddition of nitrile oxides with olefins are well documented in the chemical literature. For relevant references; see, for example, Lee, Synthesis 1982, 6, 508-509 and Kanemasa et al., Tetrahedron 2000, 56, 1057-1064 as well as references cited within. One skilled in the art knows how to select the appropriate functional groups to construct the desired heterocyclic ring J. Compounds of Formula 22 are known or can be prepared by general methods known in the art.

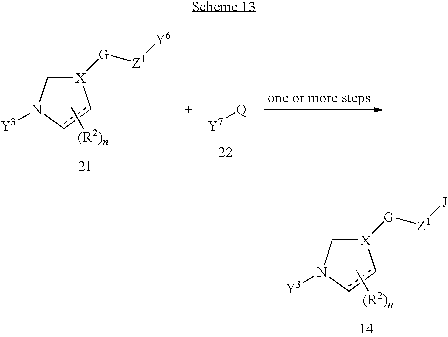

wherein Y⁶ and Y⁷ are functional groups suitable for construction of the desired heterocycle J.

An alternate preparation for the compounds of Formula 14 where Z¹ is a bond includes the well known Suzuki reaction involving Pd-catalyzed cross-coupling of an iodide or bromide of Formula 23 or 26 with a boronic acid of Formula 24 or 25, respectively, as shown in Scheme 14. Many catalysts are useful for this type of transformation; a typical catalyst is tetrakis(triphenylphosphine)palladium. Solvents such as tetrahydrofuran, acetonitrile, diethyl ether and dioxane are suitable. The Suzuki reaction and related coupling procedures offer many alternatives for creation of the G-J bond. For leading references; see, for example, C. A. Zificsak and D. J. Hlasta, Tetrahedron 2004, 60, 8991-9016. For a thorough review of palladium chemistry applicable to the synthesis of G-J bonds; see, for example, J. J. Li and G. W. Gribble, editors, Palladium in Heterocyclic Chemistry: A Guide for the Synthetic Chemist, Elsevier: Oxford, UK, 2000. Many variations of catalyst type, base and reaction conditions are known in the art for this general method.

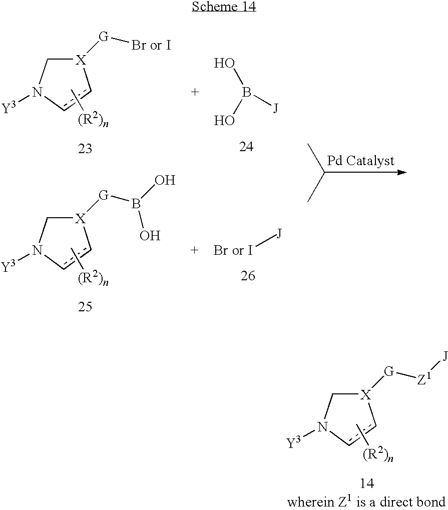

One skilled in the art will recognize that many compounds of Formula 1 can be prepared directly by methods analogous to those described in Schemes 10 through 14 above where the group Y³ is replaced by R¹AC(═W). Thus, compounds corresponding to Formulae 15, 17, 19, 21, 23 and 25 in which Y³ is replaced by R¹AC(═W) are useful intermediates for the preparation of compounds of Formula 1.

Thioamides of Formula 1Bb are particularly useful intermediates for preparing compounds of Formula 1 wherein X is X¹ using the thioamide-α-haloaryl ring-forming reaction described for the method of Scheme 10. A thioamide of Formula 1Bb can be prepared by the addition of hydrogen sulfide to the corresponding nitrile of Formula 1Ba as shown in Scheme 15.

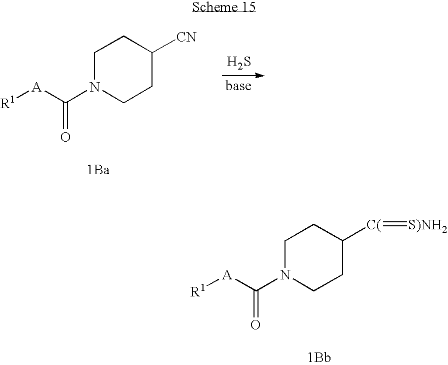

wherein R¹ and A are as defined for Formula 1.

The method of Scheme 15 can be carried out by contacting a compound of Formula 1Ba with hydrogen sulfide in the presence of an amine such as pyridine, diethylamine or diethanolamine. Alternatively, hydrogen sulfide can be used in the form of its bisulfide salt with an alkali metal or ammonia. This type of reaction is well documented in the literature (e.g., A. Jackson et al., EP 696,581 (1996)). This method is demonstrated in Example 1, Step C and Example 2, Step B.

Certain compounds of Formula 1Ba wherein R¹ is a 5-membered nitrogen-containing heteroaromatic ring linked through a nitrogen atom can be prepared by reaction of the parent heterocycle of Formula 5 and a haloacetamide of Formula 27 as shown in Scheme 16. The reaction is carried out in the presence of a base such as sodium hydride or potassium carbonate in a solvent such as tetrahydrofuran, N,N-dimethylformamide or acetonitrile at 0 to 80° C. This method is demonstrated in Example 1, Step B.

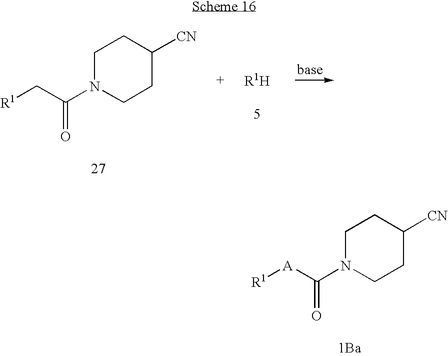

wherein in R¹H (Formula 5), R¹ is a 5-membered nitrogen-containing heteroaromatic ring unsubstituted on N (i.e. a 5-membered heteroaromatic ring comprising a ring member of the formula —(NH)—); A is CH₂; and Y¹ is Cl, Br or I.

The haloacetamides of Formula 27 can be prepared by the two methods shown in Scheme 17.

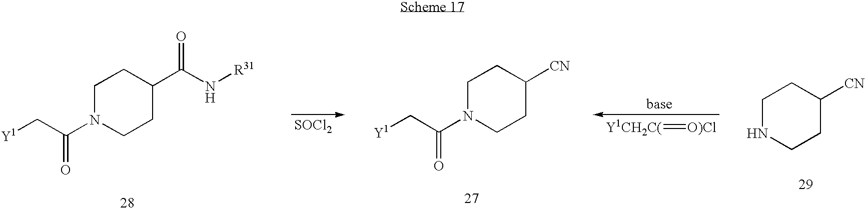

wherein Y¹ is Cl, Br, or I; and R³¹ is a tertiary alkyl group such as —C(Me)₃.

In one method, 4-cyanopiperidine of Formula 29 is haloacetylated by contact with the appropriate haloacetyl chloride typically in the presence of a base according to standard methods. Preferred conditions involve use of an aqueous solution of an inorganic base such as an alkali metal or alkaline-earth carbonate, bicarbonate or phosphate, and a non-water-miscible organic solvent such as toluene, ethyl acetate or 1,2-dichloroethane. In the second method depicted in Scheme 17, a 1-(haloacetyl)-N-substituted isonipecotamide derivative of Formula 28, wherein R³¹ is tertiary alkyl such as C(Me)₃, is dehydrated using a standard amide dehydrating agent such as thionyl chloride or phosphorus oxychloride in a suitable solvent. A particularly preferred solvent for this transformation is an N,N-dialkylamide such as N,N-dimethylformamide. The reaction is typically carried out by adding 0.9 to 2 equivalents, preferably 1.1 equivalents, of phosphorus oxychloride or thionyl chloride to a mixture of a compound of Formula 28 and 0.5 to 10 parts by weight of solvent, at a temperature at which the reaction rapidly proceeds during the addition. The addition time for this reaction is typically around 20 to 90 minutes at typical temperatures of around 35 to 55° C.

As shown in Scheme 18, the compounds of Formula 28 can be prepared from the compound of Formula 30 by analogy with the haloacetylation reaction described for Scheme 17.

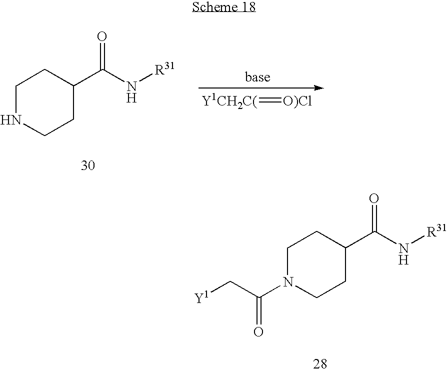

The compounds of Formula 30 are known or can be prepared from 4-cyanopyridine or isonicotinic acid using methods well-known in the art; see, for example, G. Marzolph et al., DE 3,537,762 (1986) for preparation of N-t-butyl pyridinecarboxamides from cyanopyridines and t-butanol and S. F. Nelsen et al., J. Org. Chem., 1990, 55, 3825 for hydrogenation of N-methylisonicotinamide with a platinum catalyst.

Halomethyl isoxazole ketones of Formula 35 are particularly useful intermediates for preparing certain chiral compounds of Formula 1 wherein J is, for example, selected from J-29-1 through J-29-57 as depicted in Exhibit A. Halomethyl isoxazole ketones of Formula 35 can be prepared by the multi-step reaction sequences shown in Scheme 19.

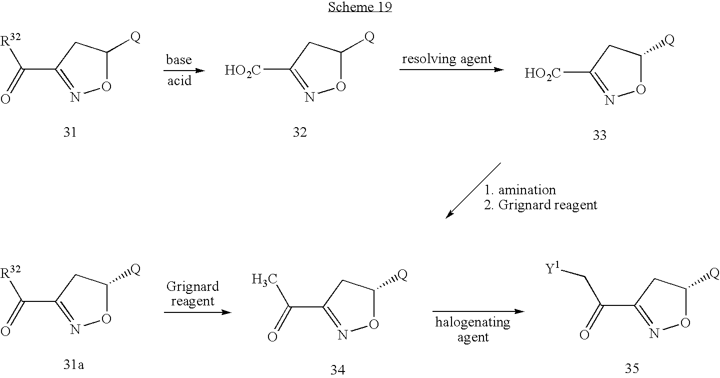

wherein R³² is C₂-C₈ dialkylamino, 1-piperidinyl, 1-pyrrolidinyl or 4-morpholinyl and Q is as defined above in the Summary of the Invention.

The preparation of the racemic carboxylic acids of Formula 32 can be accomplished according to the well-known methods of basic or acidic hydrolysis of the corresponding compounds of Formula 31, preferably using a slight excess of sodium hydroxide in a water-miscible co-solvent such as methanol or tetrahydrofuran at about 25 to 45° C. The product can be isolated by adjusting pH to about 1 to 3 and then filtration or extraction, optionally after removal of the organic solvent by evaporation. The racemic carboxylic acids of Formula 32 can be resolved by classical fractional crystallization of diastereomeric salts of suitable chiral amine bases such as cinchonine, dihydrocinchonine or a mixture thereof. A cinchonine-dihydrocinchonine mixture in about a 85:15 ratio is particularly useful, as it provides, for example, the (R)-configured carboxylic acids of Formula 33, wherein R⁵ is a substituted phenyl group, as the less soluble salt. Furthermore, these chiral amine bases are readily available on a commercial scale. The (R)-configured halomethyl ketone intermediates of Formula 35 afford the more fungicidally active final products of Formula 1 after coupling with thioamides of Formula 1Bb according to the method of Scheme 10. The halomethyl ketones of Formula 35 can be prepared by first reacting the corresponding amides of Formula 31, either as pure enantiomers (i.e. Formula 31a) or in enantiomerically enriched or racemic mixtures, with one molar equivalent of a methylmagnesium halide (Grignard reagent) in a suitable solvent or solvent mixture such as tetrahydrofuran and toluene at about 0 to 20° C., and the crude ketone products of Formula 34 can be isolated by quenching with aqueous acid, extraction, and concentration. Then the crude ketones of Formula 34 are halogenated with a reagent such as sulfuryl chloride to afford the chloromethyl ketones of Formula 35 wherein Y¹ is Cl or molecular bromine to afford the corresponding bromomethyl ketones of Formula 35 wherein Y¹ is Br. The halomethyl ketones of Formula 35 can be purified by crystallization from a solvent such as hexanes or methanol, or can be used without further purification in the condensation reaction with thioamides.

The transformation reactions depicted in Scheme 19 illustrate compounds of Formula 1A corresponding to Formulae 31 through 35, which are useful as intermediates for the preparation of certain compounds of Formula 1 wherein J is any one of J-29-1 through J-29-57 depicted in Exhibit A. R³² in Formulae 31 and 31a as well as corresponding groups in Formulae 32 through 35 correspond to M in Formula 1A. One skilled in the art recognizes that analogs of compounds of Formula 31 through 35 are useful for preparing other compounds of Formula 1 such as wherein J is any one of J-29-58 through J-29-60 depicted in Exhibit A. Furthermore one skilled in the art recognizes that for transformations shown in Scheme 19, R³² can be other groups besides C₂-C₈ dialkylamino, 1-piperidinyl, 1-pyrrolidinyl or 4-morpholinyl. For example, for the hydrolysis of a compound of Formula 31 to a compound of Formula 32 (corresponding to M in Formula 1A being hydroxy), R³² can also be C₁-C₄ alkoxy, C₁-C₂ haloalkoxy or C₁-C₄ alkylamino. Furthermore, the methyl (CH₃) group in Formula 34 and halomethyl (Y¹CH₂) group in Formula 35 are homologously representative of M in Formula 1A being C₁-C₃ alkyl and C₁-C₃ haloalkyl, respectively.

The isoxazole carboxamides of Formula 31 can be prepared by cycloaddition of the corresponding hydroxamoyl chlorides of Formula 36 with olefin derivatives of Formula 37, as shown in Scheme 20.

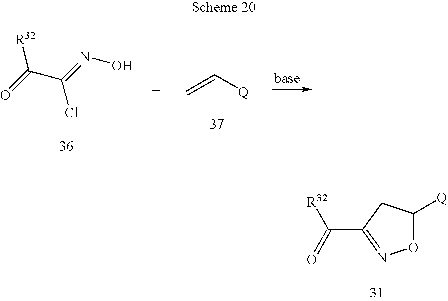

In this method, all three reacting components (the compounds of Formulae 36 and 37, and the base) are contacted so as to minimize hydrolysis or dimerization of the hydroxamoyl chloride of Formula 36. In one typical procedure, the base, which can either be a tertiary amine base such as triethylamine or an inorganic base such as an alkali metal or alkaline-earth carbonate, bicarbonate or phosphate, is mixed with the olefin derivative of Formula 37, and the hydroxamoyl chloride of Formula 36 is added gradually at a temperature at which the cycloaddition proceeds at a relatively rapid rate, typically between 5 and 25° C. Alternatively, the base can be added gradually to the other two components (the compounds of Formulae 36 and 37). This alternative procedure is preferable when the hydroxamoyl chloride of Formula 36 is substantially insoluble in the reaction medium. The solvent in the reaction medium can be water or an inert organic solvent such as toluene, hexane or even the olefin derivative used in excess. The product can be separated from the salt co-product by filtration or washing with water, followed by evaporation of the solvent. The crude product can be purified by crystallization, or the crude product can be used directly in the methods of Scheme 19. The method of Scheme 20 is demonstrated in Example 1, Step F. Also, a method analogous to Scheme 20 is demonstrated in Example 2, Step D. Compounds of Formula 31 are useful precursors to the corresponding methyl ketones of Formula 34 and halomethyl ketones of Formula 35, and are also useful for preparing the resolved enantiomers of the compounds of Formulae 34 and 35 by hydrolysis, resolution, methyl ketone synthesis and halogenation, as shown in Scheme 19.

Compounds of Formula 1f can be prepared by several methods. In one method, a compound of Formula 38 wherein Y⁸ is a leaving group such as halogen, for example iodine, is reacted with a compound of Formula 39 wherein Z³ is O, S or NH as shown in Scheme 21.

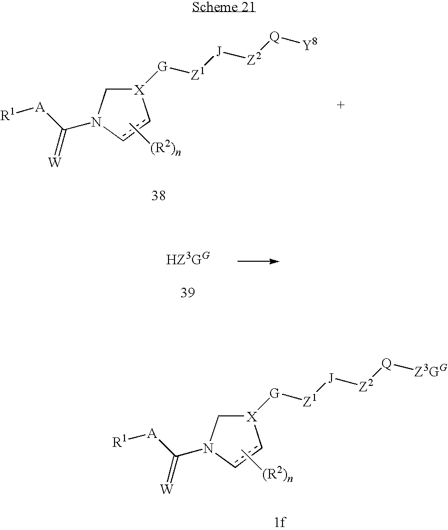

### Y⁸ is F, Cl, Br, I; Z³ is O, S or NH; G^{G} is G^{A}, G^{N} or G^{P}.

This reaction (known as the Ullmann ether synthesis when Z³ is O) is well known to one skilled in the art. The reaction is typically carried out in the presence of an inorganic base such as potassium carbonate or cesium carbonate and with a metal catalyst, for example, copper iodide. Temperatures between room temperature and 150° C. and solvents such as dimethyl sulfoxide and N,N-dimethylformamide are suitable for the reaction. Diaryl ethers of Formula 1f wherein Z³ is O can also be prepared using palladium-catalyzed Buchwald-Hartwig reaction, nucleophilic aromatic substitution or arylboronic acid diaryl ether coupling. For a recent review of these methods, including the Ullmann diaryl ether synthesis; see, for example, R. Frian and D. Kikeji, Synthesis 2006, 14, 2271-2285.

Conditions similar to those described for diaryl ethers can also be used to prepare compounds of Formula 1f where Z is S or NH. For a recent review of the preparation of sulfur and nitrogen analogs; see, for example, S. V. Ley and A. W Thomas, Angew. Chem., Int. Ed. Engl. 2003, 42, 5400.

A similar copper-catalyzed method can be used to prepare compounds of Formula 1g (i.e. Formula 1f wherein Z³ is a direct bond and G^{G} is G^{Gn} bonded through a nitrogen ring member) wherein G^{Gn} is G^{A}, G^{N} or G^{P} bonded through a nitrogen atom ring member of G^{Gn} to Q from a heterocycle HG^{Gn} in which H is connected to a nitrogen ring member, for example, triazole, or a salt thereof (e.g., sodium triazole) as shown in Scheme 22.

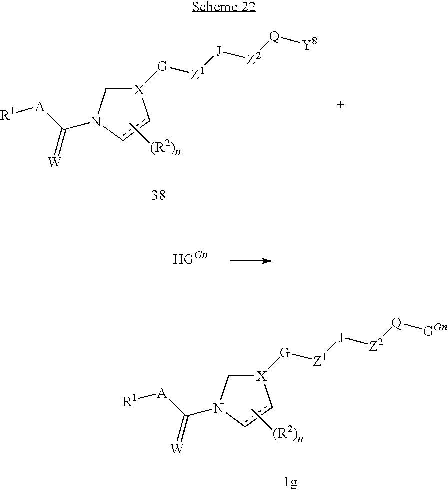

Y⁸ is F, Cl, Br, I; G^{Gn} is a G^{A}, G^{N} or G^{P} bonded through a ring nitrogen atom to Q.

A ligand such as (1R,2R)-N,N-dimethyl-1,2-cyclohexenediamine can be used to increase the solubility and reactivity of the copper catalyst. The reaction is typically carried out in a solvent such as dimethylsulfoxide or in a mixed solvent such as dimethylsulfoxide-water at temperatures between room temperature and 200° C. For leading reference; see, for example, Andersen et al., Synlett 2005, 14, 2209-2213. This method is demonstrated in Example 1, Step H.

Compounds of Formula 1h (i.e. Formula 1f wherein Z³ is a direct bond, and G^{G} is G^{Gc} bonded through a sp² carbon atom ring member) wherein G^{Gc} is G^{A}, G^{N} or G^{P} bonded through an sp² carbon atom ring member of G^{Gc} to Q can be prepared by a variety of general methods including the well known Suzuki reaction involving Pd-catalyzed cross-coupling as shown in Scheme 23.

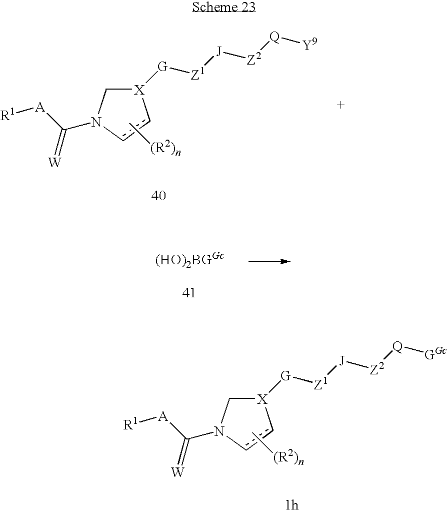

Y⁹ is Cl, Br, I, or OS(O)₂CF₃; G^{Gc} is G^{A}, G^{N} or G^{P} bonded through an sp² ring carbon atom to Q.

The conditions for coupling an iodide or bromide of Formula 40 with a boronic acid of Formula 41 wherein the boron is attached to an sp² ring carbon atom in G^{Gc} are similar to those described for the method of Scheme 14 above. Many catalysts are useful for this type of transformation; a typical catalyst is tetrakis(triphenylphosphine)palladium. Solvents such as tetrahydrofuran, acetonitrile, diethyl ether and dioxane are suitable. The Suzuki reaction and related coupling procedures offer many alternatives for creation of a direct bond between the Q and G^{Gc} rings. For leading references; see, for example, C. A. Zificsak and D. J. Hlasta, Tetrahedron 2004, 60, 8991-9016. For a thorough review of palladium chemistry applicable to the synthesis of QG^{Gc} bonds; see, for example, J. J. Li and G. W. Gribble, editors, Palladium in Heterocyclic Chemistry: A Guide for the Synthetic Chemist, Elsevier: Oxford, UK, 2000. Many variations of catalyst type, base and reaction conditions are known in the art for this general method.

As shown in Scheme 24, methods for preparing compounds of Formula 1f wherein Z³ is —C≡C— include the well-known Sonogashira reaction using Pd-catalyzed cross-coupling of a halide of Formula 40 wherein Y⁹ is a halogen such as iodine or bromide with an alkyne of Formula 42 in the presence of a metal catalyst and a base.

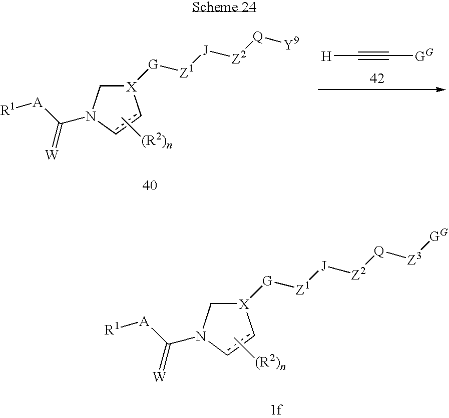

### Y⁹ is Cl, Br, I, or OS(O)₂CF₃; Z³ is —C≡C—; G^{G} is G^{A}, G^{N} or G^{P}.

Many catalysts are useful for this type of transformation; a typical catalyst is dichlorobis(tri-o-tolylphosphine)palladium (II). Suitable solvents include tetrahydrofuran, acetonitrile and ethyl acetate. Suitable metal catalysts include, for example, copper iodide. Typical bases include, for example, triethylamine or Hunig's base. For leading references; see, for example, I. B. Campbell, Organocopper Reagents 1994, 217-235.

As shown in Scheme 25, compounds of Formula 1f wherein Z³ is —C≡C— can serve as starting materials to prepare compounds of Formula 1f wherein Z³ is —CH₂CH₂— by reduction with hydrogen in the presence of a catalyst, for example, palladium on carbon.

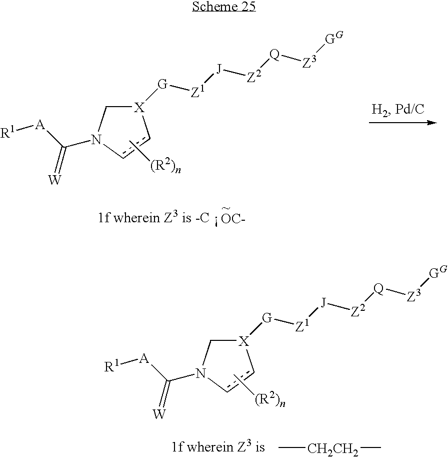

### G^{G} is G^{A}, G^{N} or G^{P}.

The reduction is typically carried out under an atmosphere of hydrogen at pressures from atmospheric to 700 kPa, preferably about 400 kPa, in a solvent such as ethyl acetate or ethanol using methods well known to one skilled in the art.

As shown in Scheme 26, preparation of the compounds of Formula 1f wherein Z³ is —C═C— includes the well-known Heck reaction using Pd-catalyzed cross-coupling of a halide of Formula 44 wherein Y¹⁰ is a halogen such as iodine or bromide with an alkene of Formula 45 in the presence of a metal catalyst and a base, such as triethylamine or sodium bicarbonate.

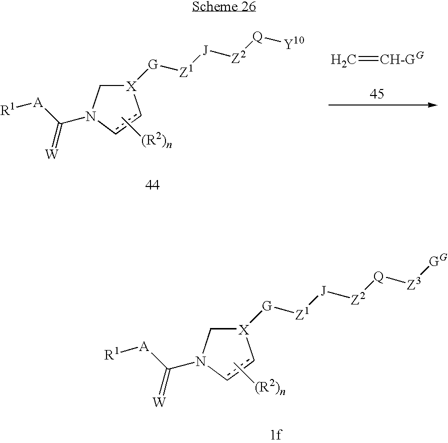

Y¹⁰ is Cl, Br, I, N₂⁺, OS(O)₂Ph or OS(O)₂CF₃; Z³ is —C═C—; G^{G} is a G^{A}, G^{N} or G^{P}.

Many catalysts are useful for this type of transformation; a typical catalyst is tris(dibenzylideneacetone)dipalladium. Suitable solvents include N,N-dimethylformamide and acetonitrile. For a review of the Heck reaction; see, for example, W. Cabri and I. Candiani, Acc. Chem. Res. 1995, 28, 2-7.

Compounds of Formula 1i (i.e. Formula 1 wherein Z³ is a direct bond and G^{G} is a tetrazole ring bonded to Q through the tetrazole ring carbon atom) can be prepared from nitriles of Formula 46 as shown in Scheme 27.

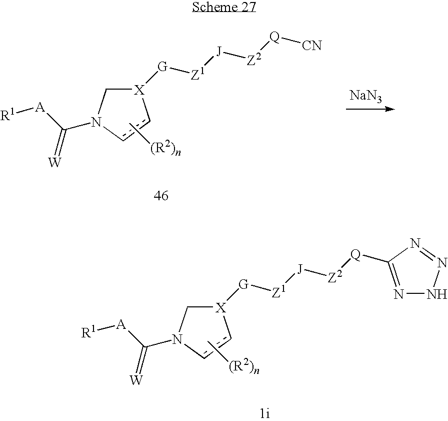

A nitrile of Formula 46 is reacted with an azide such as sodium azide or trimethylsilyl azide in a solvent such at N,N-dimethylformamide or toluene at temperatures from room temperature to 140° C. to form a compound of Formula 1i. For leading references; see, for example, B. Schmidt, D. Meid and D. Kieser, Tetrahedron 2006, 63, 492-496.

Aldehydes of Formula 47 can be used to prepare olefins of Formula 37a using the well-known Wittig (this method is demonstrated in Example 1, Step E) or Tebbe olefination reactions as shown in Scheme 28.

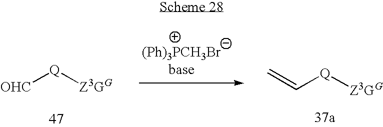

### G^{G} is G^{A}, G^{N} or G^{P}.

In the Wittig reaction, a methyltriphenylphosphonium halide such as methyltriphenylphosphonium bromide is reacted with a base such at t-BuOK. Tetrahydrofuran is a suitable solvent for this reaction. For additional leading references for the Wittig reaction; see, for example, A. Maercker Org. React. 1965, 14, 270-490; and for the Tebbe reaction; see, for example, H. Pommer, Angew. Chem. Int. Ed. Engl. 1977, 16, 423-429 and S. H. Pine, Org. React. 1993, 43 1-91. This method is demonstrated in Example 2, Step C. The olefins of Formula 37a are starting materials for the method shown in Scheme 20.

Reactions similar to those described in Scheme 21 can also be carried out on intermediates before coupling, for example, aldehydes of Formula 48 in Scheme 29 are useful starting materials to prepare the aldehydes of Formula 47.

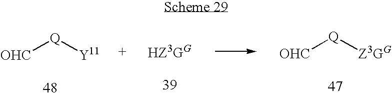

### Y¹¹ is F, Cl, Br, I; Z³ is O, S or NH; G^{G} is G^{A}, G^{N} or G^{P}.

The method of Scheme 29 using reagents and reaction conditions similar to those described for Scheme 21 provides, for example, the corresponding diaryl ether when Z³ is oxygen, (e.g., 2-phenoxybenzaldehyde is obtained starting with 2-iodobenzaldehyde and phenol). Several starting aldehydes of Formula 48 are commercially available, for example, the ortho, meta and para isomers of fluorobenzaldehyde, chlorobenzaldehyde, bromobenzaldehyde and iodobenzaldehyde.

Similarly, methods analogous to those described in Schemes 22-27 can also be used to prepare aldehydes of Formula 47; see, for example, W. Mansawat, et. al. Tetrahedron Letters 2007, 48(24), 4235-4238 for 2-(phenylthio)benzaldehyde; A. Cwik, Z. Hell, F. Figueras, Advanced Synthesis & Catalysis 2006, 348(4/5), 523-530 for 2-(2-phenylethenyl)benzaldehyde; T. Sakamoto, Y. Kondo, N. Miura, K. Hayashi, H. Yamanaka, Heterocycles 1986, 24(8), 2311-14 for 2-(phenylethynyl)benzaldehyde; and J. Rosevear, J. F. K. Wilshire, John F. K. Australian Journal of Chemistry 1991, 44(8), 1097-114 for 2-(1H-1,2,4-triazol-1-yl)benzaldehyde.

Several aldehydes of Formula 47 are also commercially available including 2-phenylbenzaldehyde, 2-phenoxybenzaldehyde 2-(furan-2-yl)benzaldehyde, 2-(thien-2-yl)benzaldehyde, 2-(imidazol-1-yl)benzaldehyde and 2-(thiazol-2-yl)benzaldehyde.

It is recognized that some reagents and reaction conditions described above for preparing compounds of Formulae 1 and 1A may not be compatible with certain functionalities present in the intermediates. In these instances, the incorporation of protection/deprotection sequences or functional group interconversions into the synthesis will aid in obtaining the desired products. The use and choice of the protecting groups will be apparent to one skilled in chemical synthesis (see, for example, T. W. Greene and P. G. M. Wuts, Protective Groups in Organic Synthesis, 2nd ed.; Wiley: New York, 1991). One skilled in the art will recognize that, in some cases, after the introduction of a given reagent as it is depicted in any individual scheme, it may be necessary to perform additional routine synthetic steps not described in detail to complete the synthesis of compounds of Formulae 1 and 1A. One skilled in the art will also recognize that it may be necessary to perform a combination of the steps illustrated in the above schemes in an order other than that implied by the particular sequence presented to prepare the compounds of Formulae 1 and 1A.

One skilled in the art will also recognize that compounds of Formulae 1 and 1A and the intermediates described herein can be subjected to various electrophilic, nucleophilic, radical, organometallic, oxidation, and reduction reactions to add substituents or modify existing substituents.

Without further elaboration, it is believed that one skilled in the art using the preceding description can utilize the present invention to its fullest extent. The following Examples are, therefore, to be construed as merely illustrative, and not limiting of the disclosure in any way whatsoever. Steps in the following Examples illustrate a procedure for each step in an overall synthetic transformation, and the starting material for each step may not have necessarily been prepared by a particular preparative run whose procedure is described in other Examples or Steps. Percentages are by weight except for chromatographic solvent mixtures or where otherwise indicated. Parts and percentages for chromatographic solvent mixtures are by volume unless otherwise indicated. ¹H NMR spectra are reported in ppm downfield from tetramethylsilane; “s” means singlet, “d” means doublet, “t” means triplet, “m” means multiplet, “q” means quartet, “dd” means doublet of doublet, “br s” means broad singlet, “br d” means broad doublet, “br t” means broad triplet, “br m” means broad multiplet.

### Example 1

### Preparation of 1-[4-[4-[4,5-dihydro-5-[3-(1H-1,2,4-triazol-1-yl)phenyl]-3-isoxazolyl]-2-thiazolyl]-1-piperidinyl]-2-[5-methyl-3-(trifluoromethyl)-1H-pyrazol-1-yl]ethanone (Compound 1)

### Step A: Preparation of 1-(2-chloroacetyl)-4-piperidinecarbonitrile

A mixture of 4-piperidinecarbonitrile (200 g, 1.80 mol) and 40% aqueous potassium carbonate solution (342 g, 0.99 mol) in dichloromethane (1 L) was cooled to −10° C., and a solution of chloroacetyl chloride (210 g, 1.86 mol) in dichloromethane (300 mL) was added over about 75 minutes while maintaining the reaction mixture at −10 to 0° C. After the addition was complete, the reaction mixture was separated, the upper aqueous phase was extracted with dichloromethane (2×300 mL), and the combined organic phases were concentrated under reduced pressure to give 312 g of the title compound as a liquid which slowly crystallized on standing. This compound was of sufficient purity to use in subsequent reactions.

¹H NMR (CDCl₃) δ 1.8-2.1 (m, 4H), 2.95 (m, 1H), 3.5-3.8 (m, 4H), 4.08 (q, 2H).

### Step A1: Alternative Preparation of 1-(2-chloroacetyl)-4-piperidinecarbonitrile

A solution of N-(1,1-dimethylethyl)-4-piperidinecarboxamide (201 g, 1.0 mol) in dichloromethane (1 L) was cooled under nitrogen to −5° C., and chloroacetyl chloride (124 g, 1.1 mol) in 300 mL of dichloromethane was added dropwise over 30 minutes while maintaining the reaction mixture at 0 to 5° C. Then 20% aqueous potassium carbonate solution (450 g, 0.65 mol) was added dropwise over 30 minutes while keeping reaction mixture between 0 and 5° C. The reaction mixture was stirred for an additional 30 minutes at 0° C., and then allowed to warm to room temperature. The layers were separated, and the aqueous layer was extracted with dichloromethane (200 mL). The combined dichloromethane layers were concentrated under reduced pressure to yield a solid, which was triturated with 400 mL of hexanes. The slurry was filtered, and the filter cake was washed with 100 mL of hexanes and dried in a vacuum oven overnight at 50° C. to give 185.5 g of 1-(2-chloroacetyl)-N-(1,1-dimethylethyl)-4-piperidinecarboxamide as a solid, melting at 140.5-141.5° C.

¹H NMR (CDCl₃) δ 1.35 (s, 9H), 1.6-2.0 (m, 4H), 2.25 (m, 1H), 2.8 (t, 1H), 3.2 (t, 1H), 3.9 (d, 1H), 4.07 (s, 2H), 4.5 (d, 1H), 5.3 (br s, 1H).

To a solution of 1-(2-chloroacetyl)-N-(1,1-dimethylethyl)-4-piperidinecarboxamide (26.1 g, 0.10 mol) in N,N-dimethylformamide (35 mL) was added phosphorus oxychloride (18.8 g, 0.123 mol) dropwise over 30 minutes while allowing the temperature of the reaction mixture to rise to 37° C. The reaction mixture was heated at 55° C. for 1 h and then was slowly added to water (about 150 g) cooled with ice to maintain a temperature of about 10° C. The pH of the reaction mixture was adjusted to 5.5 with 50% NaOH aqueous solution. The mixture was extracted with dichloromethane (4×100 mL), and the combined extract was concentrated under reduced pressure to give 18.1 g of the title compound as a solid. This compound was of sufficient purity to use in subsequent reactions.

### Step B: Preparation of 1-[2-[5-methyl-3-(trifluoromethyl)-1H-pyrazol-1-yl]acetyl]-4-piperidinecarbonitrile

A solution of 3-methyl-5-trifluoromethylpyrazole (9.3 g, 62 mmol) and 45% aqueous potassium hydroxide solution (7.79 g, 62 mmol) in N,N-dimethylformamide (25 mL) was cooled to 5° C., and 1-(2-chloroacetyl)-4-piperidinecarbonitrile (i.e. the product of Example 1, Step A or A1) (11.2 g, 60 mmol) was added. The reaction mixture was stirred for 8 h at 5-10° C., then diluted with water (100 mL), and filtered. The filter cake was washed with water and dried at 50° C. in a vacuum-oven to give 15 g of the title compound as a solid containing 3% of its regioisomer, i.e. 1-[2-[3-methyl-5-(trifluoromethyl)-1H-pyrazol-1-yl]acetyl]-4-piperidinecarbonitrile.

¹H NMR (CDCl₃) δ 1.88 (m, 4H), 2.32 (s, 3H), 2.95 (m, 1H), 3.7 (m, 4H), 5.0 (q, 2H), 6.34 (s, 1H).

### Step C: Preparation of 1-[2-[5-methyl-3-(trifluoromethyl)-1H-pyrazol-1-yl]acetyl]-4-piperidinecarbothioamide

Hydrogen sulfide gas was passed into a solution of 1-[2-[5-methyl-3-(trifluoromethyl)-1H-pyrazol-1-yl]acetyl]-4-piperidinecarbonitrile (i.e. the product of Example 1, Step B) (9.0 g, 30 mmol) and diethanolamine (3.15 g, 30 mmol) in N,N-dimethylformamide (15 mL) at 50° C. in a flask equipped with dry-ice condenser. The hydrogen sulfide feed was stopped when the reaction mixture became saturated with hydrogen sulfide, as indicated by condensation on the cold-finger. The reaction mixture was stirred for an additional 30 minutes at 50° C. Then excess hydrogen sulfide gas was sparged into the scrubber by a subsurface nitrogen flow, and water (70 mL) was gradually added. The reaction mixture was cooled to 5° C., filtered, and washed with water (2×30 mL). The filter cake was dried at 50° C. in a vacuum-oven to give 8.0 g of the title compound as a solid, melting at 185-186° C.

¹H NMR (CDCl₃) δ 1.7 (m, 2H), 2.0 (m, 2H), 2.29 (s, 3H), 2.65 (t, 1H), 3.0 (m, 1H), 3.2 (t, 1H), 4.0 (d, 1H), 4.6 (d, 1H), 4.96 (d, 1H), 5.4 (d, 1H), 6.35 (s, 1H), 7.4 (br s, 1H), 7.5 (br s, 1H).

### Step D: Preparation of 3-chloro-N-hydroxy-2-oxo-propanimidoyl chloride

To a solution of 1,3-dichloroacetone (100 g, 0.79 mol) in 2 M solution of hydrogen chloride in diethyl ether (400 mL) at 15° C. was added t-butyl nitrite (55 g, 0.534 mol) over 10 minutes. The reaction progress was monitored by ¹H NMR to obtain ˜85% conversion with no more than 3% of the bis-nitrosation side product. The reaction mixture was concentrated under reduced pressure to leave a semi-solid, which was then thoroughly rinsed with chlorobutane. The resulting solid was collected under filtration to give a 77 g of the title compound as a white solid. The filtrate was further concentrated under reduced pressure to give a semi-solid residue, which was rinsed with additional chlorobutane. The resulting solid was collected under filtration to give additional 15 g of the title compound as a white solid.

¹H NMR (DMSO-d₆) δ 4.96 (s, 2H), 13.76 (s, 1H).

### Step E: Preparation of 1-ethenyl-3-iodobenzene

A mixture of 3-iodobenzaldehyde (2.0 g, 8.6 mmol) and methyltriphenylphosphonium bromide (4.62 g, 12.9 mmol) in tetrahydrofuran (50 mL) was cooled to 0° C., and a solution of potassium tert-butoxide (1.45 g, 12.9 mmol) in tetrahydrofuran (20 mL) was added dropwise at 0° C. over 1 h. The reaction mixture was allowed to warm to room temperature and stirred for 12 h. The reaction mixture was filtered through Celite® diatomaceous filter aid with hexane, treated with DARCO® activated charcoal, and filtered a second time. The resulting oil was purified by column chromatography on silica gel using 100% hexane to 10% ethyl acetate in hexanes as eluant to give 1.82 g of the title compound as a yellow oil.

¹H NMR (CDCl₃) δ 5.28 (d, 1H), 5.74 (d, 1H), 6.60 (dd, 1H), 7.05 (t, 1H), 7.35 (d, 1H), 7.56-7.59 (m, 1H), 7.74-7.77 (m, 1H).

### Step F: Preparation of 2-chloro-1-[4,5-dihydro-5-(3-iodophenyl)-3-isoxazolyl]ethanone

To a solution of 1-ethenyl-3-iodobenzene (i.e. the product of Example 1, Step E) (1.82 g, 7.9 mmol) and 3-chloro-N-hydroxy-2-oxo-propanimidoyl chloride (i.e. the product of Example 1, Step D) (1.23 g, 7.9 mmol) in acetonitrile (32 mL) was added sodium bicarbonate (1.99 g, 23.7 mmol), and the reaction mixture was stirred at room temperature for 12 h. The reaction mixture was concentrated, taken up in water and extracted with dichloromethane, filtered through a ChemElute® diatomaceous earth-based liquid-liquid exchange cartridge, and concentrated to give 2.38 g of the title compound as a yellow oil.

¹H NMR (CDCl₃) δ 3.17 (dd, 1H), 3.62 (dd, 1H), 4.72 (s, 2H), 5.74 (dd, 1H), 7.13 (t, 1H), 7.24-7.28 (m, 1H), 7.63-7.72 (m, 2H).

### Step G: Preparation of 1-[4-[4-[4,5-dihydro-5-(3-iodophenyl)-3-isoxazolyl]-2-thiazolyl]-1-piperidinyl]-2-[5-methyl-3-(trifluoromethyl)-1H-pyrazol-1-yl]ethanone

To a mixture of 2-chloro-1-[4,5-dihydro-5-(3-iodophenyl)-3-isoxazolyl]ethanone (i.e. the product of Example 1, Step F) (2.38 g, 7.8 mmol) and tetrabutylammonium bromide (238 mg, 0.74 mmol) in acetone (50 mL) was added 1-[2-[5-methyl-3-(trifluoromethyl)-1H-pyrazol-1-yl]acetyl]-4-piperidinecarbothioamide (i.e. the product of Example 1, Step C) (2.56 g, 7.7 mmol). The reaction mixture was refluxed for 12 h. After cooling the reaction mixture was concentrated and then taken up in water. The pH was adjusted to 8 with saturated sodium bicarbonate, 1.5 mL Clorox® sodium hypochlorite bleach was added, and the mixture was extracted 2 times with ethyl acetate. The combined organic extracts were washed with brine, dried over magnesium sulfate, treated with DARCO®, filtered through Celite® diatomaceous filter aid, and concentrated. The resulting oil was purified by column chromatography on silica gel using 20% ethyl acetate in hexanes to 50% acetone in hexanes as eluant to give 2.76 g of the title compound as a light yellow solid foam.

¹H NMR (CDCl₃) δ 1.70-1.85 (m, 2H), 2.20 (br t, 2H), 2.32 (s, 3H), 2.90 (t, 1H), 3.25-3.45 (m, 4H), 3.85 (dd, 1H), 4.05 (d, 1H), 4.58 (d, 1H), 4.95-5.05 (m, 2H), 5.70 (dd, 1H), 7.11 (t, 1H), 7.35 (d, 1H), 7.60-7.70 (m, 2H) 7.75 (s, 1H).

### Step H: Preparation of 1-[4-[4-[4,5-dihydro-5-[3-(1H-1,2,4-triazol-1-yl)phenyl]-3-isoxazolyl]-2-thiazolyl]-1-piperidinyl]-2-[5-methyl-3-(trifluoromethyl)-1H-pyrazol-1-yl]ethanone

Sodium 1,2,4-triazole (63.0 mg, 0.69 mmol) was added to a mixture of 1-[4-[4-[4,5-dihydro-5-(3-iodophenyl)-3-isoxazolyl]-2-thiazolyl]-1-piperidinyl]-2-[5-methyl-3-(trifluoromethyl)-1H-pyrazol-1-yl]ethanone (i.e. the product of Example 1, Step G), (217 mg, 0.34 mmol), (+)-sodium L-ascorbate (3.4 mg, 0.017 mmol), copper iodide (6.6 mg, 0.034 mmol) and (1R,2R)-N,N-dimethyl-1,2-cyclohexenediamine (7.3 mg, 0.051 mmol) in 2 mL of an 80:20 solution of dimethylsulfoxide and water. The reaction mixture was heated at 60° C. for 20 h and then at 100° C. for 24 h. After cooling, the reaction mixture was diluted with water and extracted 2 times with ethyl acetate. The combined organic extracts were washed 5 times with water, then brine, and dried over magnesium sulfate, filtered and concentrated. The resulting oil was purified by column chromatography on silica gel using 75% ethyl acetate in hexanes as eluant to give 49 mg of the title compound, a compound of the present invention, as a pale yellow solid foam, melting at 83-85° C.

¹H NMR (CDCl₃) δ 1.68-1.89 (m, 2H), 2.19 (br t, 2H), 2.32 (s, 3H), 2.83-2.94 (m, 1H), 3.25-3.36 (m, 2H), 3.46 (dd, 1H), 3.94 (dd, 1H), 4.05 (d 1H), 4.57 (d, 1H), 4.91-5.05 (m, 2H), 5.84 (dd, 1H), 6.33 (s, 1H), 7.42-7.46 (m, 1H), 7.53 (t, 1H), 7.62-7.67 (m, 2H), 7.73-7.76 (m, 1H), 8.10 (s, 1H), 8.59 (s, 1H).

### Example 2

### Preparation of 4-[4-(5-[1,1′-biphenyl]-2-yl-4,5-dihydro-3-isoxazolyl)-2-thiazolyl]-N-(2,5-dimethylphenyl)-1-piperidinecarboxamide (Compound 17)

### Step A: Preparation of 4-cyano-N-(2,5-dimethylphenyl)-1-piperidinecarboxamide

A solution of 4-cyanopiperidine (11.0 g, 100 mmol) in diethyl ether (350 mL) was cooled to 0° C. with an ice-water bath. A solution of 2-isocyanato-1,4-dimethylbenzene (14.7 g, 100 mmol) in diethyl ether (50 mL) was added into the reaction mixture over 30 minutes to give a thick precipitate. The reaction mixture was warmed to room temperature, and the resulting solids were filtered, washed with diethyl ether and air-dried to give 25.3 g of the title compound as a white powder, melting at 187-190° C.

¹H NMR (CDCl₃): δ 1.95 (m, 4H), 2.19 (s, 3H), 2.30 (s, 3H), 2.90 (m, 1H), 3.45 (m, 2H), 3.70 (m, 2H), 6.10 (br s, 1H), 6.85 (m, 1H), 7.04 (m, 1H), 7.37 (m, 1H).

### Step B: Preparation of 4-(aminothioxomethyl)-N-(2,5-dimethylphenyl)-1-piperidine-carboxamide

A mixture of 4-cyano-N-(2,5-dimethylphenyl)-1-piperidinecarboxamide (i.e. the product of Example 2, Step A) (12.75 g, 49.6 mmol), sodium hydrosulfide hydrate (11.1 g, 150 mmol) and diethylamine hydrochloride (10.9 g, 100 mmol) in N,N-dimethylformamide (50 mL) was stirred at room temperature for 3 days. The resulting thick, green suspension was added dropwise into ice water (600 mL). The resulting solid was filtered, washed with water and air-dried to give 12.5 g of the title compound as a tan solid decomposing at 155-156° C.

¹H NMR (DMSO-d₆): δ 1.67 (m, 4H), 2.10 (s, 3H), 2.23 (s, 3H), 2.75 (m, 3H), 4.15 (m, 2H), 6.85 (m, 1H), 7.0 (m, 1H), 7.05 (m, 1H), 7.95 (br s, 1H), 9.15 (br s, 1H), 9.22 (br s, 1H).

### Step C: Preparation of 2-ethenyl-1,1′-biphenyl

A mixture of [1,1′-biphenyl]-2-carboxaldehyde (2.00 g, 11.0 mmol) and methyltriphenylphosphonium bromide (5.88 g, 16.5 mmol) in tetrahydrofuran (40 mL) was cooled to 0° C., and a solution of potassium tert-butoxide (1.85 g, 16.5 mmol) in tetrahydrofuran (20 mL) was added dropwise at 0° C. over 1 h. The reaction mixture was allowed to warm to room temperature and stirred for 12 h, then filtered through Celite® diatomaceous filter aid with hexane and concentrated under reduced pressure. The resulting oil was treated with hexanes, filtered again, concentrated under reduced pressure and purified by column chromatography on silica gel using 100% hexane to 10% ethyl acetate in hexanes as eluant to give 1.69 g of the title compound as a colorless oil.

¹H NMR (CDCl₃) δ 5.18 (dd, 1H), 5.70 (dd, 1H), 6.71 (dd, 1H), 7.27-7.44 (m, 8H), 7.62-7.66 (m, 1H).

### Step D: Preparation of 1-(5-[1,1′-biphenyl]-2-yl-4,5-dihydro-3-isoxazolyl)-2-chloroethanone

To a solution of 2-ethenyl-1,1′-biphenyl (i.e. the product of Example 2, Step C) (750 mg, 4.17 mmol) and 3-chloro-N-hydroxy-2-oxo-propanimidoyl chloride (i.e. the product of Example 1, Step D) (646 mg, 4.17 mmol) in acetonitrile (13 mL) was added sodium bicarbonate (1.05 g, 12.5 mmol), and the reaction mixture was stirred at room temperature for 2 days. The reaction mixture was concentrated under reduced pressure. The resultant residue was taken up in ethyl acetate, 2 mL of water added and eluted with ethyl acetate through a ChemElute® diatomaceous earth-based liquid-liquid exchange cartridge and concentrated to give 630 mg of the title compound as a colorless oil.

¹H NMR (CDCl₃) δ 3.14 (dd, 1H), 3.37 (dd, 1H), 4.63-4.73 (m, 2H), 5.79 (dd, 1H), 7.26-7.46 (m, 9H).

### Step E: Preparation of 4-[4-(5-[1,1′-biphenyl]-2-yl-4,5-dihydro-3-isoxazolyl)-2-thiazolyl]-N-(2,5-dimethylphenyl)-1-piperidinecarboxamide

To a mixture of 1-(5-[1,1′-biphenyl]-2-yl-4,5-dihydro-3-isoxazolyl)-2-chloroethanone (i.e. the product of Example 2, Step D) (200 mg, 0.67 mmol) and 4-(aminothioxomethyl)-N-(2,5-dimethylphenyl)-1-piperidine-carboxamide (i.e. the product of Example 2, Step B) (195 mg, 0.67 mmol) in acetonitrile (5 mL) was added sodium bromide (103 mg, 1.00 mmol). The reaction mixture was refluxed overnight and then concentrated under reduced pressure. The crude residue was added to water and sodium bicarbonate (56 mg, 0.67 mmol) and then extracted three times with ethyl acetate. The combined organic extracts were washed with brine, dried over magnesium sulfate and concentrated under reduced pressure. The resulting oil was purified by column chromatography on silica gel using 20% ethyl acetate in hexanes to 100% ethyl acetate as eluant to give 139 mg of the title compound, a compound of the present invention, as a solid white foam melting at 77-79° C.

¹H NMR (CDCl₃) δ 1.77-1.89 (m, 2H), 2.12-2.21 (m, 5H), 2.29 (s, 3H), 3.00-3.09 (m, 2H), 3.20-3.29 (m, 1H), 3.36 (dd, 1H), 3.60 (dd 1H), 4.11-4.18 (m, 2H), 5.74 (dd, 1H), 6.24 (br s, 1H), 6.82 (d 1H), 7.03 (d, 1H), 7.24-7.47 (m, 9H), 7.57 (s. 1H), 7.60 (dd, 1H).

By the procedures described herein, together with methods known in the art, the following compounds of Tables 1 to 15 can be prepared. The following abbreviations are used in the Tables which follow: t means tertiary, s means secondary, n means normal, i means iso, c means cyclo, Ac means acetyl, Me means methyl, Et means ethyl, Pr means propyl (i.e. n-propyl), i-Pr means isopropyl, c-Pr means cyclopropyl, Bu means butyl, Pen means pentyl, Hex means hexyl, Am means amyl, CN means cyano, SO₂ means sulfonyl (S(═O)₂). A dash (-) indicates no substituents.

The invention includes but is not limited to the following exemplary species.

TABLE 1

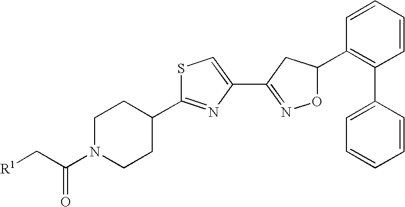

R¹
phenyl
2-methylphenyl
2-methoxyphenyl
2-chlorophenyl
2-bromophenyl
2-ethylphenyl
2-ethoxyphenyl
2-(methylthio)phenyl
2-(ethylthio)phenyl
2-(trifluoromethoxy)phenyl
3-chlorophenyl
3-bromophenyl
3-iodophenyl
3-methylphenyl
2-chloro-5-(trifluoromethyl)phenyl
2-chloro-5-(2,2,2-trifluoroethyl)phenyl
2-chloro-5-(pentafluoroethyl)phenyl
3-ethylphenyl
3-propylphenyl
3-isopropylphenyl
3-(trifluoromethyl)phenyl
3-(2,2,2-trifluoroethyl)phenyl
3-(pentafluoroethyl)phenyl
3-cyanophenyl
3-nitrophenyl
2,5-dichlorophenyl
5-bromo-2-chlorophenyl
2-chloro-5-iodophenyl
2-chloro-5-methylphenyl
2-chloro-5-ethylphenyl
2-chloro-5-propylphenyl
2-chloro-5-isopropylphenyl
5-ethyl-2-methoxyphenyl
2-methoxy-5-propylphenyl
2-chloro-5-cyanophenyl
2-chloro-5-nitrophenyl
2-bromo-5-chlorophenyl
2,5-dibromophenyl
2-bromo-5-iodophenyl
2-bromo-5-methylphenyl
2-bromo-5-ethylphenyl
2-bromo-5-propylphenyl
2-bromo-5-isopropylphenyl
2-bromo-5-(trifluoromethyl)phenyl
2-bromo-5-(2,2,2-trifluoroethyl)phenyl
2-bromo-5-(pentafluoroethyl)phenyl
2-bromo-5-cyanophenyl
2-bromo-5-nitrophenyl
5-chloro-2-methylphenyl
5-bromo-2-methylphenyl
5-iodo-2-methylphenyl
2,5-dimethylphenyl
5-ethyl-2-methylphenyl
2-methyl-5-propylphenyl
5-isopropyl-2-methylphenyl
2-methyl-5-(trifluoromethyl)phenyl
2-methyl-5-(2,2,2-trifluoroethyl)phenyl
2-methyl-5-(pentafluoroethyl)phenyl
5-cyano-2-methylphenyl
2-methyl-5-nitrophenyl
5-chloro-2-methoxyphenyl
5-bromo-2-methoxyphenyl
5-iodo-2-methoxyphenyl
2-methoxy-5-methylphenyl
3-iodo-5-methylpyrazol-1-yl
3-ethyl-5-methylpyrazol-1-yl
5-methyl-3-propylpyrazol-1-yl
3-isopropyl-5-methylpyrazol-1-yl
5-methyl-3-(trifluoromethyl)pyrazol-1-yl
5-methyl-3-(2,2,2-trifluoroethyl)pyrazol-1-yl
5-methyl-3-(pentafluoroethyl)pyrazol-1-yl
5-isopropyl-2-methoxyphenyl
2-methoxy-5-(trifluoromethyl)phenyl
2-methoxy-5-(2,2,2-trifluoroethyl)phenyl
2-methoxy-5-(pentafluoroethyl)phenyl
5-cyano-2-methoxyphenyl
2-methoxy-5-nitrophenyl
5-chloro-2-ethylphenyl
5-bromo-2-ethylphenyl
2-ethyl-5-iodophenyl
2-ethyl-5-methylphenyl
2,5-diethylphenyl
2-ethyl-5-propylphenyl
2-ethyl-5-isopropylphenyl
2-ethyl-5-(trifluoromethyl)phenyl
2-ethyl-5-(2,2,2-trifluoroethyl)phenyl
2-ethyl-5-(pentafluoroethyl)phenyl
5-cyano-2-ethylphenyl
2-ethyl-5-nitrophenyl
3-methylpyrazol-1-yl
3-chloropyrazol-1-yl
3-bromopyrazol-1-yl
3-iodopyrazol-1-yl
3-ethylpyrazol-1-yl
3-(trifluoromethyl)pyrazol-1-yl
3-(2,2,2-trifluoroethyl)pyrazol-1-yl
3-(pentafluoroethyl)pyrazol-1-yl
3-cyanopyrazol-1-yl
3-nitropyrazol-1-yl
3,5-dimethylpyrazol-1-yl
3-chloro-5-methylpyrazol-1-yl
3-bromo-5-methylpyrazol-1-yl
5-methoxy-3-methylpyrazol-1-yl
3-chloro-5-methoxypyrazol-1-yl
5-ethyl-3-methylpyrazol-1-yl
3-chloro-5-ethylpyrazol-1-yl
3-bromo-5-ethylpyrazol-1-yl
5-ethyl-3-iodopyrazol-1-yl
3-cyano-5-methylpyrazol-1-yl
5-methyl-3-nitropyrazol-1-yl
5-chloro-3-methylpyrazol-1-yl
3,5-dichloropyrazol-1-yl
5-chloro-3-bromopyrazol-1-yl
5-chloro-3-iodopyrazol-1-yl
5-chloro-3-ethylpyrazol-1-yl
5-chloro-3-propylpyrazol-1-yl
5-chloro-3-isopropylpyrazol-1-yl
5-chloro-3-(trifluoromethyl)pyrazol-1-yl
5-chloro-3-(2,2,2-trifluoroethyl)pyrazol-1-yl
5-chloro-3-(pentafluoroethyl)pyrazol-1-yl
5-chloro-3-cyanopyrazol-1-yl
5-chloro-3-nitropyrazol-1-yl
5-bromo-3-methylpyrazol-1-yl
5-bromo-3-chloropyrazol-1-yl
3,5-dibromopyrazol-1-yl
5-bromo-3-iodopyrazol-1-yl
5-bromo-3-ethylpyrazol-1-yl
5-bromo-3-propylpyrazol-1-yl
5-bromo-3-isopropylpyrazol-1-yl
5-bromo-3-(trifluoromethyl)pyrazol-1-yl
5-bromo-3-(2,2,2-trifluoroethyl)pyrazol-1-yl
5-bromo-3-(pentafluoroethyl)pyrazol-1-yl
5-bromo-3-cyanopyrazol-1-yl
5-bromo-3-nitropyrazol-1-yl
2-chloro-5-(dimethylamino)phenyl
2-chloro-5-(diethylamino)phenyl
2-chloro-5-(cyclopropylamino)phenyl
3-(methoxymethyl)phenyl
2-chloro-5-(ethoxymethyl)phenyl
2-chloro-5-(hyroxymethyl)phenyl
2-chloro-5-(methoxycarbonyl)phenyl
2-chloro-5-(ethylcarbonyl)phenyl
2-chloro-5-(methylcarbonyloxy)phenyl
2-chloro-5-(metylaminocarbonyl)phenyl
2-chloro-5-(dimethylaminocarbonyl)phenyl
3,5-diethylpyrazol-1-yl
5-ethyl-3-propylpyrazol-1-yl
5-ethyl-3-isopropylpyrazol-1-yl
5-ethyl-3-(trifluoromethyl)pyrazol-1-yl
5-ethyl-3-(2,2,2-trifluoroethyl)pyrazol-1-yl
5-ethyl-3-(pentafluoroethyl)pyrazol-1-yl
3-cyano-5-ethylpyrazol-1-yl
5-ethyl-3-nitropyrazol-1-yl
5-butyl-2-methylphenyl
5-hexyl-2-methylphenyl
5-allyl-2-methylphenyl
2-methyl-5-(4-methyl-3-pentenyl)phenyl
2-methyl-5-propargylphenyl
2-methyl-5-(3-methylpropargyl)phenyl
5-cyclopropyl-2-methylphenyl
5-cyclohexyl-2-methylphenyl
2-methyl-5-(pentafluoroisopropyl)phenyl
5-(3,3-dichloro-2-propen-1-yl)-2-methylphenyl
2-methyl-5-(4,4,4-trifluoro-2-butyn-1-yl)phenyl
5-(2,2-dichlorocyclopropan-1-yl)-2-methylphenyl
2-methyl-5-(trifluoromethoxy)phenyl
2-chloro-5-(isobutylthio)phenyl
2-chloro-5-(ethylsulfonyl)phenyl
2-chloro-5-(trifluoromethylthio)phenyl
2-chloro-5-(trifluoromethylsulfonyl)phenyl
2-chloro-5-(methylamino)phenyl
2-chloro-5-(tert-butylamino)phenyl
2,5-dimethyl-3-furyl
2,5-dimethyl-3-thienyl
2,5-dichloro-3-thienyl
1,4-dimethyl-3-pyrrolyl
1,4-dimethyl-3-pyrazolyl
1,3-dimethyl-4-pyrazolyl
2,5-dimethyl-4-oxazolyl
2,5-dimethyl-4-thiazolyl
3-bromo-4-isothiazolyl
3-bromo-4-isooxazolyl
2-methyl-5-(trimethylsilyl)phenyl
3,5-dimethyl-2-thienyl
3,5-dichloro-2-thienyl
3,5-dimethyl-2-furyl
1-methyl-2-pyrrolyl
4-methyl-2-(trifluoromethyl)-5-thiazolyl
4-(trifluoromethyl)-2-thiazolyl
4-(trifluoromethyl)-2-oxazolyl
4-methyl-2-(trifluoromethyl)-5-oxazolyl
4-bromo-5-isothiazolyl
4-bromo-5-isoxazolyl
1-methyl-5-pyrazolyl
1-methyl-5-imidazolyl
1-methyl-4-(trifluoromethyl)-2-imidazolyl
4-methyl-3-(1,3,4-triazolyl)
2-methyl-3-(1,2,4-triazolyl)
5-(trifluoromethyl)-2-(1,3,4-thiadiazolyl)
5-(trifluoromethyl)-2-(1,3,4-oxadiazolyl)
3-(trifluoromethyl)-5-(1,2,4-thiadiazolyl)
3-(trifluoromethyl)-5-(1,2,4-oxadiazolyl)
3-(trifluoromethyl)-1-(1,2,4-triazolyl)
2,5-dimethyl-1-pyrrolyl
1-methyl-3-(trifluoromethyl)pyrazol-5-yl
3-bromo-5-(trifluoromethyl)pyrazol-1-yl
3-iodo-5-(trifluoromethyl)pyrazol-1-yl
3-ethyl-5-(trifluoromethyl)-pyrazol-1-yl
3-propyl-5-(trifluoromethyl)pyrazol-1-yl
3-isopropyl-5-(trifluoromethyl)pyrazol-1-yl
3-methyl-5-(trifluoromethyl)-pyrazol-1-yl
3-methoxy-5-(trifluoromethyl)-pyrazol-1-yl
5-difluoromethoxy-3-methylpyrazol-1-yl
5-difluoromethoxy-3-chloropyrazol-1-yl
3,5-dibromopyrazol-1-yl
5-difluoromethoxy-3-iodopyrazol-1-yl
5-difluoromethoxy-3-ethylpyrazol-1-yl
5-difluoromethoxy-3-propylpyrazol-1-yl
5-difluoromethoxy-3-isopropylpyrazol-1-yl
1-methyl-4-imidazolyl
5-(trifluoromethyl)-3-(1,2,4-oxadiazolyl)
5-(trifluoromethyl)-3-(1,2,4-thiadiazolyl)
2-bromo-1-(1,3,4-triazolyl)
5-(trifluoromethyl)-3-(1,2,4-triazolyl)
2-bromo-1-imidazolyl
3,6-dimethyl-2-pyridyl
2,5-dimethyl-3-pyridyl
2,5-dimethyl-4-pyridyl
3,6-dichloro-2-pyridyl
2,5-dichloro-3-pyridyl
2,5-dichloro-4-pyridyl
4-bromo-3-pyridazinyl
4-(trifluoromethyl)-2-pyrimidinyl
3,6-dimethyl-2-pyrazinyl
2,5-dimethyl-4-pyrimidinyl
4-methoxy-5-pyrimidinyl
3,6-dimethyl-4-pyridazinyl
5-(trifluoromethyl)-3-(1,2,4-triazinyl)
5-methoxy-6-(1,2,4-triazinyl)
4-(trifluoromethyl)-2-(1,3,5-triazinyl)
3,6-dimethyl-5-(1,2,4-triazinyl)
1-methyl-4-(trifluoromethyl)imidazol-2-yl
3,5-bis-(trifluoromethyl)pyrazol-1-yl
3-(2,2,2-trifluoroethyl)-5-(trifluoromethyl)pyrazol-1-yl
3-(pentafluoroethyl)-5-(trifluoromethyl)pyrazol-1-yl
3-cyano-5-(trifluoromethyl)pyrazol-1-yl
3-nitro-5-(trifluoromethyl)pyrazol-1-yl
3-chloro-5-(trifluoromethyl)-pyrazol-1-yl
3,5-bis-(trichloromethyl)pyrazol-1-yl
3-difluoromethoxy-5-methylpyrazol-1-yl
3-difluoromethoxy-5-chloropyrazol-1-yl
3-difluoromethoxy-5-bromopyrazol-1-yl
3-difluoromethoxy-5-iodopyrazol-1-yl
3-difluoromethoxy-5-ethylpyrazol-1-yl
3-difluoromethoxy-5-(trifluoromethyl)pyrazol-1-yl
3-difluoromethoxy-5-(2,2,2-trifluoroethyl)pyrazol-1-yl
5-difluoromethoxy-3-(trifluoromethyl)pyrazol-1-yl
5-difluoromethoxy-3-(2,2,2-trifluoroethyl)pyrazol-1-yl
5-difluoromethoxy-3-(pentafluoroethyl)pyrazol-1-yl
5-difluoromethoxy-3-cyanopyrazol-1-yl
5-difluoromethoxy-3-nitropyrazol-1-yl
3-carbomethoxy-5-(trifluoromethyl)pyrazol-1-yl
5-methoxy-3-methylpyrazol-1-yl
5-methoxy-3-bromopyrazol-1-yl
5-methoxy-3-iodopyrazol-1-yl
5-methoxy-3-ethylpyrazol-1-yl
5-methoxy-3-propylpyrazol-1-yl
5-methoxy-3-isopropylpyrazol-1-yl
5-methoxy-3-(trifluoromethyl)pyrazol-1-yl
5-methoxy-3-(2,2,2-trifluoroethyl)pyrazol-1-yl
5-methoxy-3-(pentafluoroethyl)pyrazol-1-yl
5-methoxy-3-cyanopyrazol-1-yl
5-methoxy-3-nitropyrazol-1-yl
3-difluoromethoxy-5-(pentafluoroethyl)pyrazol-1-yl
3-difluoromethoxy-5-cyanopyrazol-1-yl
3-difluoromethoxy-5-nitropyrazol-1-yl
3,5-bis-(difluoromethoxy)pyrazol-1-yl
5-carbomethoxy-3-(trifluoromethyl)pyrazol-1-yl
3,5-dimethoxypyrazol-1-yl
5-ethoxy-3-methylpyrazol-1-yl
5-ethoxy-3-bromopyrazol-1-yl
5-ethoxy-3-iodopyrazol-1-yl
5-ethoxy-3-ethylpyrazol-1-yl
5-ethoxy-3-propylpyrazol-1-yl
5-ethoxy-3-isopropylpyrazol-1-yl
5-ethoxy-3-(trifluoromethyl)pyrazol-1-yl
5-ethoxy-3-(2,2,2-trifluoroethyl)pyrazol-1-yl
5-ethoxy-3-(pentafluoroethyl)pyrazol-1-yl
5-ethoxy-3-cyanopyrazol-1-yl
5-ethoxy-3-nitropyrazol-1-yl

TABLE 2

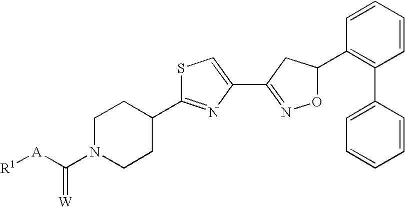

A is NH; W is O.
R¹
2-methoxyphenyl
2,5-dichlorophenyl
5-bromo-2-chlorophenyl
2-chloro-5-methylphenyl
2-chloro-5-(trifluoromethyl)phenyl
2,5-dibromophenyl
2-bromo-5-methylphenyl
2-bromo-5-(trifluoromethyl)phenyl
5-chloro-2-methylphenyl
5-bromo-2-methylphenyl
2,5-dimethylphenyl
5-ethyl-2-methylphenyl
2-methyl-5-(trifluoromethyl)phenyl
5-bromo-2-methoxyphenyl
2-methoxy-5-methylphenyl
2-methoxy-5-(trifluoromethyl)phenyl
3,5-dimethylpyrazol-1-yl
3,5-dichloropyrazol-1-yl
3,5-dibromopyrazol-1-yl
5-methyl-3-(trifluoromethyl)pyrazol-1-yl
5-chloro-3-(trifluoromethyl)pyrazol-1-yl
5-bromo-3-(trifluoromethyl)pyrazol-1-yl
5-ethyl-3-(trifluoromethyl)pyrazol-1-yl
3,5-bis-(trifluoromethyl)pyrazol-1-yl
3-methyl-5-(trifluoromethyl)pyrazol-1-yl
3-chloro-5-(trifluoromethyl)pyrazol-1-yl
3-bromo-5-(trifluoromethyl)pyrazol-1-yl
5-methoxy-3-(trifluoromethyl)pyrazol-1-yl
5-difluoromethoxy-3-(trifluoromethyl)pyrazol-1-yl
3,5-dichlorotriazol-1-yl
3,5-dibromotriazol-1-yl
A is CH₂; W is S
3-ethyl-5-methylpyrazol-1-yl
5-methyl-3-(trifluoromethyl)pyrazol-1-yl
3,5-dichloropyrazol-1-yl
5-chloro-3-(trifluoromethyl)pyrazol-1-yl
3,5-bis-(trifluoromethyl)pyrazol-1-yl
3,5-dimethylpyrazol-1-yl
3,5-dibromopyrazol-1-yl
5-bromo-3-(trifluoromethyl)pyrazol-1-yl
3,5-diethylpyrazol-1-yl
5-ethyl-3-(trifluoromethyl)pyrazol-1-yl
3,5-dichlorotriazol-1-yl
3,5-dibromotriazol-1-yl
3-methyl-5-(trifluoromethyl)pyrazol-1-yl
A is NH; W is S
2-methoxyphenyl
2,5-dichlorophenyl
5-bromo-2-chlorophenyl
2-chloro-5-methylphenyl
2-chloro-5-(trifluoromethyl)phenyl
2,5-dibromophenyl
2-bromo-5-methylphenyl
2-bromo-5-(trifluoromethyl)phenyl
5-chloro-2-methylphenyl
5-bromo-2-methylphenyl
2,5-dimethylphenyl
5-ethyl-2-methylphenyl
2-methyl-5-(trifluoromethyl)phenyl
5-bromo-2-methoxyphenyl
2-methoxy-5-methylphenyl
2-methoxy-5-(trifluoromethyl)phenyl
5-methyl-3-(trifluoromethyl)pyrazol-1-yl
R¹ is 5-methyl-3-(trifluoromethyl)pyrazol-1-yl; W is O.
A
NCH₃
NCO₂CH₃
NCH₂CO₂CH₃
NAc
CHCH₃
CHCO₂CH₃
CHCl
CHOCH₃

TABLE 3*

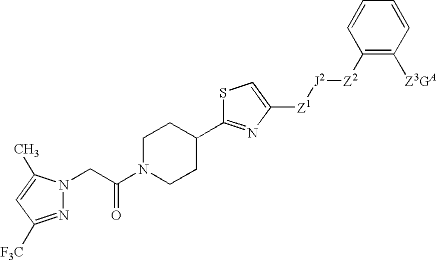

| J² | J-orientation** |
|---|---|
| J-1 | 2/4 |
| J-1 | 2/5 |
| J-1 | 4/2 |
| J-1 | 5/2 |
| J-2 | 2/4 |
| J-2 | 2/5 |
| J-2 | 4/2 |
| J-2 | 5/2 |
| J-3 | 2/4 |
| J-3 | 2/5 |
| J-3 | 4/2 |
| J-3 | 5/2 |
| J-3 | 1/4 |
| J-3 | 4/1 |
| J-4 | 2/4 |
| J-4 | 2/5 |
| J-4 | 4/2 |
| J-4 | 5/2 |
| J-4 | 3/5 |
| J-4 | 5/3 |
| J-5 | 2/4 |
| J-5 | 2/5 |
| J-5 | 4/2 |
| J-5 | 5/2 |
| J-5 | 3/5 |
| J-5 | 5/3 |
| J-6 | 2/4 |
| J-6 | 2/5 |
| J-6 | 4/2 |
| J-6 | 5/2 |
| J-6 | 3/5 |
| J-6 | 5/3 |
| J-6 | 1/3 |
| J-6 | 3/1 |
| J-7 | 5/3 |
| J-7 | 3/5 |
| J-8 | 5/3 |
| J-8 | 3/5 |
| J-9 | 5/3 |
| J-9 | 3/5 |
| J-9 | 1/4 |
| J-9 | 4/1 |
| J-10 | 3/5 |
| J-10 | 5/3 |
| J-11 | 3/5 |
| J-11 | 5/3 |
| J-12 | 3/5 |
| J-12 | 5/3 |
| J-12 | 1/3 |
| J-12 | 3/1 |
| J-13 | 1/4 |
| J-13 | 4/1 |
| J-14 | 3/5 |
| J-14 | 5/3 |
| J-15 | 2/5 |
| J-16 | 2/5 |
| J-17 | 2/4 |
| J-17 | 4/2 |
| J-18 | 2/5 |
| J-18 | 5/2 |
| J-19 | 2/4 |
| J-19 | 4/2 |
| J-20 | 2/4 |
| J-20 | 2/5 |
| J-20 | 2/6 |
| J-20 | 3/5 |
| J-20 | 4/2 |
| J-20 | 5/2 |
| J-21 | 3/5 |
| J-21 | 3/6 |
| J-21 | 5/3 |
| J-22 | 2/4 |
| J-22 | 2/5 |
| J-22 | 4/6 |
| J-22 | 4/2 |
| J-22 | 5/2 |
| J-23 | 2/5 |
| J-23 | 2/6 |
| J-24 | 2/4 |
| J-24 | 2/5 |
| J-24 | 4/2 |
| J-24 | 5/2 |
| J-25 | 2/4 |
| J-25 | 2/5 |
| J-25 | 4/2 |
| J-25 | 5/2 |
| J-26 | 2/4 |
| J-26 | 2/5 |
| J-26 | 4/2 |
| J-26 | 5/2 |
| J-26 | 1/4 |
| J-26 | 4/1 |
| J-27 | 2/4 |
| J-27 | 2/5 |
| J-27 | 3/5 |
| J-27 | 4/2 |
| J-27 | 5/2 |
| J-27 | 5/3 |
| J-28 | 3/5 |
| J-28 | 5/3 |
| J-29 | 3/5 |
| J-29 | 5/3 |
| J-30 | 3/5 |
| J-30 | 5/3 |
| J-30 | 1/3 |
| J-30 | 3/1 |
| J-30 | 1/4 |
| J-30 | 4/1 |
| J-31 | 1/3 |
| J-31 | 1/4 |
| J-31 | 2/4 |
| J-31 | 2/5 |
| J-31 | 3/5 |
| J-31 | 3/1 |
| J-31 | 4/1 |
| J-31 | 4/2 |
| J-31 | 5/2 |
| J-32 | 2/4 |
| J-32 | 2/5 |
| J-32 | 3/5 |
| J-32 | 5/3 |
| J-32 | 5/2 |
| J-32 | 4/2 |
| J-33 | 2/4 |
| J-33 | 2/5 |
| J-33 | 3/5 |
| J-33 | 5/3 |
| J-33 | 5/2 |
| J-33 | 4/2 |
| J-34 | 1/3 |
| J-34 | 1/4 |
| J-34 | 3/5 |
| J-34 | 3/1 |
| J-34 | 4/1 |
| J-35 | 1/4 |
| J-35 | 4/1 |
| J-36 | 1/3 |
| J-36 | 3/1 |
| J-36 | 3/5 |
| J-36 | 5/3 |
| J-37 | 2/5 |
| J-37 | 5/2 |
| J-37 | 2/4 |
| J-37 | 4/2 |
| J-38 | 2/5 |
| J-38 | 5/2 |
| J-38 | 2/4 |
| J-38 | 4/2 |
| J-39 | 3/5 |
| J-39 | 5/3 |
| J-40 | 3/5 |
| J-40 | 5/3 |
| J-41 | 1/3 |
| J-41 | 1/4 |
| J-42 | 1/3 |
| J-42 | 1/4 |
| J-43 | 1/4 |
| J-44 | 1/3 |
| J-44 | 2/4 |
| J-44 | 2/5 |
| J-44 | 2/6 |
| J-45 | 2/4 |
| J-45 | 2/5 |
| J-45 | 2/6 |
| J-46 | 2/4 |
| J-46 | 2/5 |
| J-46 | 4/2 |
| J-46 | 5/2 |
| J-47 | 2/4 |
| J-47 | 2/5 |
| J-47 | 4/2 |
| J-47 | 5/2 |
| J-48 | 3/5 |
| J-49 | 2/4 |
| J-49 | 2/5 |
| J-49 | 4/2 |
| J-49 | 5/2 |
| J-50 | 2/6 |
| J-51 | 2/6 |
| J-52 | 2/6 |
| J-53 | 2/3 |
| J-54 | 2/3 |
| J-55 | 2/3 |
| J-56 | 2/3 |
| J-57 | 2/4 |
| J-58 | 3/4 |
| J-59 | 2/4 |
| J-60 | 2/4 |
| J-61 | 2/4 |
| J-62 | 2/4 |
| J-63 | 3/4 |
| J-64 | 2/3 |
| J-65 | 3/4 |
| J-66 | 6/7 |
| J-67 | 2/3 |
| J-68 | 2/3 |
| J-69 | 1/3 |
| J-69 | 1/4 |
| J-70 | 1/3 |
| J-71 | 2/4 |
| J-71 | 4/2 |
| J-72 | 2/4 |
| J-72 | 4/2 |
| J-73 | 2/4 |
| J-73 | 4/2 |
| J-73 | 1/3 |
| J-73 | 1/4 |
| J-73 | 4/1 |
| J-74 | 2/4 |
| J-74 | 2/5 |
| J-74 | 4/2 |
| J-74 | 5/2 |
| J-74 | 3/5 |
| J-74 | 5/3 |
| J-75 | 3/5 |
| J-75 | 5/3 |
| J-75 | 2/4 |
| J-75 | 2/5 |
| J-75 | 3/5 |
| J-75 | 5/3 |
| J-76 | 3/6 |
| J-76 | 6/3 |
| J-77 | 3/5 |
| J-77 | 5/3 |
| J-78 | 1/3 |
| J-79 | 1/3 |
| J-79 | 3/1 |
| J-80 | 1/3 |
| J-80 | 3/1 |
| J-81 | 3/5 |
| J-81 | 5/3 |
| J-82 | 3/5 |
| J-82 | 3/6 |
| J-82 | 5/3 |
| J-82 | 6/3 |

Z¹ is a direct bond; Z² is a direct bond; Z³ is O; x is 0; G^{A} is G^{A}-49; r is 0.

| | |
|---|---|
| J-1 | 2/4 |
| J-1 | 2/5 |
| J-1 | 4/2 |
| J-1 | 5/2 |
| J-2 | 2/4 |
| J-2 | 2/5 |
| J-2 | 4/2 |
| J-2 | 5/2 |
| J-3 | 2/4 |
| J-3 | 2/5 |
| J-3 | 4/2 |
| J-3 | 5/2 |
| J-3 | 1/4 |
| J-3 | 4/1 |
| J-4 | 2/4 |
| J-4 | 2/5 |
| J-4 | 4/2 |
| J-4 | 5/2 |
| J-4 | 3/5 |
| J-4 | 5/3 |
| J-5 | 2/4 |
| J-5 | 2/5 |
| J-5 | 4/2 |
| J-5 | 5/2 |
| J-5 | 3/5 |
| J-5 | 5/3 |
| J-6 | 2/4 |
| J-6 | 2/5 |
| J-6 | 4/2 |
| J-6 | 5/2 |
| J-6 | 3/5 |
| J-6 | 5/3 |
| J-6 | 1/3 |
| J-6 | 3/1 |
| J-7 | 5/3 |
| J-7 | 3/5 |
| J-8 | 5/3 |
| J-8 | 3/5 |
| J-9 | 5/3 |
| J-9 | 3/5 |
| J-9 | 1/4 |
| J-9 | 4/1 |
| J-10 | 3/5 |
| J-10 | 5/3 |
| J-11 | 3/5 |
| J-11 | 5/3 |
| J-12 | 3/5 |
| J-12 | 5/3 |
| J-12 | 1/3 |
| J-12 | 3/1 |
| J-13 | 1/4 |
| J-13 | 4/1 |
| J-14 | 3/5 |
| J-14 | 5/3 |
| J-15 | 2/5 |
| J-16 | 2/5 |
| J-17 | 2/4 |
| J-17 | 4/2 |
| J-18 | 2/5 |
| J-18 | 5/2 |
| J-19 | 2/4 |
| J-19 | 4/2 |
| J-20 | 2/4 |
| J-20 | 2/5 |
| J-20 | 2/6 |
| J-20 | 3/5 |
| J-20 | 4/2 |
| J-20 | 5/2 |
| J-21 | 3/5 |
| J-21 | 3/6 |
| J-21 | 5/3 |
| J-22 | 2/4 |
| J-22 | 2/5 |
| J-22 | 4/6 |
| J-22 | 4/2 |
| J-22 | 5/2 |
| J-23 | 2/5 |
| J-23 | 2/6 |
| J-24 | 2/4 |
| J-24 | 2/5 |
| J-24 | 4/2 |
| J-24 | 5/2 |
| J-25 | 2/4 |
| J-25 | 2/5 |
| J-25 | 4/2 |
| J-25 | 5/2 |
| J-26 | 2/4 |
| J-26 | 2/5 |
| J-26 | 4/2 |
| J-26 | 5/2 |
| J-26 | 1/4 |
| J-26 | 4/1 |
| J-27 | 2/4 |
| J-27 | 2/5 |
| J-27 | 3/5 |
| J-27 | 4/2 |
| J-27 | 5/2 |
| J-27 | 5/3 |
| J-28 | 3/5 |
| J-28 | 5/3 |
| J-29 | 3/5 |
| J-29 | 5/3 |
| J-30 | 3/5 |
| J-30 | 5/3 |
| J-30 | 1/3 |
| J-30 | 3/1 |
| J-30 | 1/4 |
| J-30 | 4/1 |
| J-31 | 1/3 |
| J-31 | 1/4 |
| J-31 | 2/4 |
| J-31 | 2/5 |
| J-31 | 3/5 |
| J-31 | 3/1 |
| J-31 | 4/1 |
| J-31 | 4/2 |
| J-31 | 5/2 |
| J-32 | 2/4 |
| J-32 | 2/5 |
| J-32 | 3/5 |
| J-32 | 5/3 |
| J-32 | 5/2 |
| J-32 | 4/2 |
| J-33 | 2/4 |
| J-33 | 2/5 |
| J-33 | 3/5 |
| J-33 | 5/3 |
| J-33 | 5/2 |
| J-33 | 4/2 |
| J-34 | 1/3 |
| J-34 | 1/4 |
| J-34 | 3/5 |
| J-34 | 3/1 |
| J-34 | 4/1 |
| J-35 | 1/4 |
| J-35 | 4/1 |
| J-36 | 1/3 |
| J-36 | 3/1 |
| J-36 | 3/5 |
| J-36 | 5/3 |
| J-37 | 2/5 |
| J-37 | 5/2 |
| J-37 | 2/4 |
| J-37 | 4/2 |
| J-38 | 2/5 |
| J-38 | 5/2 |
| J-38 | 2/4 |
| J-38 | 4/2 |
| J-39 | 3/5 |
| J-39 | 5/3 |
| J-40 | 3/5 |
| J-40 | 5/3 |
| J-41 | 1/3 |
| J-41 | 1/4 |
| J-42 | 1/3 |
| J-42 | 1/4 |
| J-43 | 1/4 |
| J-44 | 1/3 |
| J-44 | 2/4 |
| J-44 | 2/5 |
| J-44 | 2/6 |
| J-45 | 2/4 |
| J-45 | 2/5 |
| J-45 | 2/6 |
| J-46 | 2/4 |
| J-46 | 2/5 |
| J-46 | 4/2 |
| J-46 | 5/2 |
| J-47 | 2/4 |
| J-47 | 2/5 |
| J-47 | 4/2 |
| J-47 | 5/2 |
| J-48 | 3/5 |
| J-49 | 2/4 |
| J-49 | 2/5 |
| J-49 | 4/2 |
| J-49 | 5/2 |
| J-50 | 2/6 |
| J-51 | 2/6 |
| J-52 | 2/6 |
| J-53 | 2/3 |
| J-54 | 2/3 |
| J-55 | 2/3 |
| J-56 | 2/3 |
| J-57 | 2/4 |
| J-58 | 3/4 |
| J-59 | 2/4 |
| J-60 | 2/4 |
| J-61 | 2/4 |
| J-62 | 2/4 |
| J-63 | 3/4 |
| J-64 | 2/3 |
| J-65 | 3/4 |
| J-66 | 6/7 |
| J-67 | 2/3 |
| J-68 | 2/3 |
| J-69 | 1/3 |
| J-69 | 1/4 |
| J-70 | 1/3 |
| J-71 | 2/4 |
| J-71 | 4/2 |
| J-72 | 2/4 |
| J-72 | 4/2 |
| J-73 | 2/4 |
| J-73 | 4/2 |
| J-73 | 1/3 |
| J-73 | 1/4 |
| J-73 | 4/1 |
| J-74 | 2/4 |
| J-74 | 2/5 |
| J-74 | 4/2 |
| J-74 | 5/2 |
| J-74 | 3/5 |
| J-74 | 5/3 |
| J-75 | 3/5 |
| J-75 | 5/3 |
| J-75 | 2/4 |
| J-75 | 2/5 |
| J-75 | 3/5 |
| J-75 | 5/3 |
| J-76 | 3/6 |
| J-76 | 6/3 |
| J-77 | 3/5 |
| J-77 | 5/3 |
| J-78 | 1/3 |
| J-79 | 1/3 |
| J-79 | 3/1 |
| J-80 | 1/3 |
| J-80 | 3/1 |
| J-81 | 3/5 |
| J-81 | 5/3 |
| J-82 | 3/5 |
| J-82 | 3/6 |
| J-82 | 5/3 |
| J-82 | 6/3 |

Z¹ is a direct bond; Z² is a direct bond; Z³ is CH₂; x is 0; G^{A} is G^{A}-49;
r is 0

| | |
|---|---|
| J-1 | 2/4 |
| J-1 | 2/5 |
| J-1 | 4/2 |
| J-1 | 5/2 |
| J-2 | 2/4 |
| J-2 | 2/5 |
| J-2 | 4/2 |
| J-2 | 5/2 |
| J-3 | 2/4 |
| J-3 | 2/5 |
| J-3 | 4/2 |
| J-3 | 5/2 |
| J-3 | 1/4 |
| J-3 | 4/1 |
| J-4 | 2/4 |
| J-4 | 2/5 |
| J-4 | 4/2 |
| J-4 | 5/2 |
| J-4 | 3/5 |
| J-4 | 5/3 |
| J-5 | 2/4 |
| J-5 | 2/5 |
| J-5 | 4/2 |
| J-5 | 5/2 |
| J-5 | 3/5 |
| J-5 | 5/3 |
| J-6 | 2/4 |
| J-6 | 2/5 |
| J-6 | 4/2 |
| J-6 | 5/2 |
| J-6 | 3/5 |
| J-6 | 5/3 |
| J-6 | 1/3 |
| J-6 | 3/1 |
| J-7 | 5/3 |
| J-7 | 3/5 |
| J-8 | 5/3 |
| J-8 | 3/5 |
| J-9 | 5/3 |
| J-9 | 3/5 |
| J-9 | 1/4 |
| J-9 | 4/1 |
| J-10 | 3/5 |
| J-10 | 5/3 |
| J-11 | 3/5 |
| J-11 | 5/3 |
| J-12 | 3/5 |
| J-12 | 5/3 |
| J-12 | 1/3 |
| J-12 | 3/1 |
| J-13 | 1/4 |
| J-13 | 4/1 |
| J-14 | 3/5 |
| J-14 | 5/3 |
| J-15 | 2/5 |
| J-16 | 2/5 |
| J-17 | 2/4 |
| J-17 | 4/2 |
| J-18 | 2/5 |
| J-18 | 5/2 |
| J-19 | 2/4 |
| J-19 | 4/2 |
| J-20 | 2/4 |
| J-20 | 2/5 |
| J-20 | 2/6 |
| J-20 | 3/5 |
| J-20 | 4/2 |
| J-20 | 5/2 |
| J-21 | 3/5 |
| J-21 | 3/6 |
| J-21 | 5/3 |
| J-22 | 2/4 |
| J-22 | 2/5 |
| J-22 | 4/6 |
| J-22 | 4/2 |
| J-22 | 5/2 |
| J-23 | 2/5 |
| J-23 | 2/6 |
| J-24 | 2/4 |
| J-24 | 2/5 |
| J-24 | 4/2 |
| J-24 | 5/2 |
| J-25 | 2/4 |
| J-25 | 2/5 |
| J-25 | 4/2 |
| J-25 | 5/2 |
| J-26 | 2/4 |
| J-26 | 2/5 |
| J-26 | 4/2 |
| J-26 | 5/2 |
| J-26 | 1/4 |
| J-26 | 4/1 |
| J-27 | 2/4 |
| J-27 | 2/5 |
| J-27 | 3/5 |
| J-27 | 4/2 |
| J-27 | 5/2 |
| J-27 | 5/3 |
| J-28 | 3/5 |
| J-28 | 5/3 |
| J-29 | 3/5 |
| J-29 | 5/3 |
| J-30 | 3/5 |
| J-30 | 5/3 |
| J-30 | 1/3 |
| J-30 | 3/1 |
| J-30 | 1/4 |
| J-30 | 4/1 |
| J-31 | 1/3 |
| J-31 | 1/4 |
| J-31 | 2/4 |
| J-31 | 2/5 |
| J-31 | 3/5 |
| J-31 | 3/1 |
| J-31 | 4/1 |
| J-31 | 4/2 |
| J-31 | 5/2 |
| J-32 | 2/4 |
| J-32 | 2/5 |
| J-32 | 3/5 |
| J-32 | 5/3 |
| J-32 | 5/2 |
| J-32 | 4/2 |
| J-33 | 2/4 |
| J-33 | 2/5 |
| J-33 | 3/5 |
| J-33 | 5/3 |
| J-33 | 5/2 |
| J-33 | 4/2 |
| J-34 | 1/3 |
| J-34 | 1/4 |
| J-34 | 3/5 |
| J-34 | 3/1 |
| J-34 | 4/1 |
| J-35 | 1/4 |
| J-35 | 4/1 |
| J-36 | 1/3 |
| J-36 | 3/1 |
| J-36 | 3/5 |
| J-36 | 5/3 |
| J-37 | 2/5 |
| J-37 | 5/2 |
| J-37 | 2/4 |
| J-37 | 4/2 |
| J-38 | 2/5 |
| J-38 | 5/2 |
| J-38 | 2/4 |
| J-38 | 4/2 |
| J-39 | 3/5 |
| J-39 | 5/3 |
| J-40 | 3/5 |
| J-40 | 5/3 |
| J-41 | 1/3 |
| J-41 | 1/4 |
| J-42 | 1/3 |
| J-42 | 1/4 |
| J-43 | 1/4 |
| J-44 | 1/3 |
| J-44 | 2/4 |
| J-44 | 2/5 |
| J-44 | 2/6 |
| J-45 | 2/4 |
| J-45 | 2/5 |
| J-45 | 2/6 |
| J-46 | 2/4 |
| J-46 | 2/5 |
| J-46 | 4/2 |
| J-46 | 5/2 |
| J-47 | 2/4 |
| J-47 | 2/5 |
| J-47 | 4/2 |
| J-47 | 5/2 |
| J-48 | 3/5 |
| J-49 | 2/4 |
| J-49 | 2/5 |
| J-49 | 4/2 |
| J-49 | 5/2 |
| J-50 | 2/6 |
| J-51 | 2/6 |
| J-52 | 2/6 |
| J-53 | 2/3 |
| J-54 | 2/3 |
| J-55 | 2/3 |
| J-56 | 2/3 |
| J-57 | 2/4 |
| J-58 | 3/4 |
| J-59 | 2/4 |
| J-60 | 2/4 |
| J-61 | 2/4 |
| J-62 | 2/4 |
| J-63 | 3/4 |
| J-64 | 2/3 |
| J-65 | 3/4 |
| J-66 | 6/7 |
| J-67 | 2/3 |
| J-68 | 2/3 |
| J-69 | 1/3 |
| J-69 | 1/4 |
| J-70 | 1/3 |
| J-71 | 2/4 |
| J-71 | 4/2 |
| J-72 | 2/4 |
| J-72 | 4/2 |
| J-73 | 2/4 |
| J-73 | 4/2 |
| J-73 | 1/3 |
| J-73 | 1/4 |
| J-73 | 4/1 |
| J-74 | 2/4 |
| J-74 | 2/5 |
| J-74 | 4/2 |
| J-74 | 5/2 |
| J-74 | 3/5 |
| J-74 | 5/3 |
| J-75 | 3/5 |
| J-75 | 5/3 |
| J-75 | 2/4 |
| J-75 | 2/5 |
| J-75 | 3/5 |
| J-75 | 5/3 |
| J-76 | 3/6 |
| J-76 | 6/3 |
| J-77 | 3/5 |
| J-77 | 5/3 |
| J-78 | 1/3 |
| J-79 | 1/3 |
| J-79 | 3/1 |
| J-80 | 1/3 |
| J-80 | 3/1 |
| J-81 | 3/5 |
| J-81 | 5/3 |
| J-82 | 3/5 |
| J-82 | 3/6 |
| J-82 | 5/3 |
| J-82 | 6/3 |

*J² in the above Markush structure represents the portion of the J groups defined in Exhibit 3 of the Embodiments for J-1 through J-82 excluding the substituent (Z²Q)ₛ. Furthermore J² is identified in the following table by reference to J-1 through J-82 whereby J² is understood to be the portion of J-1 through J-82 not including the substituent (Z²Q)ₛ shown in Exhibit 3. G^{A} is defined in Exhibit 5.
Z¹ is a direct bond; Z² is a direct bond; Z³ is a direct bond; xis 0; G^{A} is G^{A}-49; ris 0.
**J-orientation refers to the attachment points for Z¹ and Z² on the ring of J² (which is identified by reference to the J groups of Exhibit 3). The first number refers to the position on the ring of J² (with reference to the J groups of Exhibit 3) where Z¹ is attached, and the second number refers to the position on the ring of J² where Z² is attached.

TABLE 4*

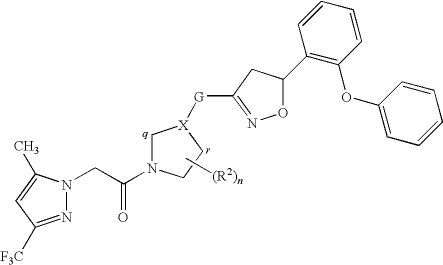

X is X¹; R^{3a} is H; R^{11a} is Me; n is 0.
G
G-1
G-2
G-3
G-4
G-5
G-6
G-7
G-8
G-9
G-10
G-11
G-12
G-13
G-14
G-15
G-16
G-17
G-18
G-19
G-20
G-21
G-22
G-23
G-24
G-25
G-26
G-27
G-28
G-29
G-30
G-31
G-32
G-33
G-34
G-35
G-36
G-37
G-38
G-39
G-40
G-41
G-42
G-43
G-44
G-45
G-46
G-47
G-48
G-49
G-50
G-51
G-52
G-53
G-54
G-55
G-56
G-57
G-58
G-59
X is X²; R^{3a} is H; R^{11a} is Me; n is 0.
G-1
G-2
G-3
G-4
G-5
G-6
G-7
G-8
G-9
G-10
G-11
G-12
G-13
G-14
G-15
G-16
G-17
G-18
G-19
G-20
G-21
G-22
G-23
G-24
G-25
G-26
G-27
G-28
G-29
G-30
G-31
G-32
G-33
G-34
G-35
G-36
G-37
G-38
G-39
G-40
G-41
G-42
G-43
G-44
G-45
G-46
G-47
G-48
G-49
G-50
G-51
G-52
G-53
G-54
G-55
G-56
G-57
G-58
G-59
X is X³; R^{3a} is H; R^{11a} is Me; n is 0.
G-1
G-2
G-3
G-4
G-5
G-6
G-7
G-8
G-9
G-10
G-11
G-12
G-13
G-14
G-15
G-16
G-17
G-18
G-19
G-20
G-21
G-22
G-23
G-24
G-25
G-26
G-27
G-28
G-29
G-30
G-31
G-32
G-33
G-34
G-35
G-36
G-37
G-38
G-39
G-40
G-41
G-42
G-43
G-44
G-45
G-46
G-47
G-48
G-49
G-50
G-51
G-52
G-53
G-54
G-55
G-56
G-57
G-58
G-59
n is 0.

| X | G | R^{3a} |
|---|---|---|
| X¹ | G-2 | Me |
| X¹ | G-2 | Cl |
| X¹ | G-2 | F |
| X¹ | G-2 | CF₃ |
| X¹ | G-14 | n-Pr |
| X¹ | G-26 | 5-Me |
| X² | G-2 | Me |
| X² | G-2 | Cl |
| X² | G-2 | F |
| X² | G-2 | CF₃ |
| X² | G-14 | n-Pr |
| X³ | G-2 | Me |
| X³ | G-2 | Cl |
| X³ | G-2 | F |
| X³ | G-2 | CF₃ |
| X³ | G-14 | n-Pr |

R^{3a} is H; n is 0.

| X | G | R^{11a} |
|---|---|---|
| X¹ | G-3 | Me |
| X¹ | G-3 | n-Pr |
| X² | G-3 | Me |
| X² | G-3 | n-Pr |
| X³ | G-3 | Me |
| X³ | G-3 | n-Pr |

G is G-1; R^{3a} is H; n is 0.
X
X⁴
X⁵
X⁶
X⁷
X⁸
X⁹
G is G-1; R^{3a} is H; n is 1.

| X | R² |
|---|---|
| X¹ | 2-Me |
| X¹ | 3-Me |
| X¹ | 2,6-di-Me |
| X¹ | 3,5-di-Me |
| X¹ | 3-n-Bu |
| X¹ | 4-MeO |
| X¹ | 4-OH |
| X¹ | 4-Cl |
| X¹ | 4-Br |
| X¹ | 4-CN |
| X² | 2-Me |
| X² | 3-Me |
| X² | 2,6-di-Me |
| X² | 3,5-di-Me |
| X² | 3-n-Bu |
| X³ | 2-Me |
| X³ | 3-Me |
| X³ | 2,6-di-Me |
| X³ | 3,5-di-Me |
| X³ | 3-n-Bu |
| X³ | 5-Me |
| X³ | 6-Me |

*The definitions of X, G, R^{3a} and R¹¹^{a} in the compounds of this table are as defined in the Summary of the Invention and Exhibit 2 in the above Embodiments.

TABLE 5*

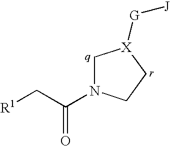

J
J-29-1
J-29-2
J-29-3
J-29-4
J-29-5
J-29-6
J-29-7
J-29-8
J-29-9
J-29-10
J-29-11
J-29-12
J-29-13
J-29-14
J-29-15
J-29-16
J-29-17
J-29-18
J-29-19
J-29-20
J-29-21
J-29-22
J-29-23
J-29-24
J-29-25
J-29-26
J-29-27
J-29-28
J-29-29
J-29-30
J-29-31
J-29-32
J-29-33
J-29-34
J-29-35
J-29-36
J-29-37
J-29-38
J-29-39
J-29-40
J-29-41
J-29-42
J-29-43
J-29-44
J-29-45
J-29-46
J-29-47
J-29-48
J-29-49
J-29-50
J-29-51
J-29-52
J-29-53
J-29-54
J-29-55
J-29-56
J-29-57
J-29-58
J-29-59
J-29-60
R¹ is 2,5-dichlorophenyl; X is X²; G is G-1; R^{3a} is H.
J-29-1
J-29-2
J-29-3
J-29-4
J-29-5
J-29-6
J-29-7
J-29-8
J-29-9
J-29-10
J-29-11
J-29-12
J-29-13
J-29-14
J-29-15
J-29-16
J-29-17
J-29-18
J-29-19
J-29-20
J-29-21
J-29-22
J-29-23
J-29-24
J-29-25
J-29-26
J-29-27
J-29-28
J-29-29
J-29-30
J-29-31
J-29-32
J-29-33
J-29-34
J-29-35
J-29-36
J-29-37
J-29-38
J-29-39
J-29-40
J-29-41
J-29-42
J-29-43
J-29-44
J-29-45
J-29-46
J-29-47
J-29-48
J-29-49
J-29-50
J-29-51
J-29-52
J-29-53
J-29-54
J-29-55
J-29-56
J-29-57
J-29-58
J-29-59
J-29-60
R¹ is 2,5-dichlorophenyl; X is X¹; G is G-2; R^{3a} is H.
J-29-1
J-29-2
J-29-3
J-29-4
J-29-5
J-29-6
J-29-7
J-29-8
J-29-9
J-29-10
J-29-11
J-29-12
J-29-13
J-29-14
J-29-15
J-29-16
J-29-17
J-29-18
J-29-19
J-29-20
J-29-21
J-29-22
J-29-23
J-29-24
J-29-25
J-29-26
J-29-27
J-29-28
J-29-29
J-29-30
J-29-31
J-29-32
J-29-33
J-29-34
J-29-35
J-29-36
J-29-37
J-29-38
J-29-39
J-29-40
J-29-41
J-29-42
J-29-43
J-29-44
J-29-45
J-29-46
J-29-47
J-29-48
J-29-49
J-29-50
J-29-51
J-29-52
J-29-53
J-29-54
J-29-55
J-29-56
J-29-57
J-29-58
J-29-59
J-29-60
R¹ is 2,5-dichlorophenyl; X is X²; G is G-2; R^{3a} is H.
J-29-1
J-29-2
J-29-3
J-29-4
J-29-5
J-29-6
J-29-7
J-29-8
J-29-9
J-29-10
J-29-11
J-29-12
J-29-13
J-29-14
J-29-15
J-29-16
J-29-17
J-29-18
J-29-19
J-29-20
J-29-21
J-29-22
J-29-23
J-29-24
J-29-25
J-29-26
J-29-27
J-29-28
J-29-29
J-29-30
J-29-31
J-29-32
J-29-33
J-29-34
J-29-35
J-29-36
J-29-37
J-29-38
J-29-39
J-29-40
J-29-41
J-29-42
J-29-43
J-29-44
J-29-45
J-29-46
J-29-47
J-29-48
J-29-49
J-29-50
J-29-51
J-29-52
J-29-53
J-29-54
J-29-55
J-29-56
J-29-57
J-29-58
J-29-59
J-29-60
R¹ is 2-chloro-5-(trifluoromethyl)phenyl; X is X¹; G is G-1; R^{3a} is H.
J-29-1
J-29-2
J-29-3
J-29-4
J-29-5
J-29-6
J-29-7
J-29-8
J-29-9
J-29-10
J-29-11
J-29-12
J-29-13
J-29-14
J-29-15
J-29-16
J-29-17
J-29-18
J-29-19
J-29-20
J-29-21
J-29-22
J-29-23
J-29-24
J-29-25
J-29-26
J-29-27
J-29-28
J-29-29
J-29-30
J-29-31
J-29-32
J-29-33
J-29-34
J-29-35
J-29-36
J-29-37
J-29-38
J-29-39
J-29-40
J-29-41
J-29-42
J-29-43
J-29-44
J-29-45
J-29-46
J-29-47
J-29-48
J-29-49
J-29-50
J-29-51
J-29-52
J-29-53
J-29-54
J-29-55
J-29-56
J-29-57
J-29-58
J-29-59
J-29-60
R¹ is 2-chloro-5-(trifluoromethyl)phenyl; X is X²; G is G-1; R^{3a} is H.
J-29-1
J-29-2
J-29-3
J-29-4
J-29-5
J-29-6
J-29-7
J-29-8
J-29-9
J-29-10
J-29-11
J-29-12
J-29-13
J-29-14
J-29-15
J-29-16
J-29-17
J-29-18
J-29-19
J-29-20
J-29-21
J-29-22
J-29-23
J-29-24
J-29-25
J-29-26
J-29-27
J-29-28
J-29-29
J-29-30
J-29-31
J-29-32
J-29-33
J-29-34
J-29-35
J-29-36
J-29-37
J-29-38
J-29-39
J-29-40
J-29-41
J-29-42
J-29-43
J-29-44
J-29-45
J-29-46
J-29-47
J-29-48
J-29-49
J-29-50
J-29-51
J-29-52
J-29-53
J-29-54
J-29-55
J-29-56
J-29-57
J-29-58
J-29-59
J-29-60
R¹ is 2-chloro-5-(trifluoromethyl)phenyl; X is X¹; G is G-2; R^{3a} is H.
J-29-1
J-29-2
J-29-3
J-29-4
J-29-5
J-29-6
J-29-7
J-29-8
J-29-9
J-29-10
J-29-11
J-29-12
J-29-13
J-29-14
J-29-15
J-29-16
J-29-17
J-29-18
J-29-19
J-29-20
J-29-21
J-29-22
J-29-23
J-29-24
J-29-25
J-29-26
J-29-27
J-29-28
J-29-29
J-29-30
J-29-31
J-29-32
J-29-33
J-29-34
J-29-35
J-29-36
J-29-37
J-29-38
J-29-39
J-29-40
J-29-41
J-29-42
J-29-43
J-29-44
J-29-45
J-29-46
J-29-47
J-29-48
J-29-49
J-29-50
J-29-51
J-29-52
J-29-53
J-29-54
J-29-55
J-29-56
J-29-57
J-29-58
J-29-59
J-29-60
R¹ is 2-chloro-5-(trifluoromethyl)phenyl; X is X²; G is G-2; R^{3a} is H.
J-29-1
J-29-2
J-29-3
J-29-4
J-29-5
J-29-6
J-29-7
J-29-8
J-29-9
J-29-10
J-29-11
J-29-12
J-29-13
J-29-14
J-29-15
J-29-16
J-29-17
J-29-18
J-29-19
J-29-20
J-29-21
J-29-22
J-29-23
J-29-24
J-29-25
J-29-26
J-29-27
J-29-28
J-29-29
J-29-30
J-29-31
J-29-32
J-29-33
J-29-34
J-29-35
J-29-36
J-29-37
J-29-38
J-29-39
J-29-40
J-29-41
J-29-42
J-29-43
J-29-44
J-29-45
J-29-46
J-29-47
J-29-48
J-29-49
J-29-50
J-29-51
J-29-52
J-29-53
J-29-54
J-29-55
J-29-56
J-29-57
J-29-58
J-29-59
J-29-60
R¹ is 2,5-dimethylphenyl; X is X¹; G is G-1; R^{3a} is H.
J-29-1
J-29-2
J-29-3
J-29-4
J-29-5
J-29-6
J-29-7
J-29-8
J-29-9
J-29-10
J-29-11
J-29-12
J-29-13
J-29-14
J-29-15
J-29-16
J-29-17
J-29-18
J-29-19
J-29-20
J-29-21
J-29-22
J-29-23
J-29-24
J-29-25
J-29-26
J-29-27
J-29-28
J-29-29
J-29-30
J-29-31
J-29-32
J-29-33
J-29-34
J-29-35
J-29-36
J-29-37
J-29-38
J-29-39
J-29-40
J-29-41
J-29-42
J-29-43
J-29-44
J-29-45
J-29-46
J-29-47
J-29-48
J-29-49
J-29-50
J-29-51
J-29-52
J-29-53
J-29-54
J-29-55
J-29-56
J-29-57
J-29-58
J-29-59
J-29-60
R¹ is 2,5-dimethylphenyl; X is X²; G is G-1; R^{3a} is H.
J-29-1
J-29-2
J-29-3
J-29-4
J-29-5
J-29-6
J-29-7
J-29-8
J-29-9
J-29-10
J-29-11
J-29-12
J-29-13
J-29-14
J-29-15
J-29-16
J-29-17
J-29-18
J-29-19
J-29-20
J-29-21
J-29-22
J-29-23
J-29-24
J-29-25
J-29-26
J-29-27
J-29-28
J-29-29
J-29-30
J-29-31
J-29-32
J-29-33
J-29-34
J-29-35
J-29-36
J-29-37
J-29-38
J-29-39
J-29-40
J-29-41
J-29-42
J-29-43
J-29-44
J-29-45
J-29-46
J-29-47
J-29-48
J-29-49
J-29-50
J-29-51
J-29-52
J-29-53
J-29-54
J-29-55
J-29-56
J-29-57
J-29-58
J-29-59
J-29-60
R¹ is 2,5-dimethylphenyl; X is X¹; G is G-2; R^{3a} is H.
J-29-1
J-29-2
J-29-3
J-29-4
J-29-5
J-29-6
J-29-7
J-29-8
J-29-9
J-29-10
J-29-11
J-29-12
J-29-13
J-29-14
J-29-15
J-29-16
J-29-17
J-29-18
J-29-19
J-29-20
J-29-21
J-29-22
J-29-23
J-29-24
J-29-25
J-29-26
J-29-27
J-29-28
J-29-29
J-29-30
J-29-31
J-29-32
J-29-33
J-29-34
J-29-35
J-29-36
J-29-37
J-29-38
J-29-39
J-29-40
J-29-41
J-29-42
J-29-43
J-29-44
J-29-45
J-29-46
J-29-47
J-29-48
J-29-49
J-29-50
J-29-51
J-29-52
J-29-53
J-29-54
J-29-55
J-29-56
J-29-57
J-29-58
J-29-59
J-29-60
R¹ is 2,5-dimethylphenyl; X is X²; G is G-2; R^{3a} is H.
J-29-1
J-29-2
J-29-3
J-29-4
J-29-5
J-29-6
J-29-7
J-29-8
J-29-9
J-29-10
J-29-11
J-29-12
J-29-13
J-29-14
J-29-15
J-29-16
J-29-17
J-29-18
J-29-19
J-29-20
J-29-21
J-29-22
J-29-23
J-29-24
J-29-25
J-29-26
J-29-27
J-29-28
J-29-29
J-29-30
J-29-31
J-29-32
J-29-33
J-29-34
J-29-35
J-29-36
J-29-37
J-29-38
J-29-39
J-29-40
J-29-41
J-29-42
J-29-43
J-29-44
J-29-45
J-29-46
J-29-47
J-29-48
J-29-49
J-29-50
J-29-51
J-29-52
J-29-53
J-29-54
J-29-55
J-29-56
J-29-57
J-29-58
J-29-59
J-29-60
R¹ is 2-methyl-5-(trifluoromethyl)phenyl; X is X¹; G is G-1; R^{3a} is H.
J-29-1
J-29-2
J-29-3
J-29-4
J-29-5
J-29-6
J-29-7
J-29-8
J-29-9
J-29-10
J-29-11
J-29-12
J-29-13
J-29-14
J-29-15
J-29-16
J-29-17
J-29-18
J-29-19
J-29-20
J-29-21
J-29-22
J-29-23
J-29-24
J-29-25
J-29-26
J-29-27
J-29-28
J-29-29
J-29-30
J-29-31
J-29-32
J-29-33
J-29-34
J-29-35
J-29-36
J-29-37
J-29-38
J-29-39
J-29-40
J-29-41
J-29-42
J-29-43
J-29-44
J-29-45
J-29-46
J-29-47
J-29-48
J-29-49
J-29-50
J-29-51
J-29-52
J-29-53
J-29-54
J-29-55
J-29-56
J-29-57
J-29-58
J-29-59
J-29-60
R¹ is 2-methyl-5-(trifluoromethyl)phenyl; X is X²; G is G-1; R^{3a} is H.
J-29-1
J-29-2
J-29-3
J-29-4
J-29-5
J-29-6
J-29-7
J-29-8
J-29-9
J-29-10
J-29-11
J-29-12
J-29-13
J-29-14
J-29-15
J-29-16
J-29-17
J-29-18
J-29-19
J-29-20
J-29-21
J-29-22
J-29-23
J-29-24
J-29-25
J-29-26
J-29-27
J-29-28
J-29-29
J-29-30
J-29-31
J-29-32
J-29-33
J-29-34
J-29-35
J-29-36
J-29-37
J-29-38
J-29-39
J-29-40
J-29-41
J-29-42
J-29-43
J-29-44
J-29-45
J-29-46
J-29-47
J-29-48
J-29-49
J-29-50
J-29-51
J-29-52
J-29-53
J-29-54
J-29-55
J-29-56
J-29-57
J-29-58
J-29-59
J-29-60
R¹ is 2-methyl-5-(trifluoromethyl)phenyl; X is X¹; G is G-2; R^{3a} is H.
J-29-1
J-29-2
J-29-3
J-29-4
J-29-5
J-29-6
J-29-7
J-29-8
J-29-9
J-29-10
J-29-11
J-29-12
J-29-13
J-29-14
J-29-15
J-29-16
J-29-17
J-29-18
J-29-19
J-29-20
J-29-21
J-29-22
J-29-23
J-29-24
J-29-25
J-29-26
J-29-27
J-29-28
J-29-29
J-29-30
J-29-31
J-29-32
J-29-33
J-29-34
J-29-35
J-29-36
J-29-37
J-29-38
J-29-39
J-29-40
J-29-41
J-29-42
J-29-43
J-29-44
J-29-45
J-29-46
J-29-47
J-29-48
J-29-49
J-29-50
J-29-51
J-29-52
J-29-53
J-29-54
J-29-55
J-29-56
J-29-57
J-29-58
J-29-59
J-29-60
R¹ is 2-methyl-5-(trifluoromethyl)phenyl; X is X²; G is G-2; R^{3a} is H.
J-29-1
J-29-2
J-29-3
J-29-4
J-29-5
J-29-6
J-29-7
J-29-8
J-29-9
J-29-10
J-29-11
J-29-12
J-29-13
J-29-14
J-29-15
J-29-16
J-29-17
J-29-18
J-29-19
J-29-20
J-29-21
J-29-22
J-29-23
J-29-24
J-29-25
J-29-26
J-29-27
J-29-28
J-29-29
J-29-30
J-29-31
J-29-32
J-29-33
J-29-34
J-29-35
J-29-36
J-29-37
J-29-38
J-29-39
J-29-40
J-29-41
J-29-42
J-29-43
J-29-44
J-29-45
J-29-46
J-29-47
J-29-48
J-29-49
J-29-50
J-29-51
J-29-52
J-29-53
J-29-54
J-29-55
J-29-56
J-29-57
J-29-58
J-29-59
J-29-60
R¹ is 3,5-dimethylpyrazol-1-yl; X is X¹; G is G-1; R^{3a} is H.
J-29-1
J-29-2
J-29-3
J-29-4
J-29-5
J-29-6
J-29-7
J-29-8
J-29-9
J-29-10
J-29-11
J-29-12
J-29-13
J-29-14
J-29-15
J-29-16
J-29-17
J-29-18
J-29-19
J-29-20
J-29-21
J-29-22
J-29-23
J-29-24
J-29-25
J-29-26
J-29-27
J-29-28
J-29-29
J-29-30
J-29-31
J-29-32
J-29-33
J-29-34
J-29-35
J-29-36
J-29-37
J-29-38
J-29-39
J-29-40
J-29-41
J-29-42
J-29-43
J-29-44
J-29-45
J-29-46
J-29-47
J-29-48
J-29-49
J-29-50
J-29-51
J-29-52
J-29-53
J-29-54
J-29-55
J-29-56
J-29-57
J-29-58
J-29-59
J-29-60
R¹ is 3,5-dimethylpyrazol-1-yl; X is X²; G is G-1; R^{3a} is H.
J-29-1
J-29-2
J-29-3
J-29-4
J-29-5
J-29-6
J-29-7
J-29-8
J-29-9
J-29-10
J-29-11
J-29-12
J-29-13
J-29-14
J-29-15
J-29-16
J-29-17
J-29-18
J-29-19
J-29-20
J-29-21
J-29-22
J-29-23
J-29-24
J-29-25
J-29-26
J-29-27
J-29-28
J-29-29
J-29-30
J-29-31
J-29-32
J-29-33
J-29-34
J-29-35
J-29-36
J-29-37
J-29-38
J-29-39
J-29-40
J-29-41
J-29-42
J-29-43
J-29-44
J-29-45
J-29-46
J-29-47
J-29-48
J-29-49
J-29-50
J-29-51
J-29-52
J-29-53
J-29-54
J-29-55
J-29-56
J-29-57
J-29-58
J-29-59
J-29-60
R¹ is 3,5-dimethylpyrazol-1-yl; X is X¹; G is G-2; R^{3a} is H.
J-29-1
J-29-2
J-29-3
J-29-4
J-29-5
J-29-6
J-29-7
J-29-8
J-29-9
J-29-10
J-29-11
J-29-12
J-29-13
J-29-14
J-29-15
J-29-16
J-29-17
J-29-18
J-29-19
J-29-20
J-29-21
J-29-22
J-29-23
J-29-24
J-29-25
J-29-26
J-29-27
J-29-28
J-29-29
J-29-30
J-29-31
J-29-32
J-29-33
J-29-34
J-29-35
J-29-36
J-29-37
J-29-38
J-29-39
J-29-40
J-29-41
J-29-42
J-29-43
J-29-44
J-29-45
J-29-46
J-29-47
J-29-48
J-29-49
J-29-50
J-29-51
J-29-52
J-29-53
J-29-54
J-29-55
J-29-56
J-29-57
J-29-58
J-29-59
J-29-60
R¹ is 3,5-dimethylpyrazol-1-yl; X is X²; G is G-2; R^{3a} is H.
J-29-1
J-29-2
J-29-3
J-29-4
J-29-5
J-29-6
J-29-7
J-29-8
J-29-9
J-29-10
J-29-11
J-29-12
J-29-13
J-29-14
J-29-15
J-29-16
J-29-17
J-29-18
J-29-19
J-29-20
J-29-21
J-29-22
J-29-23
J-29-24
J-29-25
J-29-26
J-29-27
J-29-28
J-29-29
J-29-30
J-29-31
J-29-32
J-29-33
J-29-34
J-29-35
J-29-36
J-29-37
J-29-38
J-29-39
J-29-40
J-29-41
J-29-42
J-29-43
J-29-44
J-29-45
J-29-46
J-29-47
J-29-48
J-29-49
J-29-50
J-29-51
J-29-52
J-29-53
J-29-54
J-29-55
J-29-56
J-29-57
J-29-58
J-29-59
J-29-60
R¹ is 3,5-dichloropyrazol-1-yl; X is X¹; G is G-1; R^{3a} is H.
J-29-1
J-29-2
J-29-3
J-29-4
J-29-5
J-29-6
J-29-7
J-29-8
J-29-9
J-29-10
J-29-11
J-29-12
J-29-13
J-29-14
J-29-15
J-29-16
J-29-17
J-29-18
J-29-19
J-29-20
J-29-21
J-29-22
J-29-23
J-29-24
J-29-25
J-29-26
J-29-27
J-29-28
J-29-29
J-29-30
J-29-31
J-29-32
J-29-33
J-29-34
J-29-35
J-29-36
J-29-37
J-29-38
J-29-39
J-29-40
J-29-41
J-29-42
J-29-43
J-29-44
J-29-45
J-29-46
J-29-47
J-29-48
J-29-49
J-29-50
J-29-51
J-29-52
J-29-53
J-29-54
J-29-55
J-29-56
J-29-57
J-29-58
J-29-59
J-29-60
R¹ is 3,5-dichloropyrazol-1-yl; X is X²; G is G-1; R^{3a} is H.
J-29-1
J-29-2
J-29-3
J-29-4
J-29-5
J-29-6
J-29-7
J-29-8
J-29-9
J-29-10
J-29-11
J-29-12
J-29-13
J-29-14
J-29-15
J-29-16
J-29-17
J-29-18
J-29-19
J-29-20
J-29-21
J-29-22
J-29-23
J-29-24
J-29-25
J-29-26
J-29-27
J-29-28
J-29-29
J-29-30
J-29-31
J-29-32
J-29-33
J-29-34
J-29-35
J-29-36
J-29-37
J-29-38
J-29-39
J-29-40
J-29-41
J-29-42
J-29-43
J-29-44
J-29-45
J-29-46
J-29-47
J-29-48
J-29-49
J-29-50
J-29-51
J-29-52
J-29-53
J-29-54
J-29-55
J-29-56
J-29-57
J-29-58
J-29-59
J-29-60
R¹ is 3,5-dichloropyrazol-1-yl; X is X¹; G is G-2; R^{3a} is H.
J-29-1
J-29-2
J-29-3
J-29-4
J-29-5
J-29-6
J-29-7
J-29-8
J-29-9
J-29-10
J-29-11
J-29-12
J-29-13
J-29-14
J-29-15
J-29-16
J-29-17
J-29-18
J-29-19
J-29-20
J-29-21
J-29-22
J-29-23
J-29-24
J-29-25
J-29-26
J-29-27
J-29-28
J-29-29
J-29-30
J-29-31
J-29-32
J-29-33
J-29-34
J-29-35
J-29-36
J-29-37
J-29-38
J-29-39
J-29-40
J-29-41
J-29-42
J-29-43
J-29-44
J-29-45
J-29-46
J-29-47
J-29-48
J-29-49
J-29-50
J-29-51
J-29-52
J-29-53
J-29-54
J-29-55
J-29-56
J-29-57
J-29-58
J-29-59
J-29-60
R¹ is 3,5-dichloropyrazol-1-yl; X is X²; G is G-2; R^{3a} is H.
J-29-1
J-29-2
J-29-3
J-29-4
J-29-5
J-29-6
J-29-7
J-29-8
J-29-9
J-29-10
J-29-11
J-29-12
J-29-13
J-29-14
J-29-15
J-29-16
J-29-17
J-29-18
J-29-19
J-29-20
J-29-21
J-29-22
J-29-23
J-29-24
J-29-25
J-29-26
J-29-27
J-29-28
J-29-29
J-29-30
J-29-31
J-29-32
J-29-33
J-29-34
J-29-35
J-29-36
J-29-37
J-29-38
J-29-39
J-29-40
J-29-41
J-29-42
J-29-43
J-29-44
J-29-45
J-29-46
J-29-47
J-29-48
J-29-49
J-29-50
J-29-51
J-29-52
J-29-53
J-29-54
J-29-55
J-29-56
J-29-57
J-29-58
J-29-59
J-29-60
R¹ is 3,5-dibromopyrazol-1-yl; X is X¹; G is G-1; R^{3a} is H.
J-29-1
J-29-2
J-29-3
J-29-4
J-29-5
J-29-6
J-29-7
J-29-8
J-29-9
J-29-10
J-29-11
J-29-12
J-29-13
J-29-14
J-29-15
J-29-16
J-29-17
J-29-18
J-29-19
J-29-20
J-29-21
J-29-22
J-29-23
J-29-24
J-29-25
J-29-26
J-29-27
J-29-28
J-29-29
J-29-30
J-29-31
J-29-32
J-29-33
J-29-34
J-29-35
J-29-36
J-29-37
J-29-38
J-29-39
J-29-40
J-29-41
J-29-42
J-29-43
J-29-44
J-29-45
J-29-46
J-29-47
J-29-48
J-29-49
J-29-50
J-29-51
J-29-52
J-29-53
J-29-54
J-29-55
J-29-56
J-29-57
J-29-58
J-29-59
J-29-60
R¹ is 3,5-dibromopyrazol-1-yl; X is X²; G is G-1; R^{3a} is H.
J-29-1
J-29-2
J-29-3
J-29-4
J-29-5
J-29-6
J-29-7
J-29-8
J-29-9
J-29-10
J-29-11
J-29-12
J-29-13
J-29-14
J-29-15
J-29-16
J-29-17
J-29-18
J-29-19
J-29-20
J-29-21
J-29-22
J-29-23
J-29-24
J-29-25
J-29-26
J-29-27
J-29-28
J-29-29
J-29-30
J-29-31
J-29-32
J-29-33
J-29-34
J-29-35
J-29-36
J-29-37
J-29-38
J-29-39
J-29-40
J-29-41
J-29-42
J-29-43
J-29-44
J-29-45
J-29-46
J-29-47
J-29-48
J-29-49
J-29-50
J-29-51
J-29-52
J-29-53
J-29-54
J-29-55
J-29-56
J-29-57
J-29-58
J-29-59
J-29-60
R¹ is 3,5-dibromopyrazol-1-yl; X is X¹; G is G-2; R^{3a} is H.
J-29-1
J-29-2
J-29-3
J-29-4
J-29-5
J-29-6
J-29-7
J-29-8
J-29-9
J-29-10
J-29-11
J-29-12
J-29-13
J-29-14
J-29-15
J-29-16
J-29-17
J-29-18
J-29-19
J-29-20
J-29-21
J-29-22
J-29-23
J-29-24
J-29-25
J-29-26
J-29-27
J-29-28
J-29-29
J-29-30
J-29-31
J-29-32
J-29-33
J-29-34
J-29-35
J-29-36
J-29-37
J-29-38
J-29-39
J-29-40
J-29-41
J-29-42
J-29-43
J-29-44
J-29-45
J-29-46
J-29-47
J-29-48
J-29-49
J-29-50
J-29-51
J-29-52
J-29-53
J-29-54
J-29-55
J-29-56
J-29-57
J-29-58
J-29-59
J-29-60
R¹ is 3,5-dibromopyrazol-1-yl; X is X²; G is G-2; R^{3a} is H.
J-29-1
J-29-2
J-29-3
J-29-4
J-29-5
J-29-6
J-29-7
J-29-8
J-29-9
J-29-10
J-29-11
J-29-12
J-29-13
J-29-14
J-29-15
J-29-16
J-29-17
J-29-18
J-29-19
J-29-20
J-29-21
J-29-22
J-29-23
J-29-24
J-29-25
J-29-26
J-29-27
J-29-28
J-29-29
J-29-30
J-29-31
J-29-32
J-29-33
J-29-34
J-29-35
J-29-36
J-29-37
J-29-38
J-29-39
J-29-40
J-29-41
J-29-42
J-29-43
J-29-44
J-29-45
J-29-46
J-29-47
J-29-48
J-29-49
J-29-50
J-29-51
J-29-52
J-29-53
J-29-54
J-29-55
J-29-56
J-29-57
J-29-58
J-29-59
J-29-60
R¹ is 5-methyl-3-(trifluoromethyl)pyrazol-1-yl; X is X¹; G is G-1; R^{3a} is H.
J-29-1
J-29-2
J-29-3
J-29-4
J-29-5
J-29-6
J-29-7
J-29-8
J-29-9
J-29-10
J-29-11
J-29-12
J-29-13
J-29-14
J-29-15
J-29-16
J-29-17
J-29-18
J-29-19
J-29-20
J-29-21
J-29-22
J-29-23
J-29-24
J-29-25
J-29-26
J-29-27
J-29-28
J-29-29
J-29-30
J-29-31
J-29-32
J-29-33
J-29-34
J-29-35
J-29-36
J-29-37
J-29-38
J-29-39
J-29-40
J-29-41
J-29-42
J-29-43
J-29-44
J-29-45
J-29-46
J-29-47
J-29-48
J-29-49
J-29-50
J-29-51
J-29-52
J-29-53
J-29-54
J-29-55
J-29-56
J-29-57
J-29-58
J-29-59
J-29-60
R¹ is 5-methyl-3-(trifluoromethyl)pyrazol-1-yl; X is X²; G is G-1; R^{3a} is H.
J-29-1
J-29-2
J-29-3
J-29-4
J-29-5
J-29-6
J-29-7
J-29-8
J-29-9
J-29-10
J-29-11
J-29-12
J-29-13
J-29-14
J-29-15
J-29-16
J-29-17
J-29-18
J-29-19
J-29-20
J-29-21
J-29-22
J-29-23
J-29-24
J-29-25
J-29-26
J-29-27
J-29-28
J-29-29
J-29-30
J-29-31
J-29-32
J-29-33
J-29-34
J-29-35
J-29-36
J-29-37
J-29-38
J-29-39
J-29-40
J-29-41
J-29-42
J-29-43
J-29-44
J-29-45
J-29-46
J-29-47
J-29-48
J-29-49
J-29-50
J-29-51
J-29-52
J-29-53
J-29-54
J-29-55
J-29-56
J-29-57
J-29-58
J-29-59
J-29-60
R¹ is 5-methyl-3-(trifluoromethyl)pyrazol-1-yl; X is X¹; G is G-2; R^{3a} is H.
J-29-1
J-29-2
J-29-3
J-29-4
J-29-5
J-29-6
J-29-7
J-29-8
J-29-9
J-29-10
J-29-11
J-29-12
J-29-13
J-29-14
J-29-15
J-29-16
J-29-17
J-29-18
J-29-19
J-29-20
J-29-21
J-29-22
J-29-23
J-29-24
J-29-25
J-29-26
J-29-27
J-29-28
J-29-29
J-29-30
J-29-31
J-29-32
J-29-33
J-29-34
J-29-35
J-29-36
J-29-37
J-29-38
J-29-39
J-29-40
J-29-41
J-29-42
J-29-43
J-29-44
J-29-45
J-29-46
J-29-47
J-29-48
J-29-49
J-29-50
J-29-51
J-29-52
J-29-53
J-29-54
J-29-55
J-29-56
J-29-57
J-29-58
J-29-59
J-29-60
R¹ is 5-methyl-3-(trifluoromethyl)pyrazol-1-yl; X is X²; G is G-2; R^{3a} is H.
J-29-1
J-29-2
J-29-3
J-29-4
J-29-5
J-29-6
J-29-7
J-29-8
J-29-9
J-29-10
J-29-11
J-29-12
J-29-13
J-29-14
J-29-15
J-29-16
J-29-17
J-29-18
J-29-19
J-29-20
J-29-21
J-29-22
J-29-23
J-29-24
J-29-25
J-29-26
J-29-27
J-29-28
J-29-29
J-29-30
J-29-31
J-29-32
J-29-33
J-29-34
J-29-35
J-29-36
J-29-37
J-29-38
J-29-39
J-29-40
J-29-41
J-29-42
J-29-43
J-29-44
J-29-45
J-29-46
J-29-47
J-29-48
J-29-49
J-29-50
J-29-51
J-29-52
J-29-53
J-29-54
J-29-55
J-29-56
J-29-57
J-29-58
J-29-59
J-29-60
R¹ is 5-chloro-3-(trifluoromethyl)pyrazol-1-yl; X is X¹; G is G-1; R^{3a} is H.
J-29-1
J-29-2
J-29-3
J-29-4
J-29-5
J-29-6
J-29-7
J-29-8
J-29-9
J-29-10
J-29-11
J-29-12
J-29-13
J-29-14
J-29-15
J-29-16
J-29-17
J-29-18
J-29-19
J-29-20
J-29-21
J-29-22
J-29-23
J-29-24
J-29-25
J-29-26
J-29-27
J-29-28
J-29-29
J-29-30
J-29-31
J-29-32
J-29-33
J-29-34
J-29-35
J-29-36
J-29-37
J-29-38
J-29-39
J-29-40
J-29-41
J-29-42
J-29-43
J-29-44
J-29-45
J-29-46
J-29-47
J-29-48
J-29-49
J-29-50
J-29-51
J-29-52
J-29-53
J-29-54
J-29-55
J-29-56
J-29-57
J-29-58
J-29-59
J-29-60
R¹ is 5-chloro-3-(trifluoromethyl)pyrazol-1-yl; X is X²; G is G-1; R^{3a} is H.
J-29-1
J-29-2
J-29-3
J-29-4
J-29-5
J-29-6
J-29-7
J-29-8
J-29-9
J-29-10
J-29-11
J-29-12
J-29-13
J-29-14
J-29-15
J-29-16
J-29-17
J-29-18
J-29-19
J-29-20
J-29-21
J-29-22
J-29-23
J-29-24
J-29-25
J-29-26
J-29-27
J-29-28
J-29-29
J-29-30
J-29-31
J-29-32
J-29-33
J-29-34
J-29-35
J-29-36
J-29-37
J-29-38
J-29-39
J-29-40
J-29-41
J-29-42
J-29-43
J-29-44
J-29-45
J-29-46
J-29-47
J-29-48
J-29-49
J-29-50
J-29-51
J-29-52
J-29-53
J-29-54
J-29-55
J-29-56
J-29-57
J-29-58
J-29-59
J-29-60
R¹ is 5-chloro-3-(trifluoromethyl)pyrazol-1-yl; X is X¹; G is G-2; R^{3a} is H.
J-29-1
J-29-2
J-29-3
J-29-4
J-29-5
J-29-6
J-29-7
J-29-8
J-29-9
J-29-10
J-29-11
J-29-12
J-29-13
J-29-14
J-29-15
J-29-16
J-29-17
J-29-18
J-29-19
J-29-20
J-29-21
J-29-22
J-29-23
J-29-24
J-29-25
J-29-26
J-29-27
J-29-28
J-29-29
J-29-30
J-29-31
J-29-32
J-29-33
J-29-34
J-29-35
J-29-36
J-29-37
J-29-38
J-29-39
J-29-40
J-29-41
J-29-42
J-29-43
J-29-44
J-29-45
J-29-46
J-29-47
J-29-48
J-29-49
J-29-50
J-29-51
J-29-52
J-29-53
J-29-54
J-29-55
J-29-56
J-29-57
J-29-58
J-29-59
J-29-60
R¹ is 5-chloro-3-(trifluoromethyl)pyrazol-1-yl; X is X²; G is G-2; R^{3a} is H.
J-29-1
J-29-2
J-29-3
J-29-4
J-29-5
J-29-6
J-29-7
J-29-8
J-29-9
J-29-10
J-29-11
J-29-12
J-29-13
J-29-14
J-29-15
J-29-16
J-29-17
J-29-18
J-29-19
J-29-20
J-29-21
J-29-22
J-29-23
J-29-24
J-29-25
J-29-26
J-29-27
J-29-28
J-29-29
J-29-30
J-29-31
J-29-32
J-29-33
J-29-34
J-29-35
J-29-36
J-29-37
J-29-38
J-29-39
J-29-40
J-29-41
J-29-42
J-29-43
J-29-44
J-29-45
J-29-46
J-29-47
J-29-48
J-29-49
J-29-50
J-29-51
J-29-52
J-29-53
J-29-54
J-29-55
J-29-56
J-29-57
J-29-58
J-29-59
J-29-60
R¹ is 5-bromo-3-(trifluoromethyl)pyrazol-1-yl; X is X¹; G is G-1; R^{3a} is H.
J-29-1
J-29-2
J-29-3
J-29-4
J-29-5
J-29-6
J-29-7
J-29-8
J-29-9
J-29-10
J-29-11
J-29-12
J-29-13
J-29-14
J-29-15
J-29-16
J-29-17
J-29-18
J-29-19
J-29-20
J-29-21
J-29-22
J-29-23
J-29-24
J-29-25
J-29-26
J-29-27
J-29-28
J-29-29
J-29-30
J-29-31
J-29-32
J-29-33
J-29-34
J-29-35
J-29-36
J-29-37
J-29-38
J-29-39
J-29-40
J-29-41
J-29-42
J-29-43
J-29-44
J-29-45
J-29-46
J-29-47
J-29-48
J-29-49
J-29-50
J-29-51
J-29-52
J-29-53
J-29-54
J-29-55
J-29-56
J-29-57
J-29-58
J-29-59
J-29-60
R¹ is 5-bromo-3-(trifluoromethyl)pyrazol-1-yl; X is X²; G is G-1; R^{3a} is H.
J-29-1
J-29-2
J-29-3
J-29-4
J-29-5
J-29-6
J-29-7
J-29-8
J-29-9
J-29-10
J-29-11
J-29-12
J-29-13
J-29-14
J-29-15
J-29-16
J-29-17
J-29-18
J-29-19
J-29-20
J-29-21
J-29-22
J-29-23
J-29-24
J-29-25
J-29-26
J-29-27
J-29-28
J-29-29
J-29-30
J-29-31
J-29-32
J-29-33
J-29-34
J-29-35
J-29-36
J-29-37
J-29-38
J-29-39
J-29-40
J-29-41
J-29-42
J-29-43
J-29-44
J-29-45
J-29-46
J-29-47
J-29-48
J-29-49
J-29-50
J-29-51
J-29-52
J-29-53
J-29-54
J-29-55
J-29-56
J-29-57
J-29-58
J-29-59
J-29-60
R¹ is 5-bromo-3-(trifluoromethyl)pyrazol-1-yl; X is X¹; G is G-2; R^{3a} is H.
J-29-1
J-29-2
J-29-3
J-29-4
J-29-5
J-29-6
J-29-7
J-29-8
J-29-9
J-29-10
J-29-11
J-29-12
J-29-13
J-29-14
J-29-15
J-29-16
J-29-17
J-29-18
J-29-19
J-29-20
J-29-21
J-29-22
J-29-23
J-29-24
J-29-25
J-29-26
J-29-27
J-29-28
J-29-29
J-29-30
J-29-31
J-29-32
J-29-33
J-29-34
J-29-35
J-29-36
J-29-37
J-29-38
J-29-39
J-29-40
J-29-41
J-29-42
J-29-43
J-29-44
J-29-45
J-29-46
J-29-47
J-29-48
J-29-49
J-29-50
J-29-51
J-29-52
J-29-53
J-29-54
J-29-55
J-29-56
J-29-57
J-29-58
J-29-59
J-29-60
R¹ is 5-bromo-3-(trifluoromethyl)pyrazol-1-yl; X is X²; G is G-2; R^{3a} is H.
J-29-1
J-29-2
J-29-3
J-29-4
J-29-5
J-29-6
J-29-7
J-29-8
J-29-9
J-29-10
J-29-11
J-29-12
J-29-13
J-29-14
J-29-15
J-29-16
J-29-17
J-29-18
J-29-19
J-29-20
J-29-21
J-29-22
J-29-23
J-29-24
J-29-25
J-29-26
J-29-27
J-29-28
J-29-29
J-29-30
J-29-31
J-29-32
J-29-33
J-29-34
J-29-35
J-29-36
J-29-37
J-29-38
J-29-39
J-29-40
J-29-41
J-29-42
J-29-43
J-29-44
J-29-45
J-29-46
J-29-47
J-29-48
J-29-49
J-29-50
J-29-51
J-29-52
J-29-53
J-29-54
J-29-55
J-29-56
J-29-57
J-29-58
J-29-59
J-29-60
R¹ is 5-ethyl-3-(trifluoromethyl)pyrazol-1-yl; X is X¹; G is G-1; R^{3a} is H.
J-29-1
J-29-2
J-29-3
J-29-4
J-29-5
J-29-6
J-29-7
J-29-8
J-29-9
J-29-10
J-29-11
J-29-12
J-29-13
J-29-14
J-29-15
J-29-16
J-29-17
J-29-18
J-29-19
J-29-20
J-29-21
J-29-22
J-29-23
J-29-24
J-29-25
J-29-26
J-29-27
J-29-28
J-29-29
J-29-30
J-29-31
J-29-32
J-29-33
J-29-34
J-29-35
J-29-36
J-29-37
J-29-38
J-29-39
J-29-40
J-29-41
J-29-42
J-29-43
J-29-44
J-29-45
J-29-46
J-29-47
J-29-48
J-29-49
J-29-50
J-29-51
J-29-52
J-29-53
J-29-54
J-29-55
J-29-56
J-29-57
J-29-58
J-29-59
J-29-60
R¹ is 5-ethyl-3-(trifluoromethyl)pyrazol-1-yl; X is X²; G is G-1; R^{3a} is H.
J-29-1
J-29-2
J-29-3
J-29-4
J-29-5
J-29-6
J-29-7
J-29-8
J-29-9
J-29-10
J-29-11
J-29-12
J-29-13
J-29-14
J-29-15
J-29-16
J-29-17
J-29-18
J-29-19
J-29-20
J-29-21
J-29-22
J-29-23
J-29-24
J-29-25
J-29-26
J-29-27
J-29-28
J-29-29
J-29-30
J-29-31
J-29-32
J-29-33
J-29-34
J-29-35
J-29-36
J-29-37
J-29-38
J-29-39
J-29-40
J-29-41
J-29-42
J-29-43
J-29-44
J-29-45
J-29-46
J-29-47
J-29-48
J-29-49
J-29-50
J-29-51
J-29-52
J-29-53
J-29-54
J-29-55
J-29-56
J-29-57
J-29-58
J-29-59
J-29-60
R¹ is 5-ethyl-3-(trifluoromethyl)pyrazol-1-yl; X is X¹; G is G-2; R^{3a} is H.
J-29-1
J-29-2
J-29-3
J-29-4
J-29-5
J-29-6
J-29-7
J-29-8
J-29-9
J-29-10
J-29-11
J-29-12
J-29-13
J-29-14
J-29-15
J-29-16
J-29-17
J-29-18
J-29-19
J-29-20
J-29-21
J-29-22
J-29-23
J-29-24
J-29-25
J-29-26
J-29-27
J-29-28
J-29-29
J-29-30
J-29-31
J-29-32
J-29-33
J-29-34
J-29-35
J-29-36
J-29-37
J-29-38
J-29-39
J-29-40
J-29-41
J-29-42
J-29-43
J-29-44
J-29-45
J-29-46
J-29-47
J-29-48
J-29-49
J-29-50
J-29-51
J-29-52
J-29-53
J-29-54
J-29-55
J-29-56
J-29-57
J-29-58
J-29-59
J-29-60
R¹ is 5-ethyl-3-(trifluoromethyl)pyrazol-1-yl; X is X²; G is G-2; R^{3a} is H.
J-29-1
J-29-2
J-29-3
J-29-4
J-29-5
J-29-6
J-29-7
J-29-8
J-29-9
J-29-10
J-29-11
J-29-12
J-29-13
J-29-14
J-29-15
J-29-16
J-29-17
J-29-18
J-29-19
J-29-20
J-29-21
J-29-22
J-29-23
J-29-24
J-29-25
J-29-26
J-29-27
J-29-28
J-29-29
J-29-30
J-29-31
J-29-32
J-29-33
J-29-34
J-29-35
J-29-36
J-29-37
J-29-38
J-29-39
J-29-40
J-29-41
J-29-42
J-29-43
J-29-44
J-29-45
J-29-46
J-29-47
J-29-48
J-29-49
J-29-50
J-29-51
J-29-52
J-29-53
J-29-54
J-29-55
J-29-56
J-29-57
J-29-58
J-29-59
J-29-60
R¹ is 3,5-bis-(trifluoromethyl)pyrazol-1-yl; X is X¹; G is G-1; R^{3a} is H.
J-29-1
J-29-2
J-29-3
J-29-4
J-29-5
J-29-6
J-29-7
J-29-8
J-29-9
J-29-10
J-29-11
J-29-12
J-29-13
J-29-14
J-29-15
J-29-16
J-29-17
J-29-18
J-29-19
J-29-20
J-29-21
J-29-22
J-29-23
J-29-24
J-29-25
J-29-26
J-29-27
J-29-28
J-29-29
J-29-30
J-29-31
J-29-32
J-29-33
J-29-34
J-29-35
J-29-36
J-29-37
J-29-38
J-29-39
J-29-40
J-29-41
J-29-42
J-29-43
J-29-44
J-29-45
J-29-46
J-29-47
J-29-48
J-29-49
J-29-50
J-29-51
J-29-52
J-29-53
J-29-54
J-29-55
J-29-56
J-29-57
J-29-58
J-29-59
J-29-60
R¹ is 3,5-bis-(trifluoromethyl)pyrazol-1-yl; X is X²; G is G-1; R^{3a} is H.
J-29-1
J-29-2
J-29-3
J-29-4
J-29-5
J-29-6
J-29-7
J-29-8
J-29-9
J-29-10
J-29-11
J-29-12
J-29-13
J-29-14
J-29-15
J-29-16
J-29-17
J-29-18
J-29-19
J-29-20
J-29-21
J-29-22
J-29-23
J-29-24
J-29-25
J-29-26
J-29-27
J-29-28
J-29-29
J-29-30
J-29-31
J-29-32
J-29-33
J-29-34
J-29-35
J-29-36
J-29-37
J-29-38
J-29-39
J-29-40
J-29-41
J-29-42
J-29-43
J-29-44
J-29-45
J-29-46
J-29-47
J-29-48
J-29-49
J-29-50
J-29-51
J-29-52
J-29-53
J-29-54
J-29-55
J-29-56
J-29-57
J-29-58
J-29-59
J-29-60
R¹ is 3,5-bis-(trifluoromethyl)pyrazol-1-yl; X is X¹; G is G-2; R^{3a} is H.
J-29-1
J-29-2
J-29-3
J-29-4
J-29-5
J-29-6
J-29-7
J-29-8
J-29-9
J-29-10
J-29-11
J-29-12
J-29-13
J-29-14
J-29-15
J-29-16
J-29-17
J-29-18
J-29-19
J-29-20
J-29-21
J-29-22
J-29-23
J-29-24
J-29-25
J-29-26
J-29-27
J-29-28
J-29-29
J-29-30
J-29-31
J-29-32
J-29-33
J-29-34
J-29-35
J-29-36
J-29-37
J-29-38
J-29-39
J-29-40
J-29-41
J-29-42
J-29-43
J-29-44
J-29-45
J-29-46
J-29-47
J-29-48
J-29-49
J-29-50
J-29-51
J-29-52
J-29-53
J-29-54
J-29-55
J-29-56
J-29-57
J-29-58
J-29-59
J-29-60
R¹ is 3,5-bis-(trifluoromethyl)pyrazol-1-yl; X is X²; G is G-2; R^{3a} is H.
J-29-1
J-29-2
J-29-3
J-29-4
J-29-5
J-29-6
J-29-7
J-29-8
J-29-9
J-29-10
J-29-11
J-29-12
J-29-13
J-29-14
J-29-15
J-29-16
J-29-17
J-29-18
J-29-19
J-29-20
J-29-21
J-29-22
J-29-23
J-29-24
J-29-25
J-29-26
J-29-27
J-29-28
J-29-29
J-29-30
J-29-31
J-29-32
J-29-33
J-29-34
J-29-35
J-29-36
J-29-37
J-29-38
J-29-39
J-29-40
J-29-41
J-29-42
J-29-43
J-29-44
J-29-45
J-29-46
J-29-47
J-29-48
J-29-49
J-29-50
J-29-51
J-29-52
J-29-53
J-29-54
J-29-55
J-29-56
J-29-57
J-29-58
J-29-59
J-29-60
R¹ is 3-methyl-5-(trifluoromethyl)pyrazol-1-yl; X is X¹; G is G-1; R^{3a} is H.
J-29-1
J-29-2
J-29-3
J-29-4
J-29-5
J-29-6
J-29-7
J-29-8
J-29-9
J-29-10
J-29-11
J-29-12
J-29-13
J-29-14
J-29-15
J-29-16
J-29-17
J-29-18
J-29-19
J-29-20
J-29-21
J-29-22
J-29-23
J-29-24
J-29-25
J-29-26
J-29-27
J-29-28
J-29-29
J-29-30
J-29-31
J-29-32
J-29-33
J-29-34
J-29-35
J-29-36
J-29-37
J-29-38
J-29-39
J-29-40
J-29-41
J-29-42
J-29-43
J-29-44
J-29-45
J-29-46
J-29-47
J-29-48
J-29-49
J-29-50
J-29-51
J-29-52
J-29-53
J-29-54
J-29-55
J-29-56
J-29-57
J-29-58
J-29-59
J-29-60
R¹ is 3-methyl-5-(trifluoromethyl)pyrazol-1-yl; X is X²; G is G-1; R^{3a} is H.
J-29-1
J-29-2
J-29-3
J-29-4
J-29-5
J-29-6
J-29-7
J-29-8
J-29-9
J-29-10
J-29-11
J-29-12
J-29-13
J-29-14
J-29-15
J-29-16
J-29-17
J-29-18
J-29-19
J-29-20
J-29-21
J-29-22
J-29-23
J-29-24
J-29-25
J-29-26
J-29-27
J-29-28
J-29-29
J-29-30
J-29-31
J-29-32
J-29-33
J-29-34
J-29-35
J-29-36
J-29-37
J-29-38
J-29-39
J-29-40
J-29-41
J-29-42
J-29-43
J-29-44
J-29-45
J-29-46
J-29-47
J-29-48
J-29-49
J-29-50
J-29-51
J-29-52
J-29-53
J-29-54
J-29-55
J-29-56
J-29-57
J-29-58
J-29-59
J-29-60
R¹ is 3-methyl-5-(trifluoromethyl)pyrazol-1-yl; X is X¹; G is G-2; R^{3a} is H.
J-29-1
J-29-2
J-29-3
J-29-4
J-29-5
J-29-6
J-29-7
J-29-8
J-29-9
J-29-10
J-29-11
J-29-12
J-29-13
J-29-14
J-29-15
J-29-16
J-29-17
J-29-18
J-29-19
J-29-20
J-29-21
J-29-22
J-29-23
J-29-24
J-29-25
J-29-26
J-29-27
J-29-28
J-29-29
J-29-30
J-29-31
J-29-32
J-29-33
J-29-34
J-29-35
J-29-36
J-29-37
J-29-38
J-29-39
J-29-40
J-29-41
J-29-42
J-29-43
J-29-44
J-29-45
J-29-46
J-29-47
J-29-48
J-29-49
J-29-50
J-29-51
J-29-52
J-29-53
J-29-54
J-29-55
J-29-56
J-29-57
J-29-58
J-29-59
J-29-60
R¹ is 3-methyl-5-(trifluoromethyl)pyrazol-1-yl; X is X²; G is G-2; R^{3a} is H.
J-29-1
J-29-2
J-29-3
J-29-4
J-29-5
J-29-6
J-29-7
J-29-8
J-29-9
J-29-10
J-29-11
J-29-12
J-29-13
J-29-14
J-29-15
J-29-16
J-29-17
J-29-18
J-29-19
J-29-20
J-29-21
J-29-22
J-29-23
J-29-24
J-29-25
J-29-26
J-29-27
J-29-28
J-29-29
J-29-30
J-29-31
J-29-32
J-29-33
J-29-34
J-29-35
J-29-36
J-29-37
J-29-38
J-29-39
J-29-40
J-29-41
J-29-42
J-29-43
J-29-44
J-29-45
J-29-46
J-29-47
J-29-48
J-29-49
J-29-50
J-29-51
J-29-52
J-29-53
J-29-54
J-29-55
J-29-56
J-29-57
J-29-58
J-29-59
J-29-60
R¹ is 3-chloro-5-(trifluoromethyl)pyrazol-1-yl; X is X¹; G is G-1; R^{3a} is H.
J-29-1
J-29-2
J-29-3
J-29-4
J-29-5
J-29-6
J-29-7
J-29-8
J-29-9
J-29-10
J-29-11
J-29-12
J-29-13
J-29-14
J-29-15
J-29-16
J-29-17
J-29-18
J-29-19
J-29-20
J-29-21
J-29-22
J-29-23
J-29-24
J-29-25
J-29-26
J-29-27
J-29-28
J-29-29
J-29-30
J-29-31
J-29-32
J-29-33
J-29-34
J-29-35
J-29-36
J-29-37
J-29-38
J-29-39
J-29-40
J-29-41
J-29-42
J-29-43
J-29-44
J-29-45
J-29-46
J-29-47
J-29-48
J-29-49
J-29-50
J-29-51
J-29-52
J-29-53
J-29-54
J-29-55
J-29-56
J-29-57
J-29-58
J-29-59
J-29-60
R¹ is 3-chloro-5-(trifluoromethyl)pyrazol-1-yl; X is X²; G is G-1; R^{3a} is H.
J-29-1
J-29-2
J-29-3
J-29-4
J-29-5
J-29-6
J-29-7
J-29-8
J-29-9
J-29-10
J-29-11
J-29-12
J-29-13
J-29-14
J-29-15
J-29-16
J-29-17
J-29-18
J-29-19
J-29-20
J-29-21
J-29-22
J-29-23
J-29-24
J-29-25
J-29-26
J-29-27
J-29-28
J-29-29
J-29-30
J-29-31
J-29-32
J-29-33
J-29-34
J-29-35
J-29-36
J-29-37
J-29-38
J-29-39
J-29-40
J-29-41
J-29-42
J-29-43
J-29-44
J-29-45
J-29-46
J-29-47
J-29-48
J-29-49
J-29-50
J-29-51
J-29-52
J-29-53
J-29-54
J-29-55
J-29-56
J-29-57
J-29-58
J-29-59
J-29-60
R¹ is 3-chloro-5-(trifluoromethyl)pyrazol-1-yl; X is X¹; G is G-2; R^{3a} is H.
J-29-1
J-29-2
J-29-3
J-29-4
J-29-5
J-29-6
J-29-7
J-29-8
J-29-9
J-29-10
J-29-11
J-29-12
J-29-13
J-29-14
J-29-15
J-29-16
J-29-17
J-29-18
J-29-19
J-29-20
J-29-21
J-29-22
J-29-23
J-29-24
J-29-25
J-29-26
J-29-27
J-29-28
J-29-29
J-29-30
J-29-31
J-29-32
J-29-33
J-29-34
J-29-35
J-29-36
J-29-37
J-29-38
J-29-39
J-29-40
J-29-41
J-29-42
J-29-43
J-29-44
J-29-45
J-29-46
J-29-47
J-29-48
J-29-49
J-29-50
J-29-51
J-29-52
J-29-53
J-29-54
J-29-55
J-29-56
J-29-57
J-29-58
J-29-59
J-29-60
R¹ is 3-chloro-5-(trifluoromethyl)pyrazol-1-yl; X is X²; G is G-2; R^{3a} is H.
J-29-1
J-29-2
J-29-3
J-29-4
J-29-5
J-29-6
J-29-7
J-29-8
J-29-9
J-29-10
J-29-11
J-29-12
J-29-13
J-29-14
J-29-15
J-29-16
J-29-17
J-29-18
J-29-19
J-29-20
J-29-21
J-29-22
J-29-23
J-29-24
J-29-25
J-29-26
J-29-27
J-29-28
J-29-29
J-29-30
J-29-31
J-29-32
J-29-33
J-29-34
J-29-35
J-29-36
J-29-37
J-29-38
J-29-39
J-29-40
J-29-41
J-29-42
J-29-43
J-29-44
J-29-45
J-29-46
J-29-47
J-29-48
J-29-49
J-29-50
J-29-51
J-29-52
J-29-53
J-29-54
J-29-55
J-29-56
J-29-57
J-29-58
J-29-59
J-29-60
R¹ is 3-bromo-5-(trifluoromethyl)pyrazol-1-yl; X is X¹; G is G-1; R^{3a} is H.
J-29-1
J-29-2
J-29-3
J-29-4
J-29-5
J-29-6
J-29-7
J-29-8
J-29-9
J-29-10
J-29-11
J-29-12
J-29-13
J-29-14
J-29-15
J-29-16
J-29-17
J-29-18
J-29-19
J-29-20
J-29-21
J-29-22
J-29-23
J-29-24
J-29-25
J-29-26
J-29-27
J-29-28
J-29-29
J-29-30
J-29-31
J-29-32
J-29-33
J-29-34
J-29-35
J-29-36
J-29-37
J-29-38
J-29-39
J-29-40
J-29-41
J-29-42
J-29-43
J-29-44
J-29-45
J-29-46
J-29-47
J-29-48
J-29-49
J-29-50
J-29-51
J-29-52
J-29-53
J-29-54
J-29-55
J-29-56
J-29-57
J-29-58
J-29-59
J-29-60
R¹ is 3-bromo-5-(trifluoromethyl)pyrazol-1-yl; X is X²; G is G-1; R^{3a} is H.
J-29-1
J-29-2
J-29-3
J-29-4
J-29-5
J-29-6
J-29-7
J-29-8
J-29-9
J-29-10
J-29-11
J-29-12
J-29-13
J-29-14
J-29-15
J-29-16
J-29-17
J-29-18
J-29-19
J-29-20
J-29-21
J-29-22
J-29-23
J-29-24
J-29-25
J-29-26
J-29-27
J-29-28
J-29-29
J-29-30
J-29-31
J-29-32
J-29-33
J-29-34
J-29-35
J-29-36
J-29-37
J-29-38
J-29-39
J-29-40
J-29-41
J-29-42
J-29-43
J-29-44
J-29-45
J-29-46
J-29-47
J-29-48
J-29-49
J-29-50
J-29-51
J-29-52
J-29-53
J-29-54
J-29-55
J-29-56
J-29-57
J-29-58
J-29-59
J-29-60
R¹ is 3-bromo-5-(trifluoromethyl)pyrazol-1-yl; X is X¹; G is G-2; R^{3a} is H.
J-29-1
J-29-2
J-29-3
J-29-4
J-29-5
J-29-6
J-29-7
J-29-8
J-29-9
J-29-10
J-29-11
J-29-12
J-29-13
J-29-14
J-29-15
J-29-16
J-29-17
J-29-18
J-29-19
J-29-20
J-29-21
J-29-22
J-29-23
J-29-24
J-29-25
J-29-26
J-29-27
J-29-28
J-29-29
J-29-30
J-29-31
J-29-32
J-29-33
J-29-34
J-29-35
J-29-36
J-29-37
J-29-38
J-29-39
J-29-40
J-29-41
J-29-42
J-29-43
J-29-44
J-29-45
J-29-46
J-29-47
J-29-48
J-29-49
J-29-50
J-29-51
J-29-52
J-29-53
J-29-54
J-29-55
J-29-56
J-29-57
J-29-58
J-29-59
J-29-60
R¹ is 3-bromo-5-(trifluoromethyl)pyrazol-1-yl; X is X²; G is G-2; R^{3a} is H.
J-29-1
J-29-2
J-29-3
J-29-4
J-29-5
J-29-6
J-29-7
J-29-8
J-29-9
J-29-10
J-29-11
J-29-12
J-29-13
J-29-14
J-29-15
J-29-16
J-29-17
J-29-18
J-29-19
J-29-20
J-29-21
J-29-22
J-29-23
J-29-24
J-29-25
J-29-26
J-29-27
J-29-28
J-29-29
J-29-30
J-29-31
J-29-32
J-29-33
J-29-34
J-29-35
J-29-36
J-29-37
J-29-38
J-29-39
J-29-40
J-29-41
J-29-42
J-29-43
J-29-44
J-29-45
J-29-46
J-29-47
J-29-48
J-29-49
J-29-50
J-29-51
J-29-52
J-29-53
J-29-54
J-29-55
J-29-56
J-29-57
J-29-58
J-29-59
J-29-60
R¹ is 5-methoxy-3-(trifluoromethyl)pyrazol-1-yl; X is X¹; G is G-1; R^{3a} is H.
J-29-1
J-29-2
J-29-3
J-29-4
J-29-5
J-29-6
J-29-7
J-29-8
J-29-9
J-29-10
J-29-11
J-29-12
J-29-13
J-29-14
J-29-15
J-29-16
J-29-17
J-29-18
J-29-19
J-29-20
J-29-21
J-29-22
J-29-23
J-29-24
J-29-25
J-29-26
J-29-27
J-29-28
J-29-29
J-29-30
J-29-31
J-29-32
J-29-33
J-29-34
J-29-35
J-29-36
J-29-37
J-29-38
J-29-39
J-29-40
J-29-41
J-29-42
J-29-43
J-29-44
J-29-45
J-29-46
J-29-47
J-29-48
J-29-49
J-29-50
J-29-51
J-29-52
J-29-53
J-29-54
J-29-55
J-29-56
J-29-57
J-29-58
J-29-59
J-29-60
R¹ is 5-methoxy-3-(trifluoromethyl)pyrazol-1-yl; X is X²; G is G-1; R^{3a} is H.
J-29-1
J-29-2
J-29-3
J-29-4
J-29-5
J-29-6
J-29-7
J-29-8
J-29-9
J-29-10
J-29-11
J-29-12
J-29-13
J-29-14
J-29-15
J-29-16
J-29-17
J-29-18
J-29-19
J-29-20
J-29-21
J-29-22
J-29-23
J-29-24
J-29-25
J-29-26
J-29-27
J-29-28
J-29-29
J-29-30
J-29-31
J-29-32
J-29-33
J-29-34
J-29-35
J-29-36
J-29-37
J-29-38
J-29-39
J-29-40
J-29-41
J-29-42
J-29-43
J-29-44
J-29-45
J-29-46
J-29-47
J-29-48
J-29-49
J-29-50
J-29-51
J-29-52
J-29-53
J-29-54
J-29-55
J-29-56
J-29-57
J-29-58
J-29-59
J-29-60
R¹ is 5-methoxy-3-(trifluoromethyl)pyrazol-1-yl; X is X¹; G is G-2; R^{3a} is H.
J-29-1
J-29-2
J-29-3
J-29-4
J-29-5
J-29-6
J-29-7
J-29-8
J-29-9
J-29-10
J-29-11
J-29-12
J-29-13
J-29-14
J-29-15
J-29-16
J-29-17
J-29-18
J-29-19
J-29-20
J-29-21
J-29-22
J-29-23
J-29-24
J-29-25
J-29-26
J-29-27
J-29-28
J-29-29
J-29-30
J-29-31
J-29-32
J-29-33
J-29-34
J-29-35
J-29-36
J-29-37
J-29-38
J-29-39
J-29-40
J-29-41
J-29-42
J-29-43
J-29-44
J-29-45
J-29-46
J-29-47
J-29-48
J-29-49
J-29-50
J-29-51
J-29-52
J-29-53
J-29-54
J-29-55
J-29-56
J-29-57
J-29-58
J-29-59
J-29-60
R¹ is 5-methoxy-3-(trifluoromethyl)pyrazol-1-yl; X is X²; G is G-2; R^{3a} is H.
J-29-1
J-29-2
J-29-3
J-29-4
J-29-5
J-29-6
J-29-7
J-29-8
J-29-9
J-29-10
J-29-11
J-29-12
J-29-13
J-29-14
J-29-15
J-29-16
J-29-17
J-29-18
J-29-19
J-29-20
J-29-21
J-29-22
J-29-23
J-29-24
J-29-25
J-29-26
J-29-27
J-29-28
J-29-29
J-29-30
J-29-31
J-29-32
J-29-33
J-29-34
J-29-35
J-29-36
J-29-37
J-29-38
J-29-39
J-29-40
J-29-41
J-29-42
J-29-43
J-29-44
J-29-45
J-29-46
J-29-47
J-29-48
J-29-49
J-29-50
J-29-51
J-29-52
J-29-53
J-29-54
J-29-55
J-29-56
J-29-57
J-29-58
J-29-59
J-29-60
R¹ is 5-difluoromethoxy-3-(trifluoromethyl)pyrazol-1-yl; X is X¹; G is G-1;
R^{3a} is H.
J-29-1
J-29-2
J-29-3
J-29-4
J-29-5
J-29-6
J-29-7
J-29-8
J-29-9
J-29-10
J-29-11
J-29-12
J-29-13
J-29-14
J-29-15
J-29-16
J-29-17
J-29-18
J-29-19
J-29-20
J-29-21
J-29-22
J-29-23
J-29-24
J-29-25
J-29-26
J-29-27
J-29-28
J-29-29
J-29-30
J-29-31
J-29-32
J-29-33
J-29-34
J-29-35
J-29-36
J-29-37
J-29-38
J-29-39
J-29-40
J-29-41
J-29-42
J-29-43
J-29-44
J-29-45
J-29-46
J-29-47
J-29-48
J-29-49
J-29-50
J-29-51
J-29-52
J-29-53
J-29-54
J-29-55
J-29-56
J-29-57
J-29-58
J-29-59
J-29-60
R¹ is 5-difluoromethoxy-3-(trifluoromethyl)pyrazol-1-yl; X is X²; G is G-1;
R^{3a} is H.
J-29-1
J-29-2
J-29-3
J-29-4
J-29-5
J-29-6
J-29-7
J-29-8
J-29-9
J-29-10
J-29-11
J-29-12
J-29-13
J-29-14
J-29-15
J-29-16
J-29-17
J-29-18
J-29-19
J-29-20
J-29-21
J-29-22
J-29-23
J-29-24
J-29-25
J-29-26
J-29-27
J-29-28
J-29-29
J-29-30
J-29-31
J-29-32
J-29-33
J-29-34
J-29-35
J-29-36
J-29-37
J-29-38
J-29-39
J-29-40
J-29-41
J-29-42
J-29-43
J-29-44
J-29-45
J-29-46
J-29-47
J-29-48
J-29-49
J-29-50
J-29-51
J-29-52
J-29-53
J-29-54
J-29-55
J-29-56
J-29-57
J-29-58
J-29-59
J-29-60
R¹ is 5-difluoromethoxy-3-(trifluoromethyl)pyrazol-1-yl; X is X¹; G is G-2;
R^{3a} is H.
J-29-1
J-29-2
J-29-3
J-29-4
J-29-5
J-29-6
J-29-7
J-29-8
J-29-9
J-29-10
J-29-11
J-29-12
J-29-13
J-29-14
J-29-15
J-29-16
J-29-17
J-29-18
J-29-19
J-29-20
J-29-21
J-29-22
J-29-23
J-29-24
J-29-25
J-29-26
J-29-27
J-29-28
J-29-29
J-29-30
J-29-31
J-29-32
J-29-33
J-29-34
J-29-35
J-29-36
J-29-37
J-29-38
J-29-39
J-29-40
J-29-41
J-29-42
J-29-43
J-29-44
J-29-45
J-29-46
J-29-47
J-29-48
J-29-49
J-29-50
J-29-51
J-29-52
J-29-53
J-29-54
J-29-55
J-29-56
J-29-57
J-29-58
J-29-59
J-29-60
R¹ is 5-difluoromethoxy-3-(trifluoromethyl)pyrazol-1-yl; X is X²; G is G-2;
R^{3a} is H.
J-29-1
J-29-2
J-29-3
J-29-4
J-29-5
J-29-6
J-29-7
J-29-8
J-29-9
J-29-10
J-29-11
J-29-12
J-29-13
J-29-14
J-29-15
J-29-16
J-29-17
J-29-18
J-29-19
J-29-20
J-29-21
J-29-22
J-29-23
J-29-24
J-29-25
J-29-26
J-29-27
J-29-28
J-29-29
J-29-30
J-29-31
J-29-32
J-29-33
J-29-34
J-29-35
J-29-36
J-29-37
J-29-38
J-29-39
J-29-40
J-29-41
J-29-42
J-29-43
J-29-44
J-29-45
J-29-46
J-29-47
J-29-48
J-29-49
J-29-50
J-29-51
J-29-52
J-29-53
J-29-54
J-29-55
J-29-56
J-29-57
J-29-58
J-29-59
J-29-60
R¹ is 3,5-dichlorotriazol-1-yl; X is X¹; G is G-1; R^{3a} is H.
J-29-1
J-29-2
J-29-3
J-29-4
J-29-5
J-29-6
J-29-7
J-29-8
J-29-9
J-29-10
J-29-11
J-29-12
J-29-13
J-29-14
J-29-15
J-29-16
J-29-17
J-29-18
J-29-19
J-29-20
J-29-21
J-29-22
J-29-23
J-29-24
J-29-25
J-29-26
J-29-27
J-29-28
J-29-29
J-29-30
J-29-31
J-29-32
J-29-33
J-29-34
J-29-35
J-29-36
J-29-37
J-29-38
J-29-39
J-29-40
J-29-41
J-29-42
J-29-43
J-29-44
J-29-45
J-29-46
J-29-47
J-29-48
J-29-49
J-29-50
J-29-51
J-29-52
J-29-53
J-29-54
J-29-55
J-29-56
J-29-57
J-29-58
J-29-59
J-29-60
R¹ is 3,5-dichlorotriazol-1-yl; X is X²; G is G-1; R^{3a} is H.
J-29-1
J-29-2
J-29-3
J-29-4
J-29-5
J-29-6
J-29-7
J-29-8
J-29-9
J-29-10
J-29-11
J-29-12
J-29-13
J-29-14
J-29-15
J-29-16
J-29-17
J-29-18
J-29-19
J-29-20
J-29-21
J-29-22
J-29-23
J-29-24
J-29-25
J-29-26
J-29-27
J-29-28
J-29-29
J-29-30
J-29-31
J-29-32
J-29-33
J-29-34
J-29-35
J-29-36
J-29-37
J-29-38
J-29-39
J-29-40
J-29-41
J-29-42
J-29-43
J-29-44
J-29-45
J-29-46
J-29-47
J-29-48
J-29-49
J-29-50
J-29-51
J-29-52
J-29-53
J-29-54
J-29-55
J-29-56
J-29-57
J-29-58
J-29-59
J-29-60
R¹ is 3,5-dichlorotriazol-1-yl; X is X¹; G is G-2; R^{3a} is H.
J-29-1
J-29-2
J-29-3
J-29-4
J-29-5
J-29-6
J-29-7
J-29-8
J-29-9
J-29-10
J-29-11
J-29-12
J-29-13
J-29-14
J-29-15
J-29-16
J-29-17
J-29-18
J-29-19
J-29-20
J-29-21
J-29-22
J-29-23
J-29-24
J-29-25
J-29-26
J-29-27
J-29-28
J-29-29
J-29-30
J-29-31
J-29-32
J-29-33
J-29-34
J-29-35
J-29-36
J-29-37
J-29-38
J-29-39
J-29-40
J-29-41
J-29-42
J-29-43
J-29-44
J-29-45
J-29-46
J-29-47
J-29-48
J-29-49
J-29-50
J-29-51
J-29-52
J-29-53
J-29-54
J-29-55
J-29-56
J-29-57
J-29-58
J-29-59
J-29-60
R¹ is 3,5-dichlorotriazol-1-yl; X is X²; G is G-2; R^{3a} is H.
J-29-1
J-29-2
J-29-3
J-29-4
J-29-5
J-29-6
J-29-7
J-29-8
J-29-9
J-29-10
J-29-11
J-29-12
J-29-13
J-29-14
J-29-15
J-29-16
J-29-17
J-29-18
J-29-19
J-29-20
J-29-21
J-29-22
J-29-23
J-29-24
J-29-25
J-29-26
J-29-27
J-29-28
J-29-29
J-29-30
J-29-31
J-29-32
J-29-33
J-29-34
J-29-35
J-29-36
J-29-37
J-29-38
J-29-39
J-29-40
J-29-41
J-29-42
J-29-43
J-29-44
J-29-45
J-29-46
J-29-47
J-29-48
J-29-49
J-29-50
J-29-51
J-29-52
J-29-53
J-29-54
J-29-55
J-29-56
J-29-57
J-29-58
J-29-59
J-29-60
R¹ is 3,5-dibromotriazol-1-yl; X is X¹; G is G-1; R^{3a} is H.
J-29-1
J-29-2
J-29-3
J-29-4
J-29-5
J-29-6
J-29-7
J-29-8
J-29-9
J-29-10
J-29-11
J-29-12
J-29-13
J-29-14
J-29-15
J-29-16
J-29-17
J-29-18
J-29-19
J-29-20
J-29-21
J-29-22
J-29-23
J-29-24
J-29-25
J-29-26
J-29-27
J-29-28
J-29-29
J-29-30
J-29-31
J-29-32
J-29-33
J-29-34
J-29-35
J-29-36
J-29-37
J-29-38
J-29-39
J-29-40
J-29-41
J-29-42
J-29-43
J-29-44
J-29-45
J-29-46
J-29-47
J-29-48
J-29-49
J-29-50
J-29-51
J-29-52
J-29-53
J-29-54
J-29-55
J-29-56
J-29-57
J-29-58
J-29-59
J-29-60
R¹ is 3,5-dibromotriazol-1-yl; X is X²; G is G-1; R^{3a} is H.
J-29-1
J-29-2
J-29-3
J-29-4
J-29-5
J-29-6
J-29-7
J-29-8
J-29-9
J-29-10
J-29-11
J-29-12
J-29-13
J-29-14
J-29-15
J-29-16
J-29-17
J-29-18
J-29-19
J-29-20
J-29-21
J-29-22
J-29-23
J-29-24
J-29-25
J-29-26
J-29-27
J-29-28
J-29-29
J-29-30
J-29-31
J-29-32
J-29-33
J-29-34
J-29-35
J-29-36
J-29-37
J-29-38
J-29-39
J-29-40
J-29-41
J-29-42
J-29-43
J-29-44
J-29-45
J-29-46
J-29-47
J-29-48
J-29-49
J-29-50
J-29-51
J-29-52
J-29-53
J-29-54
J-29-55
J-29-56
J-29-57
J-29-58
J-29-59
J-29-60
R¹ is 3,5-dibromotriazol-1-yl; X is X¹; G is G-2; R^{3a} is H.
J-29-1
J-29-2
J-29-3
J-29-4
J-29-5
J-29-6
J-29-7
J-29-8
J-29-9
J-29-10
J-29-11
J-29-12
J-29-13
J-29-14
J-29-15
J-29-16
J-29-17
J-29-18
J-29-19
J-29-20
J-29-21
J-29-22
J-29-23
J-29-24
J-29-25
J-29-26
J-29-27
J-29-28
J-29-29
J-29-30
J-29-31
J-29-32
J-29-33
J-29-34
J-29-35
J-29-36
J-29-37
J-29-38
J-29-39
J-29-40
J-29-41
J-29-42
J-29-43
J-29-44
J-29-45
J-29-46
J-29-47
J-29-48
J-29-49
J-29-50
J-29-51
J-29-52
J-29-53
J-29-54
J-29-55
J-29-56
J-29-57
J-29-58
J-29-59
J-29-60
R¹ is 3,5-dibromotriazol-1-yl; X is X²; G is G-2; R^{3a} is H.
J-29-1
J-29-2
J-29-3
J-29-4
J-29-5
J-29-6
J-29-7
J-29-8
J-29-9
J-29-10
J-29-11
J-29-12
J-29-13
J-29-14
J-29-15
J-29-16
J-29-17
J-29-18
J-29-19
J-29-20
J-29-21
J-29-22
J-29-23
J-29-24
J-29-25
J-29-26
J-29-27
J-29-28
J-29-29
J-29-30
J-29-31
J-29-32
J-29-33
J-29-34
J-29-35
J-29-36
J-29-37
J-29-38
J-29-39
J-29-40
J-29-41
J-29-42
J-29-43
J-29-44
J-29-45
J-29-46
J-29-47
J-29-48
J-29-49
J-29-50
J-29-51
J-29-52
J-29-53
J-29-54
J-29-55
J-29-56
J-29-57
J-29-58
J-29-59
J-29-60
R¹ is 3,5-dimethyltriazol-1-yl; X is X¹; G is G-1; R^{3a} is H.
J-29-1
J-29-2
J-29-3
J-29-4
J-29-5
J-29-6
J-29-7
J-29-8
J-29-9
J-29-10
J-29-11
J-29-12
J-29-13
J-29-14
J-29-15
J-29-16
J-29-17
J-29-18
J-29-19
J-29-20
J-29-21
J-29-22
J-29-23
J-29-24
J-29-25
J-29-26
J-29-27
J-29-28
J-29-29
J-29-30
J-29-31
J-29-32
J-29-33
J-29-34
J-29-35
J-29-36
J-29-37
J-29-38
J-29-39
J-29-40
J-29-41
J-29-42
J-29-43
J-29-44
J-29-45
J-29-46
J-29-47
J-29-48
J-29-49
J-29-50
J-29-51
J-29-52
J-29-53
J-29-54
J-29-55
J-29-56
J-29-57
J-29-58
J-29-59
J-29-60
R¹ is 3,5-dimethyltriazol-1-yl; X is X²; G is G-1; R^{3a} is H.
J-29-1
J-29-2
J-29-3
J-29-4
J-29-5
J-29-6
J-29-7
J-29-8
J-29-9
J-29-10
J-29-11
J-29-12
J-29-13
J-29-14
J-29-15
J-29-16
J-29-17
J-29-18
J-29-19
J-29-20
J-29-21
J-29-22
J-29-23
J-29-24
J-29-25
J-29-26
J-29-27
J-29-28
J-29-29
J-29-30
J-29-31
J-29-32
J-29-33
J-29-34
J-29-35
J-29-36
J-29-37
J-29-38
J-29-39
J-29-40
J-29-41
J-29-42
J-29-43
J-29-44
J-29-45
J-29-46
J-29-47
J-29-48
J-29-49
J-29-50
J-29-51
J-29-52
J-29-53
J-29-54
J-29-55
J-29-56
J-29-57
J-29-58
J-29-59
J-29-60
R¹ is 3,5-dimethyltriazol-1-yl; X is X¹; G is G-2; R^{3a} is H.
J-29-1
J-29-2
J-29-3
J-29-4
J-29-5
J-29-6
J-29-7
J-29-8
J-29-9
J-29-10
J-29-11
J-29-12
J-29-13
J-29-14
J-29-15
J-29-16
J-29-17
J-29-18
J-29-19
J-29-20
J-29-21
J-29-22
J-29-23
J-29-24
J-29-25
J-29-26
J-29-27
J-29-28
J-29-29
J-29-30
J-29-31
J-29-32
J-29-33
J-29-34
J-29-35
J-29-36
J-29-37
J-29-38
J-29-39
J-29-40
J-29-41
J-29-42
J-29-43
J-29-44
J-29-45
J-29-46
J-29-47
J-29-48
J-29-49
J-29-50
J-29-51
J-29-52
J-29-53
J-29-54
J-29-55
J-29-56
J-29-57
J-29-58
J-29-59
J-29-60
R¹ is 3,5-dimethyltriazol-1-yl; X is X²; G is G-2; R^{3a} is H.
J-29-1
J-29-2
J-29-3
J-29-4
J-29-5
J-29-6
J-29-7
J-29-8
J-29-9
J-29-10
J-29-11
J-29-12
J-29-13
J-29-14
J-29-15
J-29-16
J-29-17
J-29-18
J-29-19
J-29-20
J-29-21
J-29-22
J-29-23
J-29-24
J-29-25
J-29-26
J-29-27
J-29-28
J-29-29
J-29-30
J-29-31
J-29-32
J-29-33
J-29-34
J-29-35
J-29-36
J-29-37
J-29-38
J-29-39
J-29-40
J-29-41
J-29-42
J-29-43
J-29-44
J-29-45
J-29-46
J-29-47
J-29-48
J-29-49
J-29-50
J-29-51
J-29-52
J-29-53
J-29-54
J-29-55
J-29-56
J-29-57
J-29-58
J-29-59
J-29-60
R¹ is 5-methyl-3-(trifluoromethyl)triazol-1-yl; X is X¹; G is G-1; R^{3a} is H.
J-29-1
J-29-2
J-29-3
J-29-4
J-29-5
J-29-6
J-29-7
J-29-8
J-29-9
J-29-10
J-29-11
J-29-12
J-29-13
J-29-14
J-29-15
J-29-16
J-29-17
J-29-18
J-29-19
J-29-20
J-29-21
J-29-22
J-29-23
J-29-24
J-29-25
J-29-26
J-29-27
J-29-28
J-29-29
J-29-30
J-29-31
J-29-32
J-29-33
J-29-34
J-29-35
J-29-36
J-29-37
J-29-38
J-29-39
J-29-40
J-29-41
J-29-42
J-29-43
J-29-44
J-29-45
J-29-46
J-29-47
J-29-48
J-29-49
J-29-50
J-29-51
J-29-52
J-29-53
J-29-54
J-29-55
J-29-56
J-29-57
J-29-58
J-29-59
J-29-60
R¹ is 5-methyl-3-(trifluoromethyl)triazol-1-yl; X is X²; G is G-1; R^{3a} is H.
J-29-1
J-29-2
J-29-3
J-29-4
J-29-5
J-29-6
J-29-7
J-29-8
J-29-9
J-29-10
J-29-11
J-29-12
J-29-13
J-29-14
J-29-15
J-29-16
J-29-17
J-29-18
J-29-19
J-29-20
J-29-21
J-29-22
J-29-23
J-29-24
J-29-25
J-29-26
J-29-27
J-29-28
J-29-29
J-29-30
J-29-31
J-29-32
J-29-33
J-29-34
J-29-35
J-29-36
J-29-37
J-29-38
J-29-39
J-29-40
J-29-41
J-29-42
J-29-43
J-29-44
J-29-45
J-29-46
J-29-47
J-29-48
J-29-49
J-29-50
J-29-51
J-29-52
J-29-53
J-29-54
J-29-55
J-29-56
J-29-57
J-29-58
J-29-59
J-29-60
R¹ is 5-methyl-3-(trifluoromethyl)triazol-1-yl; X is X¹; G is G-2; R^{3a} is H.
J-29-1
J-29-2
J-29-3
J-29-4
J-29-5
J-29-6
J-29-7
J-29-8
J-29-9
J-29-10
J-29-11
J-29-12
J-29-13
J-29-14
J-29-15
J-29-16
J-29-17
J-29-18
J-29-19
J-29-20
J-29-21
J-29-22
J-29-23
J-29-24
J-29-25
J-29-26
J-29-27
J-29-28
J-29-29
J-29-30
J-29-31
J-29-32
J-29-33
J-29-34
J-29-35
J-29-36
J-29-37
J-29-38
J-29-39
J-29-40
J-29-41
J-29-42
J-29-43
J-29-44
J-29-45
J-29-46
J-29-47
J-29-48
J-29-49
J-29-50
J-29-51
J-29-52
J-29-53
J-29-54
J-29-55
J-29-56
J-29-57
J-29-58
J-29-59
J-29-60
R¹ is 5-methyl-3-(trifluoromethyl)triazol-1-yl; X is X²; G is G-2; R^{3a} is H.
J-29-1
J-29-2
J-29-3
J-29-4
J-29-5
J-29-6
J-29-7
J-29-8
J-29-9
J-29-10
J-29-11
J-29-12
J-29-13
J-29-14
J-29-15
J-29-16
J-29-17
J-29-18
J-29-19
J-29-20
J-29-21
J-29-22
J-29-23
J-29-24
J-29-25
J-29-26
J-29-27
J-29-28
J-29-29
J-29-30
J-29-31
J-29-32
J-29-33
J-29-34
J-29-35
J-29-36
J-29-37
J-29-38
J-29-39
J-29-40
J-29-41
J-29-42
J-29-43
J-29-44
J-29-45
J-29-46
J-29-47
J-29-48
J-29-49
J-29-50
J-29-51
J-29-52
J-29-53
J-29-54
J-29-55
J-29-56
J-29-57
J-29-58
J-29-59
J-29-60
R¹ is 3-methyl-5-(trifluoromethyl)triazol-1-yl; X is X¹; G is G-1; R^{3a} is H.
J-29-1
J-29-2
J-29-3
J-29-4
J-29-5
J-29-6
J-29-7
J-29-8
J-29-9
J-29-10
J-29-11
J-29-12
J-29-13
J-29-14
J-29-15
J-29-16
J-29-17
J-29-18
J-29-19
J-29-20
J-29-21
J-29-22
J-29-23
J-29-24
J-29-25
J-29-26
J-29-27
J-29-28
J-29-29
J-29-30
J-29-31
J-29-32
J-29-33
J-29-34
J-29-35
J-29-36
J-29-37
J-29-38
J-29-39
J-29-40
J-29-41
J-29-42
J-29-43
J-29-44
J-29-45
J-29-46
J-29-47
J-29-48
J-29-49
J-29-50
J-29-51
J-29-52
J-29-53
J-29-54
J-29-55
J-29-56
J-29-57
J-29-58
J-29-59
J-29-60
R¹ is 3-methyl-5-(trifluoromethyl)triazol-1-yl; X is X²; G is G-1; R^{3a} is H.
J-29-1
J-29-2
J-29-3
J-29-4
J-29-5
J-29-6
J-29-7
J-29-8
J-29-9
J-29-10
J-29-11
J-29-12
J-29-13
J-29-14
J-29-15
J-29-16
J-29-17
J-29-18
J-29-19
J-29-20
J-29-21
J-29-22
J-29-23
J-29-24
J-29-25
J-29-26
J-29-27
J-29-28
J-29-29
J-29-30
J-29-31
J-29-32
J-29-33
J-29-34
J-29-35
J-29-36
J-29-37
J-29-38
J-29-39
J-29-40
J-29-41
J-29-42
J-29-43
J-29-44
J-29-45
J-29-46
J-29-47
J-29-48
J-29-49
J-29-50
J-29-51
J-29-52
J-29-53
J-29-54
J-29-55
J-29-56
J-29-57
J-29-58
J-29-59
J-29-60
R¹ is 3-methyl-5-(trifluoromethyl)triazol-1-yl; X is X¹; G is G-2; R^{3a} is H.
J-29-1
J-29-2
J-29-3
J-29-4
J-29-5
J-29-6
J-29-7
J-29-8
J-29-9
J-29-10
J-29-11
J-29-12
J-29-13
J-29-14
J-29-15
J-29-16
J-29-17
J-29-18
J-29-19
J-29-20
J-29-21
J-29-22
J-29-23
J-29-24
J-29-25
J-29-26
J-29-27
J-29-28
J-29-29
J-29-30
J-29-31
J-29-32
J-29-33
J-29-34
J-29-35
J-29-36
J-29-37
J-29-38
J-29-39
J-29-40
J-29-41
J-29-42
J-29-43
J-29-44
J-29-45
J-29-46
J-29-47
J-29-48
J-29-49
J-29-50
J-29-51
J-29-52
J-29-53
J-29-54
J-29-55
J-29-56
J-29-57
J-29-58
J-29-59
J-29-60
R¹ is 3-methyl-5-(trifluoromethyl)triazol-1-yl; X is X²; G is G-2; R^{3a} is H.
J-29-1
J-29-2
J-29-3
J-29-4
J-29-5
J-29-6
J-29-7
J-29-8
J-29-9
J-29-10
J-29-11
J-29-12
J-29-13
J-29-14
J-29-15
J-29-16
J-29-17
J-29-18
J-29-19
J-29-20
J-29-21
J-29-22
J-29-23
J-29-24
J-29-25
J-29-26
J-29-27
J-29-28
J-29-29
J-29-30
J-29-31
J-29-32
J-29-33
J-29-34
J-29-35
J-29-36
J-29-37
J-29-38
J-29-39
J-29-40
J-29-41
J-29-42
J-29-43
J-29-44
J-29-45
J-29-46
J-29-47
J-29-48
J-29-49
J-29-50
J-29-51
J-29-52
J-29-53
J-29-54
J-29-55
J-29-56
J-29-57
J-29-58
J-29-59
J-29-60
R¹ is 3,5-bis-(trifluoromethyl)triazol-1-yl; X is X¹; G is G-1; R^{3a} is H.
J-29-1
J-29-2
J-29-3
J-29-4
J-29-5
J-29-6
J-29-7
J-29-8
J-29-9
J-29-10
J-29-11
J-29-12
J-29-13
J-29-14
J-29-15
J-29-16
J-29-17
J-29-18
J-29-19
J-29-20
J-29-21
J-29-22
J-29-23
J-29-24
J-29-25
J-29-26
J-29-27
J-29-28
J-29-29
J-29-30
J-29-31
J-29-32
J-29-33
J-29-34
J-29-35
J-29-36
J-29-37
J-29-38
J-29-39
J-29-40
J-29-41
J-29-42
J-29-43
J-29-44
J-29-45
J-29-46
J-29-47
J-29-48
J-29-49
J-29-50
J-29-51
J-29-52
J-29-53
J-29-54
J-29-55
J-29-56
J-29-57
J-29-58
J-29-59
J-29-60
R¹ is 3,5-bis-(trifluoromethyl)triazol-1-yl; X is X²; G is G-1; R^{3a} is H.
J-29-1
J-29-2
J-29-3
J-29-4
J-29-5
J-29-6
J-29-7
J-29-8
J-29-9
J-29-10
J-29-11
J-29-12
J-29-13
J-29-14
J-29-15
J-29-16
J-29-17
J-29-18
J-29-19
J-29-20
J-29-21
J-29-22
J-29-23
J-29-24
J-29-25
J-29-26
J-29-27
J-29-28
J-29-29
J-29-30
J-29-31
J-29-32
J-29-33
J-29-34
J-29-35
J-29-36
J-29-37
J-29-38
J-29-39
J-29-40
J-29-41
J-29-42
J-29-43
J-29-44
J-29-45
J-29-46
J-29-47
J-29-48
J-29-49
J-29-50
J-29-51
J-29-52
J-29-53
J-29-54
J-29-55
J-29-56
J-29-57
J-29-58
J-29-59
J-29-60
R¹ is 3,5-bis-(trifluoromethyl)triazol-1-yl; X is X¹; G is G-2; R^{3a} is H.
J-29-1
J-29-2
J-29-3
J-29-4
J-29-5
J-29-6
J-29-7
J-29-8
J-29-9
J-29-10
J-29-11
J-29-12
J-29-13
J-29-14
J-29-15
J-29-16
J-29-17
J-29-18
J-29-19
J-29-20
J-29-21
J-29-22
J-29-23
J-29-24
J-29-25
J-29-26
J-29-27
J-29-28
J-29-29
J-29-30
J-29-31
J-29-32
J-29-33
J-29-34
J-29-35
J-29-36
J-29-37
J-29-38
J-29-39
J-29-40
J-29-41
J-29-42
J-29-43
J-29-44
J-29-45
J-29-46
J-29-47
J-29-48
J-29-49
J-29-50
J-29-51
J-29-52
J-29-53
J-29-54
J-29-55
J-29-56
J-29-57
J-29-58
J-29-59
J-29-60
R¹ is 3,5-bis-(trifluoromethyl)triazol-1-yl; X is X²; G is G-2; R^{3a} is H.
J-29-1
J-29-2
J-29-3
J-29-4
J-29-5
J-29-6
J-29-7
J-29-8
J-29-9
J-29-10
J-29-11
J-29-12
J-29-13
J-29-14
J-29-15
J-29-16
J-29-17
J-29-18
J-29-19
J-29-20
J-29-21
J-29-22
J-29-23
J-29-24
J-29-25
J-29-26
J-29-27
J-29-28
J-29-29
J-29-30
J-29-31
J-29-32
J-29-33
J-29-34
J-29-35
J-29-36
J-29-37
J-29-38
J-29-39
J-29-40
J-29-41
J-29-42
J-29-43
J-29-44
J-29-45
J-29-46
J-29-47
J-29-48
J-29-49
J-29-50
J-29-51
J-29-52
J-29-53
J-29-54
J-29-55
J-29-56
J-29-57
J-29-58
J-29-59
J-29-60
*The definitions of G and J-29-1 through J-29-57 in the compounds of this table are as defined in Exhibits 2 and A in the above Embodiments.
R¹ is 2,5-dichlorophenyl; X is X¹; G is G-1; R^{3a} is H.

Table 5 above identifies particular compounds comprising a J group selected from J-29-1 through J-29-60 (i.e. particular examples of J-29). As many J-29-1 to J-29-60 include a chiral center, these J groups are illustrated in a particular enantiomeric configuration, which in some instances may provide the greatest fungicidal activity. One skilled in the art immediately recognizes the antipode (i.e. opposite enantiomer) for each of the compounds listed, and furthermore understands that the enantiomers can be present as pure enantiomers or in mixtures enriched in one enantiomer or in racemic mixtures.

TABLE 6* 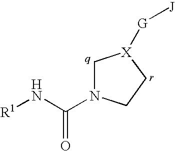
R¹ is 2,5-dimethylphenyl; X is X¹; G is G-1.
J
J-29-1
J-29-2
J-29-3
J-29-4
J-29-5
J-29-6
J-29-7
J-29-8
J-29-9
J-29-10
J-29-11
J-29-12
J-29-13
J-29-14
J-29-15
J-29-16
J-29-17
J-29-18
J-29-19
J-29-20
J-29-21
J-29-22
J-29-23
J-29-24
J-29-25
J-29-26
J-29-27
J-29-28
J-29-29
J-29-30
J-29-31
J-29-32
J-29-33
J-29-34
J-29-35
J-29-36
J-29-37
J-29-38
J-29-39
J-29-40
J-29-41
J-29-42
J-29-43
J-29-44
J-29-45
J-29-46
J-29-47
J-29-48
J-29-49
J-29-50
J-29-51
J-29-52
J-29-53
J-29-54
J-29-55
J-29-56
J-29-57
J-29-58
J-29-59
J-29-60
*The definitions of G and J-29-1 through J-29-60 in the compounds of this table are as defined in Exhibits 2 and A in the above Embodiments.

TABLE 7*

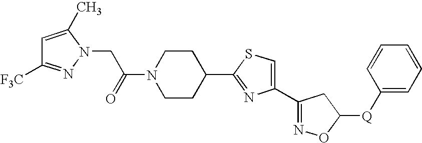

| Q | Q-orientation** |
|---|---|
| Q-1 | 2/3 |
| Q-1 | 2/4 |
| Q-1 | 2/5 |
| Q-2 | 2/3 |
| Q-2 | 2/4 |
| Q-2 | 2/5 |
| Q-3 | 2/3 |
| Q-3 | 2/4 |
| Q-3 | 2/5 |
| Q-4 | 5/4 |
| Q-4 | 5/2 |
| Q-5 | 2/4 |
| Q-5 | 2/5 |
| Q-6 | 2/4 |
| Q-6 | 2/5 |
| Q-7 | 5/2 |
| Q-7 | 5/4 |
| Q-8 | 5/3 |
| Q-8 | 5/4 |
| Q-9 | 5/3 |
| Q-9 | 5/4 |
| Q-10 | 5/3 |
| Q-10 | 5/4 |
| Q-11 | 5/2 |
| Q-11 | 5/4 |
| Q-12 | 2/4 |
| Q-12 | 2/5 |
| Q-13 | 2/5 |
| Q-14 | 5/3 |
| Q-15 | 2/5 |
| Q-16 | 2/5 |
| Q-17 | 3/5 |
| Q-18 | 5/3 |
| Q-19 | 3/2 |
| Q-19 | 3/4 |
| Q-19 | 3/5 |
| Q-20 | 3/2 |
| Q-20 | 3/4 |
| Q-20 | 3/5 |
| Q-21 | 3/2 |
| Q-21 | 3/4 |
| Q-21 | 3/5 |
| Q-22 | 3/4 |
| Q-22 | 3/5 |
| Q-23 | 4/3 |
| Q-23 | 4/5 |
| Q-24 | 4/2 |
| Q-24 | 4/5 |
| Q-25 | 4/2 |
| Q-25 | 4/5 |
| Q-26 | 4/3 |
| Q-26 | 4/5 |
| Q-27 | 4/3 |
| Q-27 | 4/5 |
| Q-28 | 4/2 |
| Q-28 | 4/5 |
| Q-29 | 3/5 |
| Q-30 | 3/4 |
| Q-31 | 3/5 |
| Q-32 | 2/3 |
| Q-32 | 2/4 |
| Q-32 | 2/5 |
| Q-32 | 2/6 |
| Q-33 | 3/2 |
| Q-33 | 3/4 |
| Q-33 | 3/5 |
| Q-33 | 3/6 |
| Q-34 | 4/2 |
| Q-34 | 4/3 |
| Q-35 | 3/4 |
| Q-35 | 3/5 |
| Q-35 | 3/6 |
| Q-36 | 2/4 |
| Q-36 | 2/5 |
| Q-37 | 2/3 |
| Q-37 | 2/5 |
| Q-37 | 2/6 |
| Q-38 | 4/2 |
| Q-38 | 4/5 |
| Q-38 | 4/6 |
| Q-39 | 5/2 |
| Q-39 | 5/4 |
| Q-39 | 5/6 |
| Q-40 | 4/3 |
| Q-40 | 4/5 |
| Q-40 | 4/6 |
| Q-41 | 3/5 |
| Q-41 | 3/6 |
| Q-42 | 6/3 |
| Q-42 | 6/5 |
| Q-43 | 2/4 |
| Q-44 | 5/3 |
| Q-44 | 5/6 |
| Q-45 | 1/2 |
| Q-45 | 1/3 |
| Q-45 | 1/4 |
| Q-46 | 1/2 |
| Q-46 | 1/3 |
| Q-46 | 1/4 |
| Q-46 | 1/5 |
| Q-47 | 1/2 |
| Q-47 | 1/3 |
| Q-47 | 1/4 |
| Q-48 | 1/1 |
| Q-48 | 1/2 |
| Q-49 | 1/1 |
| Q-49 | 1/2 |
| Q-50 | 1/1 |
| Q-50 | 1/2 |
| Q-50 | 1/3 |
| Q-51 | 1/1 |
| Q-51 | 1/2 |
| Q-51 | 1/3 |
| Q-52 | 1/1 |
| Q-52 | 1/2 |
| Q-52 | 1/3 |
| Q-53 | 1/1 |
| Q-53 | 1/2 |
| Q-53 | 1/3 |
| Q-54 | 1/1 |
| Q-54 | 1/2 |
| Q-54 | 1/3 |
| Q-54 | 1/4 |
| Q-55 | 1/1 |
| Q-55 | 1/2 |
| Q-55 | 1/3 |
| Q-55 | 1/4 |
| Q-56 | 2/3 |
| Q-56 | 2/5 |
| Q-56 | 2/6 |
| Q-57 | 3/2 |
| Q-57 | 3/5 |
| Q-57 | 3/6 |
| Q-58 | 2/3 |
| Q-58 | 2/5 |
| Q-58 | 2/6 |
| Q-59 | 3/2 |
| Q-59 | 3/5 |
| Q-59 | 3/6 |
| Q-60 | 1/3 |
| Q-61 | 1/3 |
| Q-62 | 3/5 |
| Q-63 | 2/4 |
| Q-63 | 2/5 |
| Q-64 | 2/3 |
| Q-64 | 2/4 |
| Q-64 | 2/5 |
| Q-65 | 1/3 |
| Q-65 | 1/4 |
| Q-65 | 1/5 |
| Q-66 | 2/3 |
| Q-66 | 2/4 |
| Q-66 | 2/6 |
| Q-67 | 2/4 |
| Q-67 | 2/5 |
| Q-67 | 2/6 |
| Q-68 | 3/6 |
| Q-68 | 3/8 |
| Q-69 | 1/3 |
| Q-69 | 1/4 |
| Q-69 | 1/6 |
| Q-70 | 3/4 |
| Q-70 | 3/6 |
| Q-71 | 3/4 |
| Q-71 | 3/6 |
| Q-72 | 1/4 |
| Q-72 | 1/6 |
| Q-73 | 1/4 |
| Q-73 | 1/5 |
| Q-73 | 1/6 |
| Q-73 | 1/7 |
| Q-73 | 1/8 |
| Q-74 | 1/4 |
| Q-74 | 1/6 |
| Q-74 | 1/8 |
| Q-75 | 1/4 |
| Q-75 | 1/6 |
| Q-75 | 1/7 |
| Q-75 | 1/8 |
| Q-76 | 1/6 |
| Q-76 | 1/7 |
| Q-77 | 1/6 |
| Q-77 | 1/7 |
| Q-78 | 1/5 |
| Q-78 | 1/6 |
| Q-79 | 3/6 |
| Q-79 | 3/7 |
| Q-80 | 3/6 |
| Q-80 | 3/7 |
| Q-81 | 3/4 |
| Q-81 | 3/5 |
| Q-82 | 2/5 |
| Q-82 | 2/6 |
| Q-82 | 2/7 |
| Q-83 | 3/5 |
| Q-84 | 1/5 |
| Q-85 | 3/5 |
| Q-86 | 3/5 |
| Q-87 | 3/4 |
| Q-87 | 3/5 |
| Q-88 | 1/4 |
| Q-88 | 1/5 |
| Q-89 | 3/4 |
| Q-89 | 3/5 |
| Q-90 | 1/3 |
| Q-90 | 1/4 |
| Q-90 | 1/5 |
| Q-91 | 3/4 |
| Q-91 | 3/5 |
| Q-91 | 3/6 |
| Q-92 | 1/4 |
| Q-92 | 1/5 |
| Q-92 | 1/6 |
| Q-93 | 3/4 |
| Q-93 | 3/5 |
| Q-93 | 3/6 |
| Q-94 | 1/3 |
| Q-94 | 1/4 |
| Q-94 | 1/5 |
| Q-94 | 1/6 |
| Q-95 | 2/5 |
| Q-95 | 2/6 |
| Q-96 | 2/4 |
| Q-96 | 2/6 |
| Q-97 | 2/5 |
| Q-97 | 2/6 |
| Q-98 | 2/4 |
| Q-98 | 2/7 |
| Q-99 | 2/5 |
| Q-99 | 2/6 |
| Q-100 | 3/5 |
| Q-100 | 3/6 |
| Q-101 | 3/4 |
| Q-101 | 3/7 |
| Q-102 | 1/5 |
| Q-103 | 1/2 |
| Q-103 | 1/3 |
| Q-104 | 1/3 |
| Q-104 | 1/4 |
| Q-104 | 1/5 |
| Q-105 | 1/2 |
| Q-105 | 1/4 |
| Q-105 | 1/5 |
| Q-106 | 1/3 |
| Q-106 | 1/5 |

*The definitions of Q groups (Q-1 through Q-106) in this table are as defined in Exhibit 4 in the above Embodiments; p is 1, q is 0, and R7 is phenyl depicted in the above molecular structure.
**Q-orientation refers to the attachment points for the remainder of the molecule and the R7 (phenyl) substituent on the ring of Q. The first number refers to the position on the Q ring attaching Q to the remainder of the molecule. The second number refers to the position on the Q ring where the R7 (phenyl) substituent is attached.

TABLE 8* 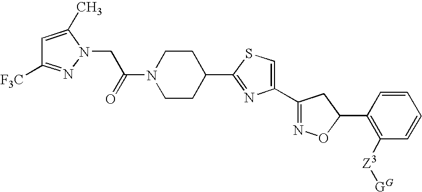
wherein G^{G} is G^{A}, G^{N} or G^{P} as indicated below.
G^{G}
Z³ is a direct bond; r is 0; R²² is Me.
G^{A}-1
G^{A}-2
G^{A}-3
G^{A}-4
G^{A}-5
G^{A}-6
G^{A}-7
G^{A}-8
G^{A}-9
G^{A}-10
G^{A}-11
G^{A}-12
G^{A}-13
G^{A}-14
G^{A}-15
G^{A}-16
G^{A}-17
G^{A}-18
G^{A}-19
G^{A}-20
G^{A}-21
G^{A}-22
G^{A}-23
G^{A}-24
G^{A}-25
G^{A}-26
G^{A}-27
G^{A}-28
G^{A}-29
G^{A}-30
G^{A}-31
G^{A}-32
G^{A}-33
G^{A}-34
G^{A}-35
G^{A}-36
G^{A}-37
G^{A}-38
G^{A}-39
G^{A}-40
G^{A}-41
G^{A}-42
G^{A}-43
G^{A}-44
G^{A}-45
G^{A}-46
G^{A}-47
G^{A}-48
G^{A}-49
G^{N}-1
G^{N}-2
G^{N}-3
G^{N}-4
G^{N}-5
G^{N}-6
G^{N}-7
G^{N}-8
G^{N}-9
G^{N}-10
G^{N}-11
G^{N}-12
G^{N}-13
G^{N}-14
G^{N}-15
G^{N}-16
G^{N}-17
G^{N}-18
G^{N}-19
G^{N}-20
G^{N}-21
G^{N}-22
G^{N}-23
G^{N}-24
G^{N}-25
G^{N}-26
G^{N}-27
G^{N}-28
G^{N}-29
G^{N}-30
G^{N}-31
G^{N}-32
G^{P}-1
G^{P}-2
G^{P}-3
G^{P}-4
G^{P}-5
G^{P}-6
G^{P}-7
G^{P}-8
G^{P}-9
G^{P}-10
G^{P}-11
G^{P}-12
G^{P}-13
G^{P}-14
G^{P}-15
G^{P}-16
G^{P}-17
G^{P}-18
G^{P}-19
G^{P}-20
G^{P}-21
G^{P}-22
G^{P}-23
G^{P}-24
G^{P}-25
G^{P}-26
G^{P}-27
G^{P}-28
G^{P}-29
G^{P}-30
G^{P}-31
G^{P}-32
G^{P}-33
G^{P}-34
G^{P}-35
Z³ is 0; r is 0; R²² is Me.
G^{A}-1
G^{A}-2
G^{A}-3
G^{A}-4
G^{A}-5
G^{A}-6
G^{A}-7
G^{A}-8
G^{A}-9
G^{A}-10
G^{A}-11
G^{A}-12
G^{A}-13
G^{A}-14
G^{A}-15
G^{A}-16
G^{A}-17
G^{A}-18
G^{A}-19
G^{A}-20
G^{A}-21
G^{A}-22
G^{A}-23
G^{A}-24
G^{A}-25
G^{A}-26
G^{A}-27
G^{A}-28
G^{A}-29
G^{A}-30
G^{A}-31
G^{A}-32
G^{A}-33
G^{A}-34
G^{A}-35
G^{A}-36
G^{A}-37
G^{A}-38
G^{A}-39
G^{A}-40
G^{A}-41
G^{A}-42
G^{A}-43
G^{A}-44
G^{A}-45
G^{A}-46
G^{A}-47
G^{A}-48
G^{A}-49
G^{N}-1
G^{N}-2
G^{N}-3
G^{N}-4
G^{N}-5
G^{N}-6
G^{N}-7
G^{N}-8
G^{N}-9
G^{N}-10
G^{N}-11
G^{N}-12
G^{N}-13
G^{N}-14
G^{N}-15
G^{N}-16
G^{N}-17
G^{N}-18
G^{N}-19
G^{N}-20
G^{N}-21
G^{N}-22
G^{N}-23
G^{N}-24
G^{N}-25
G^{N}-26
G^{N}-27
G^{N}-28
G^{N}-29
G^{N}-30
G^{N}-31
G^{N}-32
G^{P}-1
G^{P}-2
G^{P}-3
G^{P}-4
G^{P}-5
G^{P}-6
G^{P}-7
G^{P}-8
G^{P}-9
G^{P}-10
G^{P}-11
G^{P}-12
G^{P}-13
G^{P}-14
G^{P}-15
G^{P}-16
G^{P}-17
G^{P}-18
G^{P}-19
G^{P}-20
G^{P}-21
G^{P}-22
G^{P}-23
G^{P}-24
G^{P}-25
G^{P}-26
G^{P}-27
G^{P}-28
G^{P}-29
G^{P}-30
G^{P}-31
G^{P}-32
G^{P}-33
G^{P}-34
G^{P}-35
Z³ is CH₂; r is 0; R²² is Me.
G^{A}-1
G^{A}-2
G^{A}-3
G^{A}-4
G^{A}-5
G^{A}-6
G^{A}-7
G^{A}-8
G^{A}-9
G^{A}-10
G^{A}-11
G^{A}-12
G^{A}-13
G^{A}-14
G^{A}-15
G^{A}-16
G^{A}-17
G^{A}-18
G^{A}-19
G^{A}-20
G^{A}-21
G^{A}-22
G^{A}-23
G^{A}-24
G^{A}-25
G^{A}-26
G^{A}-27
G^{A}-28
G^{A}-29
G^{A}-30
G^{A}-31
G^{A}-32
G^{A}-33
G^{A}-34
G^{A}-35
G^{A}-36
G^{A}-37
G^{A}-38
G^{A}-39
G^{A}-40
G^{A}-41
G^{A}-42
G^{A}-43
G^{A}-44
G^{A}-45
G^{A}-46
G^{A}-47
G^{A}-48
G^{A}-49
G^{N}-1
G^{N}-2
G^{N}-3
G^{N}-4
G^{N}-5
G^{N}-6
G^{N}-7
G^{N}-8
G^{N}-9
G^{N}-10
G^{N}-11
G^{N}-12
G^{N}-13
G^{N}-14
G^{N}-15
G^{N}-16
G^{N}-17
G^{N}-18
G^{N}-19
G^{N}-20
G^{N}-21
G^{N}-22
G^{N}-23
G^{N}-24
G^{N}-25
G^{N}-26
G^{N}-27
G^{N}-28
G^{N}-29
G^{N}-30
G^{N}-31
G^{N}-32
G^{P}-1
G^{P}-2
G^{P}-3
G^{P}-4
G^{P}-5
G^{P}-6
G^{P}-7
G^{P}-8
G^{P}-9
G^{P}-10
G^{P}-11
G^{P}-12
G^{P}-13
G^{P}-14
G^{P}-15
G^{P}-16
G^{P}-17
G^{P}-18
G^{P}-19
G^{P}-20
G^{P}-21
G^{P}-22
G^{P}-23
G^{P}-24
G^{P}-25
G^{P}-26
G^{P}-27
G^{P}-28
G^{P}-29
G^{P}-30
G^{P}-31
G^{P}-32
G^{P}-33
G^{P}-34
G^{P}-35
Z³ is —C≡C—; r is 0; R²² is Me.
G^{A}-1
G^{A}-2
G^{A}-3
G^{A}-4
G^{A}-5
G^{A}-6
G^{A}-7
G^{A}-8
G^{A}-9
G^{A}-10
G^{A}-11
G^{A}-12
G^{A}-13
G^{A}-14
G^{A}-15
G^{A}-16
G^{A}-17
G^{A}-18
G^{A}-19
G^{A}-20
G^{A}-21
G^{A}-22
G^{A}-23
G^{A}-24
G^{A}-25
G^{A}-26
G^{A}-27
G^{A}-28
G^{A}-29
G^{A}-30
G^{A}-31
G^{A}-32
G^{A}-33
G^{A}-34
G^{A}-35
G^{A}-36
G^{A}-37
G^{A}-38
G^{A}-39
G^{A}-40
G^{A}-41
G^{A}-42
G^{A}-43
G^{A}-44
G^{A}-45
G^{A}-46
G^{A}-47
G^{A}-48
G^{A}-49
G^{N}-1
G^{N}-2
G^{N}-3
G^{N}-4
G^{N}-5
G^{N}-6
G^{N}-7
G^{N}-8
G^{N}-9
G^{N}-10
G^{N}-11
G^{N}-12
G^{N}-13
G^{N}-14
G^{N}-15
G^{N}-16
G^{N}-17
G^{N}-18
G^{N}-19
G^{N}-20
G^{N}-21
G^{N}-22
G^{N}-23
G^{N}-24
G^{N}-25
G^{N}-26
G^{N}-27
G^{N}-28
G^{N}-29
G^{N}-30
G^{N}-31
G^{N}-32
G^{P}-1
G^{P}-2
G^{P}-3
G^{P}-4
G^{P}-5
G^{P}-6
G^{P}-7
G^{P}-8
G^{P}-9
G^{P}-10
G^{P}-11
G^{P}-12
G^{P}-13
G^{P}-14
G^{P}-15
G^{P}-16
G^{P}-17
G^{P}-18
G^{P}-19
G^{P}-20
G^{P}-21
G^{P}-22
G^{P}-23
G^{P}-24
G^{P}-25
G^{P}-26
G^{P}-27
G^{P}-28
G^{P}-29
G^{P}-30
G^{P}-31
G^{P}-32
G^{P}-33
G^{P}-34
G^{P}-35
Z³ is —CH₂CH₂—; r is 0; R²² is Me.
G^{A}-1
G^{A}-2
G^{A}-3
G^{A}-4
G^{A}-5
G^{A}-6
G^{A}-7
G^{A}-8
G^{A}-9
G^{A}-10
G^{A}-11
G^{A}-12
G^{A}-13
G^{A}-14
G^{A}-15
G^{A}-16
G^{A}-17
G^{A}-18
G^{A}-19
G^{A}-20
G^{A}-21
G^{A}-22
G^{A}-23
G^{A}-24
G^{A}-25
G^{A}-26
G^{A}-27
G^{A}-28
G^{A}-29
G^{A}-30
G^{A}-31
G^{A}-32
G^{A}-33
G^{A}-34
G^{A}-35
G^{A}-36
G^{A}-37
G^{A}-38
G^{A}-39
G^{A}-40
G^{A}-41
G^{A}-42
G^{A}-43
G^{A}-44
G^{A}-45
G^{A}-46
G^{A}-47
G^{A}-48
G^{A}-49
G^{N}-1
G^{N}-2
G^{N}-3
G^{N}-4
G^{N}-5
G^{N}-6
G^{N}-7
G^{N}-8
G^{N}-9
G^{N}-10
G^{N}-11
G^{N}-12
G^{N}-13
G^{N}-14
G^{N}-15
G^{N}-16
G^{N}-17
G^{N}-18
G^{N}-19
G^{N}-20
G^{N}-21
G^{N}-22
G^{N}-23
G^{N}-24
G^{N}-25
G^{N}-26
G^{N}-27
G^{N}-28
G^{N}-29
G^{N}-30
G^{N}-31
G^{N}-32
G^{P}-1
G^{P}-2
G^{P}-3
G^{P}-4
G^{P}-5
G^{P}-6
G^{P}-7
G^{P}-8
G^{P}-9
G^{P}-10
G^{P}-11
G^{P}-12
G^{P}-13
G^{P}-14
G^{P}-15
G^{P}-16
G^{P}-17
G^{P}-18
G^{P}-19
G^{P}-20
G^{P}-21
G^{P}-22
G^{P}-23
G^{P}-24
G^{P}-25
G^{P}-26
G^{P}-27
G^{P}-28
G^{P}-29
G^{P}-30
G^{P}-31
G^{P}-32
G^{P}-33
G^{P}-34
*The definitions of G^{A}, G^{N} and G^{P} in the compounds of this table are as defined in Exhibit 5 in the above Embodiments.

TABLE 9*

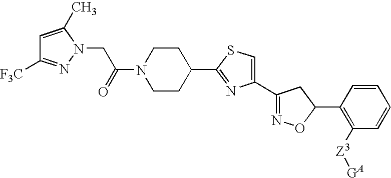

| | | | | | |
|---|---|---|---|---|---|
| Z³ | Z³ | Z³ | Z³ | Z³ | Z³ |

G^{A} is G^{A}-18; r is 0.

| | | | | | |
|---|---|---|---|---|---|
| NH | C(═O) | S | CHCH₃** | CH═C(CH₃)** | CH₂O** |
| NCH₃ | C(═S) | SO₂ | CHCF₃** | OCH₂** | |

G^{A} is G^{A}-36; r is 0.

| | | | | | |
|---|---|---|---|---|---|
| NH | C(═O) | S | CHCH₃** | CH═C(CH₃)** | CH₂O** |
| NCH₃ | C(═S) | SO₂ | CHCF₃** | OCH₂** | |

G^{A} is G^{A}-49; r is 0.

| | | | | | |
|---|---|---|---|---|---|
| NH | C(═O) | S | CHCH₃** | CH═C(CH₃)** | CH₂O** |
| NCH₃ | C(═S) | SO₂ | CHCF₃** | OCH₂** | |

*The definitions of G^{A} in the compounds of this table are as defined in Exhibit 5 in the above Embodiments.
**The left end of these substituents are connected to the phenyl group and right end of these substituent are connected to G^{A}.

TABLE 10*

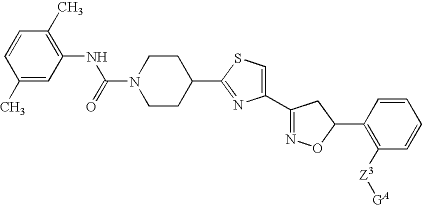

| | | | | | |
|---|---|---|---|---|---|
| Z³ | Z³ | Z³ | Z³ | Z³ | Z³ |

G^{A} is G^{A}-18; r is 0.

| | | | | | |
|---|---|---|---|---|---|
| NH | C(═O) | S | CHCH₃** | CH═C(CH₃)** | CH₂O** |
| NCH₃ | C(═S) | SO₂ | CHCF₃** | OCH₂** | |

G^{A} is G^{A}-36; r is 0.

| | | | | | |
|---|---|---|---|---|---|
| NH | C(═O) | S | CHCH₃** | CH═C(CH₃)** | CH₂O** |
| NCH₃ | C(═S) | SO₂ | CHCF₃** | OCH₂** | |

G^{A} is G^{A}-49; r is 0.

| | | | | | |
|---|---|---|---|---|---|
| NH | C(═O) | S | CHCH₃** | CH═C(CH₃)** | CH₂O** |
| NCH₃ | C(═S) | SO₂ | CHCF₃** | OCH₂** | |

*The definitions of G^{A} in the compounds of this table are as defined in Exhibit 5 in the above Embodiments.
**The left end of these substituents are connected to the phenyl group and right end of these substituent are connected to G^{A}.

TABLE 11*

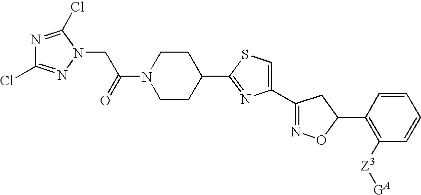

| | | | | | |
|---|---|---|---|---|---|
| Z³ | Z³ | Z³ | Z³ | Z³ | Z³ |

G^{A} is G^{A}-18; r is 0.

| | | | | | |
|---|---|---|---|---|---|
| NH | C(═O) | S | CHCH₃** | CH═C(CH₃)** | CH₂O** |
| NCH₃ | C(═S) | SO₂ | CHCF₃** | OCH₂** | |

G^{A} is G^{A}-36; r is 0.

| | | | | | |
|---|---|---|---|---|---|
| NH | C(═O) | S | CHCH₃** | CH═C(CH₃)** | CH₂O** |
| NCH₃ | C(═S) | SO₂ | CHCF₃** | OCH₂** | |

G^{A} is G^{A}-49; r is 0.

| | | | | | |
|---|---|---|---|---|---|
| NH | C(═O) | S | CHCH₃** | CH═C(CH₃)** | CH₂O** |
| NCH₃ | C(═S) | SO₂ | CHCF₃** | OCH₂** | |

*The definitions of G^{A} in the compounds of this table are as defined in Exhibit 5 in the above Embodiments.
**The left end of these substituents are connected to the phenyl group and right end of these substituent are connected to G^{A}.

TABLE 12

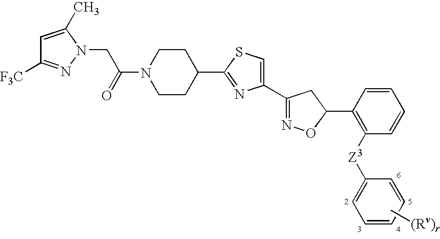

(R^{v})ᵣ
Z³ is a direct bond.
2-Cl
2-F
2,6-di-F
2-CN
2-OH
2-NH₂
2-CO₂H
2-CONH₂
3-SO₂NH₂
2-SH
2-SF₅
3-NHCN
2-CH₃
2-(c-pr-(2-c-Pr))
4-CH₃
2,6-di-CH₃
2,3-di-CH₃
2,4,6-tri-CH₃
3-CH=CH₂
3-CH≡CH
2-CF₃
3-C(=O)CH₃
2-C(=O)CF₃
2-CO₂CH₃
3-CON(CH₃)₂
2-CH=CHCl
2-cyclopropyl
2-(tetrahydrofuran-2-yl)
3-CH₂OCH₂OCH₃
3-CH₂SCH₃
3-CH₂SO₂CH₃
3-CH₂N(CH₃)₂
2-CH₂CN
4-CH₂OH
3-CH₂OCF₃
3-CHC(OCH₃)₂
3-C(OCH₃)₃
2-C(=O)CH₂OCH₃
2-CH₂CO₂CH₃
2-CO₂OCF₃
3-NHC(=O)CH₃
2-NH-c-Pr
2-NHSO₂CH₃
2-C(=S)OCH₃
2-CS₂CH₃
2-OCH₃
2-OCF₃
2-OC(=O)CH₃
2-SCH₃
2-SCF₃
2-SO₂CH₃
3-SO₂N(CH₃)₂
2-Si(CH₃)₃
3-NHCH₃
2-N(CH₃)₂
Z³ is O.
2-Cl
2-F
2,6-di-F
2-CN
2-OH
2-NH₂
2-CO₂H
2-CONH₂
3-SO₂NH₂
2-SH
2-SF₅
3-NHCN
2-CH₃
2-(c-pr-(2-c-Pr))
4-CH₃
2,6-di-CH₃
2,3-di-CH₃
2,4,6-tri-CH₃
3-CH=CH₂
3-CH≡CH
2-CF₃
3-C(=O)CH₃
2-C(=O)CF₃
2-CO₂CH₃
3-CON(CH₃)₂
2-CH=CHCl
2-cyclopropyl
2-(tetrahydrofuran-2-yl)
3-CH₂OCH₂OCH₃
3-CH₂SCH₃
3-CH₂SO₂CH₃
3-CH₂N(CH₃)₂
2-CH₂CN
4-CH₂OH
3-CH₂OCF₃
3-CHC(OCH₃)₂
3-C(OCH₃)₃
2-C(=O)CH₂OCH₃
2-CH₂CO₂CH₃
2-CO₂OCF₃
3-NHC(=O)CH₃
2-NH-c-Pr
2-NHSO₂CH₃
2-C(=S)OCH₃
2-CS₂CH₃
2-OCH₃
2-OCF₃
2-OC(=O)CH₃
2-SCH₃
2-SCF₃
2-SO₂CH₃
3-SO₂N(CH₃)₂
2-Si(CH₃)₃
3-NHCH₃
2-N(CH₃)₂
Z³ is CH₂.
2-Cl
2-F
2,6-di-F
2-CN
2-OH
2-NH₂
2-CO₂H
2-CONH₂
3-SO₂NH₂
2-SH
2-SF₅
3-NHCN
2-CH₃
2-(c-pr-(2-c-Pr))
4-CH₃
2,6-di-CH₃
2,3-di-CH₃
2,4,6-tri-CH₃
3-CH=CH₂
3-CH≡CH
2-CF₃
3-C(=O)CH₃
2-C(=O)CF₃
2-CO₂CH₃
3-CON(CH₃)₂
2-CH=CHCl
2-cyclopropyl
2-(tetrahydrofuran-2-yl)
3-CH₂OCH₂OCH₃
3-CH₂SCH₃
3-CH₂SO₂CH₃
3-CH₂N(CH₃)₂
2-CH₂CN
4-CH₂OH
3-CH₂OCF₃
3-CHC(OCH₃)₂
3-C(OCH₃)₃
2-C(=O)CH₂OCH₃
2-CH₂CO₂CH₃
2-CO₂OCF₃
3-NHC(=O)CH₃
2-NH-c-Pr
2-NHSO₂CH₃
2-C(=S)OCH₃
2-CS₂CH₃
2-OCH₃
2-OCF₃
2-OC(=O)CH₃
2-SCH₃
2-SCF₃
2-SO₂CH₃
3-SO₂N(CH₃)₂
2-Si(CH₃)₃
3-NHCH₃
2-N(CH₃)₂

TABLE 13

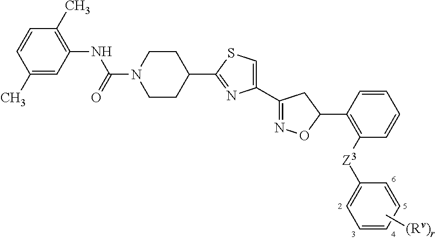

(R^{v})ᵣ
Z³ is a direct bond.
2-Cl
2-F
2,6-di-F
2-CN
2-OH
2-NH₂
2-CO₂H
2-CONH₂
3-SO₂NH₂
2-SH
2-SF₅
3-NHCN
2-CH₃
2-(c-pr-(2-c-Pr))
4-CH₃
2,6-di-CH₃
2,3-di-CH₃
2,4,6-tri-CH₃
3-CH═CH₂
3-C≡CH
2-CF₃
3-C(═O)CH₃
2-C(═O)CF₃
2-CO₂CH₃
3-CON(CH₃)₂
2-CH═CHCl
2-cyclopropyl
2-(tetrahydrofuran-2-yl)
3-CH₂OCH₂OCH₃
3-CH₂SCH₃
3-CH₂SO₂CH₃
3-CH₂N(CH₃)₂
2-CH₂CN
4-CH₂OH
3-CH₂OCF₃
3-CHC(OCH₃)₂
3-C(OCH₃)₃
2-C(═O)CH₂OCH₃
2-CH₂CO₂CH₃
2-CO₂OCF₃
3-NHC(═O)CH₃
2-NH-c-Pr
2-NHSO₂CH₃
2-C(═S)OCH₃
2-CS₂CH₃
2-OCH₃
2-OCF₃
2-OC(═O)CH₃
2-SCH₃
2-SCF₃
2-SO₂CH₃
3-SO₂N(CH₃)₂
2-Si(CH₃)₃
3-NHCH₃
2-N(CH₃)₂
Z³ is O.
2-Cl
2-F
2,6-di-F
2-CN
2-OH
2-NH₂
2-CO₂H
2-CONH₂
3-SO₂NH₂
2-SH
2-SF₅
3-NHCN
2-CH₃
2-(c-pr-(2-c-Pr))
4-CH₃
2,6-di-CH₃
2,3-di-CH₃
2,4,6-tri-CH₃
3-CH═CH2
3-C≡CH
2-CF₃
3-C(═O)CH₃
2-C(═O)CF₃
2-CO₂CH₃
3-CON(CH₃)₂
2-CH═CHCl
2-cyclopropyl
2-(tetrahydrofuran-2-yl)
3-CH₂OCH₂OCH₃
3-CH₂SCH₃
3-CH₂SO₂CH₃
3-CH₂N(CH₃)₂
2-CH₂CN
4-CH₂OH
3-CH₂OCF₃
3-CHC(OCH₃)₂
3-C(OCH₃)₃
2-C(═O)CH₂OCH₃
2-CH₂CO₂CH₃
2-CO₂OCF₃
3-NHC(═O)CH₃
2-NH-c-Pr
2-NHSO₂CH₃
2-C(═S)OCH₃
2-CS₂CH₃
2-OCH₃
2-OCF₃
2-OC(═O)CH₃
2-SCH₃
2-SCF₃
2-SO₂CH₃
3-SO₂N(CH₃)₂
2-Si(CH₃)₃
3-NHCH₃
2-N(CH₃)₂
Z3 is CH₂.
2-Cl
2-F
2,6-di-F
2-CN
2-OH
2-NH₂
2-CO₂H
2-CONH₂
3-SO₂NH₂
2-SH
2-SF₅
3-NHCN
2-CH₃
2-(c-pr-(2-c-Pr))
4-CH₃
2,6-di-CH₃
2,3-di-CH₃
2,4,6-tri-CH₃
3-CH═CH₂
3-C≡CH
2-CF₃
3-C(═O)CH₃
2-C(═O)CF₃
2-CO₂CH₃
3-CON(CH₃)₂
2-CH═CHCl
2-cyclopropyl
2-(tetrahydrofuran-2-yl)
3-CH₂OCH₂OCH₃
3-CH₂SCH₃
3-CH₂SO₂CH₃
3-CH₂N(CH₃)₂
2-CH₂CN
4-CH₂OH
3-CH₂OCF₃
3-CHC(OCH₃)₂
3-C(OCH₃)₃
2-C(═O)CH₂OCH₃
2-CH₂CO₂CH₃
2-CO₂OCF₃
3-NHC(═O)CH₃
2-NH-c-Pr
2-NHSO₂CH₃
2-C(═S)OCH₃
2-CS₂CH₃
2-OCH₃
2-OCF₃
2-OC(═O)CH₃
2-SCH₃
2-SCF₃
2-SO₂CH₃
3-SO₂N(CH₃)₂
2-Si(CH₃)₃
3-NHCH₃
2-N(CH₃)₂

TABLE 14

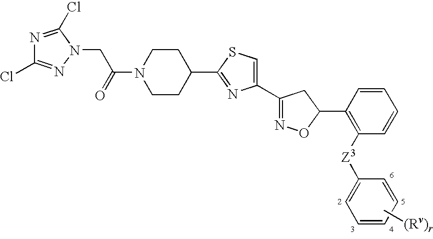

(R^{v})ᵣ
Z³ is a direct bond.
2-Cl
2-F
2,6-di-F
2-CN
2-OH
2-NH₂
2-CO₂H
2-CONH₂
3-SO₂NH₂
2-SH
2-SF₅
3-NHCN
2-CH₃
2-(c-pr-(2-c-Pr))
4-CH₃
2,6-di-CH₃
2,3-di-CH₃
2,4,6-tri-CH₃
3-CH═CH₂
3-C≡CH
2-CF₃
3-C(═O)CH₃
2-C(═O)CF₃
2-CO₂CH₃
3-CON(CH₃)₂
2-CH═CHCl
2-cyclopropyl
2-(tetrahydrofuran-2-yl)
3-CH₂OCH₂OCH₃
3-CH₂SCH₃
3-CH₂SO₂CH₃
3-CH₂N(CH₃)₂
2-CH₂CN
4-CH₂OH
3-CH₂OCF₃
3-CHC(OCH₃)₂
3-C(OCH₃)₃
2-C(═O)CH₂OCH₃
2-CH₂CO₂CH₃
2-CO₂OCF₃
3-NHC(═O)CH₃
2-NH-c-Pr
2-NHSO₂CH₃
2-C(═S)OCH₃
2-CS₂CH₃
2-OCH₃
2-OCF₃
2-OC(═O)CH₃
2-SCH₃
2-SCF₃
2-SO₂CH₃
3-SO₂N(CH₃)₂
2-Si(CH₃)₃
3-NHCH₃
2-N(CH₃)₂
Z³ is O.
2-Cl
2-F
2,6-di-F
2-CN
2-OH
2-NH₂
2-CO₂H
2-CONH₂
3-SO₂NH₂
2-SH
2-SF₅
3-NHCN
2-CH₃
2-(c-pr-(2-c-Pr))
4-CH₃
2,6-di-CH₃
2,3-di-CH₃
2,4,6-tri-CH₃
3-CH═CH₂
3-C≡CH
2-CF₃
3-C(═O)CH₃
2-C(═O)CF₃
2-CO₂CH₃
3-CON(CH₃)₂
2-CH═CHCl
2-cyclopropyl
2-(tetrahydrofuran-2-yl)
3-CH₂OCH₂OCH₃
3-CH₂SCH₃
3-CH₂SO₂CH₃
3-CH₂N(CH₃)₂
2-CH₂CN
4-CH₂OH
3-CH₂OCF₃
3-CHC(OCH₃)₂
3-C(OCH₃)₃
2-C(═O)CH₂OCH₃
2-CH₂CO₂CH₃
2-CO₂OCF₃
3-NHC(═O)CH₃
2-NH-c-Pr
2-NHSO₂CH₃
2-C(═S)OCH₃
2-CS₂CH₃
2-OCH₃
2-OCF₃
2-OC(═O)CH₃
2-SCH₃
2-SCF₃
2-SO₂CH₃
3-SO₂N(CH₃)₂
2-Si(CH₃)₃
3-NHCH₃
2-N(CH₃)2
Z³ is CH₂.
2-Cl
2-F
2,6-di-F
2-CN
2-OH
2-NH₂
2-CO₂H
2-CONH₂
3-SO₂NH₂
2-SH
2-SF₅
3-NHCN
2-CH₃
2-(c-pr-(2-c-Pr))
4-CH₃
2,6-di-CH₃
2,3-di-CH₃
2,4,6-tri-CH₃
3-CH═CH₂
3-C≡CH
2-CF₃
3-C(═O)CH₃
2-C(═O)CF₃
2-CO₂CH₃
3-CON(CH₃)₂
2-CH═CHCl
2-cyclopropyl
2-(tetrahydrofuran-2-yl)
3-CH₂OCH₂OCH₃
3-CH₂SCH₃
3-CH₂SO₂CH₃
3-CH₂N(CH₃)₂
2-CH₂CN
4-CH₂OH
3-CH₂OCF₃
3-CHC(OCH₃)₂
3-C(OCH₃)₃
2-C(═O)CH₂OCH₃
2-CH₂CO₂CH₃
2-CO₂OCF₃
3-NHC(═O)CH₃
2-NH-c-Pr
2-NHSO₂CH₃
2-C(═S)OCH₃
2-CS₂CH₃
2-OCH₃
2-OCF₃
2-OC(═O)CH₃
2-SCH₃
2-SCF₃
2-SO₂CH₃
3-SO₂N(CH₃)₂
2-Si(CH₃)₃
3-NHCH₃
2-N(CH₃)₂

TABLE 15*

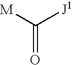

J¹
M is CH₃.
J-29-1
J-29-2
J-29-3
J-29-4
J-29-5
J-29-6
J-29-7
J-29-8
J-29-9
J-29-10
J-29-11
J-29-12
J-29-13
J-29-14
J-29-15
J-29-16
J-29-17
J-29-18
J-29-19
J-29-20
J-29-21
J-29-22
J-29-23
J-29-24
J-29-25
J-29-26
J-29-27
J-29-28
J-29-29
J-29-30
J-29-31
J-29-32
J-29-33
J-29-34
J-29-35
J-29-36
J-29-37
J-29-38
J-29-39
J-29-40
J-29-41
J-29-42
J-29-43
J-29-44
J-29-45
J-29-46
J-29-47
J-29-48
J-29-49
J-29-50
J-29-51
J-29-52
J-29-53
J-29-54
J-29-55
J-29-56
J-29-57
J-29-58
J-29-59
J-29-60
M is CH₂Cl.
J-29-1
J-29-2
J-29-3
J-29-4
J-29-5
J-29-6
J-29-7
J-29-8
J-29-9
J-29-10
J-29-11
J-29-12
J-29-13
J-29-14
J-29-15
J-29-16
J-29-17
J-29-18
J-29-19
J-29-20
J-29-21
J-29-22
J-29-23
J-29-24
J-29-25
J-29-26
J-29-27
J-29-28
J-29-29
J-29-30
J-29-31
J-29-32
J-29-33
J-29-34
J-29-35
J-29-36
J-29-37
J-29-38
J-29-39
J-29-40
J-29-41
J-29-42
J-29-43
J-29-44
J-29-45
J-29-46
J-29-47
J-29-48
J-29-49
J-29-50
J-29-51
J-29-52
J-29-53
J-29-54
J-29-55
J-29-56
J-29-57
J-29-58
J-29-59
J-29-60
M is CH₂Br.
J-29-1
J-29-2
J-29-3
J-29-4
J-29-5
J-29-6
J-29-7
J-29-8
J-29-9
J-29-10
J-29-11
J-29-12
J-29-13
J-29-14
J-29-15
J-29-16
J-29-17
J-29-18
J-29-19
J-29-20
J-29-21
J-29-22
J-29-23
J-29-24
J-29-25
J-29-26
J-29-27
J-29-28
J-29-29
J-29-30
J-29-31
J-29-32
J-29-33
J-29-34
J-29-35
J-29-36
J-29-37
J-29-38
J-29-39
J-29-40
J-29-41
J-29-42
J-29-43
J-29-44
J-29-45
J-29-46
J-29-47
J-29-48
J-29-49
J-29-50
J-29-51
J-29-52
J-29-53
J-29-54
J-29-55
J-29-56
J-29-57
J-29-58
J-29-59
J-29-60
M is CH₂I.
J-29-1
J-29-2
J-29-3
J-29-4
J-29-5
J-29-6
J-29-7
J-29-8
J-29-9
J-29-10
J-29-11
J-29-12
J-29-13
J-29-14
J-29-15
J-29-16
J-29-17
J-29-18
J-29-19
J-29-20
J-29-21
J-29-22
J-29-23
J-29-24
J-29-25
J-29-26
J-29-27
J-29-28
J-29-29
J-29-30
J-29-31
J-29-32
J-29-33
J-29-34
J-29-35
J-29-36
J-29-37
J-29-38
J-29-39
J-29-40
J-29-41
J-29-42
J-29-43
J-29-44
J-29-45
J-29-46
J-29-47
J-29-48
J-29-49
J-29-50
J-29-51
J-29-52
J-29-53
J-29-54
J-29-55
J-29-56
J-29-57
J-29-58
J-29-59
J-29-60
M is OH.
J-29-1
J-29-2
J-29-3
J-29-4
J-29-5
J-29-6
J-29-7
J-29-8
J-29-9
J-29-10
J-29-11
J-29-12
J-29-13
J-29-14
J-29-15
J-29-16
J-29-17
J-29-18
J-29-19
J-29-20
J-29-21
J-29-22
J-29-23
J-29-24
J-29-25
J-29-26
J-29-27
J-29-28
J-29-29
J-29-30
J-29-31
J-29-32
J-29-33
J-29-34
J-29-35
J-29-36
J-29-37
J-29-38
J-29-39
J-29-40
J-29-41
J-29-42
J-29-43
J-29-44
J-29-45
J-29-46
J-29-47
J-29-48
J-29-49
J-29-50
J-29-51
J-29-52
J-29-53
J-29-54
J-29-55
J-29-56
J-29-57
J-29-58
J-29-59
J-29-60
M is OMe.
J-29-1
J-29-2
J-29-3
J-29-4
J-29-5
J-29-6
J-29-7
J-29-8
J-29-9
J-29-10
J-29-11
J-29-12
J-29-13
J-29-14
J-29-15
J-29-16
J-29-17
J-29-18
J-29-19
J-29-20
J-29-21
J-29-22
J-29-23
J-29-24
J-29-25
J-29-26
J-29-27
J-29-28
J-29-29
J-29-30
J-29-31
J-29-32
J-29-33
J-29-34
J-29-35
J-29-36
J-29-37
J-29-38
J-29-39
J-29-40
J-29-41
J-29-42
J-29-43
J-29-44
J-29-45
J-29-46
J-29-47
J-29-48
J-29-49
J-29-50
J-29-51
J-29-52
J-29-53
J-29-54
J-29-55
J-29-56
J-29-57
J-29-58
J-29-59
J-29-60
M is OEt.
J-29-1
J-29-2
J-29-3
J-29-4
J-29-5
J-29-6
J-29-7
J-29-8
J-29-9
J-29-10
J-29-11
J-29-12
J-29-13
J-29-14
J-29-15
J-29-16
J-29-17
J-29-18
J-29-19
J-29-20
J-29-21
J-29-22
J-29-23
J-29-24
J-29-25
J-29-26
J-29-27
J-29-28
J-29-29
J-29-30
J-29-31
J-29-32
J-29-33
J-29-34
J-29-35
J-29-36
J-29-37
J-29-38
J-29-39
J-29-40
J-29-41
J-29-42
J-29-43
J-29-44
J-29-45
J-29-46
J-29-47
J-29-48
J-29-49
J-29-50
J-29-51
J-29-52
J-29-53
J-29-54
J-29-55
J-29-56
J-29-57
J-29-58
J-29-59
J-29-60
M is OPr.
J-29-1
J-29-2
J-29-3
J-29-4
J-29-5
J-29-6
J-29-7
J-29-8
J-29-9
J-29-10
J-29-11
J-29-12
J-29-13
J-29-14
J-29-15
J-29-16
J-29-17
J-29-18
J-29-19
J-29-20
J-29-21
J-29-22
J-29-23
J-29-24
J-29-25
J-29-26
J-29-27
J-29-28
J-29-29
J-29-30
J-29-31
J-29-32
J-29-33
J-29-34
J-29-35
J-29-36
J-29-37
J-29-38
J-29-39
J-29-40
J-29-41
J-29-42
J-29-43
J-29-44
J-29-45
J-29-46
J-29-47
J-29-48
J-29-49
J-29-50
J-29-51
J-29-52
J-29-53
J-29-54
J-29-55
J-29-56
J-29-57
J-29-58
J-29-59
J-29-60
M is O-i-Pr.
J-29-1
J-29-2
J-29-3
J-29-4
J-29-5
J-29-6
J-29-7
J-29-8
J-29-9
J-29-10
J-29-11
J-29-12
J-29-13
J-29-14
J-29-15
J-29-16
J-29-17
J-29-18
J-29-19
J-29-20
J-29-21
J-29-22
J-29-23
J-29-24
J-29-25
J-29-26
J-29-27
J-29-28
J-29-29
J-29-30
J-29-31
J-29-32
J-29-33
J-29-34
J-29-35
J-29-36
J-29-37
J-29-38
J-29-39
J-29-40
J-29-41
J-29-42
J-29-43
J-29-44
J-29-45
J-29-46
J-29-47
J-29-48
J-29-49
J-29-50
J-29-51
J-29-52
J-29-53
J-29-54
J-29-55
J-29-56
J-29-57
J-29-58
J-29-59
J-29-60
M is O-n-Bu.
J-29-1
J-29-2
J-29-3
J-29-4
J-29-5
J-29-6
J-29-7
J-29-8
J-29-9
J-29-10
J-29-11
J-29-12
J-29-13
J-29-14
J-29-15
J-29-16
J-29-17
J-29-18
J-29-19
J-29-20
J-29-21
J-29-22
J-29-23
J-29-24
J-29-25
J-29-26
J-29-27
J-29-28
J-29-29
J-29-30
J-29-31
J-29-32
J-29-33
J-29-34
J-29-35
J-29-36
J-29-37
J-29-38
J-29-39
J-29-40
J-29-41
J-29-42
J-29-43
J-29-44
J-29-45
J-29-46
J-29-47
J-29-48
J-29-49
J-29-50
J-29-51
J-29-52
J-29-53
J-29-54
J-29-55
J-29-56
J-29-57
J-29-58
J-29-59
J-29-60
M is O-t-Bu.
J-29-1
J-29-2
J-29-3
J-29-4
J-29-5
J-29-6
J-29-7
J-29-8
J-29-9
J-29-10
J-29-11
J-29-12
J-29-13
J-29-14
J-29-15
J-29-16
J-29-17
J-29-18
J-29-19
J-29-20
J-29-21
J-29-22
J-29-23
J-29-24
J-29-25
J-29-26
J-29-27
J-29-28
J-29-29
J-29-30
J-29-31
J-29-32
J-29-33
J-29-34
J-29-35
J-29-36
J-29-37
J-29-38
J-29-39
J-29-40
J-29-41
J-29-42
J-29-43
J-29-44
J-29-45
J-29-46
J-29-47
J-29-48
J-29-49
J-29-50
J-29-51
J-29-52
J-29-53
J-29-54
J-29-55
J-29-56
J-29-57
J-29-58
J-29-59
J-29-60
M is NMe₂.
J-29-1
J-29-2
J-29-3
J-29-4
J-29-5
J-29-6
J-29-7
J-29-8
J-29-9
J-29-10
J-29-11
J-29-12
J-29-13
J-29-14
J-29-15
J-29-16
J-29-17
J-29-18
J-29-19
J-29-20
J-29-21
J-29-22
J-29-23
J-29-24
J-29-25
J-29-26
J-29-27
J-29-28
J-29-29
J-29-30
J-29-31
J-29-32
J-29-33
J-29-34
J-29-35
J-29-36
J-29-37
J-29-38
J-29-39
J-29-40
J-29-41
J-29-42
J-29-43
J-29-44
J-29-45
J-29-46
J-29-47
J-29-48
J-29-49
J-29-50
J-29-51
J-29-52
J-29-53
J-29-54
J-29-55
J-29-56
J-29-57
J-29-58
J-29-59
J-29-60
M is NEt₂.
J-29-1
J-29-2
J-29-3
J-29-4
J-29-5
J-29-6
J-29-7
J-29-8
J-29-9
J-29-10
J-29-11
J-29-12
J-29-13
J-29-14
J-29-15
J-29-16
J-29-17
J-29-18
J-29-19
J-29-20
J-29-21
J-29-22
J-29-23
J-29-24
J-29-25
J-29-26
J-29-27
J-29-28
J-29-29
J-29-30
J-29-31
J-29-32
J-29-33
J-29-34
J-29-35
J-29-36
J-29-37
J-29-38
J-29-39
J-29-40
J-29-41
J-29-42
J-29-43
J-29-44
J-29-45
J-29-46
J-29-47
J-29-48
J-29-49
J-29-50
J-29-51
J-29-52
J-29-53
J-29-54
J-29-55
J-29-56
J-29-57
J-29-58
J-29-59
J-29-60
M is N(n-Pr)₂.
J-29-1
J-29-2
J-29-3
J-29-4
J-29-5
J-29-6
J-29-7
J-29-8
J-29-9
J-29-10
J-29-11
J-29-12
J-29-13
J-29-14
J-29-15
J-29-16
J-29-17
J-29-18
J-29-19
J-29-20
J-29-21
J-29-22
J-29-23
J-29-24
J-29-25
J-29-26
J-29-27
J-29-28
J-29-29
J-29-30
J-29-31
J-29-32
J-29-33
J-29-34
J-29-35
J-29-36
J-29-37
J-29-38
J-29-39
J-29-40
J-29-41
J-29-42
J-29-43
J-29-44
J-29-45
J-29-46
J-29-47
J-29-48
J-29-49
J-29-50
J-29-51
J-29-52
J-29-53
J-29-54
J-29-55
J-29-56
J-29-57
J-29-58
J-29-59
J-29-60
M is 1-piperdinyl.
J-29-1
J-29-2
J-29-3
J-29-4
J-29-5
J-29-6
J-29-7
J-29-8
J-29-9
J-29-10
J-29-11
J-29-12
J-29-13
J-29-14
J-29-15
J-29-16
J-29-17
J-29-18
J-29-19
J-29-20
J-29-21
J-29-22
J-29-23
J-29-24
J-29-25
J-29-26
J-29-27
J-29-28
J-29-29
J-29-30
J-29-31
J-29-32
J-29-33
J-29-34
J-29-35
J-29-36
J-29-37
J-29-38
J-29-39
J-29-40
J-29-41
J-29-42
J-29-43
J-29-44
J-29-45
J-29-46
J-29-47
J-29-48
J-29-49
J-29-50
J-29-51
J-29-52
J-29-53
J-29-54
J-29-55
J-29-56
J-29-57
J-29-58
J-29-59
J-29-60
M is 1-pyrrolidinyl.
J-29-1
J-29-2
J-29-3
J-29-4
J-29-5
J-29-6
J-29-7
J-29-8
J-29-9
J-29-10
J-29-11
J-29-12
J-29-13
J-29-14
J-29-15
J-29-16
J-29-17
J-29-18
J-29-19
J-29-20
J-29-21
J-29-22
J-29-23
J-29-24
J-29-25
J-29-26
J-29-27
J-29-28
J-29-29
J-29-30
J-29-31
J-29-32
J-29-33
J-29-34
J-29-35
J-29-36
J-29-37
J-29-38
J-29-39
J-29-40
J-29-41
J-29-42
J-29-43
J-29-44
J-29-45
J-29-46
J-29-47
J-29-48
J-29-49
J-29-50
J-29-51
J-29-52
J-29-53
J-29-54
J-29-55
J-29-56
J-29-57
J-29-58
J-29-59
J-29-60
M is 4-morpholinyl.
J-29-1
J-29-2
J-29-3
J-29-4
J-29-5
J-29-6
J-29-7
J-29-8
J-29-9
J-29-10
J-29-11
J-29-12
J-29-13
J-29-14
J-29-15
J-29-16
J-29-17
J-29-18
J-29-19
J-29-20
J-29-21
J-29-22
J-29-23
J-29-24
J-29-25
J-29-26
J-29-27
J-29-28
J-29-29
J-29-30
J-29-31
J-29-32
J-29-33
J-29-34
J-29-35
J-29-36
J-29-37
J-29-38
J-29-39
J-29-40
J-29-41
J-29-42
J-29-43
J-29-44
J-29-45
J-29-46
J-29-47
J-29-48
J-29-49
J-29-50
J-29-51
J-29-52
J-29-53
J-29-54
J-29-55
J-29-56
J-29-57
J-29-58
J-29-59
J-29-60
* J-29-1 through J-29-60 specified for J¹ in the compounds of this table are as defined in Exhibit A in the above Embodiments.

Table 15 above identifies particular compounds comprising a J¹ group selected from J-29-1 through J-29-60. As many J-29-1 through J-29-60 include a chiral center, these J¹ groups are illustrated in a particular enantiomeric configuration, which in some instances may provide the greatest fungicidal activity for compounds of Formula 1. One skilled in the art immediately recognizes the antipode (i.e. opposite enantiomer) for each of the compounds listed, and furthermore understands that the enantiomers can be present as pure enantiomers or in mixtures enriched in one enantiomer or in racemic mixtures.

### Formulation/Utility

A compound of Formula 1 (or an N-oxide or salt thereof) according to this invention will generally be used as a fungicidal active ingredient in a composition, i.e. formulation, with at least one additional component selected from the group consisting of surfactants, solid diluents and liquid diluents, which serve as a carrier. The formulation or composition ingredients are selected to be consistent with the physical properties of the active ingredient, mode of application and environmental factors such as soil type, moisture and temperature.

Useful formulations include both liquid and solid compositions. Liquid compositions include solutions (including emulsifiable concentrates), suspensions, emulsions (including microemulsions and/or suspoemulsions) and the like, which optionally can be thickened into gels. The general types of aqueous liquid compositions are soluble concentrate, suspension concentrate, capsule suspension, concentrated emulsion, microemulsion and suspo-emulsion. The general types of nonaqueous liquid compositions are emulsifiable concentrate, microemulsifiable concentrate, dispersible concentrate and oil dispersion.

The general types of solid compositions are dusts, powders, granules, pellets, pills, pastilles, tablets, filled films (including seed coatings) and the like, which can be water-dispersible (“wettable”) or water-soluble. Films and coatings formed from film-forming solutions or flowable suspensions are particularly useful for seed treatment. Active ingredient can be (micro)encapsulated and further formed into a suspension or solid formulation; alternatively the entire formulation of active ingredient can be encapsulated (or “overcoated”). Encapsulation can control or delay release of the active ingredient. An emulsifiable granule combines the advantages of both an emulsifiable concentrate formulation and a dry granular formulation. High-strength compositions are primarily used as intermediates for further formulation.

Sprayable formulations are typically extended in a suitable medium before spraying. Such liquid and solid formulations are formulated to be readily diluted in the spray medium, usually water. Spray volumes can range from about from about one to several thousand liters per hectare, but more typically are in the range from about ten to several hundred liters per hectare. Sprayable formulations can be tank mixed with water or another suitable medium for foliar treatment by aerial or ground application, or for application to the growing medium of the plant. Liquid and dry formulations can be metered directly into drip irrigation systems or metered into the furrow during planting. Liquid and solid formulations can be applied onto vegetable seeds as seed treatments before planting to protect developing roots and other subterranean plant parts and/or foliage through systemic uptake.

The formulations will typically contain effective amounts of active ingredient, diluent and surfactant within the following approximate ranges which add up to 100 percent by weight.

| | Weight Percent | | |
|---|---|---|---|
| | Active | | |
| | Ingredient | Diluent | Surfactant |
| Water-Dispersible and Water- | 0.001-90 | 0-99.999 | 0-15 |
| soluble Granules, Tablets | | | |
| and Powders. | | | |
| Oil Dispersions, Suspensions, | 1-50 | 40-99 | 0-50 |
| Emulsions, Solutions (including | | | |
| Emulsifiable Concentrates) | | | |
| Dusts | 1-25 | 70-99 | 0-5 |
| Granules and Pellets | 0.001-95 | 5-99.999 | 0-15 |
| High Strength Compositions | 90-99 | 0-10 | 0-2 |

Solid diluents include, for example, clays such as bentonite, montmorillonite, attapulgite and kaolin, gypsum, cellulose, titanium dioxide, zinc oxide, starch, dextrin, sugars (e.g., lactose, sucrose), silica, talc, mica, diatomaceous earth, urea, calcium carbonate, sodium carbonate and bicarbonate, and sodium sulfate. Typical solid diluents are described in Watkins et al., Handbook of Insecticide Dust Diluents and Carriers, 2nd Ed., Dorland Books, Caldwell, N.J.

Liquid diluents include, for example, water, N,N-dimethylalkanamides (e.g., N,N-dimethylformamide), limonene, dimethyl sulfoxide, N-alkylpyrrolidones (e.g., N-methylpyrrolidinone), ethylene glycol, triethylene glycol, propylene glycol, dipropylene glycol, polypropylene glycol, propylene carbonate, butylene carbonate, paraffins (e.g., white mineral oils, normal paraffins, isoparaffins), alkylbenzenes, alkylnaphthalenes, glycerine, glycerol triacetate, sorbitol, triacetin, aromatic hydrocarbons, dearomatized aliphatics, alkylbenzenes, alkylnaphthalenes, ketones such as cyclohexanone, 2-heptanone, isophorone and 4-hydroxy-4-methyl-2-pentanone, acetates such as isoamyl acetate, hexyl acetate, heptyl acetate, octyl acetate, nonyl acetate, tridecyl acetate and isobornyl acetate, other esters such as alkylated lactate esters, dibasic esters and γ-butyrolactone, and alcohols, which can be linear, branched, saturated or unsaturated, such as methanol, ethanol, n-propanol, isopropyl alcohol, n-butanol, isobutyl alcohol, n-hexanol, 2-ethylhexanol, n-octanol, decanol, isodecyl alcohol, isooctadecanol, cetyl alcohol, lauryl alcohol, tridecyl alcohol, oleyl alcohol, cyclohexanol, tetrahydrofurfuryl alcohol, diacetone alcohol and benzyl alcohol. Liquid diluents also include glycerol esters of saturated and unsaturated fatty acids (typically C₆-C₂₂), such as plant seed and fruit oils (e.g., oils of olive, castor, linseed, sesame, corn (maize), peanut, sunflower, grapeseed, safflower, cottonseed, soybean, rapeseed, coconut and palm kernel), animal-sourced fats (e.g., beef tallow, pork tallow, lard, cod liver oil, fish oil), and mixtures thereof. Liquid diluents also include alkylated fatty acids (e.g., methylated, ethylated, butylated) wherein the fatty acids may be obtained by hydrolysis of glycerol esters from plant and animal sources, and can be purified by distillation. Typical liquid diluents are described in Marsden, Solvents Guide, 2nd Ed., Interscience, New York, 1950.

The solid and liquid compositions of the present invention often include one or more surfactants. Surfactants can be classified as nonionic, anionic or cationic. Nonionic surfactants useful for the present compositions include, but are not limited to: alcohol alkoxylates such as alcohol alkoxylates based on natural and synthetic alcohols (which may be branched or linear) and prepared from the alcohols and ethylene oxide, propylene oxide, butylene oxide or mixtures thereof; amine ethoxylates, alkanolamides and ethoxylated alkanolamides; alkoxylated triglycerides such as ethoxylated soybean, castor and rapeseed oils; alkylphenol alkoxylates such as octylphenol ethoxylates, nonylphenol ethoxylates, dinonyl phenol ethoxylates and dodecyl phenol ethoxylates (prepared from the phenols and ethylene oxide, propylene oxide, butylene oxide or mixtures thereof); block polymers prepared from ethylene oxide or propylene oxide and reverse block polymers where the terminal blocks are prepared from propylene oxide; ethoxylated fatty acids; ethoxylated fatty esters and oils; ethoxylated methyl esters; ethoxylated tristyrylphenol (including those prepared from ethylene oxide, propylene oxide, butylene oxide or mixtures thereof); fatty acid esters, glycerol esters, lanolin-based derivatives, polyethoxylate esters such as polyethoxylated sorbitan fatty acid esters, polyethoxylated sorbitol fatty acid esters and polyethoxylated glycerol fatty acid esters; other sorbitan derivatives such as sorbitan esters; polymeric surfactants such as random copolymers, block copolymers, alkyd peg (polyethylene glycol) resins, graft or comb polymers and star polymers; polyethylene glycols (pegs); polyethylene glycol fatty acid esters; silicone-based surfactants; and sugar-derivatives such as sucrose esters, alkyl polyglycosides and alkyl polysaccharides.

Useful anionic surfactants include, but are not limited to: alkylaryl sulfonic acids and their salts; carboxylated alcohol or alkylphenol ethoxylates; diphenyl sulfonate derivatives; lignin and lignin derivatives such as lignosulfonates; maleic or succinic acids or their anhydrides; olefin sulfonates; phosphate esters such as phosphate esters of alcohol alkoxylates, phosphate esters of alkylphenol alkoxylates and phosphate esters of styryl phenol ethoxylates; protein-based surfactants; sarcosine derivatives; styryl phenol ether sulfate; sulfates and sulfonates of oils and fatty acids; sulfates and sulfonates of ethoxylated alkylphenols; sulfates of alcohols; sulfates of ethoxylated alcohols; sulfonates of amines and amides such as N,N-alkyltaurates; sulfonates of benzene, cumene, toluene, xylene, and dodecyl and tridecylbenzenes; sulfonates of condensed naphthalenes; sulfonates of naphthalene and alkyl naphthalene; sulfonates of fractionated petroleum; sulfosuccinamates; and sulfosuccinates and their derivatives such as dialkyl sulfosuccinate salts.

Useful cationic surfactants include, but are not limited to: amides and ethoxylated amides; amines such as N-alkyl propanediamines, tripropylenetriamines and dipropylenetetramines, and ethoxylated amines, ethoxylated diamines and propoxylated amines (prepared from the amines and ethylene oxide, propylene oxide, butylene oxide or mixtures thereof); amine salts such as amine acetates and diamine salts; quaternary ammonium salts such as quaternary salts, ethoxylated quaternary salts and diquaternary salts; and amine oxides such as alkyldimethylamine oxides and bis-(2-hydroxyethyl)-alkylamine oxides.

Also useful for the present compositions are mixtures of nonionic and anionic surfactants or mixtures of nonionic and cationic surfactants. Nonionic, anionic and cationic surfactants and their recommended uses are disclosed in a variety of published references including McCutcheon's Emulsifiers and Detergents, annual American and International Editions published by McCutcheon's Division, The Manufacturing Confectioner Publishing Co.; Sisely and Wood, Encyclopedia of Surface Active Agents, Chemical Publ. Co., Inc., New York, 1964; and A. S. Davidson and B. Milwidsky, Synthetic Detergents, Seventh Edition, John Wiley and Sons, New York, 1987.

Compositions of this invention may also contain formulation auxiliaries and additives, known to those skilled in the art as formulation aids. Such formulation auxiliaries and additives may control: pH (buffers), foaming during processing (antifoams such polyorganosiloxanes (e.g., Rhodorsil® 416)), sedimentation of active ingredients (suspending agents), viscosity (thixotropic thickeners), in-container microbial growth (antimicrobials), product freezing (antifreezes), color (dyes/pigment dispersions (e.g., Prolzed® Colorant Red)), wash-off (film formers or stickers), evaporation (evaporation retardants), and other formulation attributes. Film formers include, for example, polyvinyl acetates, polyvinyl acetate copolymers, polyvinylpyrrolidone-vinyl acetate copolymer, polyvinyl alcohols, polyvinyl alcohol copolymers and waxes. Examples of formulation auxiliaries and additives include those listed in McCutcheon's Volume 2: Functional Materials, annual International and North American editions published by McCutcheon's Division, The Manufacturing Confectioner Publishing Co.; and PCT Publication WO 03/024222.

Solutions, including emulsifiable concentrates, can be prepared by simply mixing the ingredients. If the solvent of a liquid composition intended for use as an emulsifiable concentrate is water-immiscible, an emulsifier is typically added to emulsify the active-containing solvent upon dilution with water. Active ingredient slurries, with particle diameters of up to 2,000 μm can be wet milled using media mills to obtain particles with average diameters below 3 μm. Aqueous slurries can be made into finished suspension concentrates (see, for example, U.S. Pat. No. 3,060,084) or further processed by spray drying to form water-dispersible granules. Dry formulations usually require dry milling processes, which produce average particle diameters in the 2 to 10 μm range. Dusts and powders can be prepared by blending and, usually, grinding as in a hammer mill or fluid-energy mill. Granules and pellets can be prepared by spraying the active material upon preformed granular carriers or by agglomeration techniques. See Browning, “Agglomeration”, Chemical Engineering, Dec. 4, 1967, pp 147-48, Perry's Chemical Engineer's Handbook, 4th Ed., McGraw-Hill, New York, 1963, pages 8-57 and following, and WO 91/13546. Pellets can be prepared as described in U.S. Pat. No. 4,172,714. Water-dispersible and water-soluble granules can be prepared as taught in U.S. Pat. No. 4,144,050, U.S. Pat. No. 3,920,442 and DE 3,246,493. Tablets can be prepared as taught in U.S. Pat. No. 5,180,587, U.S. Pat. No. 5,232,701 and U.S. Pat. No. 5,208,030. Films can be prepared as taught in GB 2,095,558 and U.S. Pat. No. 3,299,566.

For further information regarding the art of formulation, see T. S. Woods, “The Formulator's Toolbox—Product Forms for Modern Agriculture” in Pesticide Chemistry and Bioscience, The Food-Environment Challenge, T. Brooks and T. R. Roberts, Eds., Proceedings of the 9th International Congress on Pesticide Chemistry, The Royal Society of Chemistry, Cambridge, 1999, pp. 120-133. See also U.S. Pat. No. 3,235,361, Col. 6, line 16 through Col. 7, line 19 and Examples 10-41; U.S. Pat. No. 3,309,192, Col. 5, line 43 through Col. 7, line 62 and Examples 8, 12, 15, 39, 41, 52, 53, 58, 132, 138-140, 162-164, 166, 167 and 169-182; U.S. Pat. No. 2,891,855, Col. 3, line 66 through Col. 5, line 17 and Examples 1-4; Klingman, Weed Control as a Science, John Wiley and Sons, Inc., New York, 1961, pp 81-96; Hance et al., Weed Control Handbook, 8th Ed., Blackwell Scientific Publications, Oxford, 1989; and Developments in formulation technology, PJB Publications, Richmond, UK, 2000.

In the following Examples, all percentages are by weight and all formulations are prepared in conventional ways. Compound numbers refer to compounds in Index Table A.

### Example A

High Strength Concentrate

| | |
|---|---|
| Compound 1 | 98.5% |
| silica aerogel | 0.5% |
| synthetic amorphous fine silica | 1.0%. |

### Example B

Wettable Powder

| | |
|---|---|
| Compound 2 | 65.0% |
| dodecylphenol polyethylene glycol ether | 2.0% |
| sodium ligninsulfonate | 4.0% |
| sodium silicoaluminate | 6.0% |
| montmorillonite (calcined) | 23.0%. |

### Example C

Granule

| | |
|---|---|
| Compound 1 | 10.0% |
| attapulgite granules (low volatile matter, | 90.0%. |
| 0.71/0.30 mm; U.S.S. No. 25-50 sieves) | |

### Example D

Aqueous Suspension

| | |
|---|---|
| Compound 2 | 25.0% |
| hydrated attapulgite | 3.0% |
| crude calcium ligninsulfonate | 10.0% |
| sodium dihydrogen phosphate | 0.5% |
| water | 61.5%. |

### Example E

Extruded Pellet

| | |
|---|---|
| Compound 1 | 25.0% |
| anhydrous sodium sulfate | 10.0% |
| crude calcium ligninsulfonate | 5.0% |
| sodium alkylnaphthalenesulfonate | 1.0% |
| calcium/magnesium bentonite | 59.0%. |

### Example F

Microemulsion

| | |
|---|---|
| Compound 2 | 1.0% |
| triacetine | 30.0% |
| C₈-C₁₀ alkylpolyglycoside | 30.0% |
| glyceryl monooleate | 19.0% |
| water | 20.0%. |

### Example G

Emulsifiable Concentrate

| | |
|---|---|
| Compound 1 | 10.0% |
| C₈-C₁₀ fatty acid methyl ester | 70.0% |
| polyoxyethylene sorbitol hexoleate | 20.0%. |

Compounds of this invention (i.e. compounds of Formula 1, N-oxides, and salts thereof) are useful as plant disease control agents. The present invention therefore further comprises a method for controlling plant diseases caused by fungal plant pathogens comprising applying to the plant or portion thereof to be protected, or to the plant seed to be protected, an effective amount of a compound of the invention or a fungicidal composition containing said compound. The compounds and/or compositions of this invention provide control of diseases caused by a broad spectrum of fungal plant pathogens in the Basidiomycete, Ascomycete, Oomycete and Deuteromycete classes. They are effective in controlling a broad spectrum of plant diseases, particularly foliar pathogens of ornamental, turf, vegetable, field, cereal, and fruit crops. These pathogens include: Oomycetes, including Phytophthora diseases such as Phytophthora infestans, Phytophthora megasperma, Phytophthora parasitica, Phytophthora cinnamomi and Phytophthora capsici, Pythium diseases such as Pythium aphanidermatum, and diseases in the Peronosporaceae family such as Plasmopara viticola, Peronospora spp. (including Peronospora tabacina and Peronospora parasitica), Pseudoperonospora spp. (including Pseudoperonospora cubensis) and Bremia lactucae; Ascomycetes, including Alternaria diseases such as Alternaria solani and Alternaria brassicae, Guignardia diseases such as Guignardia bidwell, Venturia diseases such as Venturia inaequalis, Septoria diseases such as Septoria nodorum and Septoria tritici, powdery mildew diseases such as Erysiphe spp. (including Erysiphe graminis and Erysiphe polygoni), Uncinula necatur, Sphaerotheca fuligena and Podosphaera leucotricha, Pseudocercosporella herpotrichoides, Botrytis diseases such as Botrytis cinerea, Monilinia fructicola, Sclerotinia diseases such as Sclerotinia sclerotiorum, Magnaporthe grisea, Phomopsis viticola, Helminthosporium diseases such as Helminthosporium tritici repentis, Pyrenophora teres, anthracnose diseases such as Glomerella or Colletotrichum spp. (such as Colletotrichum graminicola and Colletotrichum orbiculare), and Gaeumannomyces graminis; Basidiomycetes, including rust diseases caused by Puccinia spp. (such as Puccinia recondite, Puccinia striiformis, Puccinia hordei, Puccinia graminis and Puccinia arachidis), Hemileia vastatrix and Phakopsora pachyrhizi; other pathogens including Rhizoctonia spp. (such as Rhizoctonia solani); Fusarium diseases such as Fusarium roseum, Fusarium graminearum and Fusarium oxysporum; Verticillium dahliae; Sclerotium rolfsii; Rynchosporium secalis; Cercosporidium personatum, Cercospora arachidicola and Cercospora beticola; and other genera and species closely related to these pathogens. In addition to their fungicidal activity, the compositions or combinations also have activity against bacteria such as Erwinia amylovora, Xanthomonas campestris, Pseudomonas syringae, and other related species. Of note is control provided of disease caused by the Ascomycete and Oomycete classes. Of particular note is control provided of disease caused by the Oomycete class.

Plant disease control is ordinarily accomplished by applying an effective amount of a compound of this invention either pre- or post-infection, to the portion of the plant to be protected such as the roots, stems, foliage, fruit, seeds, tubers or bulbs, or to the media (soil or sand) in which the plants to be protected are growing. The compounds can also be applied to seeds to protect the seeds and seedlings developing from the seeds. The compounds can also be applied through irrigation water to treat plants.

Rates of application for these compounds can be influenced by many factors of the environment and should be determined under actual use conditions. Foliage can normally be protected when treated at a rate of from less than about 1 g/ha to about 5,000 g/ha of active ingredient. Seed and seedlings can normally be protected when seed is treated at a rate of from about 0.1 to about 10 g per kilogram of seed.

Compounds of this invention can also be mixed with one or more other biologically active compounds or agents including fungicides, insecticides, nematocides, bactericides, acaricides, herbicides, herbicide safeners, growth regulators such as insect molting inhibitors and rooting stimulants, chemosterilants, semiochemicals, repellents, attractants, pheromones, feeding stimulants, plant nutrients, other biologically active compounds or entomopathogenic bacteria, virus or fungi to form a multi-component pesticide giving an even broader spectrum of agricultural protection. Thus the present invention also pertains to a composition comprising a fungicidally effective amount of a compound of Formula 1 and a biologically effective amount of at least one additional biologically active compound or agent and can further comprise at least one of a surfactant, a solid diluent or a liquid diluent. The other biologically active compounds or agents can be formulated in compositions comprising at least one of a surfactant, solid or liquid diluent. For mixtures of the present invention, one or more other biologically active compounds or agents can be formulated together with a compound of Formula 1, to form a premix, or one or more other biologically active compounds or agents can be formulated separately from the compound of Formula 1, and the formulations combined together before application (e.g., in a spray tank) or, alternatively, applied in succession.

Of note is a composition which in addition to the compound of Formula 1 include at least one fungicidal compound selected from the group consisting of the classes (1) methyl benzimidazole carbamate (MBC) fungicides; (2) dicarboximide fungicides; (3) demethylation inhibitor (DMI) fungicides; (4) phenylamide fungicides; (5) amine/morpholine fungicides; (6) phospholipid biosynthesis inhibitor fungicides; (7) carboxamide fungicides; (8) hydroxy(2-amino-)pyrimidine fungicides; (9) anilinopyrimidine fungicides; (10) N-phenyl carbamate fungicides; (11) quinone outside inhibitor (QoI) fungicides; (12) phenylpyrrole fungicides; (13) quinoline fungicides; (14) lipid peroxidation inhibitor fungicides; (15) melanin biosynthesis inhibitors-reductase (MBI-R) fungicides; (16) melanin biosynthesis inhibitors-dehydratase (MBI-D) fungicides; (17) hydroxyanilide fungicides; (18) squalene-epoxidase inhibitor fungicides; (19) polyoxin fungicides; (20) phenylurea fungicides; (21) quinone inside inhibitor (QiI) fungicides; (22) benzamide fungicides; (23) enopyranuronic acid antibiotic fungicides; (24) hexopyranosyl antibiotic fungicides; (25) glucopyranosyl antibiotic: protein synthesis fungicides; (26) glucopyranosyl antibiotic: trehalase and inositol biosynthesis fungicides; (27) cyanoacetamideoxime fungicides; (28) carbamate fungicides; (29) oxidative phosphorylation uncoupling fungicides; (30) organo tin fungicides; (31) carboxylic acid fungicides; (32) heteroaromatic fungicides; (33) phosphonate fungicides; (34) phthalamic acid fungicides; (35) benzotriazine fungicides; (36) benzene-sulfonamide fungicides; (37) pyridazinone fungicides; (38) thiophene-carboxamide fungicides; (39) pyrimidinamide fungicides; (40) carboxylic acid amide (CAA) fungicides; (41) tetracycline antibiotic fungicides; (42) thiocarbamate fungicides; (43) benzamide fungicides; (44) host plant defense induction fungicides; (45) multi-site contact activity fungicides; (46) fungicides other than classes (1) through (45); and salts of compounds of classes (1) through (46).

Further descriptions of these classes of fungicidal compounds are provided below.

(1) “Methyl benzimidazole carbamate (MBC) fungicides” (Fungicide Resistance Action Committee (FRAC) code 1) inhibit mitosis by binding to β-tubulin during microtubule assembly. Inhibition of microtubule assembly can disrupt cell division, transport within the cell and cell structure. Methyl benzimidazole carbamate fungicides include benzimidazole and thiophanate fungicides. The benzimidazoles include benomyl, carbendazim, fuberidazole and thiabendazole. The thiophanates include thiophanate and thiophanate-methyl.

(2) “Dicarboximide fungicides” (Fungicide Resistance Action Committee (FRAC) code 2) are proposed to inhibit a lipid peroxidation in fungi through interference with NADH cytochrome c reductase. Examples include chlozolinate, iprodione, procymidone and vinclozolin.

(3) “Demethylation inhibitor (DMI) fungicides” (Fungicide Resistance Action Committee (FRAC) code 3) inhibit C14-demethylase, which plays a role in sterol production. Sterols, such as ergosterol, are needed for membrane structure and function, making them essential for the development of functional cell walls. Therefore, exposure to these fungicides results in abnormal growth and eventually death of sensitive fungi. DMI fungicides are divided between several chemical classes: azoles (including triazoles and imidazoles), pyrimidines, piperazines and pyridines. The triazoles include azaconazole, bitertanol, bromuconazole, cyproconazole, difenoconazole, diniconazole (including diniconazole-M), epoxiconazole, fenbuconazole, fluquinconazole, flusilazole, flutriafol, hexaconazole, imibenconazole, ipconazole, metconazole, myclobutanil, penconazole, propiconazole, prothioconazole, simeconazole, tebuconazole, tetraconazole, triadimefon, triadimenol, triticonazole and uniconazole. The imidazoles include clotrimazole, imazalil, oxpoconazole, prochloraz, pefurazoate and triflumizole. The pyrimidines include fenarimol and nuarimol. The piperazines include triforine. The pyridines include pyrifenox. Biochemical investigations have shown that all of the above mentioned fungicides are DMI fungicides as described by K. H. Kuck et al. in Modern Selective Fungicides—Properties, Applications and Mechanisms of Action, H. Lyr (Ed.), Gustav Fischer Verlag: New York, 1995, 205-258.

(4) “Phenylamide fungicides” (Fungicide Resistance Action Committee (FRAC) code 4) are specific inhibitors of RNA polymerase in Oomycete fungi. Sensitive fungi exposed to these fungicides show a reduced capacity to incorporate uridine into rRNA. Growth and development in sensitive fungi is prevented by exposure to this class of fungicide. Phenylamide fungicides include acylalanine, oxazolidinone and butyrolactone fungicides. The acylalanines include benalaxyl, benalaxyl-M, furalaxyl, metalaxyl and metalaxyl-M/mefenoxam. The oxazolidinones include oxadixyl. The butyrolactones include ofurace.

(5) “Amine/morpholine fungicides” (Fungicide Resistance Action Committee (FRAC) code 5) inhibit two target sites within the sterol biosynthetic pathway, Δ⁸→>Δ⁷ isomerase and Δ¹⁴ reductase. Sterols, such as ergosterol, are needed for membrane structure and function, making them essential for the development of functional cell walls. Therefore, exposure to these fungicides results in abnormal growth and eventually death of sensitive fungi. Amine/morpholine fungicides (also known as non-DMI sterol biosynthesis inhibitors) include morpholine, piperidine and spiroketal-amine fungicides. The morpholines include aldimorph, dodemorph, fenpropimorph, tridemorph and trimorphamide. The piperidines include fenpropidin and piperalin. The spiroketal-amines include spiroxamine.

(6) “Phospholipid biosynthesis inhibitor fungicides” (Fungicide Resistance Action Committee (FRAC) code 6) inhibit growth of fungi by affecting phospholipid biosynthesis. Phospholipid biosynthesis fungicides include phosphorothiolate and dithiolane fungicides. The phosphorothiolates include edifenphos, iprobenfos and pyrazophos. The dithiolanes include isoprothiolane.

(7) “Carboxamide fungicides” (Fungicide Resistance Action Committee (FRAC) code 7) inhibit Complex II (succinate dehydrogenase) fungal respiration by disrupting a key enzyme in the Krebs Cycle (TCA cycle) named succinate dehydrogenase. Inhibiting respiration prevents the fungus from making ATP, and thus inhibits growth and reproduction. Carboxamide fungicides include benzamides, furan carboxamides, oxathiin carboxamides, thiazole carboxamides, pyrazole carboxamides and pyridine carboxamides. The benzamides include benodanil, flutolanil and mepronil. The furan carboxamides include fenfuram. The oxathiin carboxamides include carboxin and oxycarboxin. The thiazole carboxamides include thifluzamide. The pyrazole carboxamides include furametpyr, penthiopyrad, bixafen, N-[2-(1S,2R)-[1,1′-bicyclopropyl]-2-ylphenyl]-3-(difluoromethyl)-1-methyl-1H-pyrazole-4-carboxamide and N-[2-(1,3-dimethylbutyl)phenyl]-5-fluoro-1,3-dimethyl-1H-pyrazole-4-carboxamide. The pyridine carboxamides include boscalid.

(8) “Hydroxy(2-amino-)pyrimidine fungicides” (Fungicide Resistance Action Committee (FRAC) code 8) inhibit nucleic acid synthesis by interfering with adenosine deaminase. Examples include bupirimate, dimethirimol and ethirimol.

(9) “Anilinopyrimidine fungicides” (Fungicide Resistance Action Committee (FRAC) code 9) are proposed to inhibit biosynthesis of the amino acid methionine and to disrupt the secretion of hydrolytic enzymes that lyse plant cells during infection. Examples include cyprodinil, mepanipyrim and pyrimethanil.

(10) “N-Phenyl carbamate fungicides” (Fungicide Resistance Action Committee (FRAC) code 10) inhibit mitosis by binding to β-tubulin and disrupting microtubule assembly. Inhibition of microtubule assembly can disrupt cell division, transport within the cell and cell structure. Examples include diethofencarb.

(11) “Quinone outside inhibitor (QoI) fungicides” (Fungicide Resistance Action Committee (FRAC) code 11) inhibit Complex III mitochondrial respiration in fungi by affecting ubiquinol oxidase. Oxidation of ubiquinol is blocked at the “quinone outside” (Q_{O}) site of the cytochrome bc₁ complex, which is located in the inner mitochondrial membrane of fungi. Inhibiting mitochondrial respiration prevents normal fungal growth and development. Quinone outside inhibitor fungicides (also known as strobilurin fungicides) include methoxyacrylate, methoxycarbamate, oximinoacetate, oximinoacetamide, oxazolidinedione, dihydrodioxazine, imidazolinone and benzylcarbamate fungicides. The methoxyacrylates include azoxystrobin, enestroburin (SYP-Z071) and picoxystrobin. The methoxycarbamates include pyraclostrobin. The oximinoacetates include kresoxim-methyl and trifloxystrobin. The oximinoacetamides include dimoxystrobin, metominostrobin, orysastrobin, α-[methoxyimino]-N-methyl-2-[[[1-[3-(trifluoromethyl)phenyl]ethoxy]imino]-methyl]benzeneacetamide and 2-[[[3-(2,6-dichlorophenyl)-1-methyl-2-propen-1-ylidene]-amino]oxy]methyl]-α-(methoxyimino)-N-methylbenzeneacetamide. The oxazolidinediones include famoxadone. The dihydrodioxazines include fluoxastrobin. The imidazolinones include fenamidone. The benzylcarbamates include pyribencarb.

(12) “Phenylpyrrole fungicides” (Fungicide Resistance Action Committee (FRAC) code 12) inhibit a MAP protein kinase associated with osmotic signal transduction in fungi. Fenpiclonil and fludioxonil are examples of this fungicide class.

(13) “Quinoline fungicides” (Fungicide Resistance Action Committee (FRAC) code 13) are proposed to inhibit signal transduction by affecting G-proteins in early cell signaling. They have been shown to interfere with germination and/or appressorium formation in fungi that cause powder mildew diseases. Quinoxyfen is an example of this class of fungicide.

(14) “Lipid peroxidation inhibitor fungicides” (Fungicide Resistance Action Committee (FRAC) code 14) are proposed to inhibit lipid peroxidation which affects membrane synthesis in fungi. Members of this class, such as etridiazole, may also affect other biological processes such as respiration and melanin biosynthesis. Lipid peroxidation fungicides include aromatic carbon and 1,2,4-thiadiazole fungicides. The aromatic carbon fungicides include biphenyl, chloroneb, dicloran, quintozene, tecnazene and tolclofos-methyl. The 1,2,4-thiadiazole fungicides include etridiazole.

(15) “Melanin biosynthesis inhibitors-reductase (MBI-R) fungicides” (Fungicide Resistance Action Committee (FRAC) code 16.1) inhibit the naphthal reduction step in melanin biosynthesis. Melanin is required for host plant infection by some fungi. Melanin biosynthesis inhibitors-reductase fungicides include isobenzofuranone, pyrroloquinolinone and triazolobenzothiazole fungicides. The isobenzofuranones include phthalide. The pyrroloquinolinones include pyroquilon. The triazolobenzothiazoles include tricyclazole.

(16) “Melanin biosynthesis inhibitors-dehydratase (MBI-D) fungicides” (Fungicide Resistance Action Committee (FRAC) code 16.2) inhibit scytalone dehydratase in melanin biosynthesis. Melanin in required for host plant infection by some fungi. Melanin biosynthesis inhibitors-dehydratase fungicides include cyclopropanecarboxamide, carboxamide and propionamide fungicides. The cyclopropanecarboxamides include carpropamid. The carboxamides include diclocymet. The propionamides include fenoxanil.

(17) “Hydroxyanilide fungicides (Fungicide Resistance Action Committee (FRAC) code 17) inhibit C4-demethylase which plays a role in sterol production. Examples include fenhexamid.

(18) “Squalene-epoxidase inhibitor fungicides” (Fungicide Resistance Action Committee (FRAC) code 18) inhibit squalene-epoxidase in ergosterol biosynthesis pathway. Sterols such as ergosterol are needed for membrane structure and function, making them essential for the development of functional cell walls. Therefore exposure to these fungicides results in abnormal growth and eventually death of sensitive fungi. Squalene-epoxidase inhibitor fungicides include thiocarbamate and allylamine fungicides. The thiocarbamates include pyributicarb. The allylamines include naftifine and terbinafine.

(19) “Polyoxin fungicides” (Fungicide Resistance Action Committee (FRAC) code 19) inhibit chitin synthase. Examples include polyoxin.

(20) “Phenylurea fungicides” (Fungicide Resistance Action Committee (FRAC) code 20) are proposed to affect cell division. Examples include pencycuron.

(21) “Quinone inside inhibitor (QiI) fungicides” (Fungicide Resistance Action Committee (FRAC) code 21) inhibit Complex III mitochondrial respiration in fungi by affecting ubiquinol reductase. Reduction of ubiquinol is blocked at the “quinone inside” (Qᵢ) site of the cytochrome bc₁ complex, which is located in the inner mitochondrial membrane of fungi. Inhibiting mitochondrial respiration prevents normal fungal growth and development. Quinone inside inhibitor fungicides include cyanoimidazole and sulfamoyltriazole fungicides. The cyanoimidazoles include cyazofamid. The sulfamoyltriazoles include amisulbrom.

(22) “Benzamide fungicides” (Fungicide Resistance Action Committee (FRAC) code 22) inhibit mitosis by binding to β-tubulin and disrupting microtubule assembly. Inhibition of microtubule assembly can disrupt cell division, transport within the cell and cell structure. Examples include zoxamide.

(23) “Enopyranuronic acid antibiotic fungicides” (Fungicide Resistance Action Committee (FRAC) code 23) inhibit growth of fungi by affecting protein biosynthesis. Examples include blasticidin-S.

(24) “Hexopyranosyl antibiotic fungicides” (Fungicide Resistance Action Committee (FRAC) code 24) inhibit growth of fungi by affecting protein biosynthesis. Examples include kasugamycin.

(25) “Glucopyranosyl antibiotic: protein synthesis fungicides” (Fungicide Resistance Action Committee (FRAC) code 25) inhibit growth of fungi by affecting protein biosynthesis. Examples include streptomycin.

(26) “Glucopyranosyl antibiotic: trehalase and inositol biosynthesis fungicides” (Fungicide Resistance Action Committee (FRAC) code 26) inhibit trehalase in inositol biosynthesis pathway. Examples include validamycin.

(27) “Cyanoacetamideoxime fungicides (Fungicide Resistance Action Committee (FRAC) code 27) include cymoxanil.

(28) “Carbamate fungicides” (Fungicide Resistance Action Committee (FRAC) code 28) are considered multi-site inhibitors of fungal growth. They are proposed to interfere with the synthesis of fatty acids in cell membranes, which then disrupts cell membrane permeability. Propamacarb, propamacarb-hydrochloride, iodocarb, and prothiocarb are examples of this fungicide class.

(29) “Oxidative phosphorylation uncoupling fungicides” (Fungicide Resistance Action Committee (FRAC) code 29) inhibit fungal respiration by uncoupling oxidative phosphorylation. Inhibiting respiration prevents normal fungal growth and development. This class includes 2,6-dinitroanilines such as fluazinam, pyrimidonehydrazones such as ferimzone and dinitrophenyl crotonates such as dinocap, meptyldinocap and binapacryl.

(30) “Organo tin fungicides” (Fungicide Resistance Action Committee (FRAC) code 30) inhibit adenosine triphosphate (ATP) synthase in oxidative phosphorylation pathway. Examples include fentin acetate, fentin chloride and fentin hydroxide.

(31) “Carboxylic acid fungicides” (Fungicide Resistance Action Committee (FRAC) code 31) inhibit growth of fungi by affecting deoxyribonucleic acid (DNA) topoisomerase type II (gyrase). Examples include oxolinic acid.

(32) “Heteroaromatic fungicides” (Fungicide Resistance Action Committee (FRAC) code 32) are proposed to affect DNA/ribonucleic acid (RNA) synthesis. Heteroaromatic fungicides include isoxazole and isothiazolone fungicides. The isoxazoles include hymexazole and the isothiazolones include octhilinone.

(33) “Phosphonate fungicides” (Fungicide Resistance Action Committee (FRAC) code 33) include phosphorous acid and its various salts, including fosetyl-aluminum.

(34) “Phthalamic acid fungicides” (Fungicide Resistance Action Committee (FRAC) code 34) include teclofthalam.

(35) “Benzotriazine fungicides” (Fungicide Resistance Action Committee (FRAC) code 35) include triazoxide.

(36) “Benzene-sulfonamide fungicides” (Fungicide Resistance Action Committee (FRAC) code 36) include flusulfamide.

(37) “Pyridazinone fungicides” (Fungicide Resistance Action Committee (FRAC) code 37) include diclomezine.

(38) “Thiophene-carboxamide fungicides” (Fungicide Resistance Action Committee (FRAC) code 38) are proposed to affect ATP production. Examples include silthiofam.

(39) “Pyrimidinamide fungicides” (Fungicide Resistance Action Committee (FRAC) code 39) inhibit growth of fungi by affecting phospholipid biosynthesis and include diflumetorim.

(40) “Carboxylic acid amide (CAA) fungicides” (Fungicide Resistance Action Committee (FRAC) code 40) are proposed to inhibit phospholipid biosynthesis and cell wall deposition. Inhibition of these processes prevents growth and leads to death of the target fungus. Carboxylic acid amide fungicides include cinnamic acid amide, valinamide carbamate and mandelic acid amide fungicides. The cinnamic acid amides include dimethomorph and flumorph. The valinamide carbamates include benthiavalicarb, benthiavalicarb-isopropyl, iprovalicarb and valiphenal. The mandelic acid amides include mandipropamid, N-[2-[4-[[3-(4-chlorophenyl)-2-propyn-1-yl]oxy]-3-methoxyphenyl]ethyl]-3-methyl-2-[(methylsulfonyl)amino]butanamide and N-[2-[4-[[3-(4-chlorophenyl)-2-propyn-1-yl]oxy]-3-methoxyphenyl]ethyl]-3-methyl-2-[(ethylsulfonyl)amino]butanamide.

(41) “Tetracycline antibiotic fungicides” (Fungicide Resistance Action Committee (FRAC) code 41) inhibit growth of fungi by affecting complex 1 nicotinamide adenine dinucleotide (NADH) oxidoreductase. Examples include oxytetracycline.

(42) “Thiocarbamate fungicides (b42)” (Fungicide Resistance Action Committee (FRAC) code 42) include methasulfocarb.

(43) “Benzamide fungicides” (Fungicide Resistance Action Committee (FRAC) code 43) inhibit growth of fungi by delocalization of spectrin-like proteins. Examples include acylpicolide fungicides such as fluopicolide and fluopyram.

(44) “Host plant defense induction fungicides” (Fungicide Resistance Action Committee (FRAC) code P) induce host plant defense mechanisms. Host plant defense induction fungicides include benzo-thiadiazole, benzisothiazole and thiadiazole-carboxamide fungicides. The benzo-thiadiazoles include acibenzolar-S-methyl. The benzisothiazoles include probenazole. The thiadiazole-carboxamides include tiadinil and isotianil.

(45) “Multi-site contact fungicides” inhibit fungal growth through multiple sites of action and have contact/preventive activity. This class of fungicides includes: (45.1) “copper fungicides” (Fungicide Resistance Action Committee (FRAC) code M1)”, (45.2) “sulfur fungicides” (Fungicide Resistance Action Committee (FRAC) code M2), (45.3) “dithiocarbamate fungicides” (Fungicide Resistance Action Committee (FRAC) code M3), (45.4) “phthalimide fungicides” (Fungicide Resistance Action Committee (FRAC) code M4), (45.5) “chloronitrile fungicides” (Fungicide Resistance Action Committee (FRAC) code M5), (45.6) “sulfamide fungicides” (Fungicide Resistance Action Committee (FRAC) code M6), (45.7) “guanidine fungicides” (Fungicide Resistance Action Committee (FRAC) code M7), (45.8) “triazine fungicides” (Fungicide Resistance Action Committee (FRAC) code M8) and (45.9) “quinone fungicides” (Fungicide Resistance Action Committee (FRAC) code M9). “Copper fungicides” are inorganic compounds containing copper, typically in the copper(II) oxidation state; examples include copper oxychloride, copper sulfate and copper hydroxide, including compositions such as Bordeaux mixture (tribasic copper sulfate). “Sulfur fungicides” are inorganic chemicals containing rings or chains of sulfur atoms; examples include elemental sulfur. “Dithiocarbamate fungicides” contain a dithiocarbamate molecular moiety; examples include mancozeb, metiram, propineb, ferbam, maneb, thiram, zineb and ziram. “Phthalimide fungicides” contain a phthalimide molecular moiety; examples include folpet, captan and captafol. “Chloronitrile fungicides” contain an aromatic ring substituted with chloro and cyano; examples include chlorothalonil. “Sulfamide fungicides” include dichlofluanid and tolyfluanid. “Guanidine fungicides” include dodine, guazatine, iminoctadine albesilate and iminoctadine triacetate. “Triazine fungicides” include anilazine. “Quinone fungicides” include dithianon.

(46) “Fungicides other than fungicides of classes (1) through (45)” include certain fungicides whose mode of action may be unknown. These include: (46.1) “thiazole carboxamide fungicides” (Fungicide Resistance Action Committee (FRAC) code U5), (46.2) “phenyl-acetamide fungicides” (Fungicide Resistance Action Committee (FRAC) code U6), (46.3) “quinazolinone fungicides” (Fungicide Resistance Action Committee (FRAC) code U7) and (46.4) “benzophenone fungicides” (Fungicide Resistance Action Committee (FRAC) code U8). The thiazole carboxamides include ethaboxam. The phenyl-acetamides include cyflufenamid and N-[[(cyclopropylmethoxy)amino][6-(difluoromethoxy)-2,3-difluorophenyl]-methylene]benzeneacetamide. The quinazolinones include proquinazid and 2-butoxy-6-iodo-3-propyl-4H-1-benzopyran-4-one. The benzophenones include metrafenone. The (b46) class also includes bethoxazin, neo-asozin (ferric methanearsonate), pyrrolnitrin, quinomethionate, N-[2-[4-[[3-(4-chlorophenyl)-2-propyn-1-yl]oxy]-3-methoxy-phenyl]ethyl]-3-methyl-2-[(methylsulfonyl)amino]butanamide, N-[2-[4-[[3-(4-chloro-phenyl)-2-propyn-1-yl]oxy]-3-methoxyphenyl]ethyl]-3-methyl-2-[(ethylsulfonyl)amino]-butanamide, 2-[[2-fluoro-5-(trifluoromethyl)phenyl]thio]-2-[3-(2-methoxyphenyl)-2-thiazo-lidinylidene]acetonitrile, 3-[5-(4-chlorophenyl)-2,3-dimethyl-3-isoxazolidinyl]pyridine, 4-fluorophenyl N-[1-[[[1-(4-cyanophenyl)ethyl]sulfonyl]methyl]propyl]carbamate, 5-chloro-6-(2,4,6-trifluorophenyl)-7-(4-methylpiperidin-1-yl)[1,2,4]triazolo[1,5-a]pyrimidine, N-(4-chloro-2-nitrophenyl)-N-ethyl-4-methylbenzenesulfonamide, N-[[(cyclopropylmethoxy)-amino][6-(difluoromethoxy)-2,3-difluorophenyl]methylene]benzeneacetamide, N′-[4-[4-chloro-3-(trifluoromethyl)phenoxy]-2,5-dimethylphenyl]-N-ethyl-N-methylmethanimid-amide and 1-[(2-propenylthio)carbonyl]-2-(1-methylethyl)-4-(2-methylphenyl)-5-amino-1H-pyrazol-3-one.

Therefore of note is a mixture (i.e. composition) comprising a compound of Formula 1 and at least one fungicidal compound selected from the group consisting of the aforedescribed classes (1) through (46). Also of note is a composition comprising said mixture (in fungicidally effective amount) and further comprising at least one additional component selected from the group consisting of surfactants, solid diluents and liquid diluents. Of particular note is a mixture (i.e. composition) comprising a compound of Formula 1 and at least one fungicidal compound selected from the group of specific compounds listed above in connection with classes (1) through (46). Also of particular note is a composition comprising said mixture (in fungicidally effective amount) and further comprising at least one additional surfactant selected from the group consisting of surfactants, solid diluents and liquid diluents.

Examples of other biologically active compounds or agents with which compounds of this invention can be formulated are: insecticides such as abamectin, acephate, acetamiprid, acetoprole, aldicarb, amidoflumet, amitraz, avermectin, azadirachtin, azinphos-methyl, bifenthrin, bifenazate, bistrifluoron, buprofezin, carbofuran, cartap, chinomethionat, chlorfenapyr, chlorfluazuron, chlorantraniliprole, 3-bromo-1-(3-chloro-2-pyridinyl)-N-[4-cyano-2-methyl-6-[[(1-methylethyl)amino]carbonyl]phenyl]-1H-pyrazole-5-carboxamide, 3-bromo-1-(3-chloro-2-pyridinyl)-N-[4-cyano-2-methyl-6-[(methylamino)carbonyl]phenyl]-1H-pyrazole-5-carboxamide, 3-chloro-1-(3-chloro-2-pyridinyl)-N-[4-cyano-2-methyl-6-[(methylamino)carbonyl]phenyl]-1H-pyrazole-5-carboxamide, 3-chloro-1-(3-chloro-2-pyridinyl)-N-[4-cyano-2-methyl-6-[[(1-methylethyl)amino]carbonyl]phenyl]-1H-pyrazole-5-carboxamide, chlorpyrifos, chlorpyrifos-methyl, chlorobenzilate, chromafenozide, clothianidin, cyflumetofen, cyfluthrin, beta-cyfluthrin, cyhalothrin, gamma-cyhalothrin, lambda-cyhalothrin, cyhexatin, cypermethrin, cyromazine, deltamethrin, diafenthiuron, diazinon, dicofol, dieldrin, dienochlor, diflubenzuron, dimefluthrin, dimethoate, dinotefuran, diofenolan, emamectin, endosulfan, esfenvalerate, ethiprole, etoxazole, fenamiphos, fenazaquin, fenbutatin oxide, fenothiocarb, fenoxycarb, fenpropathrin, fenpyroximate, fenvalerate, fipronil, flonicamid, flubendiamide, flucythrinate, tau-fluvalinate, flufenerim, flufenoxuron, fonophos, halofenozide, hexaflumuron, hexythiazox, hydramethylnon, imicyafos, imidacloprid, indoxacarb, isofenphos, lufenuron, malathion, metaflumizone, metaldehyde, methamidophos, methidathion, methomyl, methoprene, methoxychlor, methoxyfenozide, metofluthrin, monocrotophos, nitenpyram, nithiazine, novaluron, noviflumuron, oxamyl, parathion, parathion-methyl, permethrin, phorate, phosalone, phosmet, phosphamidon, pirimicarb, profenofos, profluthrin, propargite, protrifenbute, pymetrozine, pyrafluprole, pyrethrin, pyridaben, pyridalyl, pyrifluquinazon, pyriprole, pyriproxyfen, rotenone, ryanodine, spinetoram, spinosad, spiridiclofen, spiromesifen, spirotetramat, sulprofos, tebufenozide, tebufenpyrad, teflubenzuron, tefluthrin, terbufos, tetrachlorvinphos, thiacloprid, thiamethoxam, thiodicarb, thiosultap-sodium, tolfenpyrad, tralomethrin, triazamate, trichlorfon, triflumuron; and biological agents including entomopathogenic bacteria, such as Bacillus thuringiensis subsp. aizawai, Bacillus thuringiensis subsp. kurstaki, and the encapsulated delta-endotoxins of Bacillus thuringiensis (e.g., Cellcap, MPV, MPVII); entomopathogenic fungi, such as green muscardine fungus; and entomopathogenic virus including baculovirus, nucleopolyhedro virus (NPV) such as HzNPV, AfNPV; and granulosis virus (GV) such as CpGV.

Compounds of this invention and compositions thereof can be applied to plants genetically transformed to express proteins toxic to invertebrate pests (such as Bacillus thuringiensis delta-endotoxins). The effect of the exogenously applied fungicidal compounds of this invention may be synergistic with the expressed toxin proteins.

General references for agricultural protectants (i.e. insecticides, fungicides, nematocides, acaricides, herbicides and biological agents) include The Pesticide Manual, 13th Edition, C. D. S. Tomlin, Ed., British Crop Protection Council, Farnham, Surrey, U.K., 2003 and The BioPesticide Manual, 2nd Edition, L. G. Copping, Ed., British Crop Protection Council, Farnham, Surrey, U.K., 2001.

For embodiments where one or more of these various mixing partners are used, the weight ratio of these various mixing partners (in total) to the compound of Formula 1 (or an N-oxide or salt thereof) is typically between about 1:3000 and about 3000:1. Of note are weight ratios between about 1:300 and about 300:1 (for example, ratios between about 1:30 and about 30:1). One skilled in the art can easily determine through simple experimentation the biologically effective amounts of active ingredients necessary for the desired spectrum of biological activity. It will be evident that including these additional components may expand the spectrum of diseases controlled beyond the spectrum controlled by the compound of Formula 1 alone.

In certain instances, combinations of a compound of this invention with other biologically active (particularly fungicidal) compounds or agents (i.e. active ingredients) can result in a greater-than-additive (i.e. synergistic) effect. Reducing the quantity of active ingredients released in the environment while ensuring effective pest control is always desirable. When synergism of fungicidal active ingredients occurs at application rates giving agronomically satisfactory levels of fungal control, such combinations can be advantageous for reducing crop production cost and decreasing environmental load.

Of note is a combination of a compound of Formula 1 (or an N-oxide or salt thereof) with at least one other fungicidal active ingredient. Of particular note is such a combination where the other fungicidal active ingredient has different site of action from the compound of Formula 1. In certain instances, a combination with at least one other fungicidal active ingredient having a similar spectrum of control but a different site of action will be particularly advantageous for resistance management. Thus, a composition of the present invention can further comprise a biologically effective amount of at least one additional fungicidal active ingredient having a similar spectrum of control but a different site of action.

Of note are these methods where plant diseases caused by Oomycete fungal plant pathogens are controlled.

The following TESTS demonstrate the control efficacy of compounds of this invention on specific pathogens. The pathogen control protection afforded by the compounds is not limited, however, to these species. See Index Tables A and B for compound descriptions. The abbreviation “Ex.” stands for “Example” and is followed by a number indicating in which example the compound is prepared. Index Tables A and B lists the molecular weight of the highest isotopic abundance parent ion (M+1) formed by addition of H⁺ (molecular weight of 1) to the molecule, observed by mass spectrometry using atmospheric pressure chemical ionization (AP⁺). The group G^{G} in Index Tables A and B can be either G^{A}, G^{N} or G^{P} as defined in the Summary of the Invention. The wavy line indicates the point of attachment of each QZ³G^{G} group to the J ring (isoxazoline). Z² is a direct bond and thus is depicted as a line between Q and the isoxazoline ring.

INDEX TABLE A

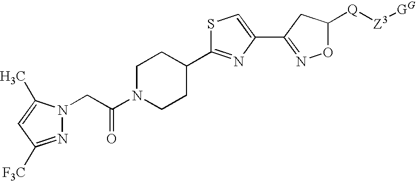

| | | m.p. | AP+ |
|---|---|---|---|
| Cmpd. | QZ³G^{G} | (° C.) | (M + 1) |
| 1 (Ex. 1) | 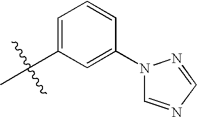 | * | |
| 2 | 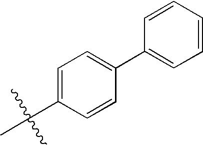 | ** | |
| 3 | 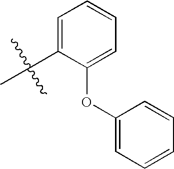 | | 596 |
| 4 | 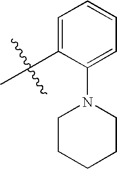 | | 587 |
| 5 | 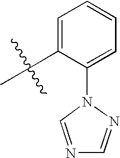 | | 571 |
| 6 | 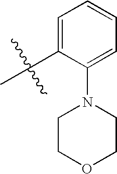 | | 589 |
| 7 | 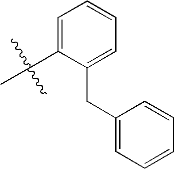 | | 594 |
| 8 | 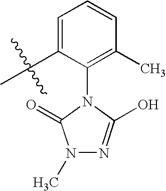 | | 631 |
| 9 | 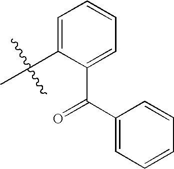 | | 608 |
| 10 | 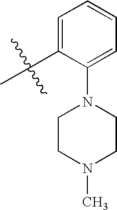 | | 602 |
| 11 | 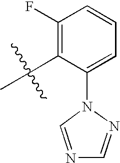 | | 589 |
| 12 | 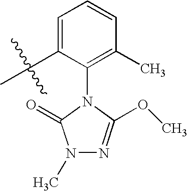 | | 645 |
| 13 | 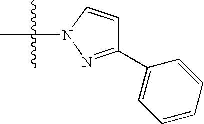 | | 570 |
| 14# | 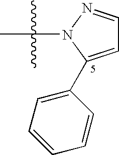 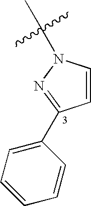 | | 570 |
| 15 | 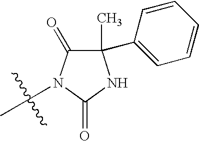 | | 616 |
| 16 | 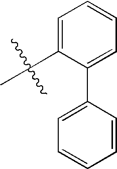 | | 580 |

*See synthesis example for ¹H NMR data.
**See Index Table B for ¹H NMR data.
#This compound is a 3 to 1 mixture of the 3-phenyl and 5-phenyl regioisomers.

INDEX TABLE B

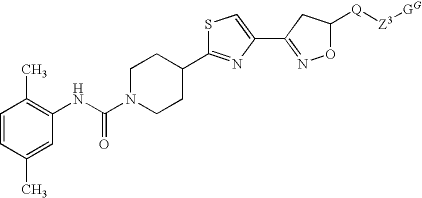

| Cmpd. | QZ³G^{G} | m.p. (° C.) | AP+ (M + 1) |
|---|---|---|---|
| 17 (Ex. 2) | 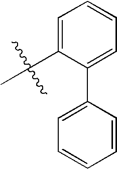 | * | |
| 18 | 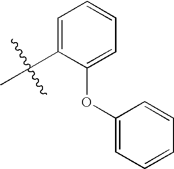 | | 553 |
| 19 | 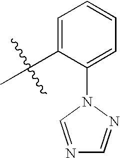 | | 528 |

*See synthesis example for ¹H NMR data.

INDEX TABLE C

| Cmpd. | ¹H NMR Data (CDCl₃ solution unless indicated otherwise)^{a} |
|---|---|
| 2 | δ 1.70-1.90 (m, 2H), 2.20 (t, 2H), 2.32 (s, 3H), 2,89 (t, 1H), |
| | 3.25-3.40 (m, 2H), 3.45 (dd, 1H), 3.88 (dd, 1H), 4.04 (d, 1H), |
| | 4.57 (br d, 1H), 4.92-5.05 (m, 2H), 5.80 (dd, 1H), 6.33 (s, 1H), |
| | 7.35 (t, 1H), 7.40-7.50 (m, 4H), 7.55-7.68 (m, 5H). |

^{a}¹H NMR data are in ppm downfield from tetramethylsilane.
Couplings are designated by (s)—singlet, (d)—doublet, (t)—triplet, (m)—multiplet, (dd)—doublet of doublets, (br d)—broad doublet.

### Biological Examples of the Invention

General protocol for preparing test suspensions for Test A-C: The test compounds were first dissolved in acetone in an amount equal to 3% of the final volume and then suspended at the desired concentration (in ppm) in acetone and purified water (50/50 mix by volume) containing 250 ppm of the surfactant Trem® 014 (polyhydric alcohol esters). The resulting test suspensions were then used in Tests A-C. Spraying a 40 ppm test suspension to the point of run-off on the test plants was equivalent to a rate of 100 g/ha.

### Test A

Grape seedlings were inoculated with a spore suspension of Plasmopara viticola (the causal agent of grape downy mildew) and incubated in a saturated atmosphere at 20° C. for 24 h. After a short drying period, the test suspension was sprayed to the point of run-off on the grape seedlings, which were then moved to a growth chamber at 20° C. for 5 days, after which time the grape seedling were placed back into a saturated atmosphere at 20° C. for 24 h. Upon removal, visual disease ratings were made.

### Test B

The test suspension was sprayed to the point of run-off on tomato seedlings. The following day the seedlings were inoculated with a spore suspension of Phytophthora infestans (the causal agent of tomato late blight) and incubated in a saturated atmosphere at 20° C. for 24 h, and then moved to a growth chamber at 20° C. for 5 days, after which time visual disease ratings were made.

### Test C

Tomato seedlings were inoculated with a spore suspension of Phytophthora infestans (the causal agent of tomato late blight) and incubated in a saturated atmosphere at 20° C. for 17 h. After a short drying period, the test suspension was sprayed to the point of run-off on the tomato seedlings, which were then moved to a growth chamber at 20° C. for 4 days, after which time visual disease ratings were made.

In addition to Tests A-C, the compounds were also sprayed on tomato plants, which were inoculated with Alternaria solani 24 h after treatment, and wheat plants, which were inoculated with Erysiphe graminis f. sp. tritici 24 h after treatment. Test compounds did not show noticeable activity against these additional pathogens under the test conditions at the application rates tested.

Results for Tests A-C are given in Table A. In the table, a rating of 100 indicates 100% disease control and a rating of 0 indicates no disease control (relative to the controls).

TABLE A

| | Percent Disease Control | | |
|---|---|---|---|
| Compound | Test A | Test B | Test C |
| 1 | 91 | 100 | 99 |
| 2 | 76 | 90 | 32 |
| 3 | 97 | 100 | 93 |
| 4 | 58 | 99 | 83 |
| 5 | 98 | 100 | 99 |
| 6 | 87 | 100 | 99 |
| 7 | 73 | 99 | 86 |
| 8 | 0 | 100 | 53 |
| 9 | 0 | 100 | 17 |
| 10 | 10 | 100 | 93 |
| 11 | 99 | 100 | 99 |
| 12 | 31 | 100 | 99 |
| 13 | 56 | 100 | 93 |
| 14 | 82 | 100 | 99 |
| 15 | 92 | 100 | 97 |
| 16 | 99 | 100 | 99 |
| 17 | 98 | 100 | 99 |
| 18 | 67 | 93 | 58 |
| 19 | 99 | 100 | 99 |

What is claimed is:

1. A compound selected from the compounds of Formula 1 and N-oxides and salts thereof, 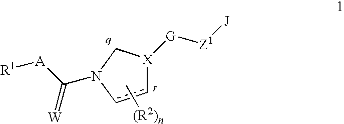
wherein
R¹ is an optionally substituted phenyl or 5- or 6-membered heteroaromatic ring or optionally substituted naphthalenyl;
A is CHR¹⁵ or NR¹⁶;
R¹⁵ is H, halogen, cyano, hydroxy, —CHO, C₁-C₄ alkyl, C₂-C₄ alkenyl, C₂-C₄ alkynyl, C₁-C₄ haloalkyl, C₂-C₄ haloalkenyl, C₂-C₄ haloalkynyl, C₂-C₄ alkoxyalkyl, C₂-C₄ alkylthioalkyl, C₂-C₄ alkylsulfinylalkyl, C₂-C₄ alkylsulfonylalkyl, C₂-C₄ alkylcarbonyl, C₂-C₄ haloalkylcarbonyl, C₂-C₅ alkoxycarbonyl, C₃-C₅ alkoxycarbonylalkyl, C₂-C₅ alkylaminocarbonyl, C₃-C₅ dialkylaminocarbonyl, C₁-C₄ alkoxy, C₁-C₄ haloalkoxy, C₁-C₄ alkylthio, C₁-C₄ haloalkylthio, C₁-C₄ alkylsulfinyl, C₁-C₄ haloalkylsulfinyl, C₁-C₄ alkylsulfonyl or C₁-C₄ haloalkylsulfonyl;
R¹⁶ is H, C₁-C₄ alkyl, C₂-C₄ alkenyl, C₂-C₄ alkynyl, C₁-C₄ haloalkyl, C₂-C₄ haloalkenyl, C₂-C₄ haloalkynyl, C₂-C₄ alkoxyalkyl, C₂-C₄ alkylthioalkyl, C₂-C₄ alkylsulfinylalkyl, C₂-C₄ alkylsulfonylalkyl, C₂-C₄ alkylcarbonyl, C₂-C₄ haloalkylcarbonyl, C₂-C₅ alkoxycarbonyl, C₃-C₅ alkoxycarbonylalkyl, C₂-C₅ alkylaminocarbonyl, C₃-C₅ dialkylaminocarbonyl, C₁-C₄ alkylsulfonyl or C₁-C₄ haloalkylsulfonyl;
W is O or S;
X is a radical selected from 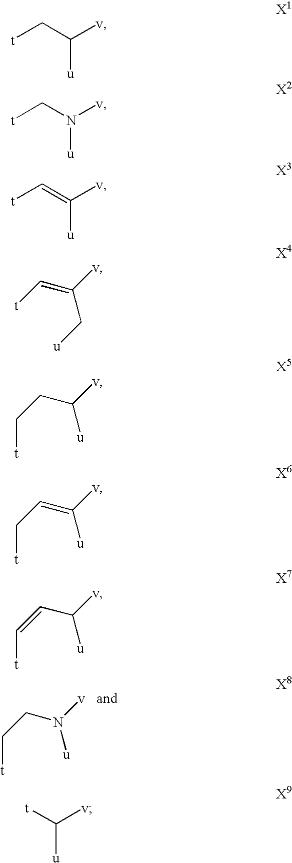
wherein the bond of X¹, X², X³, X⁴, X⁵, X⁶, X⁷, X⁸ or X⁹ which is identified with “t” is connected to the carbon atom identified with “q” of Formula 1, the bond which is identified with “u” is connected to the carbon atom identified with “r” of Formula 1, and the bond which is identified with “v” is connected to G;
each R² is independently C₁-C₄ alkyl, C₁-C₄ alkenyl, C₁-C₄ haloalkyl, C₁-C₄ alkoxy, halogen, cyano or hydroxy; or
two R² are taken together as C₁-C₄ alkylene or C₂-C₄ alkenylene to form a bridged bicyclic or fused bicyclic ring system; or
two R² attached to adjacent ring carbon atoms joined by a double bond are taken together as —CH═CH—CH═CH— optionally substituted with 1 to 3 substituents selected from C₁-C₄ alkyl, C₁-C₄ haloalkyl, C₁-C₄ alkoxy, C₁-C₄ haloalkoxy, halogen, hydroxy, amino, cyano and nitro;
G is an optionally substituted 5-membered heterocyclic ring;
J is a 5-, 6- or 7-membered ring, a 8- to 11-membered bicyclic ring system or a 7- to 11-membered spirocyclic ring system, each ring or ring system containing ring members selected from carbon, up to 4 heteroatoms selected from up to 2 O, up to 2 S and up to 4 N, and up to 3 ring members selected from C(═O), C(═S), S(═O)ₐ(═NR²³)_{b} and SiR¹⁷R¹⁸, each ring or ring system substituted with 1 to 2 substituents independently selected from —Z²Q and optionally substituted with 1 to 5 substituents independently selected from R⁵;
each R⁵ is independently H, halogen, cyano, hydroxy, amino, nitro, —CHO, —C(═O)OH, —C(═O)NH₂, —NR²⁵R²⁶, C₁-C₆ alkyl, C₂-C₆ alkenyl, C₂-C₆ alkynyl, C₁-C₆ haloalkyl, C₂-C₆ haloalkenyl, C₂-C₆ haloalkynyl, C₃-C₈ cycloalkyl, C₃-C₈ halocycloalkyl, C₄-C₁₀ alkylcycloalkyl, C₄-C₁₀ cycloalkylalkyl, C₆-C₁₄ cycloalkylcycloalkyl, C₄-C₁₀ halocycloalkylalkyl, C₅-C₁₀ alkylcycloalkylalkyl, C₃-C₈ cycloalkenyl, C₃-C₈ halocycloalkenyl, C₂-C₆ alkoxyalkyl, C₄-C₁₀ cycloalkoxyalkyl, C₃-C₈ alkoxyalkoxyalkyl, C₂-C₆ alkylthioalkyl, C₂-C₆ alkylsulfinylalkyl, C₂-C₆ alkylsulfonylalkyl, C₂-C₆ alkylaminoalkyl, C₃-C₈ dialkylaminoalkyl, C₂-C₆ haloalkylaminoalkyl, C₄-C₁₀ cycloalkylaminoalkyl, C₂-C₆ alkylcarbonyl, C₂-C₆ haloalkylcarbonyl, C₄-C₈ cycloalkylcarbonyl, C₂-C₆ alkoxycarbonyl, C₄-C₈ cycloalkoxycarbonyl, C₅-C₁₀ cycloalkylalkoxycarbonyl, C₂-C₆ alkylaminocarbonyl, C₃-C₈ dialkylaminocarbonyl, C₄-C₈ cycloalkylaminocarbonyl, C₂-C₆ haloalkoxyalkyl, C₁-C₆ hydroxyalkyl, C₁-C₆ alkoxy, C₁-C₆ haloalkoxy, C₃-C₈ cycloalkoxy, C₃-C₈ halocycloalkoxy, C₄-C₁₀ cycloalkylalkoxy, C₂-C₆ alkenyloxy, C₂-C₆ haloalkenyloxy, C₂-C₆ alkynyloxy, C₂-C₆ haloalkynyloxy, C₂-C₆ alkoxyalkoxy, C₂-C₆ alkylcarbonyloxy, C₂-C₆ haloalkylcarbonyloxy, C₄-C₈ cycloalkylcarbonyloxy, C₃-C₆ alkylcarbonylalkoxy, C₁-C₆ alkylthio, C₁-C₆ haloalkylthio, C₃-C₈ cycloalkylthio, C₁-C₆ alkylsulfinyl, C₁-C₆ haloalkylsulfinyl, C₁-C₆ alkylsulfonyl, C₁-C₆ haloalkylsulfonyl, C₃-C₈ cycloalkylsulfonyl, C₃-C₁₀ trialkylsilyl, C₁-C₆ alkylsulfonylamino or C₁-C₆ haloalkylsulfonylamino;
R²⁵ is H, C₁-C₆ alkyl, C₁-C₆ haloalkyl, C₃-C₈ cycloalkyl, C₂-C₆ alkylcarbonyl, C₂-C₆ haloalkylcarbonyl, C₂-C₆ alkoxycarbonyl or C₂-C₆ haloalkoxycarbonyl;
R²⁶ is C₁-C₆ alkyl, C₁-C₆ haloalkyl, C₃-C₈ cycloalkyl, C₂-C₆ alkylcarbonyl, C₂-C₆ haloalkylcarbonyl, C₂-C₆ alkoxycarbonyl, C₂-C₆ haloalkoxycarbonyl or —Z⁴Q;
each R¹⁷ and R¹⁸ is independently C₁-C₅ alkyl, C₂-C₅ alkenyl, C₂-C₅ alkynyl, C₃-C₅ cycloalkyl, C₃-C₆ halocycloalkyl, C₄-C₁₀ cycloalkylalkyl, C₄-C₇ alkylcycloalkyl, C₅-C₇ alkylcycloalkylalkyl, C₁-C₅ haloalkyl, C₁-C₅ alkoxy or C₁-C₅ haloalkoxy;
each Q is independently phenyl, benzyl, naphthalenyl, a 5- or 6-membered heteroaromatic ring or an 8- to 11-membered heteroaromatic bicyclic ring system, each substituted with 1 to 2 substituents independently selected from R⁷ on carbon or nitrogen atom ring members, and each optionally substituted with 1 to 5 substituents independently selected from R^{7a} on carbon atom ring members and R¹² on nitrogen atom ring members; or
a 3- to 7-membered nonaromatic carbocyclic ring, a 5-, 6- or 7-membered nonaromatic heterocyclic ring or an 8- to 11-membered nonaromatic bicyclic ring system, each optionally including ring members selected from C(═O), C(═S), S(═O)ₐ(═NR²³)_{b} and SiR¹⁷R¹⁸, and each ring or ring system substituted with 1 to 2 substituents independently selected from R⁷ on carbon or nitrogen atom ring members, and each optionally substituted with 1 to 5 substituents independently selected from R^{7a} on carbon atom ring members and R¹² on nitrogen atom ring members;
each R⁷ is independently —Z³G^{A}, —Z³G^{N} or —Z³G^{P};
each G^{A} is independently a phenyl or 5- or 6-membered heteroaromatic ring, each ring substituted with up to 5 substituents independently selected from R^{v} on carbon atom ring members and R²² on nitrogen atom ring members;
each G^{N} is independently a 3- to 7-membered nonaromatic ring including ring members selected from (CR^{v})₂, O, S, NR²², —C(R^{v})═C(R^{v})—, —C(R^{v})═N—, —N═N—, C(═O), C(═S), C(═NR²³), S(═O)ₐ(═NR²³)_{b} and SiR¹⁷R¹⁸;
each G^{P} is independently an 8- to 10-membered aromatic or 7- to 11-membered nonaromatic bicyclic ring system, said ring system including ring members selected from (CR^{v})₂, O, S, NR²², —C(R^{v})═C(R^{v})—, —C(R^{v})═N—, —N═N—, C(═O), C(═S), C(═NR²³), S(═O)ₐ(═NR²³)_{b} and SiR¹⁷R¹⁸;
each R^{v} is independently H, halogen, cyano, hydroxy, amino, nitro, —CHO, —C(═O)OH, —C(═O)NH₂, —SO₂NH₂, —C(═S)NH₂, —C(═O)NHCN, —C(═O)NHOH, —SH, —SO₂NHCN, —SO₂NHOH, —OCN, —SCN, —SF₅, —NHCHO, —NHNH₂, —N₃, —NHOH, —NHCN, —NHC(═O)NH₂, —N═C═O, —N═C═S, C₁-C₆ alkyl, C₂-C₆ alkenyl, C₂-C₆ alkynyl, C₁-C₆ haloalkyl, C₂-C₈ alkylcarbonyl, C₂-C₈ haloalkylcarbonyl, C₂-C₈ alkoxycarbonyl, C₄-C₁₀ cycloalkoxycarbonyl, C₅-C₁₂ cycloalkylalkoxycarbonyl, C₂-C₈ alkylaminocarbonyl, C₃-C₁₀ dialkylaminocarbonyl, C₂-C₆ haloalkenyl, C₂-C₆ haloalkynyl, C₃-C₈ cycloalkyl, C₃-C₈ halocycloalkyl, C₄-C₁₀ alkylcycloalkyl, C₄-C₁₀ cycloalkylalkyl, C₆-C₁₄ cycloalkylcycloalkyl, C₄-C₁₀ halocycloalkylalkyl, C₅-C₁₂ alkylcycloalkylalkyl, C₃-C₈ cycloalkenyl, C₃-C₈ halocycloalkenyl, C₂-C₈ alkoxyalkyl, C₄-C₁₀ cycloalkoxyalkyl, C₃-C₁₀ alkoxyalkoxyalkyl, C₂-C₈ alkylthioalkyl, C₂-C₈ alkylsulfinylalkyl, C₂-C₈ alkylsulfonylalkyl, C₂-C₈ alkylaminoalkyl, C₃-C₁₀ dialkylaminoalkyl, C₂-C₈ haloalkylaminoalkyl, C₄-C₁₀ cycloalkylaminoalkyl, C₄-C₁₀ cycloalkylcarbonyl, C₄-C₁₀ cycloalkylaminocarbonyl, C₂-C₇ cyanoalkyl, C₁-C₆ hydroxyalkyl, C₄-C₁₀ cycloalkenylalkyl, C₂-C₈ haloalkoxyalkyl, C₂-C₈ alkoxyhaloalkyl, C₂-C₈ haloalkoxyhaloalkyl, C₄-C₁₀ halocycloalkoxyalkyl, C₄-C₁₀ cycloalkenyloxyalkyl, C₄-C₁₀ halocycloalkenyloxyalkyl, C₃-C₁₀ dialkoxyalkyl, C₄-C₁₂ trialkoxyalkyl, C₃-C₈ alkoxyalkenyl, C₃-C₈ alkoxyalkynyl, C₃-C₁₀ halodialkylaminoalkyl, C₅-C₁₂ cycloalkyl(alkyl)aminoalkyl, C₂-C₈ alkyl(thiocarbonyl), C₃-C₁₀ alkoxyalkylcarbonyl, C₃-C₁₀ alkoxycarbonylalkyl, C₂-C₈ haloalkoxycarbonyl, C₃-C₁₀ alkoxyalkoxycarbonyl, C₂-C₈ (alkylthio)carbonyl, C₂-C₈ alkoxy(thiocarbonyl), C₂-C₈ alkylthio(thiocarbonyl), C₂-C₈ alkylamino(thiocarbonyl), C₃-C₁₀ dialkylamino(thiocarbonyl), C₃-C₁₀ alkoxy(alkyl)aminocarbonyl, C₂-C₈ alkylsulfonylaminocarbonyl, C₂-C₈ haloalkylsulfonylaminocarbonyl, C₂-C₈ alkylamidino, C₃-C₁₀ dialkylamidino, C₁-C₆ alkoxy, C₁-C₆ haloalkoxy, C₂-C₈ alkylcarbonyloxy, C₁-C₆ alkylthio, C₁-C₆ haloalkylthio, C₁-C₆ alkylsulfinyl, C₁-C₆ haloalkylsulfinyl, C₁-C₆ alkylsulfonyl, C₁-C₆ haloalkylsulfonyl, C₁-C₆ alkylaminosulfonyl, C₂-C₈ dialkylaminosulfonyl, C₃-C₁₀ trialkylsilyl, C₃-C₈ cycloalkoxy, C₃-C₈ halocycloalkoxy, C₄-C₁₀ cycloalkylalkoxy, C₂-C₆ alkenyloxy, C₂-C₆ haloalkenyloxy, C₂-C₆ alkynyloxy, C₃-C₆ haloalkynyloxy, C₂-C₈ alkoxyalkoxy, C₂-C₈ haloalkylcarbonyloxy, C₄-C₁₀ cycloalkylcarbonyloxy, C₃-C₁₀ alkylcarbonylalkoxy, C₃-C₈ cycloalkylthio, C₃-C₈ cycloalkylsulfonyl, C₃-C₈ cycloalkenyloxy, C₃-C₈ halocycloalkenyloxy, C₂-C₈ haloalkoxyalkoxy, C₂-C₈ alkoxyhaloalkoxy, C₂-C₈ haloalkoxyhaloalkoxy, C₃-C₁₀ alkoxycarbonylalkoxy, C₂-C₈ alkyl(thiocarbonyl)oxy, C₂-C₈ alkylcarbonylthio, C₂-C₈ alkyl(thiocarbonyl)thio, C₃-C₈ cycloalkylsulfinyl, C₃-C₁₀ halotrialkylsilyl, C₁-C₆ alkylamino, C₂-C₈ dialkylamino, C₂-C₈ alkylcarbonylamino, C₁-C₆ alkylsulfonylamino, C₁-C₆ haloalkylamino, C₂-C₈ halodialkylamino, C₃-C₈ cycloalkylamino, C₂-C₈ haloalkylcarbonylamino, C₁-C₆ haloalkylsulfonylamino, C₄-C₁₀ cycloalkylalkylamino, C₄-C₁₀ cycloalkyl(alkyl)amino, C₃-C₁₀ alkoxycarbonylalkylamino, C₁-C₆ alkoxyamino, C₁-C₆ haloalkoxyamino, C₄-C₁₂ dialkylimido, C₂-C₈ alkoxycarbonylamino, C₂-C₈ haloalkoxycarbonylamino, C₂-C₈ alkylaminocarbonylamino, C₃-C₁₀ dialkylaminocarbonylamino, C₃-C₁₀ alkylaminocarbonylalkylamino, C₄-C₁₂ dialkylaminocarbonylalkylamino, C₂-C₈ alkylamino(thiocarbonyl)amino, C₃-C₁₀ dialkylamino(thiocarbonyl)amino, C₃-C₁₀ alkylamino(thiocarbonyl)alkylamino or C₄-C₁₂ dialkylamino(thiocarbonyl)alkylamino;
each R^{7a} is independently C₁-C₆ alkyl, C₂-C₆ alkenyl, C₂-C₆ alkynyl, C₃-C₆ cycloalkyl, C₄-C₁₀ cycloalkylalkyl, C₄-C₁₀ alkylcycloalkyl, C₅-C₁₀ alkylcycloalkylalkyl, C₁-C₆ haloalkyl, C₂-C₆ haloalkenyl, C₂-C₆ haloalkynyl, C₃-C₆ halocycloalkyl, halogen, hydroxy, amino, cyano, nitro, C₁-C₄ alkoxy, C₁-C₄ haloalkoxy, C₁-C₄ alkylthio, C₁-C₄ alkylsulfinyl, C₁-C₄ alkylsulfonyl, C₁-C₄ haloalkylthio, C₁-C₄ haloalkylsulfinyl, C₁-C₄ haloalkylsulfonyl, C₁-C₄ alkylamino, C₂-C₈ dialkylamino, C₃-C₆ cycloalkylamino, C₂-C₄ alkoxyalkyl, C₁-C₄ hydroxyalkyl, C₂-C₄ alkylcarbonyl, C₂-C₆ alkoxycarbonyl, C₂-C₆ alkylcarbonyloxy, C₂-C₆ alkylcarbonylthio, C₂-C₆ alkylaminocarbonyl, C₃-C₈ dialkylaminocarbonyl or C₃-C₆ trialkylsilyl; or
R⁵ and R^{7a} are taken together with the atoms linking R⁵ and R^{7a} to form an optionally substituted 5- to 7-membered ring containing ring members selected from carbon, up to 3 heteroatoms selected from up to 1 O, up to 1 S and up to 1 N, and up to 3 ring members selected from C(═O), C(═S), S(═O)ₐ(═NR²³)_{b} and SiR¹⁷R¹⁸;
R¹² is H, C₁-C₃ alkyl, C₁-C₃ alkylcarbonyl, C₁-C₃ alkoxy or C₁-C₃ alkoxycarbonyl;
each Z¹ and Z² is independently a direct bond, O, C(═O), S(O)ₘ, CHR²⁰ or NR²¹;
each Z³ is independently a direct bond, O, NR²², C(═O), C(═S), S(O)ₘ, CHR²⁰, CHR²⁰—CHR²⁰, CR²⁴═CR²⁷, C≡C, OCHR²⁰ or CHR²⁰O;
each Z⁴ is independently O, C(═O), S(O)ₘ or CHR²⁰;
each R²⁰ is independently H, C₁-C₄ alkyl or C₁-C₄ haloalkyl;
each R²¹ is independently H, C₁-C₆ alkyl, C₁-C₆ haloalkyl, C₃-C₈ cycloalkyl, C₂-C₆ alkylcarbonyl, C₂-C₆ haloalkylcarbonyl, C₂-C₆ alkoxycarbonyl or C₂-C₆ haloalkoxycarbonyl;
each R²² is independently H, C₁-C₄ alkyl or C₁-C₄ haloalkyl;
each R²³ is independently H, cyano, C₁-C₆ alkyl, C₁-C₆ haloalkyl, C₃-C₈ cycloalkyl, C₃-C₈ halocycloalkyl, C₁-C₆ alkoxy, C₁-C₆ haloalkoxy, C₁-C₆ alkylamino, C₂-C₈ dialkylamino, C₁-C₆ haloalkylamino or phenyl;
each R²⁴ and R²⁷ is independently H, C₁-C₄ alkyl or C₁-C₄ haloalkyl;
each m is independently 0, 1 or 2;
n is 0, 1 or 2; and
a and b are independently 0, 1 or 2 in each instance of S(═O)ₐ(═NR²³)_{b}, provided that the sum of a and b is 1 or 2.

2. A compound of claim 1 wherein
R¹ is a phenyl or 5- or 6-membered heteroaromatic ring optionally substituted with 1-3 substituents independently selected from R^{4a} on carbon ring members and R^{4b} on nitrogen ring members;
G is a 5-membered heterocyclic ring optionally substituted with up to 2 substituents selected from R³ on carbon ring members and selected from R¹¹ on nitrogen ring members;
J is one of J-1 through J-82 (as depicted in Exhibit 3) wherein the bond shown projecting to the left is bonded to Z¹; 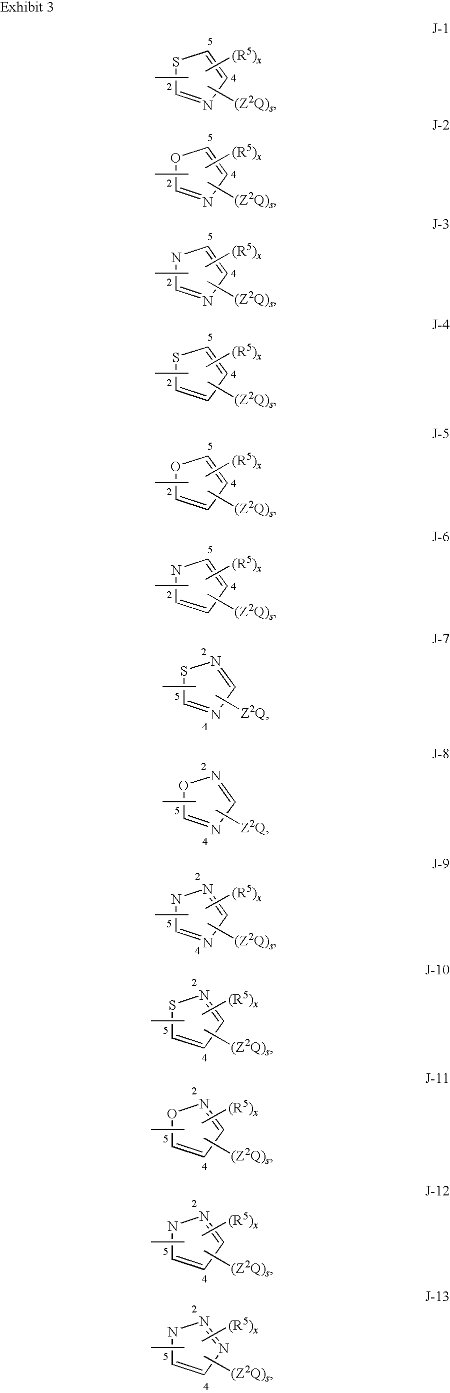 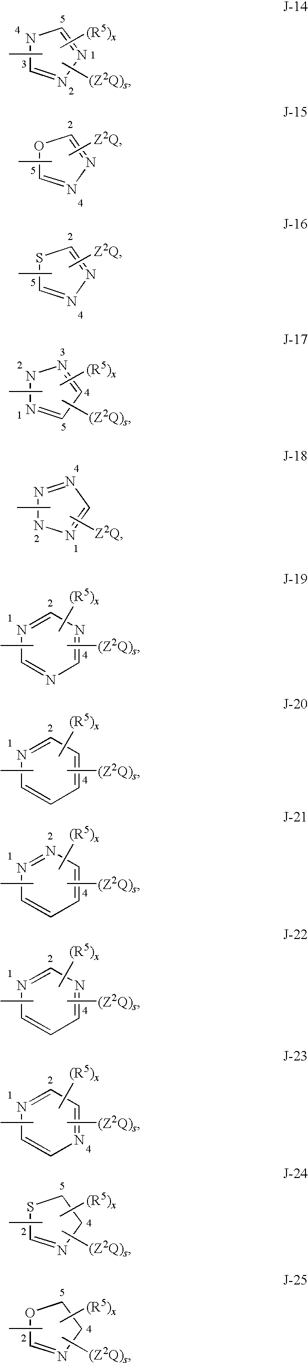 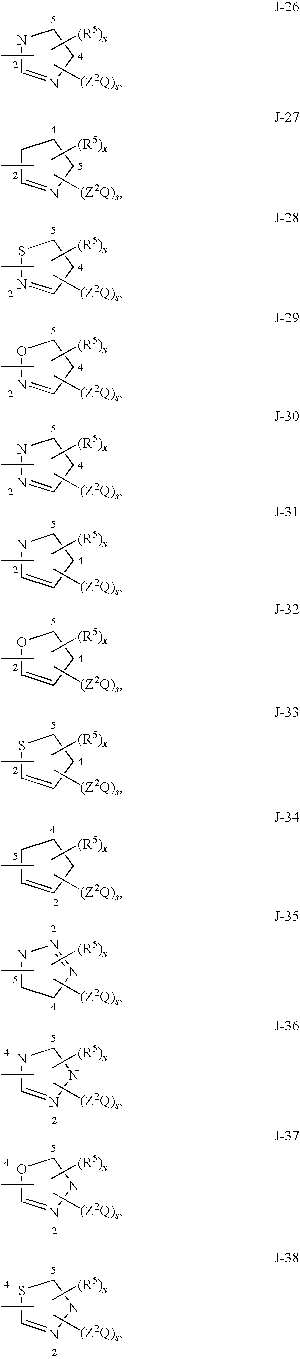 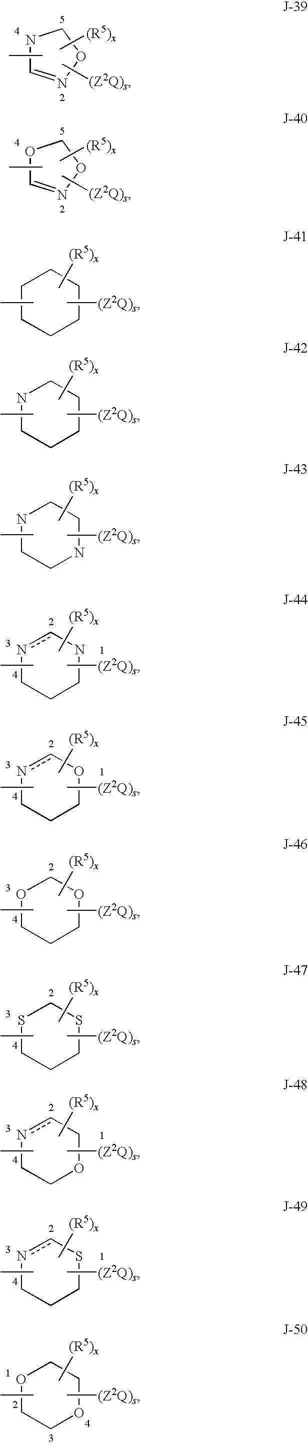 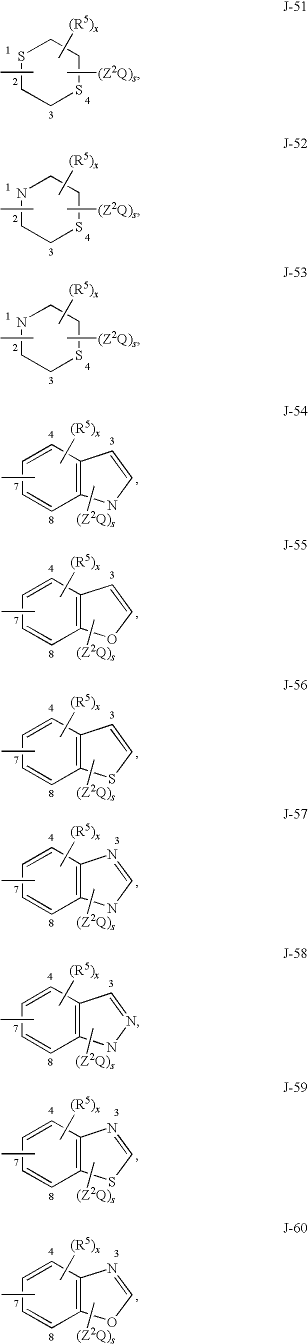 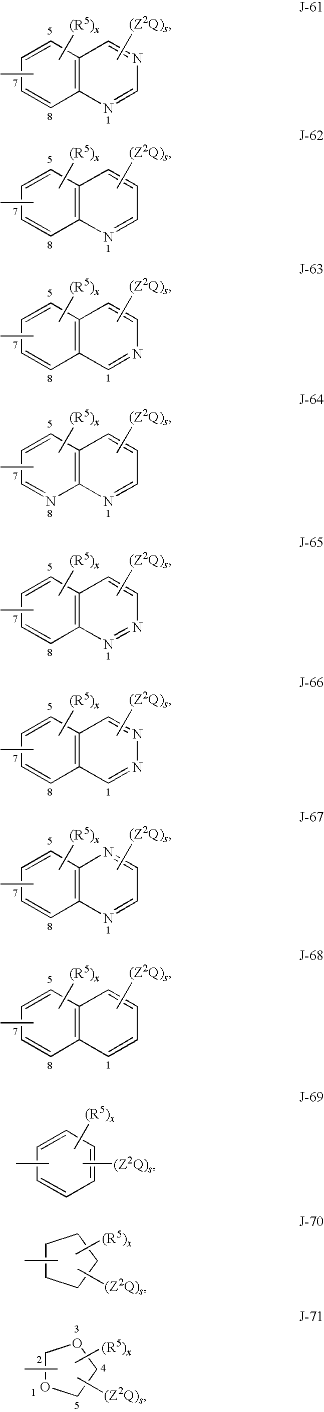 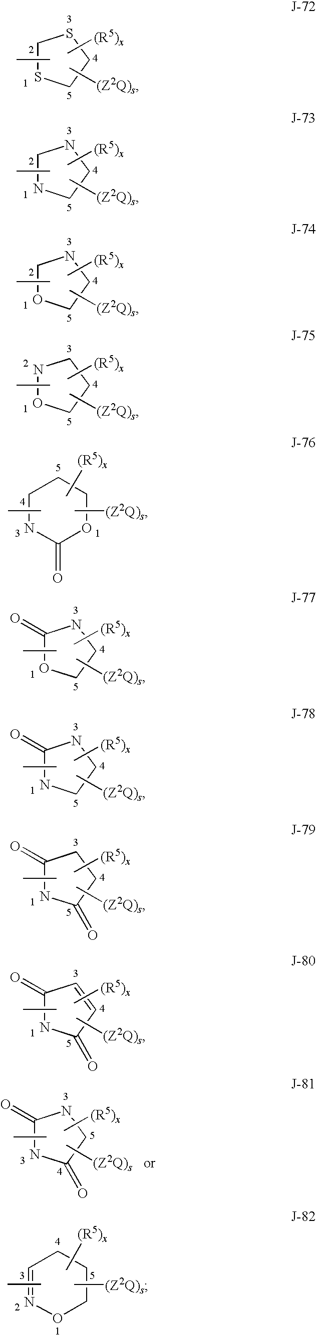
each R² is independently C₁-C₂ alkyl, C₁-C₂ haloalkyl, C₁-C₂ alkoxy, halogen, cyano or hydroxy;
each R³ is independently C₁-C₃ alkyl, C₁-C₃ haloalkyl or halogen;
each R^{4a} is independently C₁-C₆ alkyl, C₂-C₆ alkenyl, C₂-C₆ alkynyl, C₃-C₆ cycloalkyl, C₄-C₁₀ cycloalkylalkyl, C₄-C₁₀ alkylcycloalkyl, C₅-C₁₀ alkylcycloalkylalkyl, C₁-C₆ haloalkyl, C₂-C₆ haloalkenyl, C₂-C₆ haloalkynyl, C₃-C₆ halocycloalkyl, halogen, hydroxy, amino, cyano, nitro, C₁-C₄ alkoxy, C₁-C₄ haloalkoxy, C₁-C₄ alkylthio, C₁-C₄ alkylsulfinyl, C₁-C₄ alkylsulfonyl, C₁-C₄ haloalkylthio, C₁-C₄ haloalkylsulfinyl, C₁-C₄ haloalkylsulfonyl, C₁-C₄ alkylamino, C₂-C₈ dialkylamino, C₃-C₆ cycloalkylamino, C₂-C₄ alkoxyalkyl, C₁-C₄ hydroxyalkyl, C₂-C₄ alkylcarbonyl, C₂-C₆ alkoxycarbonyl, C₂-C₆ alkylcarbonyloxy, C₂-C₆ alkylcarbonylthio, C₂-C₆ alkylaminocarbonyl, C₃-C₈ dialkylaminocarbonyl or C₃-C₆ trialkylsilyl;
each R^{4b} is independently C₁-C₆ alkyl, C₃-C₆ alkenyl, C₃-C₆ alkynyl, C₃-C₆ cycloalkyl, C₁-C₆ haloalkyl, C₃-C₆ haloalkenyl, C₃-C₆ haloalkynyl, C₃-C₆ halocycloalkyl or C₂-C₄ alkoxyalkyl;
each R¹¹ is independently C₁-C₃ alkyl;
R¹⁵ is H, halogen, cyano, hydroxy, —CHO, C₁-C₄ alkyl, C₁-C₄ haloalkyl or C₂-C₅ alkoxycarbonyl;
R¹⁶ is H, C₁-C₄ alkyl, C₁-C₄ haloalkyl, C₂-C₄ alkylcarbonyl, C₂-C₄ haloalkylcarbonyl or C₂-C₄ alkoxycarbonyl;
x is an integer from 0 to 5; and
s is an integer from 1 to 2.

3. A compound of claim 2 wherein
G is one of G-1 through G-59 (as depicted in Exhibit 2) wherein the bond projecting to the left is bonded to X, and bond projecting to the right is bonded to Z¹; 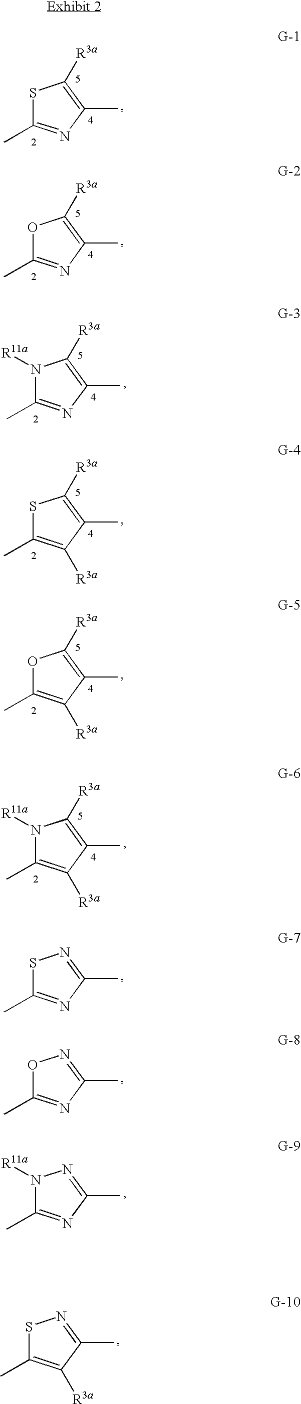 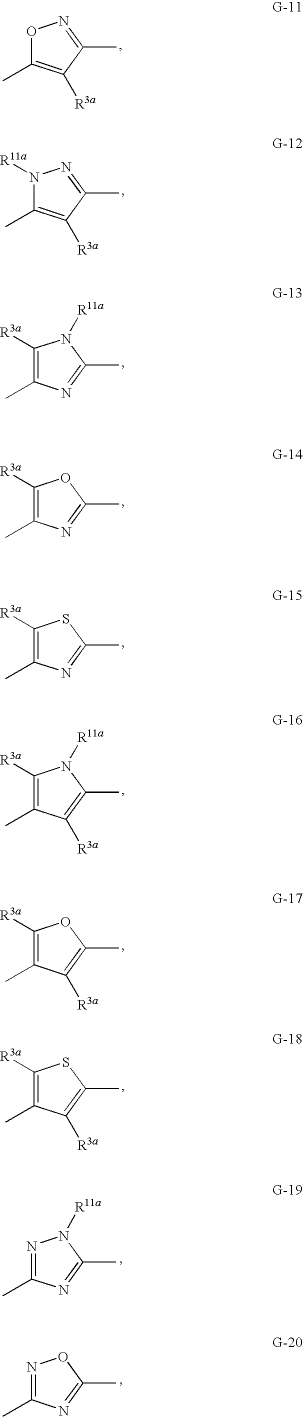 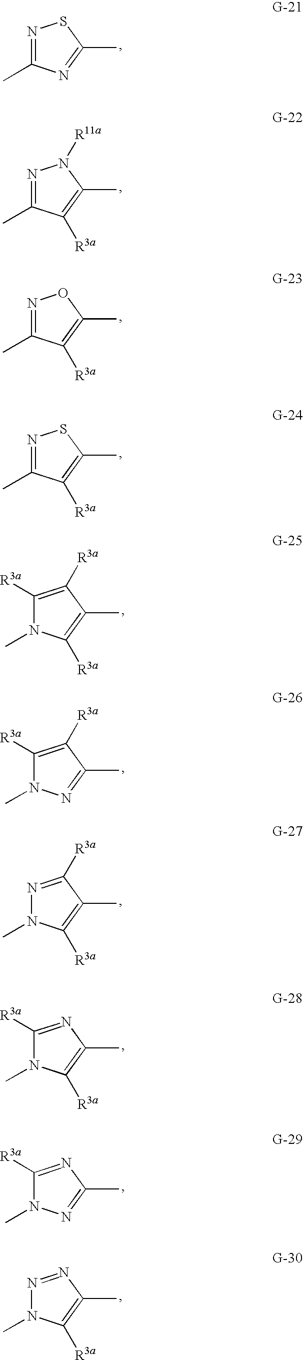 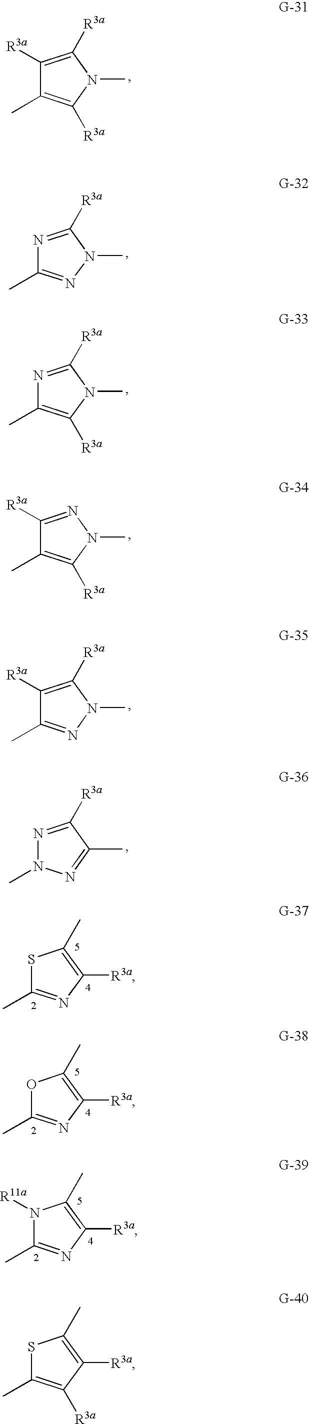 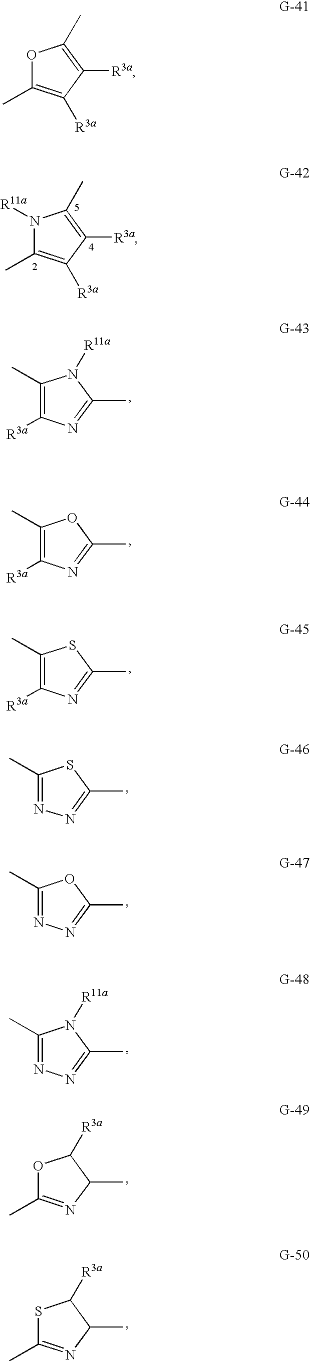 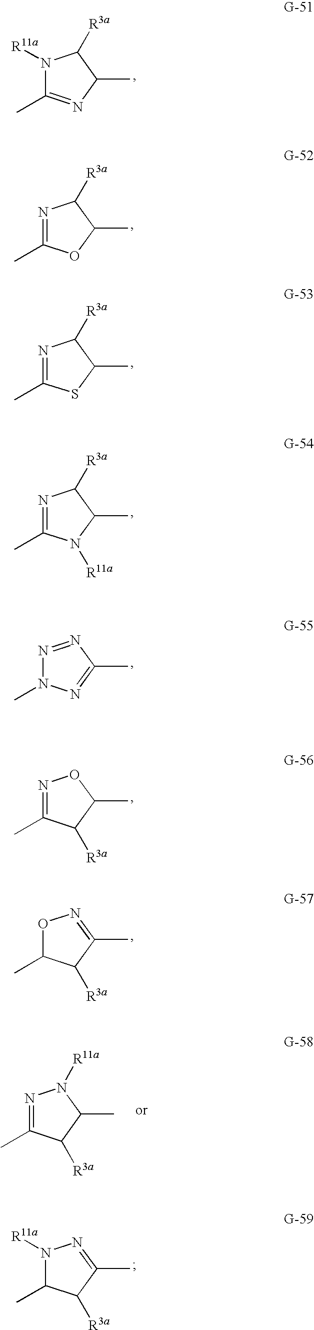
J is selected from J-1, J-2, J-3, J-4, J-5, J-7, J-8, J-9, J-10, J-11, J-12, J-14, J-15, J-16, J-20, J-24, J-25, J-26, J-29, J-30, J-37, J-38, J-45 and J-69;
Q is one of Q-1 through Q-106 (as depicted in Exhibit 4); 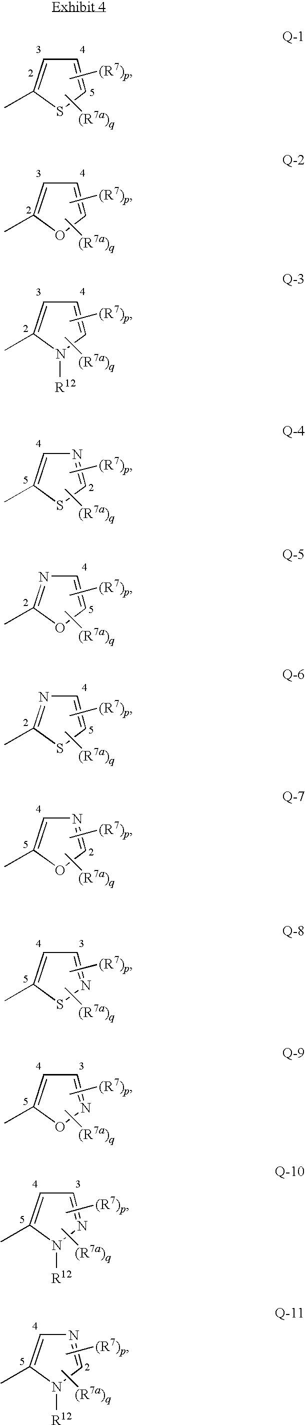 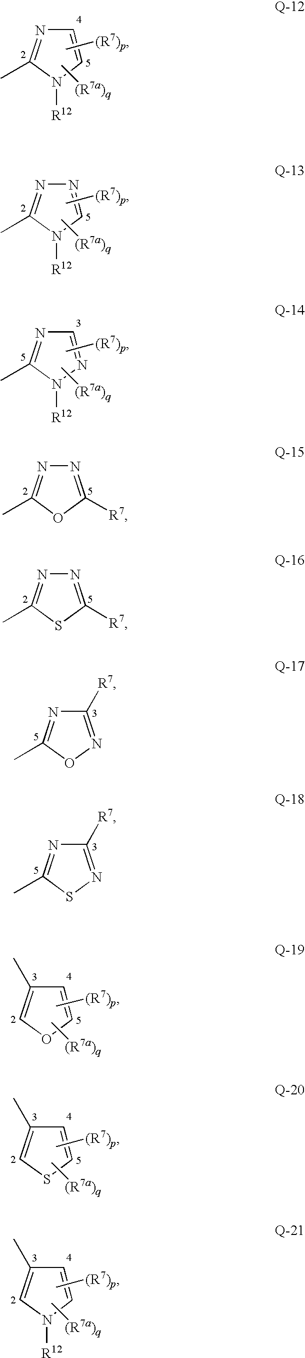 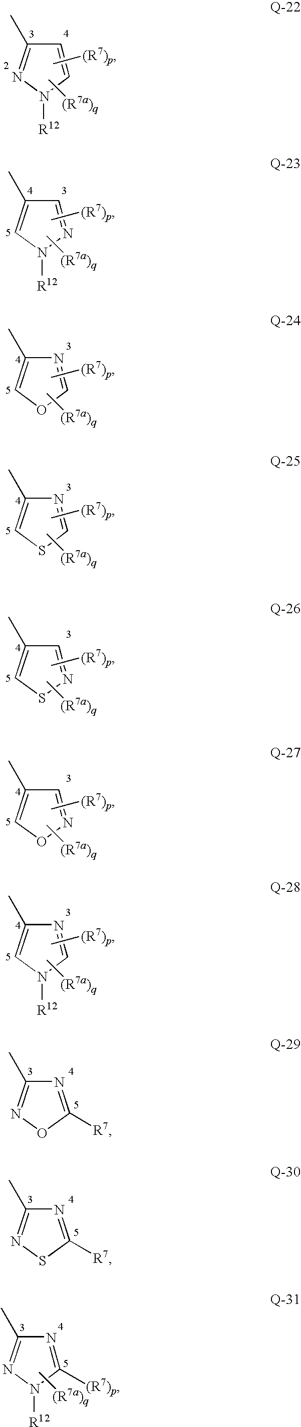 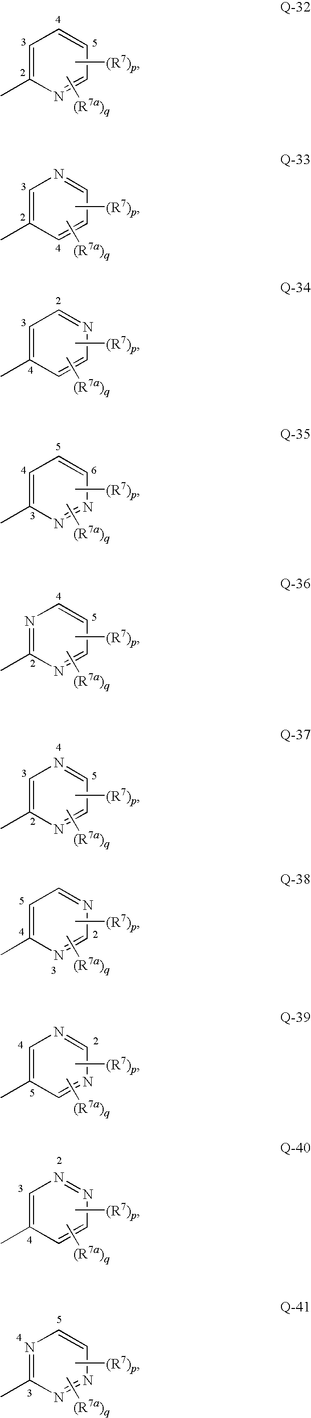 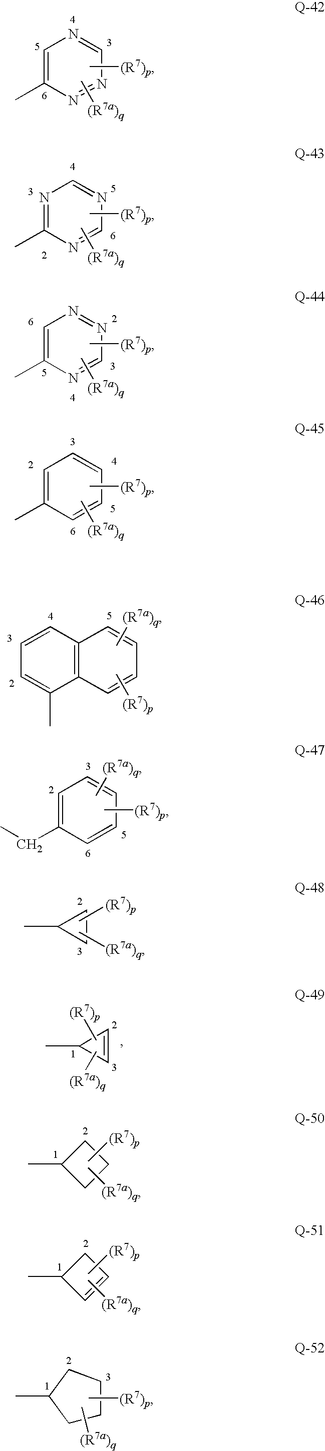 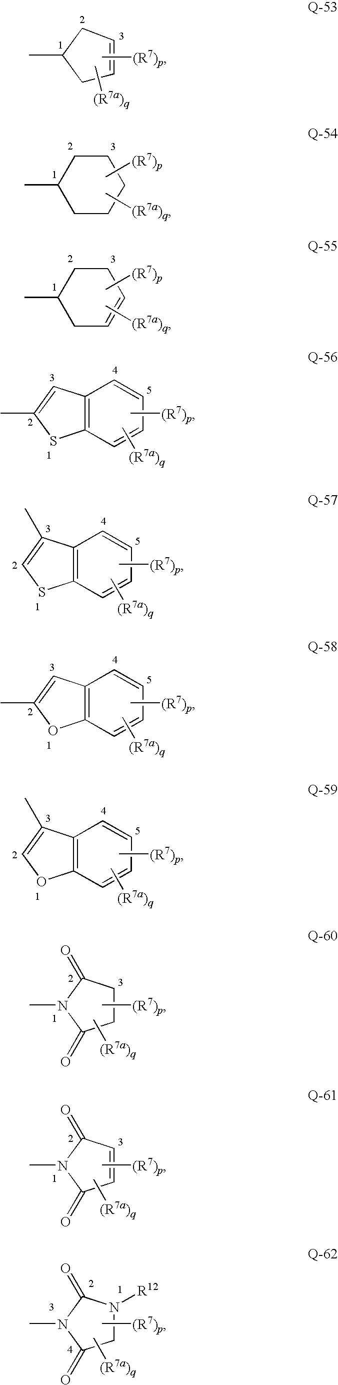 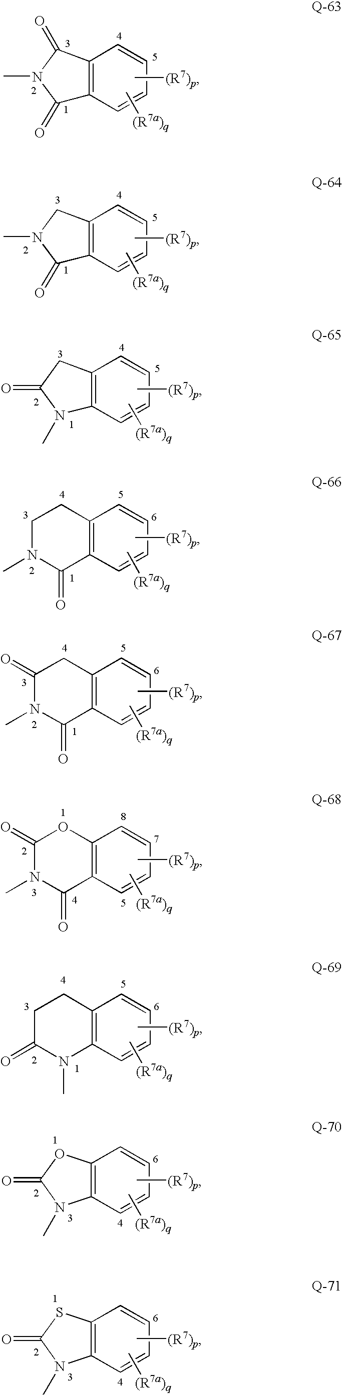 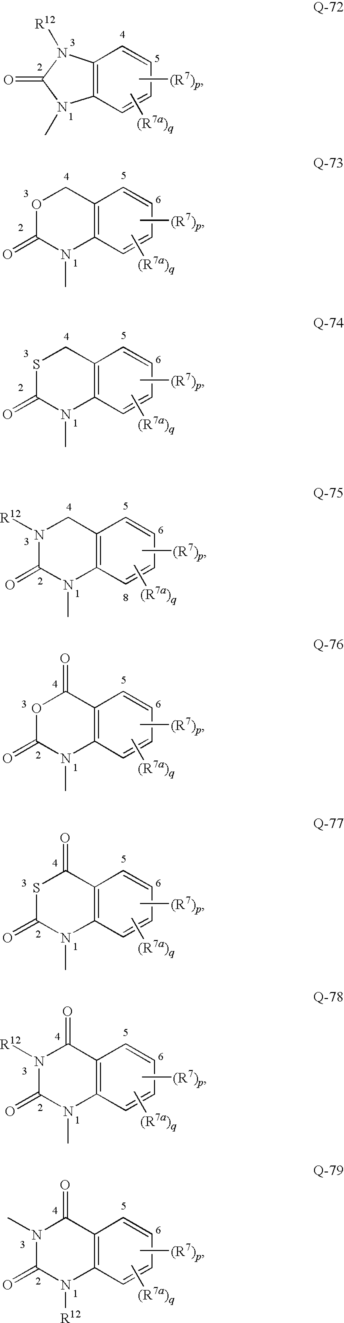 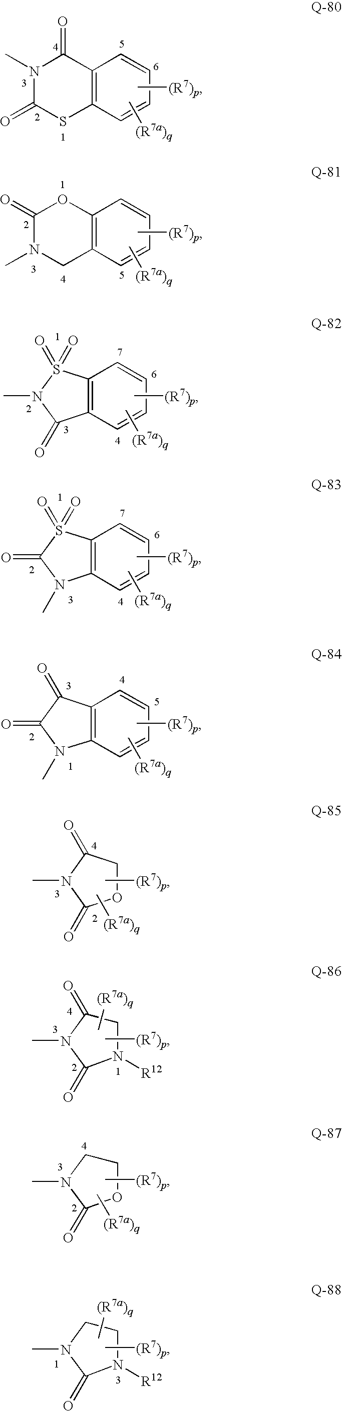 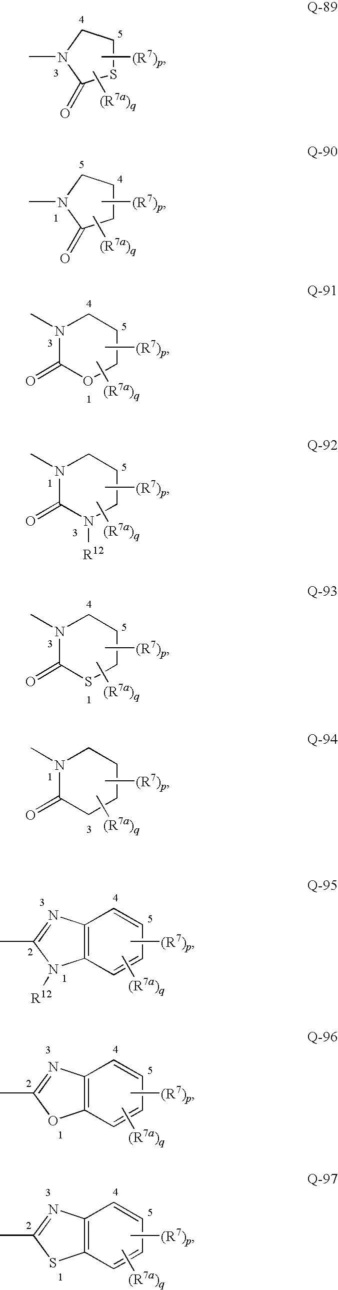 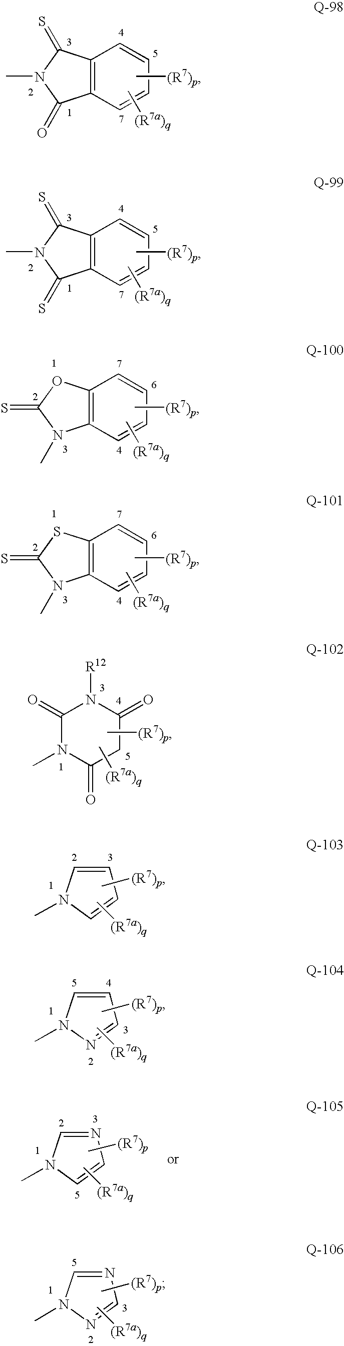
R¹ is one of U-1 through U-50 (as depicted in Exhibit 1); 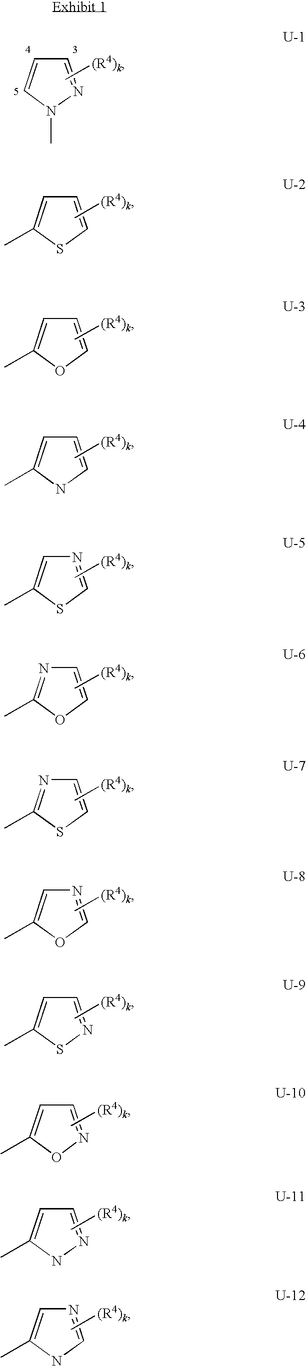 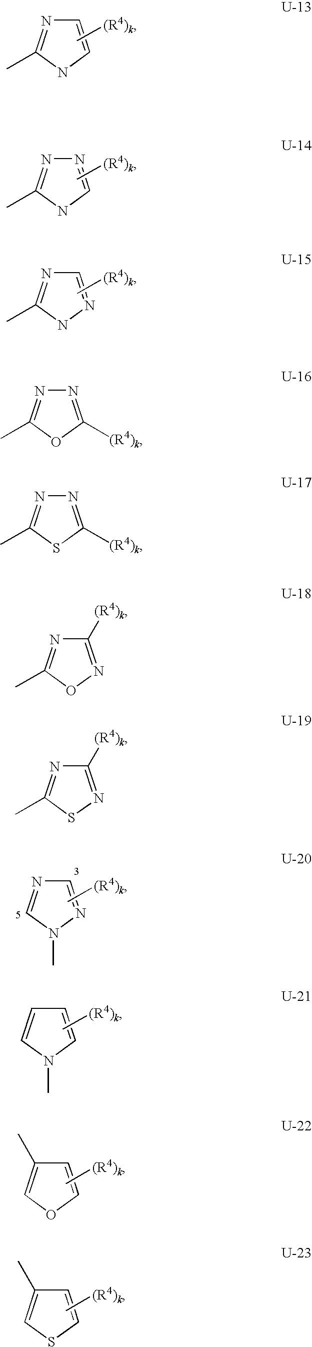 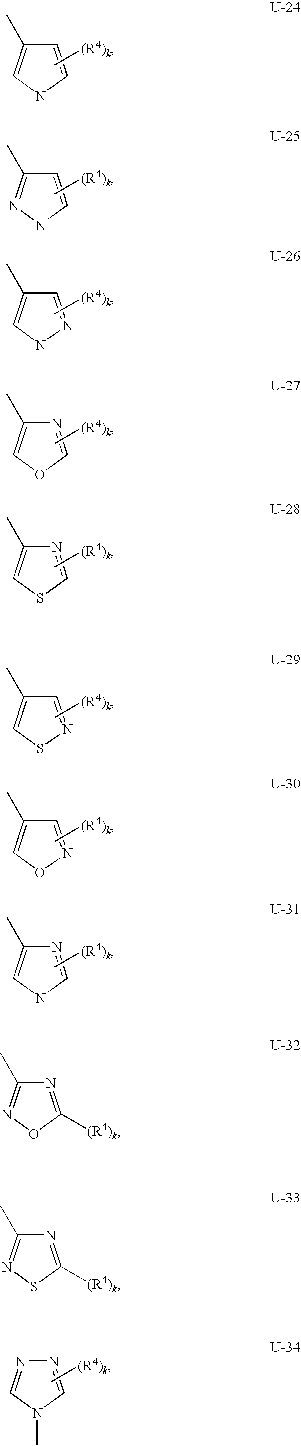 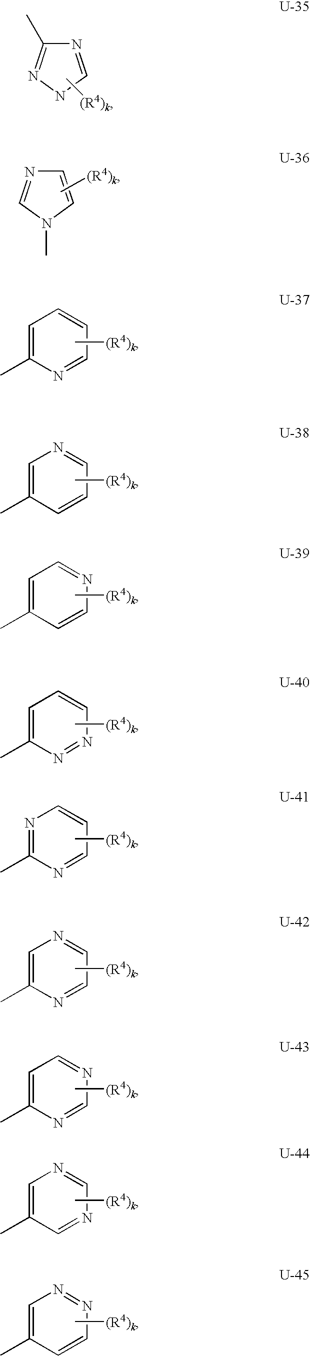 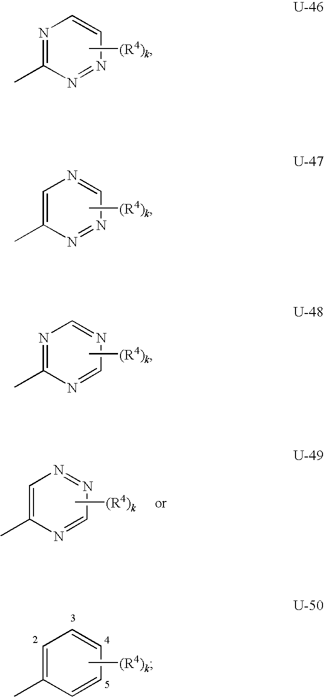
wherein
when R⁴ is attached to a carbon ring member, said R⁴ is selected from R^{4a}, and when R⁴ is attached to a nitrogen ring member (e.g., in U-4, U-11 through U-15, U-24 through U-26, U-31 or U-35), said R⁴ is selected from R^{4b};
each R² is independently methyl, methoxy, cyano or hydroxy;
each R^{1a} is independently selected from H and R³;
each R⁵ is independently H, cyano, C₁-C₆ alkyl, C₁-C₆ haloalkyl, C₃-C₈ cycloalkyl, C₃-C₈ halocycloalkyl, C₂-C₆ alkoxyalkyl, C₁-C₆ alkoxy, C₁-C₆ haloalkoxy, C₃-C₈ cycloalkoxy, C₂-C₆ alkenyloxy, C₂-C₆ haloalkenyloxy, C₂-C₆ alkynyloxy, C₂-C₆ alkoxyalkoxy, C₂-C₆ alkylcarbonyloxy, C₂-C₆ haloalkylcarbonyloxy, C₁-C₆ alkylthio, C₁-C₆ haloalkylthio, C₃-C₁₀ trialkylsilyl or —NR²⁵R²⁶;
R^{11a} is selected from H and R¹¹;
R¹⁵ is H, cyano, hydroxy, methyl or methoxycarbonyl;
R¹⁶ is H, methyl, methylcarbonyl or methoxycarbonyl;
each Z⁴ is C(═O);
k is 0, 1 or 2;
p is 1 or 2;
q is 0, 1, 2, 3, 4 or 5; and
s is 1.

4. A compound of claim 3 wherein
G is selected from G-1, G-2, G-7, G-8, G-14, G-15, G-23, G-24, G-26, G-27, G-36, G-37, G-38, G-49, G-50 and G-55;
J is selected from J-4, J-5, J-8, J-11, J-15, J-16, J-20, J-29, J-30, J-37, J-38 and J-69;
each Q is independently Q-1, Q-20, Q-32 through Q-34, Q-45 through Q-47, Q-60 through Q-73, Q-76 through Q-79, Q-84 through Q-94 and Q-98 through Q-106;
A is CH₂ or NH;
W is O;
X is X¹, X² or X³;
Z¹ is a direct bond;
Z² is a direct bond or NR²¹;
R¹ is selected from U-1 through U-3, U-11, U-13, U-20, U-22, U-23, U-36 through U-39 and U-50;
each R³ is independently methyl or halogen;
each R^{4a} is independently C₁-C₂ alkyl, C₁-C₂ haloalkyl, halogen, C₁-C₂ alkoxy or C₁-C₂ haloalkoxy;
each R^{4b} is independently C₁-C₂ alkyl or C₁-C₂ haloalkyl;
each R^{7a} is independently C₁-C₆ alkyl, C₃-C₆ cycloalkyl, C₁-C₆ haloalkyl, halogen, cyano, C₁-C₄ alkoxy, C₁-C₄ haloalkoxy or C₂-C₆ alkoxycarbonyl;
k is 1 or 2; and
n is 0.

5. A compound of claim 4 wherein
A is CH₂;
G is selected from G-1, G-2, G-15, G-26, G-27, G-36, G-37 and G-38; and G is unsubstituted;
J is J-29;
Q is selected from Q-1, Q-45, Q-63, Q-64, Q-65, Q-68, Q-69, Q-70, Q-71, Q-72, Q-73, Q-76, Q-78, Q-79, Q-84, Q-85, Q-98, Q-99, Q-100 and Q-101 through Q-106;
X is X¹ or X²; and the ring comprising X is saturated;
R¹ is U-1, U-20 or U-50;
each R^{4a} is independently C₁-C₂ alkyl, trifluoromethyl, Cl, Br, I or methoxy;
each R^{4b} is independently C₁-C₂ alkyl or trifluoromethyl; and
each R⁵ is independently H, cyano, C₁-C₆ alkyl, C₁-C₆ haloalkyl, C₁-C₆ alkoxy, C₁-C₆ haloalkoxy or —NR²⁵R²⁶.

6. A compound of claim 5 wherein
G is selected from G-1, G-2, G-15, G-26 and G-36;
J is any one of J-29-1 to J-29-60 (depicted with Exhibit A); 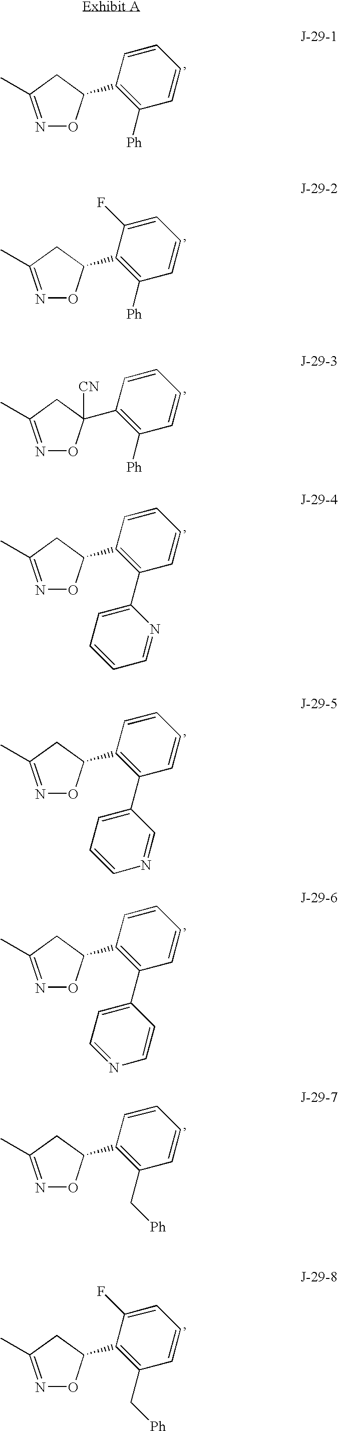 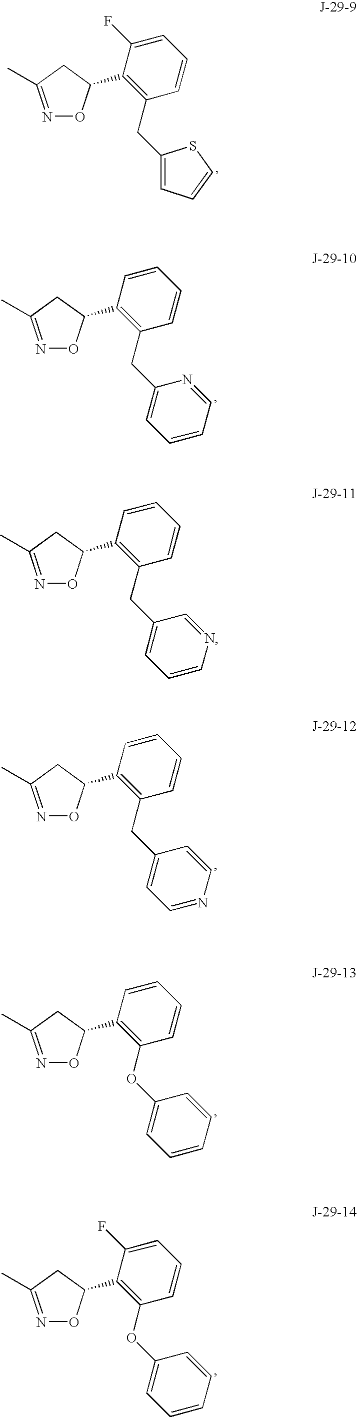 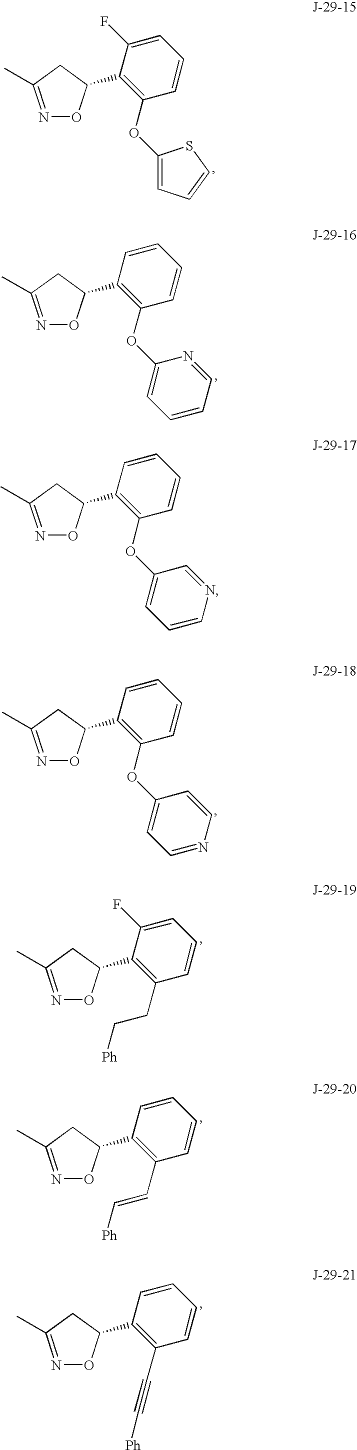 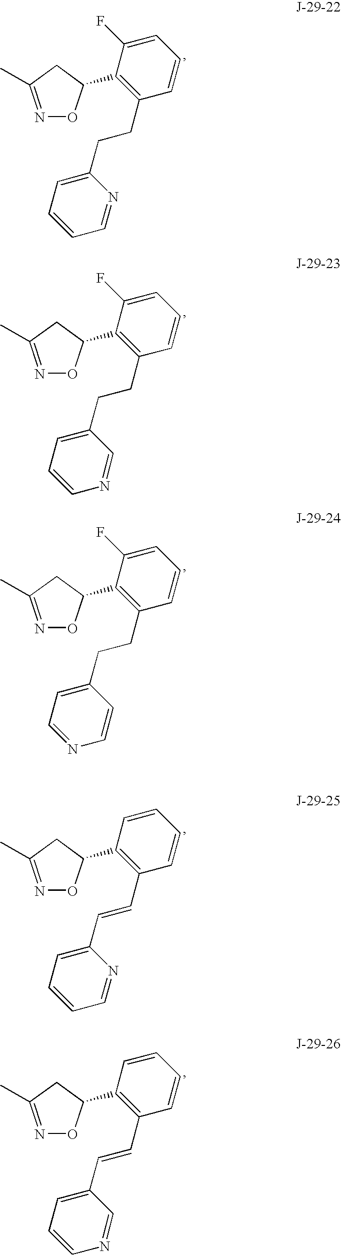 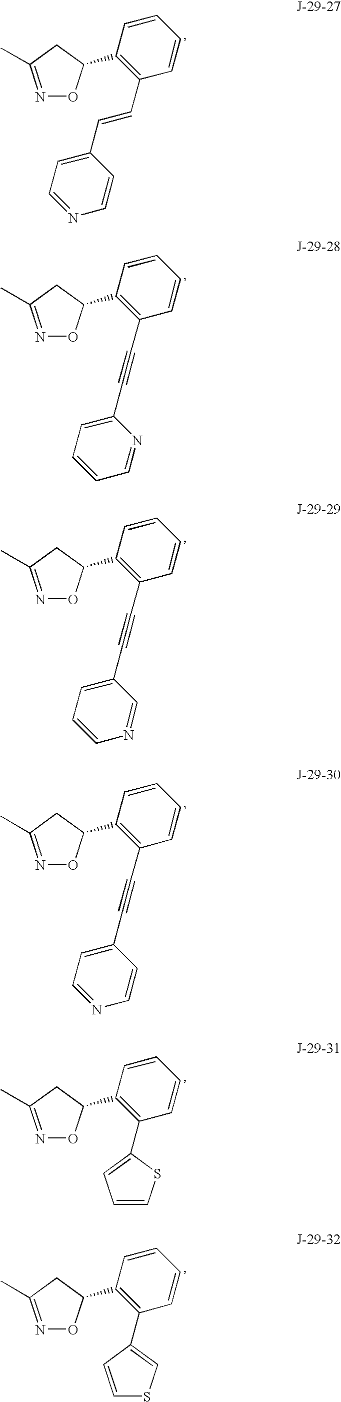 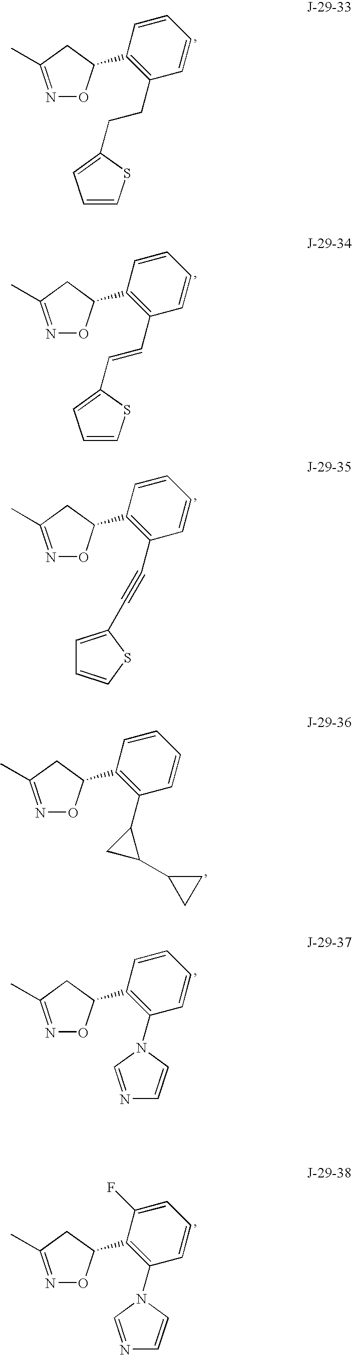 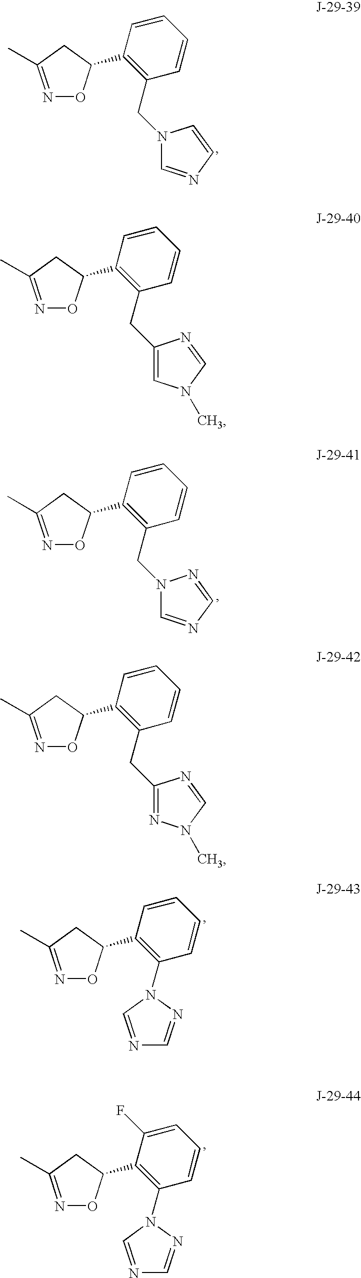 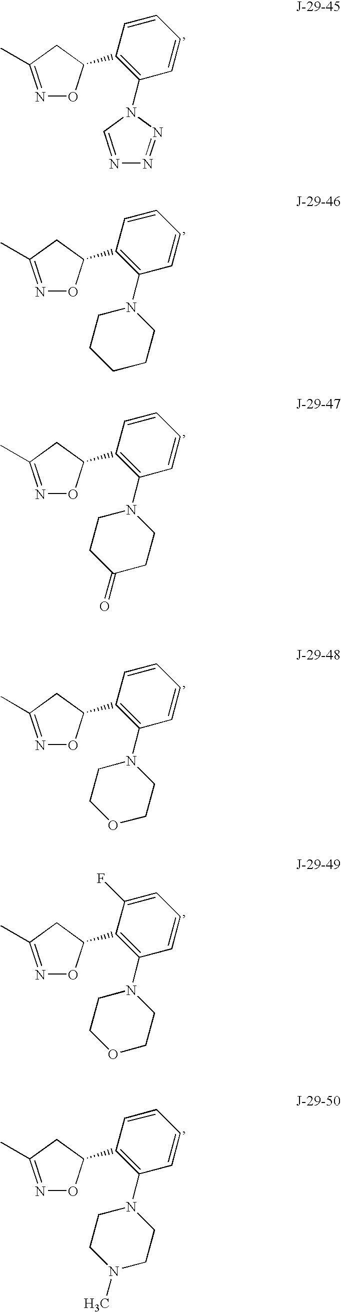 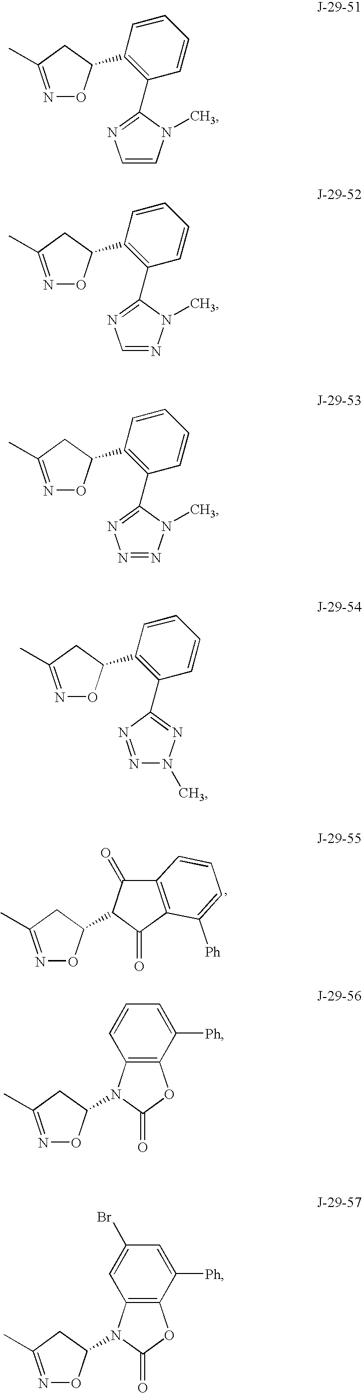 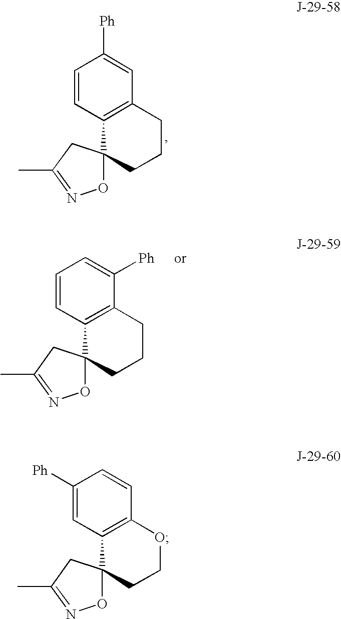
wherein the bond shown projecting to the left is bonded to Z¹ and Ph is phenyl.
Q is selected from Q-45, Q-63, Q-64, Q-65, Q-68, Q-69, Q-70, Q-71, Q-72 and Q-85; and
X is X¹.

7. A compound of claim 1 selected from the group consisting of:
1-[4-[4-[4,5-dihydro-5-[3-(1H-1,2,4-triazol-1-yl)phenyl]-3-isoxazolyl]-2-thiazolyl]-1-piperidinyl]-2-[5-methyl-3-(trifluoromethyl)-1H-pyrazol-1-yl]ethanone, and
1-[4-[4-(5-[1,1′-biphenyl]-4-yl-4,5-dihydro-3-isoxazolyl)-2-thiazolyl]-1-piperidinyl]-2-[5-methyl-3-(trifluoromethyl)-1H-pyrazol-1-yl]ethanone.
4-[4-(5-[1,1′-biphenyl]-2-yl-4,5-dihydro-3-isoxazolyl)-2-thiazolyl]-N-(2,5-dimethylphenyl)-1-piperidinecarboxamide,
4-[4-(4,5-dihydro-5-[2-(1H-1,2,4-triazol-1-yl)phenyl]-3-isoxazolyl)-2-thiazolyl]-N-(2,5-dimethylphenyl)-1-piperidinecarboxamide,
1-[4-[4-[4,5-dihydro-5-[2-(1H-1,2,4-triazol-1-yl)phenyl]-3-isoxazolyl]-2-thiazolyl]-1-piperidinyl]-2-[5-methyl-3-(trifluoromethyl)-1H-pyrazol-1-yl]ethanone,
1-[4-[4-[5-[2-fluoro-6-(1H-1,2,4-triazol-1-yl)phenyl]-4,5-dihydro-3-isoxazolyl]-2-thiazolyl]-1-piperidinyl]-2-[5-methyl-3-(trifluoromethyl)-1H-pyrazol-1-yl]ethanone, and
1-[4-[4-(5-[1,1′-biphenyl]-2-yl-4,5-dihydro-3-isoxazolyl)-2-thiazolyl]-1-piperidinyl]-2-[5-methyl-3-(trifluoromethyl)-1H-pyrazol-1-yl]ethanone.

8. A compound selected from the compounds of Formula 1A and N-oxides and salts thereof 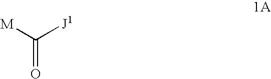
wherein
M is C₁-C₃ alkyl, C₁-C₃ haloalkyl, hydroxy, C₁-C₄ alkoxy, C₁-C₂ haloalkoxy, C₁-C₄ alkylamino, C₂-C₈ dialkylamino, 1-piperidinyl, 1-pyrrolidinyl or 4-morpholinyl; and
J¹ is any one of J-29-1 through J-29-60 as depicted in claim 6 wherein the bond shown projecting to the left is bonded to —C(═O)M of Formula 1A.

9. A method for controlling plant diseases caused by Oomycete fungal plant pathogens comprising applying to the plant or portion thereof, or to the plant seed or seedling, a fungicidally effective amount of a compound of claim 1.

10. A fungicidal composition comprising (1) a compound of claims 1; and (2) at least one other fungicide.

11. A fungicidal composition comprising (1) a compound of claims 1; and (2) at least one additional component selected from the group consisting of surfactants, solid diluents and liquid diluents.
